SEQUENCE LISTING (797 sequences)

SEQ ID NO: 1 - DNA, 23 bp, Artificial Sequence
  Synthetic Oligo U1 (U1 primer)
ggtgtgtgtgggtagttggtgat

SEQ ID NO: 2 - DNA, 24 bp, Artificial Sequence
  Synthetic Oligo U1-A
ggtgtgtgtgggtagttggtgata

SEQ ID NO: 3 - DNA, 24 bp, Artificial Sequence
  Synthetic Oligo U1-C
ggtgtgtgtgggtagttggtgatc

SEQ ID NO: 4 - DNA, 24 bp, Artificial Sequence
  Synthetic Oligo U1-G
ggtgtgtgtgggtagttggtgatg

SEQ ID NO: 5 - DNA, 24 bp, Artificial Sequence
  Synthetic Oligo U1-T
ggtgtgtgtgggtagttggtgatt

SEQ ID NO: 6 - DNA, 23 bp, Artificial Sequence
  Synthetic Oligo U2
  misc_feature: Modified with a 5Phos, 3SpC3 and Phosphorothioate Bonds
agagaatgaggaaggtggggtag

SEQ ID NO: 7 - DNA, 23 bp, Artificial Sequence
  Synthetic U2 primer
ctaccccaccttcctcattctct

SEQ ID NO: 8 - DNA, 200 bp, Artificial Sequence
  Synthetic 200-A oligo
cggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcagg

SEQ ID NO: 9 - DNA, 200 bp, Artificial Sequence
  Synthetic 200-C oligo
cggtcacagcttgtctgtaagcggatgccgggagcagacaagcccgtcagggcgcgtcagcgggtgttggcgggtgtcggggctggcttaactatgcggcatcagagcagattgtactgagagtgcaccctatgcggtgtgaaataccgcacagatgcgtaaggagaaaataccgcatcaggcgccattcgccattcagg

SEQ ID NO: 10 - DNA, 200 bp, Artificial Sequence
  Synthetic 200-G oligo
cggtcacagcttgtctgtaagcggatgccgggagcagacaagcccgtcagggcgcgtcagcgggtgttggcgggtgtcggggctggcttaactatgcggcatcagagcagattgtactgagagtgcaccgtatgcggtgtgaaataccgcacagatgcgtaaggagaaaataccgcatcaggcgccattcgccattcagg

SEQ ID NO: 11 - DNA, 200 bp, Artificial Sequence
  Synthetic 200-T oligo
cggtcacagcttgtctgtaagcggatgccgggagcagacaagcccgtcagggcgcgtcagcgggtgttggcgggtgtcggggctggcttaactatgcggcatcagagcagattgtactgagagtgcaccttatgcggtgtgaaataccgcacagatgcgtaaggagaaaataccgcatcaggcgccattcgccattcagg

SEQ ID NO: 12 - DNA, 53 bp, Artificial Sequence
  Synthetic 200 oligo 1-A
cgcatctgtgcggtatttcacaccgcatatatcaccaactacccacacacacc

SEQ ID NO: 13 - DNA, 53 bp, Artificial Sequence
  Synthetic 200 oligo 1-C
cgcatctgtgcggtatttcacaccgcatagatcaccaactacccacacacacc

SEQ ID NO: 14 - DNA, 53 bp, Artificial Sequence
  Synthetic 200 oligo 1-G
cgcatctgtgcggtatttcacaccgcatacatcaccaactacccacacacacc

SEQ ID NO: 15 - DNA, 53 bp, Artificial Sequence
  Synthetic 200 oligo 1-T
cgcatctgtgcggtatttcacaccgcataaatcaccaactacccacacacacc

SEQ ID NO: 16 - DNA, 80 bp, Artificial Sequence
  Synthetic Unmethylated 80-nt oligo
gtctgaactatgcagcatcagagcagattgtaagatacagtgcatgcgcgtgccgatgcgaatacacagacgtgtcagga

SEQ ID NO: 17 - DNA, 80 bp, Artificial Sequence; identical to SEQ ID NO: 16
  Synthetic Methylated 80-nt oligo
  modified_base (43)..(43): 5-Methylcytosine
  modified_base (47)..(47): 5-Methylcytosine
  modified_base (49)..(49): 5-Methylcytosine
  modified_base (53)..(54): 5-Methylcytosine

SEQ ID NO: 18 - DNA, 44 bp, Artificial Sequence
  Synthetic 80 oligo 1-A
tattcgcatcggcacgcgcatatcaccaactacccacacacacc

SEQ ID NO: 19 - DNA, 44 bp, Artificial Sequence
  Synthetic 80 oligo 1-C
gtgtattcgcatcggcacgcgatcaccaactacccacacacacc

SEQ ID NO: 20 - DNA, 44 bp, Artificial Sequence
  Synthetic 80 oligo 1-G
tgtattcgcatcggcacgcgcatcaccaactacccacacacacc

SEQ ID NO: 21 - DNA, 44 bp, Artificial Sequence
  Synthetic 80 oligo 1-T
gtattcgcatcggcacgcgcaatcaccaactacccacacacacc

SEQ ID NO: 22 - DNA, 24 bp, Artificial Sequence
  Synthetic 80 qPCR-F primer
gtctgaactatgcagcatcagagc

SEQ ID NO: 23 - DNA, 22 bp, Artificial Sequence
  Synthetic 80 qPCR-R primer
tcctgacacgtctgtgtattcg

SEQ ID NO: 24 - DNA, 47 bp, Artificial Sequence
  Synthetic pGEM-3Z/601b NdeI oligo 1-T
ctgtgcggtatttcacaccgcataatcaccaactacccacacacacc

SEQ ID NO: 25 - DNA, 43 bp, Artificial Sequence
  Synthetic pGEM-3Z/601b HindIII oligo
ctaccccaccttcctcattctcttgcatgcctgcaggtccggg

SEQ ID NO: 26 - DNA, 47 bp, Artificial Sequence
  Synthetic Methyl test pGEM-3Z/601b oligo 1-C
tcagtacaatctgctctgatgccgatcaccaactacccacacacacc

SEQ ID NO: 27 - DNA, 46 bp, Artificial Sequence
  Synthetic Methyl test pGEM-3Z/601b oligo 1-G
agtacaatctgctctgatgccgcatcaccaactacccacacacacc

SEQ ID NO: 28 - DNA, 48 bp, Artificial Sequence
  Synthetic Methyl test pGEM-3Z/601b oligo 1-T
gtacaatctgctctgatgccgcataatcaccaactacccacacacacc

SEQ ID NO: 29 - DNA, 17 bp, Artificial Sequence
  Synthetic pGEM-3Z/601b mutagenesis to C oligo 1
tgcaccctatgcggtgt

SEQ ID NO: 30 - DNA, 17 bp, Artificial Sequence
  Synthetic pGEM-3Z/601b mutagenesis to G oligo 1
tgcaccgtatgcggtgt

SEQ ID NO: 31 - DNA, 17 bp, Artificial Sequence
  Synthetic pGEM-3Z/601b mutagenesis to T oligo 1
tgcaccttatgcggtgt

SEQ ID NO: 32 - DNA, 22 bp, Artificial Sequence
  Synthetic pGEM-3Z/601b mutagenesis oligo 2
ctctcagtacaatctgctctga

SEQ ID NO: 33 - DNA, 53 bp, Artificial Sequence; identical to SEQ ID NO: 12
  Synthetic pGEM-3Z/601b NdeI-2 oligo 1-A

SEQ ID NO: 34 - DNA, 53 bp, Artificial Sequence; identical to SEQ ID NO: 13
  Synthetic pGEM-3Z/601b NdeI-2 oligo 1-C

SEQ ID NO: 35 - DNA, 53 bp, Artificial Sequence; identical to SEQ ID NO: 14
  Synthetic pGEM-3Z/601b NdeI-2 oligo 1-G

SEQ ID NO: 36 - DNA, 53 bp, Artificial Sequence; identical to SEQ ID NO: 15
  Synthetic pGEM-3Z/601b NdeI-2 oligo 1-T

SEQ ID NO: 37 - DNA, 24 bp, Artificial Sequence
  Synthetic MGO3791
tttatagagttgagaaatttgatt

SEQ ID NO: 38 - DNA, 32 bp, Artificial Sequence
  Synthetic MGO3792
taaatataaacaaataatttctaaaataaata

SEQ ID NO: 39 - DNA, 50 bp, Artificial Sequence
  Synthetic 300-nt-EPM2AIP1-oligo 1-T
gcgccactacgaggctgagcacgaataatcaccaactacccacacacacc

SEQ ID NO: 40 - DNA, 44 bp, Artificial Sequence
  Synthetic 300-nt-EXO1-oligo 1-T
agccccagttctggaggctgaatcaccaactacccacacacacc

SEQ ID NO: 41 - DNA, 44 bp, Artificial Sequence
  Synthetic 300-nt-HSPA5-oligo 1-T
gtcggagtaggtccagcaggaatcaccaactacccacacacacc

SEQ ID NO: 42 - DNA, 48 bp, Artificial Sequence
  Synthetic 300-nt-MLH1-oligo 1-T
ggaaacgtctagatgctcaacggaaatcaccaactacccacacacacc

SEQ ID NO: 43 - DNA, 45 bp, Artificial Sequence
  Synthetic 300-nt-MLH3-oligo 1-T
ctggcctatggtaggcaccgaaatcaccaactacccacacacacc

SEQ ID NO: 44 - DNA, 50 bp, Artificial Sequence
  Synthetic 300-nt-MSH2-oligo 1-T
aaactagccttcctcctcctcccagtaatcaccaactacccacacacacc

SEQ ID NO: 45 - DNA, 42 bp, Artificial Sequence
  Synthetic 300-nt-MSH3-oligo 1-T
ccgactcccggcgagaggaatcaccaactacccacacacacc

SEQ ID NO: 46 - DNA, 44 bp, Artificial Sequence
  Synthetic 300-nt-MSH6-oligo 1-T
gataaccggggggacccgaaaatcaccaactacccacacacacc

SEQ ID NO: 47 - DNA, 49 bp, Artificial Sequence
  Synthetic 300-nt-PMS1-oligo 1-T
gctttgccttccagctgagatgtaaaatcaccaactacccacacacacc

SEQ ID NO: 48 - DNA, 44 bp, Artificial Sequence
  Synthetic 300-nt-PMS2-oligo 1-T
ctctccgtgtggagcttcccaatcaccaactacccacacacacc

SEQ ID NO: 49 - DNA, 58 bp, Artificial Sequence
  Synthetic 300-nt-TDG-oligo 1-T
ctgtgttgcattatcaggtaatgagatgtgagggaatcaccaactacccacacacacc

SEQ ID NO: 50 - DNA, 48 bp, Artificial Sequence
  Synthetic 300-nt-EPM2AIP1-oligo 2-T
aaccgggcttgtgtgcctctgctgaatcaccaactacccacacacacc

SEQ ID NO: 51 - DNA, 43 bp, Artificial Sequence
  Synthetic 300-nt-EXO1-oligo 2-T
gccccgcccagaggatgtgaatcaccaactacccacacacacc

SEQ ID NO: 52 - DNA, 41 bp, Artificial Sequence
  Synthetic 300-nt-HSPA5-oligo 2-T
cgtcgtggaggccgccgaatcaccaactacccacacacacc

SEQ ID NO: 53 - DNA, 48 bp, Artificial Sequence
  Synthetic 300-nt-MLH1-oligo 2-T
aggagaaggcctgactggcacgtcaatcaccaactacccacacacacc

SEQ ID NO: 54 - DNA, 47 bp, Artificial Sequence
  Synthetic 300-nt-MLH3-oligo 2-T
caaggatccgcatcccagggttcaatcaccaactacccacacacacc

SEQ ID NO: 55 - DNA, 44 bp, Artificial Sequence
  Synthetic 300-nt-MSH2-oligo 2-T
gtctgccgcggcaaccaatcaatcaccaactacccacacacacc

SEQ ID NO: 56 - DNA, 41 bp, Artificial Sequence
  Synthetic 300-nt-MSH3-oligo 2-T
cgtctgcgggcgcgagcaatcaccaactacccacacacacc

SEQ ID NO: 57 - DNA, 38 bp, Artificial Sequence
  Synthetic 300-nt-MSH6-oligo 2-T
gggcggtgcgcgccaatcaccaactacccacacacacc

SEQ ID NO: 58 - DNA, 43 bp, Artificial Sequence
  Synthetic 300-nt-PMS1-oligo 2-T
gtgacgtgcggccgcgagaaatcaccaactacccacacacacc

SEQ ID NO: 59 - DNA, 47 bp, Artificial Sequence
  Synthetic 300-nt-PMS2-oligo 2-T
ggctgctttcgacgttgtgctccaatcaccaactacccacacacacc

SEQ ID NO: 60 - DNA, 45 bp, Artificial Sequence
  Synthetic 300-nt-TDG-oligo 2-T
acgcgtactggccgtcacgtgaatcaccaactacccacacacacc

SEQ ID NO: 61 - DNA, 47 bp, Artificial Sequence
  Synthetic 300-nt-EPM2AIP1-oligo 3-T
ctaccccaccttcctcattctctaggtgatctggcgcagagcggagg

SEQ ID NO: 62 - DNA, 47 bp, Artificial Sequence
  Synthetic 300-nt-EXO1-oligo 3-T
ctaccccaccttcctcattctctacgtcaacgcgtatcccgcaaccc

SEQ ID NO: 63 - DNA, 47 bp, Artificial Sequence
  Synthetic 300-nt-HSPA5-oligo 3-T
ctaccccaccttcctcattctctattggtgaaggccctgctcgttgg

SEQ ID NO: 64 - DNA, 41 bp, Artificial Sequence
  Synthetic 300-nt-MLH1-oligo 3-T
ctaccccaccttcctcattctctagggaacccggcggcctc

SEQ ID NO: 65 - DNA, 58 bp, Artificial Sequence
  Synthetic 300-nt-MLH3-oligo 3-T
ctaccccaccttcctcattctctagtgaaccggtacctacagtctcaatccttattcc

SEQ ID NO: 66 - DNA, 43 bp, Artificial Sequence
  Synthetic 300-nt-MSH2-oligo 3-T
ctaccccaccttcctcattctctataagcagacgcggcccagc

SEQ ID NO: 67 - DNA, 41 bp, Artificial Sequence
  Synthetic 300-nt-MSH3-oligo 3-T
ctaccccaccttcctcattctctacgccgcgaccctgcgtg

SEQ ID NO: 68 - DNA, 38 bp, Artificial Sequence
  Synthetic 300-nt-MSH6-oligo 3-T
ctaccccaccttcctcattctctaggccccgcccccgg

SEQ ID NO: 69 - DNA, 42 bp, Artificial Sequence
  Synthetic 300-nt-PMS1-oligo 3-T
ctaccccaccttcctcattctctaggaaggggcgggttgggg

SEQ ID NO: 70 - DNA, 41 bp, Artificial Sequence
  Synthetic 300-nt-PMS2-oligo 3-T
ctaccccaccttcctcattctctaccctttccgggcggggc

SEQ ID NO: 71 - DNA, 50 bp, Artificial Sequence
  Synthetic 300-nt-TDG-oligo 3-T
ctaccccaccttcctcattctctatgtaggacagcccaatcacgctcacc

SEQ ID NO: 72 - DNA, 47 bp, Artificial Sequence
  Synthetic 450-nt-ALKBH1-oligo 1-T
cagcgtggtggctgaattaggagaatcaccaactacccacacacacc

SEQ ID NO: 73 - DNA, 50 bp, Artificial Sequence
  Synthetic 450-nt-ALKBH2-oligo 1-T
aggagttctgcagtcggatatcgtggaatcaccaactacccacacacacc

SEQ ID NO: 74 - DNA, 49 bp, Artificial Sequence
  Synthetic 450-nt-ALKBH3-oligo 1-T
gcatcggcatctttgtggcagaaagaatcaccaactacccacacacacc

SEQ ID NO: 75 - DNA, 40 bp, Artificial Sequence
  Synthetic 450-nt-APEX1-oligo 1-T
ggtctccgctcgcgggaatcaccaactacccacacacacc

SEQ ID NO: 76 - DNA, 50 bp, Artificial Sequence
  Synthetic 450-nt-APEX2-oligo 1-T
gccactttttaagcgacggaaccctgaatcaccaactacccacacacacc

SEQ ID NO: 77 - DNA, 48 bp, Artificial Sequence
  Synthetic 450-nt-ATM-oligo 1-T
gtggttcctgctgtggttttgatcaatcaccaactacccacacacacc

SEQ ID NO: 78 - DNA, 63 bp, Artificial Sequence
  Synthetic 450-nt-ATRX-oligo 1-T
cggcactacactaatcctttatccgggttatctcatttaaatcaccaactacccacacacacc

SEQ ID NO: 79 - DNA, 53 bp, Artificial Sequence
  Synthetic 450-nt-ATXN3-oligo 1-T
ccgcttcccacccatttctatggattcttaatcaccaactacccacacacacc

SEQ ID NO: 80 - DNA, 48 bp, Artificial Sequence
  Synthetic 450-nt-AXIN2-oligo 1-T
gtttccctgggaatttgggcttggaatcaccaactacccacacacacc

SEQ ID NO: 81 - DNA, 57 bp, Artificial Sequence
  Synthetic 450-nt-BRCA1-oligo 1-T
ccctaatggaggtctccagtttcggtaaatataaatcaccaactacccacacacacc

SEQ ID NO: 82 - DNA, 39 bp, Artificial Sequence
  Synthetic 450-nt-BRCA2-oligo 1-T
gtgggcgccctcgggaatcaccaactacccacacacacc

SEQ ID NO: 83 - DNA, 52 bp, Artificial Sequence
  Synthetic 450-nt-CCN4-oligo 1-T
cctgcctaggtctttcactactgtagggaatcaccaactacccacacacacc

SEQ ID NO: 84 - DNA, 54 bp, Artificial Sequence
  Synthetic 450-nt-CD44-oligo 1-T
ggggagttggtgaatcttccaagtggaaagaatcaccaactacccacacacacc

SEQ ID NO: 85 - DNA, 48 bp, Artificial Sequence
  Synthetic 450-nt-CDH13-oligo 1-T
tggtgcttccctgccatctcacaaaatcaccaactacccacacacacc

SEQ ID NO: 86 - DNA, 48 bp, Artificial Sequence
  Synthetic 450-nt-CDH1-oligo 1-T
agtgcagtggtgcgatcacagctcaatcaccaactacccacacacacc

SEQ ID NO: 87 - DNA, 42 bp, Artificial Sequence
  Synthetic 450-nt-CDH2-oligo 1-T
ccgcaagggggtgaggcgaatcaccaactacccacacacacc

SEQ ID NO: 88 - DNA, 43 bp, Artificial Sequence
  Synthetic 450-nt-CDKN2A-oligo 1-T
ggcagctcgccccctagctaatcaccaactacccacacacacc

SEQ ID NO: 89 - DNA, 49 bp, Artificial Sequence
  Synthetic 450-nt-CDKN2D-oligo 1-T
gcaggcgggaaagaaggctggtttaaatcaccaactacccacacacacc

SEQ ID NO: 90 - DNA, 52 bp, Artificial Sequence
  Synthetic 450-nt-CETN1-oligo 1-T
gaccccagttttataggtttctttagggaatcaccaactacccacacacacc

SEQ ID NO: 91 - DNA, 51 bp, Artificial Sequence
  Synthetic 450-nt-CHEK1-oligo 1-T
tggggaacatagggagaccccatctctaatcaccaactacccacacacacc

SEQ ID NO: 92 - DNA, 43 bp, Artificial Sequence
  Synthetic 450-nt-CHEK2-oligo 1-T
gccagatgtggcgggggagaatcaccaactacccacacacacc

SEQ ID NO: 93 - DNA, 56 bp, Artificial Sequence
  Synthetic 450-nt-CSDE1-oligo 1-T
ggccaacaattctgatggcatattcaggagtcaatcaccaactacccacacacacc

SEQ ID NO: 94 - DNA, 50 bp, Artificial Sequence
  Synthetic 450-nt-DDB2-oligo 1-T
cagcctcctaagtagatgggactacaaatcaccaactacccacacacacc

SEQ ID NO: 95 - DNA, 53 bp, Artificial Sequence
  Synthetic 450-nt-DRD3-oligo 1-T
tgatgtcaatatccttacctgcctatttaaatcaccaactacccacacacacc

SEQ ID NO: 96 - DNA, 44 bp, Artificial Sequence
  Synthetic 450-nt-DRD4-oligo 1-T
cgcgacccgagaaaccgacaaatcaccaactacccacacacacc

SEQ ID NO: 97 - DNA, 62 bp, Artificial Sequence
  Synthetic 450-nt-EGFR-oligo 1-T
ccccctttcccttcttttgttttacaaagcaaacttgtaatcaccaactacccacacacacc

SEQ ID NO: 98 - DNA, 50 bp, Artificial Sequence; identical to SEQ ID NO: 39
  Synthetic 450-nt-EPM2AIP1-oligo 1-T

SEQ ID NO: 99 - DNA, 54 bp, Artificial Sequence
  Synthetic 450-nt-EPSTI1-oligo 1-T
caggcaactctaatttgaggttaccgctggaatcaccaactacccacacacacc

SEQ ID NO: 100 - DNA, 54 bp, Artificial Sequence
  Synthetic 450-nt-ERCC1-oligo 1-T
ctcagacattgacttggcttcagtttcctcaatcaccaactacccacacacacc

SEQ ID NO: 101 - DNA, 53 bp, Artificial Sequence
  Synthetic 450-nt-ERCC2-oligo 1-T
cccaggagttcgagatcagcttgagcaagaatcaccaactacccacacacacc

SEQ ID NO: 102 - DNA, 47 bp, Artificial Sequence
  Synthetic 450-nt-ERCC8-oligo 1-T
atcccgcagtacaagaactggagaatcaccaactacccacacacacc

SEQ ID NO: 103 - DNA, 44 bp, Artificial Sequence; identical to SEQ ID NO: 40
  Synthetic 450-nt-EXO1-oligo 1-T

SEQ ID NO: 104 - DNA, 58 bp, Artificial Sequence
  Synthetic 450-nt-FANCC-oligo 1-T
gtaggggaggtaaaatggaaattagttcttttgaaatcaccaactacccacacacacc

SEQ ID NO: 105 - DNA, 58 bp, Artificial Sequence
  Synthetic 450-nt-FAT4-oligo 1-T
gagttcccagaaaagtttctcaagcctcttatataatcaccaactacccacacacacc

SEQ ID NO: 106 - DNA, 41 bp, Artificial Sequence
  Synthetic 450-nt-FEN1-oligo 1-T
cgagaggcgtggggcgaaatcaccaactacccacacacacc

SEQ ID NO: 107 - DNA, 48 bp, Artificial Sequence
  Synthetic 450-nt-FHIT-oligo 1-T
acgtgtaacgaaaggctgtaccgcaatcaccaactacccacacacacc

SEQ ID NO: 108 - DNA, 39 bp, Artificial Sequence
  Synthetic 450-nt-FOXC2-oligo 1-T
tcggcccgcgcgaggaatcaccaactacccacacacacc

SEQ ID NO: 109 - DNA, 39 bp, Artificial Sequence
  Synthetic 450-nt-FZD7-oligo 1-T
cgcaccgcagcgcgcaatcaccaactacccacacacacc

SEQ ID NO: 110 - DNA, 40 bp, Artificial Sequence
  Synthetic 450-nt-FZD8-oligo 1-T
ggcagggcccttccgcaatcaccaactacccacacacacc

SEQ ID NO: 111 - DNA, 48 bp, Artificial Sequence
  Synthetic 450-nt-GPR158-oligo 1-T
cgctctcattgaggcgcgttcagaaatcaccaactacccacacacacc

SEQ ID NO: 112 - DNA, 56 bp, Artificial Sequence
  Synthetic 450-nt-GSTA1-oligo 1-T
ccatgtaaatgtgaatttcaggcaaacaatcaaatcaccaactacccacacacacc

SEQ ID NO: 113 - DNA, 61 bp, Artificial Sequence
  Synthetic 450-nt-HIST1H1B-oligo 1-T
catacttgtttgtgttttcaagaatcttgtgcaattgaatcaccaactacccacacacacc

SEQ ID NO: 114 - DNA, 43 bp, Artificial Sequence
  Synthetic 450-nt-HMGA2-oligo 1-T
cggcggcggggttaaagggaatcaccaactacccacacacacc

SEQ ID NO: 115 - DNA, 53 bp, Artificial Sequence
  Synthetic 450-nt-HOTAIR-oligo 1-T
ccgagccttaatgtaggagctaaagccagaatcaccaactacccacacacacc

SEQ ID NO: 116 - DNA, 49 bp, Artificial Sequence
  Synthetic 450-nt-HOXB2-oligo 1-T
gcctccgtttctccagaaaagctagaatcaccaactacccacacacacc

SEQ ID NO: 117 - DNA, 44 bp, Artificial Sequence; identical to SEQ ID NO: 41
  Synthetic 450-nt-HSPA5-oligo 1-T

SEQ ID NO: 118 - DNA, 53 bp, Artificial Sequence
  Synthetic 450-nt-INO80-oligo 1-T
cgcaggagaaaatgtcgcgagagctttccaatcaccaactacccacacacacc

SEQ ID NO: 119 - DNA, 57 bp, Artificial Sequence
  Synthetic 450-nt-KLF8-oligo 1-T
gggctgtctaagagggaaaggagaaaacaaaaaaatcaccaactacccacacacacc

SEQ ID NO: 120 - DNA, 42 bp, Artificial Sequence
  Synthetic 450-nt-LIG1-oligo 1-T
gcagctggacgtggggggaatcaccaactacccacacacacc

SEQ ID NO: 121 - DNA, 41 bp, Artificial Sequence
  Synthetic 450-nt-MBD4-oligo 1-T
gcttgctgcctgggccgaatcaccaactacccacacacacc

SEQ ID NO: 122 - DNA, 52 bp, Artificial Sequence
  Synthetic 450-nt-MCM9-oligo 1-T
ccccaaatttccttcatattaaccctgcaatcaccaactacccacacacacc

SEQ ID NO: 123 - DNA, 47 bp, Artificial Sequence
  Synthetic 450-nt-MECP2-oligo 1-T
gcaagtgaaggcctaggcttgggaatcaccaactacccacacacacc

SEQ ID NO: 124 - DNA, 48 bp, Artificial Sequence
  Synthetic 450-nt-MEG3-oligo 1-T
attcaggcacaatgtgtggggggaaatcaccaactacccacacacacc

SEQ ID NO: 125 - DNA, 45 bp, Artificial Sequence
  Synthetic 450-nt-MGMT-oligo 1-T
gatggcccttcggccggtacaaatcaccaactacccacacacacc

SEQ ID NO: 126 - DNA, 48 bp, Artificial Sequence; identical to SEQ ID NO: 42
  Synthetic 450-nt-MLH1-oligo 1-T

SEQ ID NO: 127 - DNA, 45 bp, Artificial Sequence; identical to SEQ ID NO: 43
  Synthetic 450-nt-MLH3-oligo 1-T

SEQ ID NO: 128 - DNA, 59 bp, Artificial Sequence
  Synthetic 450-nt-MNAT1-oligo 1-T
gatttatgagcggtgtacttactttcttttggaccaatcaccaactacccacacacacc

SEQ ID NO: 129 - DNA, 46 bp, Artificial Sequence
  Synthetic 450-nt-MPG-oligo 1-T
acgtgttttccggtggggtgccaatcaccaactacccacacacacc

SEQ ID NO: 130 - DNA, 44 bp, Artificial Sequence
  Synthetic 450-nt-MRE11-oligo 1-T
ccgcttcgtggtccccaggtaatcaccaactacccacacacacc

SEQ ID NO: 131 - DNA, 50 bp, Artificial Sequence; identical to SEQ ID NO: 44
  Synthetic 450-nt-MSH2-oligo 1-T

SEQ ID NO: 132 - DNA, 42 bp, Artificial Sequence; identical to SEQ ID NO: 45
  Synthetic 450-nt-MSH3-oligo 1-T

SEQ ID NO: 133 - DNA, 62 bp, Artificial Sequence
  Synthetic 450-nt-MSH4-oligo 1-T
gcacactagcttatctttaaatgtagaaaccacccaacaatcaccaactacccacacacacc

SEQ ID NO: 134 - DNA, 53 bp, Artificial Sequence
  Synthetic 450-nt-MSH5-oligo 1-T
gggttttgagggggaggccaaacttaaagaatcaccaactacccacacacacc

SEQ ID NO: 135 - DNA, 44 bp, Artificial Sequence; identical to SEQ ID NO: 46
  Synthetic 450-nt-MSH6-oligo 1-T

SEQ ID NO: 136 - DNA, 51 bp, Artificial Sequence
  Synthetic 450-nt-MUTYH-oligo 1-T
caagccctacaaaatgtttcctgcaggaatcaccaactacccacacacacc

SEQ ID NO: 137 - DNA, 59 bp, Artificial Sequence
  Synthetic 450-nt-NEIL2-oligo 1-T
ctggcgcccggctaatttttgtatttttagtagagaatcaccaactacccacacacacc

SEQ ID NO: 138 - DNA, 50 bp, Artificial Sequence
  Synthetic 450-nt-NEIL3-oligo 1-T
cctcttaatctgtccttgtctgtgacaatcaccaactacccacacacacc

SEQ ID NO: 139 - DNA, 42 bp, Artificial Sequence
  Synthetic 450-nt-NKX2-5-oligo 1-T
gggtgggaagcggtggggaatcaccaactacccacacacacc

SEQ ID NO: 140 - DNA, 45 bp, Artificial Sequence
  Synthetic 450-nt-OGG1-oligo 1-T
cgttctcccccgagcctttgcaatcaccaactacccacacacacc

SEQ ID NO: 141 - DNA, 43 bp, Artificial Sequence
  Synthetic 450-nt-OLIG2-oligo 1-T
ggggatggagggagggctgaatcaccaactacccacacacacc

SEQ ID NO: 142 - DNA, 46 bp, Artificial Sequence
  Synthetic 450-nt-PARP1-oligo 1-T
aggcgctcaccacaactggctaaatcaccaactacccacacacacc

SEQ ID NO: 143 - DNA, 41 bp, Artificial Sequence
  Synthetic 450-nt-PCNA-oligo 1-T
agccctgctcccgggagaatcaccaactacccacacacacc

SEQ ID NO: 144 - DNA, 42 bp, Artificial Sequence
  Synthetic 450-nt-PELO-oligo 1-T
accaccccgacaggctccaatcaccaactacccacacacacc

SEQ ID NO: 145 - DNA, 49 bp, Artificial Sequence; identical to SEQ ID NO: 47
  Synthetic 450-nt-PMS1-oligo 1-T

SEQ ID NO: 146 - DNA, 44 bp, Artificial Sequence; identical to SEQ ID NO: 48
  Synthetic 450-nt-PMS2-oligo 1-T

SEQ ID NO: 147 - DNA, 52 bp, Artificial Sequence
  Synthetic 450-nt-PMS2P11-oligo 1-T
cccatcaccatgcctggctaatttttgtaatcaccaactacccacacacacc

SEQ ID NO: 148 - DNA, 46 bp, Artificial Sequence
  Synthetic 450-nt-PMS2P1-oligo 1-T
gagttcgagaccagtctggccaaatcaccaactacccacacacacc

SEQ ID NO: 149 - DNA, 46 bp, Artificial Sequence
  Synthetic 450-nt-PMS2P2-oligo 1-T
ccttggtgtctggcggtttccaaatcaccaactacccacacacacc

SEQ ID NO: 150 - DNA, 47 bp, Artificial Sequence
  Synthetic 450-nt-PMS2P3-oligo 1-T
ctggagcccaagtcctctgggttaatcaccaactacccacacacacc

SEQ ID NO: 151 - DNA, 47 bp, Artificial Sequence
  Synthetic 450-nt-PMS2P5-oligo 1-T
gcgttggtgtctggcggtttctgaatcaccaactacccacacacacc

SEQ ID NO: 152 - DNA, 41 bp, Artificial Sequence
  Synthetic 450-nt-POLD1-oligo 1-T
tcgggtgttgcgcgcgcaatcaccaactacccacacacacc

SEQ ID NO: 153 - DNA, 45 bp, Artificial Sequence
  Synthetic 450-nt-POLD4-oligo 1-T
gcttcgctaatccccgtggccaatcaccaactacccacacacacc

SEQ ID NO: 154 - DNA, 57 bp, Artificial Sequence
  Synthetic 450-nt-POLG-oligo 1-T
gccgcggtcgaagggaggttatgattaacttttaatcaccaactacccacacacacc

SEQ ID NO: 155 - DNA, 42 bp, Artificial Sequence
  Synthetic 450-nt-POLK-oligo 1-T
aacgcaggggtcacggcgaatcaccaactacccacacacacc

SEQ ID NO: 156 - DNA, 52 bp, Artificial Sequence
  Synthetic 450-nt-POLQ-oligo 1-T
aggtagatcagggaagtagctcttcttaaatcaccaactacccacacacacc

SEQ ID NO: 157 - DNA, 44 bp, Artificial Sequence
  Synthetic 450-nt-POLR2B-oligo 1-T
agggtcaccacgcccgggtaaatcaccaactacccacacacacc

SEQ ID NO: 158 - DNA, 51 bp, Artificial Sequence
  Synthetic 450-nt-POLR2G-oligo 1-T
tgtgccaccacacctgactaattttgtaatcaccaactacccacacacacc

SEQ ID NO: 159 - DNA, 51 bp, Artificial Sequence
  Synthetic 450-nt-PRDM2-oligo 1-T
cagacaagctgtgccaagaagtaattcaatcaccaactacccacacacacc

SEQ ID NO: 160 - DNA, 50 bp, Artificial Sequence
  Synthetic 450-nt-PRKCG-oligo 1-T
gggcgctaaggttctcactttctccgaatcaccaactacccacacacacc

SEQ ID NO: 161 - DNA, 44 bp, Artificial Sequence
  Synthetic 450-nt-PRUNE2-oligo 1-T
ggaggtgcaggggtatggcgaatcaccaactacccacacacacc

SEQ ID NO: 162 - DNA, 40 bp, Artificial Sequence
  Synthetic 450-nt-PXMP4-oligo 1-T
ggaggagcagggccccaatcaccaactacccacacacacc

SEQ ID NO: 163 - DNA, 58 bp, Artificial Sequence
  Synthetic 450-nt-PYCARD-oligo 1-T
catgtcgtagaatcttggaatcatgacagctagaaatcaccaactacccacacacacc

SEQ ID NO: 164 - DNA, 47 bp, Artificial Sequence
  Synthetic 450-nt-RNASEH2A-oligo 1-T
cgtagcttcgaagacccagcctaaatcaccaactacccacacacacc

SEQ ID NO: 165 - DNA, 60 bp, Artificial Sequence
  Synthetic 450-nt-RNF111-oligo 1-T
caacatttaagccaaatcttgactacatcttggttcaatcaccaactacccacacacacc

SEQ ID NO: 166 - DNA, 53 bp, Artificial Sequence
  Synthetic 450-nt-RPA2-oligo 1-T
ggtcacattctcttcgtgtaatgccaccaaatcaccaactacccacacacacc

SEQ ID NO: 167 - DNA, 56 bp, Artificial Sequence
  Synthetic 450-nt-RPA3-oligo 1-T
ggggaaatgtatgtgtgttttaaagcttcccaaatcaccaactacccacacacacc

SEQ ID NO: 168 - DNA, 47 bp, Artificial Sequence
  Synthetic 450-nt-RPA4-oligo 1-T
ctttgggaggctgaggcaggttgaatcaccaactacccacacacacc

SEQ ID NO: 169 - DNA, 50 bp, Artificial Sequence
  Synthetic 450-nt-SH3TC1-oligo 1-T
cttccatttactgcacacacacacacaatcaccaactacccacacacacc

SEQ ID NO: 170 - DNA, 39 bp, Artificial Sequence
  Synthetic 450-nt-SIM2-oligo 1-T
gcggcagggagggcgaatcaccaactacccacacacacc

SEQ ID NO: 171 - DNA, 45 bp, Artificial Sequence
  Synthetic 450-nt-SMAD4-oligo 1-T
gcgtagagtgggcgtccagtaaatcaccaactacccacacacacc

SEQ ID NO: 172 - DNA, 56 bp, Artificial Sequence
  Synthetic 450-nt-SMUG1-oligo 1-T
cattcactattttcccaaagggattgcaaatcaatcaccaactacccacacacacc

SEQ ID NO: 173 - DNA, 41 bp, Artificial Sequence
  Synthetic 450-nt-SNAI1-oligo 1-T
gggcggggactcctccgaatcaccaactacccacacacacc

SEQ ID NO: 174 - DNA, 46 bp, Artificial Sequence
  Synthetic 450-nt-SNAI2-oligo 1-T
gcctggcttccagatgtggtgcaatcaccaactacccacacacacc

SEQ ID NO: 175 - DNA, 48 bp, Artificial Sequence
  Synthetic 450-nt-TAF1-oligo 1-T
ggagctgctcgagtcacgtggcttaatcaccaactacccacacacacc

SEQ ID NO: 176 - DNA, 58 bp, Artificial Sequence; identical to SEQ ID NO: 49
  Synthetic 450-nt-TDG-oligo 1-T

SEQ ID NO: 177 - DNA, 58 bp, Artificial Sequence
  Synthetic 450-nt-TP53-oligo 1-T
ctctcagctggatcctttcttctcatcagttaaaaatcaccaactacccacacacacc

SEQ ID NO: 178 - DNA, 44 bp, Artificial Sequence
  Synthetic 450-nt-TREX1-oligo 1-T
catgcccacctgtgggtcccaatcaccaactacccacacacacc

SEQ ID NO: 179 - DNA, 47 bp, Artificial Sequence
  Synthetic 450-nt-TRIM28-oligo 1-T
ttcgccgccgtagagtcgtctccaatcaccaactacccacacacacc

SEQ ID NO: 180 - DNA, 42 bp, Artificial Sequence
  Synthetic 450-nt-TWIST1-oligo 1-T
gcagcgcccccgaaccctaatcaccaactacccacacacacc

SEQ ID NO: 181 - DNA, 45 bp, Artificial Sequence
  Synthetic 450-nt-TWIST2-oligo 1-T
agtcccagatgcgctcccagaaatcaccaactacccacacacacc

SEQ ID NO: 182 - DNA, 44 bp, Artificial Sequence
  Synthetic 450-nt-UBB-oligo 1-T
acctccgcctcccgggttcaaatcaccaactacccacacacacc

SEQ ID NO: 183 - DNA, 58 bp, Artificial Sequence
  Synthetic 450-nt-USP47-oligo 1-T
cttctctagtagtaagcatggtgctttgtgttataatcaccaactacccacacacacc

SEQ ID NO: 184 - DNA, 53 bp, Artificial Sequence
  Synthetic 450-nt-WDR48-oligo 1-T
ggcccaagcaaaacgaaagcactgaaagaaatcaccaactacccacacacacc

SEQ ID NO: 185 - DNA, 44 bp, Artificial Sequence
  Synthetic 450-nt-WNT1-oligo 1-T
tccggagcccgtcgaggactaatcaccaactacccacacacacc

SEQ ID NO: 186 - DNA, 51 bp, Artificial Sequence
  Synthetic 450-nt-XPC-oligo 1-T
aggctgctttttccagactcaggaaaaaatcaccaactacccacacacacc

SEQ ID NO: 187 - DNA, 51 bp, Artificial Sequence
  Synthetic 450-nt-XRCC2-oligo 1-T
ggcccctttgtccgttctcgggttaaaaatcaccaactacccacacacacc

SEQ ID NO: 188 - DNA, 40 bp, Artificial Sequence
  Synthetic 450-nt-XRCC3-oligo 1-T
tccgcgcgcggctgacaatcaccaactacccacacacacc

SEQ ID NO: 189 - DNA, 45 bp, Artificial Sequence
  Synthetic 450-nt-ZEB1-oligo 1-T
gggcagggagggatctggctgaatcaccaactacccacacacacc

SEQ ID NO: 190 - DNA, 43 bp, Artificial Sequence
  Synthetic 450-nt-ZNF830-oligo 1-T
gacagcggttggagcggccaatcaccaactacccacacacacc

SEQ ID NO: 191 - DNA, 39 bp, Artificial Sequence
  Synthetic 450-nt-ALKBH1-oligo 2-T
ctgcgtgggccgccgaatcaccaactacccacacacacc

SEQ ID NO: 192 - DNA, 56 bp, Artificial Sequence
  Synthetic 450-nt-ALKBH2-oligo 2-T
cgattcagaagcgagacgcaaagaggttaaacaatcaccaactacccacacacacc

SEQ ID NO: 193 - DNA, 43 bp, Artificial Sequence
  Synthetic 450-nt-ALKBH3-oligo 2-T
gggtggtactcgcacccccaatcaccaactacccacacacacc

SEQ ID NO: 194 - DNA, 52 bp, Artificial Sequence
  Synthetic 450-nt-APEX1-oligo 2-T
cctcttatgagcaaaagagcaaccccgtaatcaccaactacccacacacacc

SEQ ID NO: 195 - DNA, 46 bp, Artificial Sequence
  Synthetic 450-nt-APEX2-oligo 2-T
cggcacagttgctgggttcctgaatcaccaactacccacacacacc

SEQ ID NO: 196 - DNA, 42 bp, Artificial Sequence
  Synthetic 450-nt-ATM-oligo 2-T
gccaagcagccgcagagcaatcaccaactacccacacacacc

SEQ ID NO: 197 - DNA, 46 bp, Artificial Sequence
  Synthetic 450-nt-ATRX-oligo 2-T
cgtcactgtagctgctgctggaaatcaccaactacccacacacacc

SEQ ID NO: 198 - DNA, 40 bp, Artificial Sequence
  Synthetic 450-nt-ATXN3-oligo 2-T
cccctcgccgggcgagaatcaccaactacccacacacacc

SEQ ID NO: 199 - DNA, 50 bp, Artificial Sequence
  Synthetic 450-nt-AXIN2-oligo 2-T
gagttgccaggaccttatcaaagcgcaatcaccaactacccacacacacc

SEQ ID NO: 200 - DNA, 48 bp, Artificial Sequence
  Synthetic 450-nt-BRCA1-oligo 2-T
gggcccagttatctgagaaaccccaatcaccaactacccacacacacc

SEQ ID NO: 201 - DNA, 50 bp, Artificial Sequence
  Synthetic 450-nt-BRCA2-oligo 2-T
ggtgcagttcttttttggccggagtaaatcaccaactacccacacacacc

SEQ ID NO: 202 - DNA, 44 bp, Artificial Sequence
  Synthetic 450-nt-CCN4-oligo 2-T
aggcaggactcaccgtggccaatcaccaactacccacacacacc

SEQ ID NO: 203 - DNA, 45 bp, Artificial Sequence
  Synthetic 450-nt-CD44-oligo 2-T
ggctgtgactaagcagcccgcaatcaccaactacccacacacacc

SEQ ID NO: 204 - DNA, 51 bp, Artificial Sequence
  Synthetic 450-nt-CDH13-oligo 2-T
tggtcatcagcctctacccaatgctgcaatcaccaactacccacacacacc

SEQ ID NO: 205 - DNA, 40 bp, Artificial Sequence
  Synthetic 450-nt-CDH1-oligo 2-T
ggctggagcgggctggaatcaccaactacccacacacacc

SEQ ID NO: 206 - DNA, 46 bp, Artificial Sequence
  Synthetic 450-nt-CDH2-oligo 2-T
cccagagatcaaggagctggggaatcaccaactacccacacacacc

SEQ ID NO: 207 - DNA, 44 bp, Artificial Sequence
  Synthetic 450-nt-CDKN2A-oligo 2-T
accatgttctcgccgcctccaatcaccaactacccacacacacc

SEQ ID NO: 208 - DNA, 40 bp, Artificial Sequence
  Synthetic 450-nt-CDKN2D-oligo 2-T
acggcgaggggctgggaatcaccaactacccacacacacc

SEQ ID NO: 209 - DNA, 51 bp, Artificial Sequence
  Synthetic 450-nt-CETN1-oligo 2-T
aaatgcttcccgaacttcttgcttctgaatcaccaactacccacacacacc

SEQ ID NO: 210 - DNA, 53 bp, Artificial Sequence
  Synthetic 450-nt-CHEK1-oligo 2-T
gaaagcgagcagtttatgttggagcttgaaatcaccaactacccacacacacc

SEQ ID NO: 211 - DNA, 53 bp, Artificial Sequence
  Synthetic 450-nt-CHEK2-oligo 2-T
cacacactcccccttcagctcaaaactacaatcaccaactacccacacacacc

SEQ ID NO: 212 - DNA, 53 bp, Artificial Sequence
  Synthetic 450-nt-CSDE1-oligo 2-T
ggcttctcgttagccttggtcttattctcaatcaccaactacccacacacacc

SEQ ID NO: 213 - DNA, 49 bp, Artificial Sequence
  Synthetic 450-nt-DDB2-oligo 2-T
gggtactgtgctctatgcgaagatcaatcaccaactacccacacacacc

SEQ ID NO: 214 - DNA, 55 bp, Artificial Sequence
  Synthetic 450-nt-DRD3-oligo 2-T
cctgagactggataatttataatgaacagaaaatcaccaactacccacacacacc

SEQ ID NO: 215 - DNA, 43 bp, Artificial Sequence
  Synthetic 450-nt-DRD4-oligo 2-T
cacgagcgagttccccgcgaatcaccaactacccacacacacc

SEQ ID NO: 216 - DNA, 39 bp, Artificial Sequence
  Synthetic 450-nt-EGFR-oligo 2-T
ccgcgtcgggcgctcaatcaccaactacccacacacacc

SEQ ID NO: 217 - DNA, 49 bp, Artificial Sequence
  Synthetic 450-nt-EPM2AIP1-oligo 2-T
gctgggtctcttcgtccctccctgaaatcaccaactacccacacacacc

SEQ ID NO: 218 - DNA, 46 bp, Artificial Sequence
  Synthetic 450-nt-EPSTI1-oligo 2-T
cgagcccggagttcaccactctaatcaccaactacccacacacacc

SEQ ID NO: 219 - DNA, 43 bp, Artificial Sequence
  Synthetic 450-nt-ERCC1-oligo 2-T
ggcgccaatctccacccgcaatcaccaactacccacacacacc

SEQ ID NO: 220 - DNA, 38 bp, Artificial Sequence
  Synthetic 450-nt-ERCC2-oligo 2-T
acccaggcgcgcccaatcaccaactacccacacacacc

SEQ ID NO: 221 - DNA, 55 bp, Artificial Sequence
  Synthetic 450-nt-ERCC8-oligo 2-T
ggcctgttagccaaaaagtaaggttttctttaatcaccaactacccacacacacc

SEQ ID NO: 222 - DNA, 44 bp, Artificial Sequence
  Synthetic 450-nt-EXO1-oligo 2-T
ctcctcctccccttgagcggaatcaccaactacccacacacacc

SEQ ID NO: 223 - DNA, 43 bp, Artificial Sequence
  Synthetic 450-nt-FANCC-oligo 2-T
tcctcgcgggagctgcttcaatcaccaactacccacacacacc

SEQ ID NO: 224 - DNA, 45 bp, Artificial Sequence
  Synthetic 450-nt-FAT4-oligo 2-T
gctgctggaagagcaacctccaatcaccaactacccacacacacc

SEQ ID NO: 225 - DNA, 46 bp, Artificial Sequence
  Synthetic 450-nt-FEN1-oligo 2-T
gcaaccagtccctccagggagaaatcaccaactacccacacacacc

SEQ ID NO: 226 - DNA, 43 bp, Artificial Sequence
  Synthetic 450-nt-FHIT-oligo 2-T
ctccccgcccctaccttccaatcaccaactacccacacacacc

SEQ ID NO: 227 - DNA, 43 bp, Artificial Sequence
  Synthetic 450-nt-FOXC2-oligo 2-T
tagctgcccgcagcccggtaatcaccaactacccacacacacc

SEQ ID NO: 228 - DNA, 42 bp, Artificial Sequence
  Synthetic 450-nt-FZD7-oligo 2-T
gccagcaccagggcacagaatcaccaactacccacacacacc

SEQ ID NO: 229 - DNA, 45 bp, Artificial Sequence
  Synthetic 450-nt-FZD8-oligo 2-T
ggcgagcactggatctccaccaatcaccaactacccacacacacc

SEQ ID NO: 230 - DNA, 41 bp, Artificial Sequence
  Synthetic 450-nt-GPR158-oligo 2-T
tcgcggctggcgccaacaatcaccaactacccacacacacc

SEQ ID NO: 231 - DNA, 44 bp, Artificial Sequence
  Synthetic 450-nt-GSTA1-oligo 2-T
gtcaccgtcctggctcgacaaatcaccaactacccacacacacc

SEQ ID NO: 232 - DNA, 48 bp, Artificial Sequence
  Synthetic 450-nt-HIST1H1B-oligo 2-T
ttagaagcagccacagccttggtgaatcaccaactacccacacacacc

SEQ ID NO: 233 - DNA, 47 bp, Artificial Sequence
  Synthetic 450-nt-HMGA2-oligo 2-T
cagaggacagagtagtgggtggcaatcaccaactacccacacacacc

SEQ ID NO: 234 - DNA, 44 bp, Artificial Sequence
  Synthetic 450-nt-HOTAIR-oligo 2-T
ggggcaaggaggcataggccaatcaccaactacccacacacacc

SEQ ID NO: 235 - DNA, 47 bp, Artificial Sequence
  Synthetic 450-nt-HOXB2-oligo 2-T
gagcgacggctggctgtttataaaatcaccaactacccacacacacc

SEQ ID NO: 236 - DNA, 44 bp, Artificial Sequence
  Synthetic 450-nt-HSPA5-oligo 2-T
accaggcgaagggcaggtctaatcaccaactacccacacacacc

SEQ ID NO: 237 - DNA, 42 bp, Artificial Sequence
  Synthetic 450-nt-INO80-oligo 2-T
gcggctcctgggaccccaaatcaccaactacccacacacacc

SEQ ID NO: 238 - DNA, 46 bp, Artificial Sequence
  Synthetic 450-nt-KLF8-oligo 2-T
ccccacgcgagtcagaacctaaaatcaccaactacccacacacacc

SEQ ID NO: 239 - DNA, 47 bp, Artificial Sequence
  Synthetic 450-nt-LIG1-oligo 2-T
cactgggagtcgtagtctcccgaaatcaccaactacccacacacacc

SEQ ID NO: 240 - DNA, 49 bp, Artificial Sequence
  Synthetic 450-nt-MBD4-oligo 2-T
gccatttccacgctccaccaatcaaaatcaccaactacccacacacacc

SEQ ID NO: 241 - DNA, 52 bp, Artificial Sequence
  Synthetic 450-nt-MCM9-oligo 2-T
gatttccatgttggtctcaaacagagtcaatcaccaactacccacacacacc

SEQ ID NO: 242 - DNA, 42 bp, Artificial Sequence
  Synthetic 450-nt-MECP2-oligo 2-T
gcggcggccattttccggaatcaccaactacccacacacacc

SEQ ID NO: 243 - DNA, 45 bp, Artificial Sequence
  Synthetic 450-nt-MEG3-oligo 2-T
cagagcccatcccagaagcggaatcaccaactacccacacacacc

SEQ ID NO: 244 - DNA, 42 bp, Artificial Sequence
  Synthetic 450-nt-MGMT-oligo 2-T
gaggctgccaccgtcccgaatcaccaactacccacacacacc

SEQ ID NO: 245 - DNA, 56 bp, Artificial Sequence
  Synthetic 450-nt-MLH1-oligo 2-T
gcatacataattttaaattaagtggcttccttaatcaccaactacccacacacacc

SEQ ID NO: 246 - DNA, 48 bp, Artificial Sequence
  Synthetic 450-nt-MLH3-oligo 2-T
gagtcccgcttaacaaattctccgaatcaccaactacccacacacacc

SEQ ID NO: 247 - DNA, 48 bp, Artificial Sequence
  Synthetic 450-nt-MNAT1-oligo 2-T
ggggttccgatatttggtggtcttaatcaccaactacccacacacacc

SEQ ID NO: 248 - DNA, 40 bp, Artificial Sequence
  Synthetic 450-nt-MPG-oligo 2-T
ccagcagccgccgaccaatcaccaactacccacacacacc

SEQ ID NO: 249 - DNA, 46 bp, Artificial Sequence
  Synthetic 450-nt-MRE11-oligo 2-T
gaagccccacaatcgcgacccaaatcaccaactacccacacacacc

SEQ ID NO: 250 - DNA, 48 bp, Artificial Sequence
  Synthetic 450-nt-MSH2-oligo 2-T
gcaccgccatgtcgaaacctcctcaatcaccaactacccacacacacc

SEQ ID NO: 251 - DNA, 43 bp, Artificial Sequence
  Synthetic 450-nt-MSH3-oligo 2-T
gcttgcctcgcaggggctgaatcaccaactacccacacacacc

SEQ ID NO: 252 - DNA, 40 bp, Artificial Sequence
  Synthetic 450-nt-MSH4-oligo 2-T
gggaaaccgccggggcaatcaccaactacccacacacacc

SEQ ID NO: 253 - DNA, 41 bp, Artificial Sequence
  Synthetic 450-nt-MSH5-oligo 2-T
ctcccccagctgccgccaatcaccaactacccacacacacc

SEQ ID NO: 254 - DNA, 44 bp, Artificial Sequence
  Synthetic 450-nt-MSH6-oligo 2-T
aggcccaaccgttctgtcggaatcaccaactacccacacacacc

SEQ ID NO: 255 - DNA, 42 bp, Artificial Sequence
  Synthetic 450-nt-MUTYH-oligo 2-T
gacggtgagcggcttcccaatcaccaactacccacacacacc

SEQ ID NO: 256 - DNA, 43 bp, Artificial Sequence
  Synthetic 450-nt-NEIL2-oligo 2-T
atgcggcctccgtccccttaatcaccaactacccacacacacc

SEQ ID NO: 257 - DNA, 44 bp, Artificial Sequence
  Synthetic 450-nt-NEIL3-oligo 2-T
cgcgcgcgaatcttctctccaatcaccaactacccacacacacc

SEQ ID NO: 258 - DNA, 43 bp, Artificial Sequence
  Synthetic 450-nt-NKX2-5-oligo 2-T
cccctggcagcttccctgcaatcaccaactacccacacacacc

SEQ ID NO: 259 - DNA, 44 bp, Artificial Sequence
  Synthetic 450-nt-OGG1-oligo 2-T
tgcctcgccctttgcgggtcaatcaccaactacccacacacacc

SEQ ID NO: 260 - DNA, 48 bp, Artificial Sequence
  Synthetic 450-nt-OLIG2-oligo 2-T
gaggagagtctgtccctgggatctaatcaccaactacccacacacacc

SEQ ID NO: 261 - DNA, 42 bp, Artificial Sequence
  Synthetic 450-nt-PARP1-oligo 2-T
ccagagccgccaccgaacaatcaccaactacccacacacacc

SEQ ID NO: 262 - DNA, 46 bp, Artificial Sequence
  Synthetic 450-nt-PCNA-oligo 2-T
accgcttggcaaagcaaagcagaatcaccaactacccacacacacc

SEQ ID NO: 263 - DNA, 48 bp, Artificial Sequence
  Synthetic 450-nt-PELO-oligo 2-T
cgcgctcttggtctctgaaatctcaatcaccaactacccacacacacc

SEQ ID NO: 264 - DNA, 44 bp, Artificial Sequence
  Synthetic 450-nt-PMS1-oligo 2-T
tagggaactgctggcggcctaatcaccaactacccacacacacc

SEQ ID NO: 265 - DNA, 44 bp, Artificial Sequence
  Synthetic 450-nt-PMS2-oligo 2-T
ggggacaccggaagactgcgaatcaccaactacccacacacacc

SEQ ID NO: 266 - DNA, 66 bp, Artificial Sequence
  Synthetic 450-nt-PMS2P11-oligo 2-T
ccacatggagaaaacatacattgtatctctcaaattaccaaaaatcaccaactacccacacacacc

SEQ ID NO: 267 - DNA, 44 bp, Artificial Sequence
  Synthetic 450-nt-PMS2P1-oligo 2-T
gtggcctggcgtggattccgaatcaccaactacccacacacacc

SEQ ID NO: 268 - DNA, 40 bp, Artificial Sequence
  Synthetic 450-nt-PMS2P2-oligo 2-T
cacgaggcggcctcccaatcaccaactacccacacacacc

SEQ ID NO: 269 - DNA, 43 bp, Artificial Sequence
  Synthetic 450-nt-PMS2P3-oligo 2-T
cgcgcgaaccctggtctgcaatcaccaactacccacacacacc

SEQ ID NO: 270 - DNA, 43 bp, Artificial Sequence
  Synthetic 450-nt-PMS2P5-oligo 2-T
cctcggccatgttccccccaatcaccaactacccacacacacc

SEQ ID NO: 271 - DNA, 44 bp, Artificial Sequence
  Synthetic 450-nt-POLD1-oligo 2-T
ggccaaagggtgccctcgcaaatcaccaactacccacacacacc

SEQ ID NO: 272 - DNA, 45 bp, Artificial Sequence
  Synthetic 450-nt-POLD4-oligo 2-T
gaggcaactgcggggaagaggaatcaccaactacccacacacacc

SEQ ID NO: 273 - DNA, 40 bp, Artificial Sequence
  Synthetic 450-nt-POLG-oligo 2-T
accccaggcccacggcaatcaccaactacccacacacacc

SEQ ID NO: 274 - DNA, 48 bp, Artificial Sequence
  Synthetic 450-nt-POLK-oligo 2-T
gggatactcacccgttacctcaggaatcaccaactacccacacacacc

SEQ ID NO: 275 - DNA, 51 bp, Artificial Sequence
  Synthetic 450-nt-POLQ-oligo 2-T
cgagaacgaatctgagcctgattctgaaatcaccaactacccacacacacc

SEQ ID NO: 276 - DNA, 48 bp, Artificial Sequence
  Synthetic 450-nt-POLR2B-oligo 2-T
aggatctgcgcctgcgcaaattgtaatcaccaactacccacacacacc

SEQ ID NO: 277 - DNA, 47 bp, Artificial Sequence
  Synthetic 450-nt-POLR2G-oligo 2-T
ggccttgctcttcctttccacccaatcaccaactacccacacacacc

SEQ ID NO: 278 - DNA, 52 bp, Artificial Sequence
  Synthetic 450-nt-PRDM2-oligo 2-T
gccttctaacaagtggtaacagagcttcaatcaccaactacccacacacacc

SEQ ID NO: 279 - DNA, 43 bp, Artificial Sequence
  Synthetic 450-nt-PRKCG-oligo 2-T
agcgggcgagtggggaggaaatcaccaactacccacacacacc

SEQ ID NO: 280 - DNA, 54 bp, Artificial Sequence
  Synthetic 450-nt-PRUNE2-oligo 2-T
cctcctctgctgcaacaccttttcctctccaatcaccaactacccacacacacc

SEQ ID NO: 281 - DNA, 46 bp, Artificial Sequence
  Synthetic 450-nt-PXMP4-oligo 2-T
cagtgcgttgacgactacgagcaatcaccaactacccacacacacc

SEQ ID NO: 282 - DNA, 40 bp, Artificial Sequence
  Synthetic 450-nt-PYCARD-oligo 2-T
ccgctcaccccgctgcaatcaccaactacccacacacacc

SEQ ID NO: 283 - DNA, 42 bp, Artificial Sequence
  Synthetic 450-nt-RNASEH2A-oligo 2-T
cccgcctcatcgacgcccaatcaccaactacccacacacacc

SEQ ID NO: 284 - DNA, 44 bp, Artificial Sequence
  Synthetic 450-nt-RNF111-oligo 2-T
gcctggactcggcccagctaaatcaccaactacccacacacacc

SEQ ID NO: 285 - DNA, 47 bp, Artificial Sequence
  Synthetic 450-nt-RPA2-oligo 2-T
gccaatcggtgctcacggatgctaatcaccaactacccacacacacc

SEQ ID NO: 286 - DNA, 62 bp, Artificial Sequence
  Synthetic 450-nt-RPA3-oligo 2-T
cttgactatgtgtgtcttcaaaataatacctttaagttaatcaccaactacccacacacacc

SEQ ID NO: 287 - DNA, 46 bp, Artificial Sequence
  Synthetic 450-nt-RPA4-oligo 2-T
ggccaccttctcacctgatcgcaatcaccaactacccacacacacc

SEQ ID NO: 288 - DNA, 45 bp, Artificial Sequence
  Synthetic 450-nt-SH3TC1-oligo 2-T
catacctgcggcgtgttcctcaatcaccaactacccacacacacc

SEQ ID NO: 289 - DNA, 41 bp, Artificial Sequence
  Synthetic 450-nt-SIM2-oligo 2-T
ggagcccagcggaggggaatcaccaactacccacacacacc

SEQ ID NO: 290 - DNA, 38 bp, Artificial Sequence
  Synthetic 450-nt-SMAD4-oligo 2-T
ctgggcggccgcggaatcaccaactacccacacacacc

SEQ ID NO: 291 - DNA, 46 bp, Artificial Sequence
  Synthetic 450-nt-SMUG1-oligo 2-T
cttaccccatacggagggagggaatcaccaactacccacacacacc

SEQ ID NO: 292 - DNA, 40 bp, Artificial Sequence
  Synthetic 450-nt-SNAI1-oligo 2-T
gcaggccgcctccctcaatcaccaactacccacacacacc

SEQ ID NO: 293 - DNA, 43 bp, Artificial Sequence
  Synthetic 450-nt-SNAI2-oligo 2-T
cggataacggtccggcgggaatcaccaactacccacacacacc

SEQ ID NO: 294 - DNA, 44 bp, Artificial Sequence
  Synthetic 450-nt-TAF1-oligo 2-T
aatgggccgcctccagcggaaatcaccaactacccacacacacc

SEQ ID NO: 295 - DNA, 46 bp, Artificial Sequence
  Synthetic 450-nt-TDG-oligo 2-T
ctcactgcggtaagacaaccccaatcaccaactacccacacacacc

SEQ ID NO: 296 - DNA, 47 bp, Artificial Sequence
  Synthetic 450-nt-TP53-oligo 2-T
ggaagcgtgtcaccgtcgtggaaaatcaccaactacccacacacacc

SEQ ID NO: 297 - DNA, 44 bp, Artificial Sequence
  Synthetic 450-nt-TREX1-oligo 2-T
ccctcccctagtgcccccttaatcaccaactacccacacacacc

SEQ ID NO: 298 - DNA, 41 bp, Artificial Sequence
  Synthetic 450-nt-TRIM28-oligo 2-T
ccgccgaaacccgcaccaatcaccaactacccacacacacc

SEQ ID NO: 299 - DNA, 40 bp, Artificial Sequence
  Synthetic 450-nt-TWIST1-oligo 2-T
gcgggagggggaggggaatcaccaactacccacacacacc

SEQ ID NO: 300 - DNA, 47 bp, Artificial Sequence
  Synthetic 450-nt-TWIST2-oligo 2-T
acacgctctcggagttgctggaaaatcaccaactacccacacacacc

SEQ ID NO: 301 - DNA, 51 bp, Artificial Sequence
  Synthetic 450-nt-UBB-oligo 2-T
agtttaggcttttcaactgagccccaaaatcaccaactacccacacacacc

SEQ ID NO: 302 - DNA, 45 bp, Artificial Sequence
  Synthetic 450-nt-USP47-oligo 2-T
cctggacgtcatcccctcttcaatcaccaactacccacacacacc

SEQ ID NO: 303 - DNA, 42 bp, Artificial Sequence
  Synthetic 450-nt-WDR48-oligo 2-T
tggcgccgggcaccttctaatcaccaactacccacacacacc

SEQ ID NO: 304 - DNA, 45 bp, Artificial Sequence
  Synthetic 450-nt-WNT1-oligo 2-T
ctctgtgctgcagttgcggcgaatcaccaactacccacacacacc

SEQ ID NO: 305 - DNA, 45 bp, Artificial Sequence
  Synthetic 450-nt-XPC-oligo 2-T
gggccttgctcttggccttggaatcaccaactacccacacacacc

SEQ ID NO: 306 - DNA, 43 bp, Artificial Sequence
  Synthetic 450-nt-XRCC2-oligo 2-T
tcccaacccggcggctctcaatcaccaactacccacacacacc

SEQ ID NO: 307 - DNA, 39 bp, Artificial Sequence
  Synthetic 450-nt-XRCC3-oligo 2-T
cgcccccgccccttgaatcaccaactacccacacacacc

SEQ ID NO: 308 - DNA, 48 bp, Artificial Sequence
  Synthetic 450-nt-ZEB1-oligo 2-T
cgggttcgcctgctttctgcgcttaatcaccaactacccacacacacc

SEQ ID NO: 309 - DNA, 51 bp, Artificial Sequence
  Synthetic 450-nt-ZNF830-oligo 2-T
cggttgtacttcgcgaatggagattctaatcaccaactacccacacacacc

SEQ ID NO: 310 - DNA, 47 bp, Artificial Sequence
  Synthetic 450-nt-ALKBH1-oligo 3-T
ctaccccaccttcctcattctctagaagtcgatgaccccttccaggt

SEQ ID NO: 311 - DNA, 49 bp, Artificial Sequence
  Synthetic 450-nt-ALKBH2-oligo 3-T
ctaccccaccttcctcattctctacgggtaaaccgcacgcaaaattctg

SEQ ID NO: 312 - DNA, 38 bp, Artificial Sequence
  Synthetic 450-nt-ALKBH3-oligo 3-T
ctaccccaccttcctcattctctagccccgcgcaccca

SEQ ID NO: 313 - DNA, 41 bp, Artificial Sequence
  Synthetic 450-nt-APEX1-oligo 3-T
ctaccccaccttcctcattctctatctgcaccggcccgatg

SEQ ID NO: 314 - DNA, 47 bp, Artificial Sequence
  Synthetic 450-nt-APEX2-oligo 3-T
ctaccccaccttcctcattctctatttgccaccccttgcaggggtct

SEQ ID NO: 315 - DNA, 47 bp, Artificial Sequence
  Synthetic 450-nt-ATM-oligo 3-T
ctaccccaccttcctcattctctacggtatgcccatgcgcatccagt

SEQ ID NO: 316 - DNA, 44 bp, Artificial Sequence
  Synthetic 450-nt-ATRX-oligo 3-T
ctaccccaccttcctcattctctacctccccacagctcaaaggc

SEQ ID NO: 317 - DNA, 43 bp, Artificial Sequence
  Synthetic 450-nt-ATXN3-oligo 3-T
ctaccccaccttcctcattctctatcggcatgggggcgactcg

SEQ ID NO: 318 - DNA, 41 bp, Artificial Sequence
  Synthetic 450-nt-AXIN2-oligo 3-T
ctaccccaccttcctcattctctagccggctccagccgact

SEQ ID NO: 319 - DNA, 42 bp, Artificial Sequence
  Synthetic 450-nt-BRCA1-oligo 3-T
ctaccccaccttcctcattctctacagcctgtcccccgtcca

SEQ ID NO: 320 - DNA, 44 bp, Artificial Sequence
  Synthetic 450-nt-BRCA2-oligo 3-T
ctaccccaccttcctcattctctagctgacaaaaaccgcgccgg

SEQ ID NO: 321 - DNA, 41 bp, Artificial Sequence
  Synthetic 450-nt-CCN4-oligo 3-T
ctaccccaccttcctcattctctaggacggtgctggcggct

SEQ ID NO: 322 - DNA, 51 bp, Artificial Sequence
  Synthetic 450-nt-CD44-oligo 3-T
ctaccccaccttcctcattctctagaggttttaagaagtagcagccctccc

SEQ ID NO: 323 - DNA, 54 bp, Artificial Sequence
  Synthetic 450-nt-CDH13-oligo 3-T
ctaccccaccttcctcattctctattgggtagaggctgatgaccaggaccaatg

SEQ ID NO: 324 - DNA, 50 bp, Artificial Sequence
  Synthetic 450-nt-CDH1-oligo 3-T
ctaccccaccttcctcattctctagtctgaactgacttccgcaagctcac

SEQ ID NO: 325 - DNA, 41 bp, Artificial Sequence
  Synthetic 450-nt-CDH2-oligo 3-T
ctaccccaccttcctcattctctagggggcgggggaacagg

SEQ ID NO: 326 - DNA, 41 bp, Artificial Sequence
  Synthetic 450-nt-CDKN2A-oligo 3-T
ctaccccaccttcctcattctctagggccgagctcggcagc

SEQ ID NO: 327 - DNA, 39 bp, Artificial Sequence
  Synthetic 450-nt-CDKN2D-oligo 3-T
ctaccccaccttcctcattctctagccgggcccaaccct

SEQ ID NO: 328 - DNA, 45 bp, Artificial Sequence
  Synthetic 450-nt-CETN1-oligo 3-T
ctaccccaccttcctcattctctatcctcagtgagctcgggctta

SEQ ID NO: 329 - DNA, 56 bp, Artificial Sequence
  Synthetic 450-nt-CHEK1-oligo 3-T
ctaccccaccttcctcattctctagtcaggataaccagttttgccttttttctgtg

SEQ ID NO: 330 - DNA, 49 bp, Artificial Sequence
  Synthetic 450-nt-CHEK2-oligo 3-T
ctaccccaccttcctcattctctagacaaagcgaagctcaggagactcc

SEQ ID NO: 331 - DNA, 47 bp, Artificial Sequence
  Synthetic 450-nt-CSDE1-oligo 3-T
ctaccccaccttcctcattctctagcccttcccctgggtttctgcta

SEQ ID NO: 332 - DNA, 47 bp, Artificial Sequence
  Synthetic 450-nt-DDB2-oligo 3-T
ctaccccaccttcctcattctctatggagggaggattgagaggcctc

SEQ ID NO: 333 - DNA, 50 bp, Artificial Sequence
  Synthetic 450-nt-DRD3-oligo 3-T
ctaccccaccttcctcattctctatttatttggctcacagttctggagtc

SEQ ID NO: 334 - DNA, 42 bp, Artificial Sequence
  Synthetic 450-nt-DRD4-oligo 3-T
ctaccccaccttcctcattctctagcaccgcgccgatgagca

SEQ ID NO: 335 - DNA, 39 bp, Artificial Sequence
  Synthetic 450-nt-EGFR-oligo 3-T
ctaccccaccttcctcattctctacaccgtgcggggggc

SEQ ID NO: 336 - DNA, 48 bp, Artificial Sequence
  Synthetic 450-nt-EPM2AIP1-oligo 3-T
ctaccccaccttcctcattctctagcggctactgcccgctacctagaa

SEQ ID NO: 337 - DNA, 48 bp, Artificial Sequence
  Synthetic 450-nt-EPSTI1-oligo 3-T
ctaccccaccttcctcattctctattgcgggtgttcatggttcacagc

SEQ ID NO: 338 - DNA, 43 bp, Artificial Sequence
  Synthetic 450-nt-ERCC1-oligo 3-T
ctaccccaccttcctcattctctagtctccgcctcccgcacct

SEQ ID NO: 339 - DNA, 48 bp, Artificial Sequence
  Synthetic 450-nt-ERCC2-oligo 3-T
ctaccccaccttcctcattctctaccgatgaccccatcttcaagaccc

SEQ ID NO: 340 - DNA, 45 bp, Artificial Sequence
  Synthetic 450-nt-ERCC8-oligo 3-T
ctaccccaccttcctcattctctacctccgtgttgactctgctct

SEQ ID NO: 341 - DNA, 46 bp, Artificial Sequence
  Synthetic 450-nt-EXO1-oligo 3-T
ctaccccaccttcctcattctctagccgattgcggttggagaaacc

SEQ ID NO: 342 - DNA, 46 bp, Artificial Sequence
  Synthetic 450-nt-FANCC-oligo 3-T
ctaccccaccttcctcattctctagcagtttgggcagtggcggaaa

SEQ ID NO: 343 - DNA, 43 bp, Artificial Sequence
  Synthetic 450-nt-FAT4-oligo 3-T
ctaccccaccttcctcattctctagcttggaacacctggcgcg

SEQ ID NO: 344 - DNA, 45 bp, Artificial Sequence
  Synthetic 450-nt-FEN1-oligo 3-T
ctaccccaccttcctcattctctacagagtctctcagcgaccctg

SEQ ID NO: 345 - DNA, 53 bp, Artificial Sequence
  Synthetic 450-nt-FHIT-oligo 3-T
ctaccccaccttcctcattctctaggatgttgacagctgggaatgaaaggcag

SEQ ID NO: 346 - DNA, 48 bp, Artificial Sequence
  Synthetic 450-nt-FOXC2-oligo 3-T
ctaccccaccttcctcattctctagtaattctgctcgctcaggtaggg

SEQ ID NO: 347 - DNA, 44 bp, Artificial Sequence
  Synthetic 450-nt-FZD7-oligo 3-T
ctaccccaccttcctcattctctaggcccagggacgaaagcgga

SEQ ID NO: 348 - DNA, 45 bp, Artificial Sequence
  Synthetic 450-nt-FZD8-oligo 3-T
ctaccccaccttcctcattctctagcggccagaactggtgcacct

SEQ ID NO: 349 - DNA, 46 bp, Artificial Sequence
  Synthetic 450-nt-GPR158-oligo 3-T
ctaccccaccttcctcattctctagctcccaatcccagctgagcaa

SEQ ID NO: 350 - DNA, 55 bp, Artificial Sequence
  Synthetic 450-nt-GSTA1-oligo 3-T
ctaccccaccttcctcattctctactgaattccaggtcctaatgtatttataagc

SEQ ID NO: 351 - DNA, 44 bp, Artificial Sequence
  Synthetic 450-nt-HIST1H1B-oligo 3-T
ctaccccaccttcctcattctctatcagctctgagactgggggc

SEQ ID NO: 352 - DNA, 42 bp, Artificial Sequence
  Synthetic 450-nt-HMGA2-oligo 3-T
ctaccccaccttcctcattctctaccgcgcctcctagggtgg

SEQ ID NO: 353 - DNA, 46 bp, Artificial Sequence
  Synthetic 450-nt-HOTAIR-oligo 3-T
ctaccccaccttcctcattctctacaacactcaccccacggagcag

SEQ ID NO: 354 - DNA, 57 bp, Artificial Sequence
  Synthetic 450-nt-HOXB2-oligo 3-T
ctaccccaccttcctcattctctacccaatctccctctcaaattcaaaattcatggc

SEQ ID NO: 355 - DNA, 44 bp, Artificial Sequence
  Synthetic 450-nt-HSPA5-oligo 3-T
ctaccccaccttcctcattctctagaaatacaggccgcggcgct

SEQ ID NO: 356 - DNA, 41 bp, Artificial Sequence
  Synthetic 450-nt-INO80-oligo 3-T
ctaccccaccttcctcattctctagatggcggcggcgctga

SEQ ID NO: 357 - DNA, 45 bp, Artificial Sequence
  Synthetic 450-nt-KLF8-oligo 3-T
ctaccccaccttcctcattctctagtgctctctgggcaacctgag

SEQ ID NO: 358 - DNA, 44 bp, Artificial Sequence
  Synthetic 450-nt-LIG1-oligo 3-T
ctaccccaccttcctcattctctatgggaggagggcgggaaaag

SEQ ID NO: 359 - DNA, 48 bp, Artificial Sequence
  Synthetic 450-nt-MBD4-oligo 3-T
ctaccccaccttcctcattctctatccattctcgaggaagacgcaccg

SEQ ID NO: 360 - DNA, 50 bp, Artificial Sequence
  Synthetic 450-nt-MCM9-oligo 3-T
ctaccccaccttcctcattctctatggcattaaccacaactgggtaatga

SEQ ID NO: 361 - DNA, 47 bp, Artificial Sequence
  Synthetic 450-nt-MECP2-oligo 3-T
ctaccccaccttcctcattctctacggcttttaccacagccctctct

SEQ ID NO: 362 - DNA, 49 bp, Artificial Sequence
  Synthetic 450-nt-MEG3-oligo 3-T
ctaccccaccttcctcattctctatgccgtcttccttttgcacatcctt

SEQ ID NO: 363 - DNA, 44 bp, Artificial Sequence
  Synthetic 450-nt-MGMT-oligo 3-T
ctaccccaccttcctcattctctagggagagctccgcactcttc

SEQ ID NO: 364 - DNA, 51 bp, Artificial Sequence
  Synthetic 450-nt-MLH1-oligo 3-T
ctaccccaccttcctcattctctacttagttaacggtaccattaagacaac

SEQ ID NO: 365 - DNA, 43 bp, Artificial Sequence
  Synthetic 450-nt-MLH3-oligo 3-T
ctaccccaccttcctcattctctacaccaaccgcctcggacgc

SEQ ID NO: 366 - DNA, 44 bp, Artificial Sequence
  Synthetic 450-nt-MNAT1-oligo 3-T
ctaccccaccttcctcattctctacaccgagggcaaccctgatc

SEQ ID NO: 367 - DNA, 43 bp, Artificial Sequence
  Synthetic 450-nt-MPG-oligo 3-T
ctaccccaccttcctcattctctacggaagcacccctaggacc

SEQ ID NO: 368 - DNA, 50 bp, Artificial Sequence
  Synthetic 450-nt-MRE11-oligo 3-T
ctaccccaccttcctcattctctaggctgtcttcttttcgggaagaaagg

SEQ ID NO: 369 - DNA, 51 bp, Artificial Sequence
  Synthetic 450-nt-MSH2-oligo 3-T
ctaccccaccttcctcattctctacctcctggttgaagaaaatgcgcgacc

SEQ ID NO: 370 - DNA, 42 bp, Artificial Sequence
  Synthetic 450-nt-MSH3-oligo 3-T
ctaccccaccttcctcattctctagctggaggcagcgaggcc

SEQ ID NO: 371 - DNA, 48 bp, Artificial Sequence
  Synthetic 450-nt-MSH4-oligo 3-T
ctaccccaccttcctcattctctagaaggcgaggttgatgagatctca

SEQ ID NO: 372 - DNA, 47 bp, Artificial Sequence
  Synthetic 450-nt-MSH5-oligo 3-T
ctaccccaccttcctcattctctatcgcagcgttttcccgccttttc

SEQ ID NO: 373 - DNA, 47 bp, Artificial Sequence
  Synthetic 450-nt-MSH6-oligo 3-T
ctaccccaccttcctcattctctacggagctcctaaaagcaccgcat

SEQ ID NO: 374 - DNA, 48 bp, Artificial Sequence
  Synthetic 450-nt-MUTYH-oligo 3-T
ctaccccaccttcctcattctctagaggtagccttcaaagcctctgcg

SEQ ID NO: 375 - DNA, 42 bp, Artificial Sequence
  Synthetic 450-nt-NEIL2-oligo 3-T
ctaccccaccttcctcattctctaggggacgcccctgtaggt

SEQ ID NO: 376 - DNA, 46 bp, Artificial Sequence
  Synthetic 450-nt-NEIL3-oligo 3-T
ctaccccaccttcctcattctctattcagagtacagcctggtcctt

SEQ ID NO: 377 - DNA, 39 bp, Artificial Sequence
  Synthetic 450-nt-NKX2-5-oligo 3-T
ctaccccaccttcctcattctctatggtgccgccgcccg

SEQ ID NO: 378 - DNA, 48 bp, Artificial Sequence
  Synthetic 450-nt-OGG1-oligo 3-T
ctaccccaccttcctcattctctatcaaagccccgcttcgtgccccaa

SEQ ID NO: 379 - DNA, 41 bp, Artificial Sequence
  Synthetic 450-nt-OLIG2-oligo 3-T
ctaccccaccttcctcattctctaggctgcggctgctgtgg

SEQ ID NO: 380 - DNA, 38 bp, Artificial Sequence
  Synthetic 450-nt-PARP1-oligo 3-T
ctaccccaccttcctcattctctacgccgcaccggcca

SEQ ID NO: 381 - DNA, 55 bp, Artificial Sequence
  Synthetic 450-nt-PCNA-oligo 3-T
ctaccccaccttcctcattctctactgaagcgtgggtttcatttctcatcatgtg

SEQ ID NO: 382 - DNA, 45 bp, Artificial Sequence
  Synthetic 450-nt-PELO-oligo 3-T
ctaccccaccttcctcattctctactggcagcttcccaggctctg

SEQ ID NO: 383 - DNA, 45 bp, Artificial Sequence
  Synthetic 450-nt-PMS1-oligo 3-T
ctaccccaccttcctcattctctaggggtcgtcctgagagggaaa

SEQ ID NO: 384 - DNA, 42 bp, Artificial Sequence
  Synthetic 450-nt-PMS2-oligo 3-T
ctaccccaccttcctcattctctagccccgctcacctcgagc

SEQ ID NO: 385 - DNA, 51 bp, Artificial Sequence
  Synthetic 450-nt-PMS2P11-oligo 3-T
ctaccccaccttcctcattctctacctttggcaacaatggtgtcttctttc

SEQ ID NO: 386 - DNA, 45 bp, Artificial Sequence
  Synthetic 450-nt-PMS2P1-oligo 3-T
ctaccccaccttcctcattctctagcgttggtgtctggcggtttc

SEQ ID NO: 387 - DNA, 42 bp, Artificial Sequence
  Synthetic 450-nt-PMS2P2-oligo 3-T
ctaccccaccttcctcattctctacgcgcctcggccatgttc

SEQ ID NO: 388 - DNA, 43 bp, Artificial Sequence
  Synthetic 450-nt-PMS2P3-oligo 3-T
ctaccccaccttcctcattctctacagctccgcgtccgcctag

SEQ ID NO: 389 - DNA, 48 bp, Artificial Sequence
  Synthetic 450-nt-PMS2P5-oligo 3-T
ctaccccaccttcctcattctctatttccagggaggttggaagccgtg

SEQ ID NO: 390 - DNA, 51 bp, Artificial Sequence
  Synthetic 450-nt-POLD1-oligo 3-T
ctaccccaccttcctcattctctaccgtctctcagatctatgtgcctggct

SEQ ID NO: 391 - DNA, 41 bp, Artificial Sequence
  Synthetic 450-nt-POLD4-oligo 3-T
ctaccccaccttcctcattctctagagaccggccagtgggc

SEQ ID NO: 392 - DNA, 40 bp, Artificial Sequence
  Synthetic 450-nt-POLG-oligo 3-T
ctaccccaccttcctcattctctacggcgtcccccttcgc

SEQ ID NO: 393 - DNA, 45 bp, Artificial Sequence
  Synthetic 450-nt-POLK-oligo 3-T
ctaccccaccttcctcattctctatcgcgactacaactcccagga

SEQ ID NO: 394 - DNA, 41 bp, Artificial Sequence
  Synthetic 450-nt-POLQ-oligo 3-T
ctaccccaccttcctcattctctacgccgccgtttcccact

SEQ ID NO: 395 - DNA, 45 bp, Artificial Sequence
  Synthetic 450-nt-POLR2B-oligo 3-T
ctaccccaccttcctcattctctaggtcaagcttgggccggggtt

SEQ ID NO: 396 - DNA, 39 bp, Artificial Sequence
  Synthetic 450-nt-POLR2G-oligo 3-T
ctaccccaccttcctcattctctagcccttgccgccacc

SEQ ID NO: 397 - DNA, 56 bp, Artificial Sequence
  Synthetic 450-nt-PRDM2-oligo 3-T
ctaccccaccttcctcattctctacaattaagaatctaaatttatcccaagagcca

SEQ ID NO: 398 - DNA, 42 bp, Artificial Sequence
  Synthetic 450-nt-PRKCG-oligo 3-T
ctaccccaccttcctcattctctagagccaaaggcaggggcg

SEQ ID NO: 399 - DNA, 48 bp, Artificial Sequence
  Synthetic 450-nt-PRUNE2-oligo 3-T
ctaccccaccttcctcattctctacctttccattagcatacgcgcgcg

SEQ ID NO: 400 - DNA, 41 bp, Artificial Sequence
  Synthetic 450-nt-PXMP4-oligo 3-T
ctaccccaccttcctcattctctagagcccttagctgcggc

SEQ ID NO: 401 - DNA, 45 bp, Artificial Sequence
  Synthetic 450-nt-PYCARD-oligo 3-T
ctaccccaccttcctcattctctagccgccgaccaggaggaagtc

SEQ ID NO: 402 - DNA, 43 bp, Artificial Sequence
  Synthetic 450-nt-RNASEH2A-oligo 3-T
ctaccccaccttcctcattctctaggacgcaaggctccttgcg

SEQ ID NO: 403 - DNA, 52 bp, Artificial Sequence
  Synthetic 450-nt-RNF111-oligo 3-T
ctaccccaccttcctcattctctagccggcgtgggttcagaatgcctaattt

SEQ ID NO: 404 - DNA, 46 bp, Artificial Sequence
  Synthetic 450-nt-RPA2-oligo 3-T
ctaccccaccttcctcattctctacgcttgttcctgtctcctctgc

SEQ ID NO: 405 - DNA, 62 bp, Artificial Sequence
  Synthetic 450-nt-RPA3-oligo 3-T
ctaccccaccttcctcattctctattttgaaggaaaaaactatgtatccaacaaattcaaac

SEQ ID NO: 406 - DNA, 52 bp, Artificial Sequence
  Synthetic 450-nt-RPA4-oligo 3-T
ctaccccaccttcctcattctctataatccttccctgtgtgtcagtggttgc

SEQ ID NO: 407 - DNA, 42 bp, Artificial Sequence
  Synthetic 450-nt-SH3TC1-oligo 3-T
ctaccccaccttcctcattctctagaggcagctcccaggccc

SEQ ID NO: 408 - DNA, 42 bp, Artificial Sequence
  Synthetic 450-nt-SIM2-oligo 3-T
ctaccccaccttcctcattctctagcgagtcgcaggcggctg

SEQ ID NO: 409 - DNA, 44 bp, Artificial Sequence
  Synthetic 450-nt-SMAD4-oligo 3-T
ctaccccaccttcctcattctctagcctaggggagagcaggaag

SEQ ID NO: 410 - DNA, 59 bp, Artificial Sequence
  Synthetic 450-nt-SMUG1-oligo 3-T
ctaccccaccttcctcattctctaccccaaaatatagaaattctaaccaccctctcggc

SEQ ID NO: 411 - DNA, 40 bp, Artificial Sequence
  Synthetic 450-nt-SNAI1-oligo 3-T
ctaccccaccttcctcattctctaggtgccccccacgcag

SEQ ID NO: 412 - DNA, 47 bp, Artificial Sequence
  Synthetic 450-nt-SNAI2-oligo 3-T
ctaccccaccttcctcattctctaggacacggcggtccctacagcat

SEQ ID NO: 413 - DNA, 49 bp, Artificial Sequence
  Synthetic 450-nt-TAF1-oligo 3-T
ctaccccaccttcctcattctctatcttcgtcgctgtccgtgtctgaca

SEQ ID NO: 414 - DNA, 44 bp, Artificial Sequence
  Synthetic 450-nt-TDG-oligo 3-T
ctaccccaccttcctcattctctagagacctgggcccgatggct

SEQ ID NO: 415 - DNA, 43 bp, Artificial Sequence
  Synthetic 450-nt-TP53-oligo 3-T
ctaccccaccttcctcattctctagcacgctcccagcccgaac

SEQ ID NO: 416 - DNA, 44 bp, Artificial Sequence
  Synthetic 450-nt-TREX1-oligo 3-T
ctaccccaccttcctcattctctagtctggggtgggggaaccca

SEQ ID NO: 417 - DNA, 46 bp, Artificial Sequence
  Synthetic 450-nt-TRIM28-oligo 3-T
ctaccccaccttcctcattctctacgcgccacaagcacaagcacaa

SEQ ID NO: 418 - DNA, 42 bp, Artificial Sequence
  Synthetic 450-nt-TWIST1-oligo 3-T
ctaccccaccttcctcattctctacggtgtggatggccccga

SEQ ID NO: 419 - DNA, 49 bp, Artificial Sequence
  Synthetic 450-nt-TWIST2-oligo 3-T
ctaccccaccttcctcattctctaggctctgatttcaaggccggcttgt

SEQ ID NO: 420 - DNA, 51 bp, Artificial Sequence
  Synthetic 450-nt-UBB-oligo 3-T
ctaccccaccttcctcattctctattcctcatagccgtaagaaaggctcct

SEQ ID NO: 421 - DNA, 44 bp, Artificial Sequence
  Synthetic 450-nt-USP47-oligo 3-T
ctaccccaccttcctcattctctagataccccaggcctcgtcct

SEQ ID NO: 422 - DNA, 41 bp, Artificial Sequence
  Synthetic 450-nt-WDR48-oligo 3-T
ctaccccaccttcctcattctctagctggagtgggcccgga

SEQ ID NO: 423 - DNA, 46 bp, Artificial Sequence
  Synthetic 450-nt-WNT1-oligo 3-T
ctaccccaccttcctcattctctactttggttgttgcccgcgacgg

SEQ ID NO: 424 - DNA, 46 bp, Artificial Sequence
  Synthetic 450-nt-XPC-oligo 3-T
ctaccccaccttcctcattctctatttctggctgcgcagttcgcgt

SEQ ID NO: 425 - DNA, 43 bp, Artificial Sequence
  Synthetic 450-nt-XRCC2-oligo 3-T
ctaccccaccttcctcattctctacctcggtcccagactcagc

SEQ ID NO: 426 - DNA, 43 bp, Artificial Sequence
  Synthetic 450-nt-XRCC3-oligo 3-T
ctaccccaccttcctcattctctaccctgcctcacccctcacg

SEQ ID NO: 427 - DNA, 44 bp, Artificial Sequence
  Synthetic 450-nt-ZEB1-oligo 3-T
ctaccccaccttcctcattctctacacctggggccatccgccat

SEQ ID NO: 428 - DNA, 48 bp, Artificial Sequence
  Synthetic 450-nt-ZNF830-oligo 3-T
ctaccccaccttcctcattctctatccgtttccgactggtgctcagac

SEQ ID NO: 429 - DNA, 52 bp, Artificial Sequence
  Synthetic 940-nt-ALKBH2-oligo 1-T
ctcccaaattgttgggattacaggcgtgaatcaccaactacccacacacacc

SEQ ID NO: 430 - DNA, 53 bp, Artificial Sequence
  Synthetic 940-nt-APEX1-oligo 1-T
actgagcgcattttatgtgctaagtattcaatcaccaactacccacacacacc

SEQ ID NO: 431 - DNA, 60 bp, Artificial Sequence
  Synthetic 940-nt-APEX2-oligo 1-T
gagacgtcgaatgaattcatttcattctcacagttcaatcaccaactacccacacacacc

SEQ ID NO: 432 - DNA, 52 bp, Artificial Sequence
  Synthetic 940-nt-ATM-oligo 1-T
gagagaccttcctggcgcccttctaataaatcaccaactacccacacacacc

SEQ ID NO: 433 - DNA, 53 bp, Artificial Sequence
  Synthetic 940-nt-ATRX-oligo1
cagaggaagcttggctggatgatctgtaaaatcaccaactacccacacacacc

SEQ ID NO: 434 - DNA, 45 bp, Artificial Sequence
  Synthetic 940-nt-BRCA1-oligo 1-T
tccgtcgacgcaatcgccaccaatcaccaactacccacacacacc

SEQ ID NO: 435 - DNA, 45 bp, Artificial Sequence
  Synthetic 940-nt-BRCA2-oligo 1-T
accacgcacggctaactttgcaatcaccaactacccacacacacc

SEQ ID NO: 436 - DNA, 54 bp, Artificial Sequence
  Synthetic 940-nt-CCN4-oligo 1-T
gcattgcgcccaaccctacttctggttttcaatcaccaactacccacacacacc

SEQ ID NO: 437 - DNA, 63 bp, Artificial Sequence
  Synthetic 940-nt-CD44-oligo 1-T
cccatccacccacatatctgtttattgatatatccactcaatcaccaactacccacacacacc

SEQ ID NO: 438 - DNA, 57 bp, Artificial Sequence
  Synthetic 940-nt-CETN1-oligo 1-T
ccatgtgttcaacttgacataaatgggaaacagaatcaccaactacccacacacacc

SEQ ID NO: 439 - DNA, 44 bp, Artificial Sequence
  Synthetic 940-nt-CHEK1-oligo 1-T
agaggctgcagtgagcagagaatcaccaactacccacacacacc

SEQ ID NO: 440 - DNA, 48 bp, Artificial Sequence
  Synthetic 940-nt-CHEK2-oligo 1-T
gaggctagaggggagggagatttgaatcaccaactacccacacacacc

SEQ ID NO: 441 - DNA, 56 bp, Artificial Sequence
  Synthetic 940-nt-CSDE1-oligo 1-T
cccagattcaatgcttctgttttcacagattcaatcaccaactacccacacacacc

SEQ ID NO: 442 - DNA, 47 bp, Artificial Sequence
  Synthetic 940-nt-DDB2-oligo 1-T
gcagtgtctctcttccgttacccaatcaccaactacccacacacacc

SEQ ID NO: 443 - DNA, 51 bp, Artificial Sequence
  Synthetic 940-nt-EPM2AIP1-MLH1-oligo 1-T
acgaatactacgagcggtatgtggcggaatcaccaactacccacacacacc

SEQ ID NO: 444 - DNA, 56 bp, Artificial Sequence
  Synthetic 940-nt-EPSTI1-oligo 1-T
gctacctgcattctaggagtatcctagtagacaatcaccaactacccacacacacc

SEQ ID NO: 445 - DNA, 59 bp, Artificial Sequence
  Synthetic 940-nt-EXO1-oligo
gataatgcagactcaattcaaggaattgaaggggaaatcaccaactacccacacacacc

SEQ ID NO: 446 - DNA, 56 bp, Artificial Sequence
  Synthetic 940-nt-FEN1-oligo 1-T
ggctgaccggtgacaatgaagttaacatttgtaatcaccaactacccacacacacc

SEQ ID NO: 447 - DNA, 51 bp, Artificial Sequence
  Synthetic 940-nt-GSTA1-oligo 1-T
tggaacacagaggcagaaattagagtgaatcaccaactacccacacacacc

SEQ ID NO: 448 - DNA, 58 bp, Artificial Sequence
  Synthetic 940-nt-HIST1H1B-oligo 1-T
gagtgttttctctttttagttcctgggcaaatccaatcaccaactacccacacacacc

SEQ ID NO: 449 - DNA, 42 bp, Artificial Sequence
  Synthetic 940-nt-HOTAIR-oligo 1-T
tggggtgtcccgggtgcaaatcaccaactacccacacacacc

SEQ ID NO: 450 - DNA, 53 bp, Artificial Sequence
  Synthetic 940-nt-HOXB2-oligo 1-T
ccttatgtacccctgctttcctcactttcaatcaccaactacccacacacacc

SEQ ID NO: 451 - DNA, 46 bp, Artificial Sequence
  Synthetic 940-nt-HSPA5-oligo 1-T
tttactttcccctccgctgcccaatcaccaactacccacacacacc

SEQ ID NO: 452 - DNA, 56 bp, Artificial Sequence
  Synthetic 940-nt-LIG1-oligo 1-T
ggaaggaggagttccagatgaatacgagagttaatcaccaactacccacacacacc

SEQ ID NO: 453 - DNA, 56 bp, Artificial Sequence
  Synthetic 940-nt-MBD4-oligo 1-T
gagatctcgtttctctaaaactggagatgggcaatcaccaactacccacacacacc

SEQ ID NO: 454 - DNA, 57 bp, Artificial Sequence
  Synthetic 940-nt-MCM9-oligo 1-T
ggttctagcttttgtttatatgaacacagtgtcaatcaccaactacccacacacacc

SEQ ID NO: 455 - DNA, 53 bp, Artificial Sequence
  Synthetic 940-nt-MECP2-oligo 1-T
ctgttagctaaccaggtgttcaggacaggaatcaccaactacccacacacacc

SEQ ID NO: 456 - DNA, 56 bp, Artificial Sequence
  Synthetic 940-nt-MRE11-oligo 1-T
gtttcttaactgtaagtcgggtagatgcgcctaatcaccaactacccacacacacc

SEQ ID NO: 457 - DNA, 49 bp, Artificial Sequence
  Synthetic 940-nt-MSH2-oligo 1-T
agggctgaggcgaaaggatcacctgaatcaccaactacccacacacacc

SEQ ID NO: 458 - DNA, 49 bp, Artificial Sequence
  Synthetic 940-nt-MSH4-oligo 1-T
cggtttacccacatgcagtgacaacaatcaccaactacccacacacacc

SEQ ID NO: 459 - DNA, 61 bp, Artificial Sequence
  Synthetic 940-nt-MSH5-oligo 1-T
gcggtgaacataatgaagtcactgctccataaagcttaatcaccaactacccacacacacc

SEQ ID NO: 460 - DNA, 53 bp, Artificial Sequence
  Synthetic 940-nt-OGG1-oligo 1-T
ctgggccattctcaccttttagcatagggaatcaccaactacccacacacacc

SEQ ID NO: 461 - DNA, 53 bp, Artificial Sequence
  Synthetic 940-nt-OLIG2-oligo 1-T
ctctcctttaggttgaggatggtgtgggtaatcaccaactacccacacacacc

SEQ ID NO: 462 - DNA, 62 bp, Artificial Sequence
  Synthetic 940-nt-PELO-oligo 1-T
gcggttttgagaatttcaaagacagaaagaataagtggaatcaccaactacccacacacacc

SEQ ID NO: 463 - DNA, 46 bp, Artificial Sequence
  Synthetic 940-nt-PMS2-oligo 1-T
ccattgcactcgagcctgggcaaatcaccaactacccacacacacc

SEQ ID NO: 464 - DNA, 50 bp, Artificial Sequence
  Synthetic 940-nt-PMS2P5-oligo 1-T
ggccaacatggtgaaaccccgtgtctaatcaccaactacccacacacacc

SEQ ID NO: 465 - DNA, 59 bp, Artificial Sequence
  Synthetic 940-nt-POLD4-oligo 1-T
ccatgtgccaagtactgttctaagcactttcccacaatcaccaactacccacacacacc

SEQ ID NO: 466 - DNA, 53 bp, Artificial Sequence
  Synthetic 940-nt-PRUNE2-oligo 1-T
taggtgcccttatgtcagccctgggactaaatcaccaactacccacacacacc

SEQ ID NO: 467 - DNA, 47 bp, Artificial Sequence
  Synthetic 940-nt-RPA2-oligo 1-T
tgtccaggctgcagtgagctgtgaatcaccaactacccacacacacc

SEQ ID NO: 468 - DNA, 57 bp, Artificial Sequence
  Synthetic 940-nt-RPA4-oligo 1-T
caaggctctgtggacaggacagaaaagtacaagaatcaccaactacccacacacacc

SEQ ID NO: 469 - DNA, 66 bp, Artificial Sequence
  Synthetic 940-nt-TAF1-oligo 1-T
cggaaaatgtgaaagtttagtaaaatgaacagctgacccctcaatcaccaactacccacacacacc

SEQ ID NO: 470 - DNA, 57 bp, Artificial Sequence
  Synthetic 940-nt-TP53-oligo 1-T
gcttgatgggaacgggaaaccttctaacctttcaatcaccaactacccacacacacc

SEQ ID NO: 471 - DNA, 49 bp, Artificial Sequence
  Synthetic 940-nt-UBB-oligo 1-T
cttgaacctaggaggcagagattgcaatcaccaactacccacacacacc

SEQ ID NO: 472 - DNA, 62 bp, Artificial Sequence
  Synthetic 940-nt-XRCC2-oligo 1-T
cccacctagtttagttttcaaaaacgctaggaaagagcaatcaccaactacccacacacacc

SEQ ID NO: 473 - DNA, 59 bp, Artificial Sequence
  Synthetic 940-nt-ZNF830-oligo 1-T
gcacccttttctatagacaagagtgaggaaaagagaatcaccaactacccacacacacc

SEQ ID NO: 474 - DNA, 56 bp, Artificial Sequence; identical to SEQ ID NO: 192
  Synthetic 940-nt-ALKBH2-oligo 2-T

SEQ ID NO: 475 - DNA, 52 bp, Artificial Sequence; identical to SEQ ID NO: 194
  Synthetic 940-nt-APEX1-oligo 2-T

SEQ ID NO: 476 - DNA, 46 bp, Artificial Sequence; identical to SEQ ID NO: 195
  Synthetic 940-nt-APEX2-oligo 2-T

SEQ ID NO: 477 - DNA, 42 bp, Artificial Sequence; identical to SEQ ID NO: 196
  Synthetic 940-nt-ATM-oligo 2-T

SEQ ID NO: 478 - DNA, 46 bp, Artificial Sequence; identical to SEQ ID NO: 197
  Synthetic 940-nt-ATRX-oligo 2-T

SEQ ID NO: 479 - DNA, 48 bp, Artificial Sequence; identical to SEQ ID NO: 200
  Synthetic 940-nt-BRCA1-oligo 2-T

SEQ ID NO: 480 - DNA, 50 bp, Artificial Sequence; identical to SEQ ID NO: 201
  Synthetic 940-nt-BRCA2-oligo 2-T

SEQ ID NO: 481 - DNA, 44 bp, Artificial Sequence; identical to SEQ ID NO: 202
  Synthetic 940-nt-CCN4-oligo 2-T

SEQ ID NO: 482 - DNA, 45 bp, Artificial Sequence; identical to SEQ ID NO: 203
  Synthetic 940-nt-CD44-oligo 2-T

SEQ ID NO: 483 - DNA, 51 bp, Artificial Sequence; identical to SEQ ID NO: 209
  Synthetic 940-nt-CETN1-oligo 2-T

SEQ ID NO: 484 - DNA, 53 bp, Artificial Sequence; identical to SEQ ID NO: 210
  Synthetic 940-nt-CHEK1-oligo 2-T

SEQ ID NO: 485 - DNA, 53 bp, Artificial Sequence; identical to SEQ ID NO: 211
  Synthetic 940-nt-CHEK2-oligo 2-T

SEQ ID NO: 486 - DNA, 53 bp, Artificial Sequence; identical to SEQ ID NO: 212
  Synthetic 940-nt-CSDE1-oligo 2-T

SEQ ID NO: 487 - DNA, 49 bp, Artificial Sequence; identical to SEQ ID NO: 213
  Synthetic 940-nt-DDB2-oligo 2-T

SEQ ID NO: 488 - DNA, 48 bp, Artificial Sequence; identical to SEQ ID NO: 50
  Synthetic 940-nt-EPM2AIP1-MLH1-oligo 2-T

SEQ ID NO: 489 - DNA, 46 bp, Artificial Sequence; identical to SEQ ID NO: 218
  Synthetic 940-nt-EPSTI1-oligo 2-T

SEQ ID NO: 490 - DNA, 44 bp, Artificial Sequence; identical to SEQ ID NO: 222
  Synthetic 940-nt-EXO1-oligo 2-T

SEQ ID NO: 491 - DNA, 46 bp, Artificial Sequence; identical to SEQ ID NO: 225
  Synthetic 940-nt-FEN1-oligo 2-T

SEQ ID NO: 492 - DNA, 44 bp, Artificial Sequence; identical to SEQ ID NO: 231
  Synthetic 940-nt-GSTA1-oligo 2-T

SEQ ID NO: 493 - DNA, 48 bp, Artificial Sequence; identical to SEQ ID NO: 232
  Synthetic 940-nt-HIST1H1B-oligo 2-T

SEQ ID NO: 494 - DNA, 44 bp, Artificial Sequence; identical to SEQ ID NO: 234
  Synthetic 940-nt-HOTAIR-oligo 2-T

SEQ ID NO: 495 - DNA, 47 bp, Artificial Sequence; identical to SEQ ID NO: 235
  Synthetic 940-nt-HOXB2-oligo 2-T

SEQ ID NO: 496 - DNA, 44 bp, Artificial Sequence; identical to SEQ ID NO: 236
  Synthetic 940-nt-HSPA5-oligo 2-T

SEQ ID NO: 497 - DNA, 47 bp, Artificial Sequence; identical to SEQ ID NO: 239
  Synthetic 940-nt-LIG1-oligo 2-T

SEQ ID NO: 498 - DNA, 49 bp, Artificial Sequence; identical to SEQ ID NO: 240
  Synthetic 940-nt-MBD4-oligo 2-T

SEQ ID NO: 499 - DNA, 52 bp, Artificial Sequence; identical to SEQ ID NO: 241
  Synthetic 940-nt-MCM9-oligo 2-T

SEQ ID NO: 500 - DNA, 42 bp, Artificial Sequence; identical to SEQ ID NO: 242
  Synthetic 940-nt-MECP2-oligo 2-T

SEQ ID NO: 501 - DNA, 46 bp, Artificial Sequence; identical to SEQ ID NO: 249
  Synthetic 940-nt-MRE11-oligo 2-T

SEQ ID NO: 502 - DNA, 48 bp, Artificial Sequence; identical to SEQ ID NO: 250
  Synthetic 940-nt-MSH2-oligo 2-T

SEQ ID NO: 503 - DNA, 40 bp, Artificial Sequence; identical to SEQ ID NO: 252
  Synthetic 940-nt-MSH4-oligo 2-T

SEQ ID NO: 504 - DNA, 41 bp, Artificial Sequence; identical to SEQ ID NO: 253
  Synthetic 940-nt-MSH5-oligo 2-T

SEQ ID NO: 505 - DNA, 44 bp, Artificial Sequence; identical to SEQ ID NO: 259
  Synthetic 940-nt-OGG1-oligo 2-T

SEQ ID NO: 506 - DNA, 48 bp, Artificial Sequence; identical to SEQ ID NO: 260
  Synthetic 940-nt-OLIG2-oligo 2-T

SEQ ID NO: 507 - DNA, 48 bp, Artificial Sequence; identical to SEQ ID NO: 263
  Synthetic 940-nt-PELO-oligo 2-T

SEQ ID NO: 508 - DNA, 44 bp, Artificial Sequence; identical to SEQ ID NO: 265
  Synthetic 940-nt-PMS2-oligo 2-T

SEQ ID NO: 509 - DNA, 43 bp, Artificial Sequence; identical to SEQ ID NO: 270
  Synthetic 940-nt-PMS2P5-oligo 2-T

SEQ ID NO: 510 - DNA, 45 bp, Artificial Sequence; identical to SEQ ID NO: 272
  Synthetic 940-nt-POLD4-oligo 2-T

SEQ ID NO: 511 - DNA, 54 bp, Artificial Sequence; identical to SEQ ID NO: 280
  Synthetic 940-nt-PRUNE2-oligo 2-T

SEQ ID NO: 512 - DNA, 47 bp, Artificial Sequence; identical to SEQ ID NO: 285
  Synthetic 940-nt-RPA2-oligo 2-T

SEQ ID NO: 513 - DNA, 46 bp, Artificial Sequence; identical to SEQ ID NO: 287
  Synthetic 940-nt-RPA4-oligo 2-T

SEQ ID NO: 514 - DNA, 44 bp, Artificial Sequence; identical to SEQ ID NO: 294
  Synthetic 940-nt-TAF1-oligo 2-T

SEQ ID NO: 515 - DNA, 47 bp, Artificial Sequence; identical to SEQ ID NO: 296
  Synthetic 940-nt-TP53-oligo 2-T

SEQ ID NO: 516 - DNA, 51 bp, Artificial Sequence; identical to SEQ ID NO: 301
  Synthetic 940-nt-UBB-oligo 2-T

SEQ ID NO: 517 - DNA, 43 bp, Artificial Sequence; identical to SEQ ID NO: 306
  Synthetic 940-nt-XRCC2-oligo 2-T

SEQ ID NO: 518 - DNA, 51 bp, Artificial Sequence; identical to SEQ ID NO: 309
  Synthetic 940-nt-ZNF830-oligo 2-T

SEQ ID NO: 519 - DNA, 49 bp, Artificial Sequence; identical to SEQ ID NO: 311
  Synthetic 940-nt-ALKBH2-oligo 3-T

SEQ ID NO: 520 - DNA, 41 bp, Artificial Sequence; identical to SEQ ID NO: 313
  Synthetic 940-nt-APEX1-oligo 3-T

SEQ ID NO: 521 - DNA, 47 bp, Artificial Sequence; identical to SEQ ID NO: 314
  Synthetic 940-nt-APEX2-oligo 3-T

SEQ ID NO: 522 - DNA, 47 bp, Artificial Sequence; identical to SEQ ID NO: 315
  Synthetic 940-nt-ATM-oligo 3-T

SEQ ID NO: 523 - DNA, 44 bp, Artificial Sequence; identical to SEQ ID NO: 316
  Synthetic 940-nt-ATRX-oligo 3-T

SEQ ID NO: 524 - DNA, 42 bp, Artificial Sequence; identical to SEQ ID NO: 319
  Synthetic 940-nt-BRCA1-oligo 3-T

SEQ ID NO: 525 - DNA, 44 bp, Artificial Sequence; identical to SEQ ID NO: 320
  Synthetic 940-nt-BRCA2-oligo 3-T

SEQ ID NO: 526 - DNA, 41 bp, Artificial Sequence; identical to SEQ ID NO: 321
  Synthetic 940-nt-CCN4-oligo 3-T

SEQ ID NO: 527 - DNA, 51 bp, Artificial Sequence; identical to SEQ ID NO: 322
  Synthetic 940-nt-CD44-oligo 3-T

SEQ ID NO: 528 - DNA, 45 bp, Artificial Sequence; identical to SEQ ID NO: 328
  Synthetic 940-nt-CETN1-oligo 3-T

SEQ ID NO: 529 - DNA, 56 bp, Artificial Sequence; identical to SEQ ID NO: 329
  Synthetic 940-nt-CHEK1-oligo 3-T

SEQ ID NO: 530 - DNA, 49 bp, Artificial Sequence; identical to SEQ ID NO: 330
  Synthetic 940-nt-CHEK2-oligo 3-T

SEQ ID NO: 531 - DNA, 47 bp, Artificial Sequence; identical to SEQ ID NO: 331
  Synthetic 940-nt-CSDE1-oligo 3-T

SEQ ID NO: 532 - DNA, 47 bp, Artificial Sequence; identical to SEQ ID NO: 332
  Synthetic 940-nt-DDB2-oligo 3-T

SEQ ID NO: 533 - DNA, 47 bp, Artificial Sequence; identical to SEQ ID NO: 61
  Synthetic 940-nt-EPM2AIP1-MLH1-oligo 3-T

SEQ ID NO: 534 - DNA, 48 bp, Artificial Sequence; identical to SEQ ID NO: 337
  Synthetic 940-nt-EPSTI1-oligo 3-T

SEQ ID NO: 535 - DNA, 46 bp, Artificial Sequence; identical to SEQ ID NO: 341
  Synthetic 940-nt-EXO1-oligo 3-T

SEQ ID NO: 536 - DNA, 45 bp, Artificial Sequence; identical to SEQ ID NO: 344
  Synthetic 940-nt-FEN1-oligo 2-T

SEQ ID NO: 537 - DNA, 55 bp, Artificial Sequence; identical to SEQ ID NO: 350
  Synthetic 940-nt-GSTA1-oligo 3-T

SEQ ID NO: 538 - DNA, 44 bp, Artificial Sequence; identical to SEQ ID NO: 351
  Synthetic 940-nt-HIST1H1B-oligo 3-T

SEQ ID NO: 539 - DNA, 46 bp, Artificial Sequence; identical to SEQ ID NO: 353
  Synthetic 940-nt-HOTAIR-oligo 3-T

SEQ ID NO: 540 - DNA, 57 bp, Artificial Sequence; identical to SEQ ID NO: 354
  Synthetic 940-nt-HOXB2-oligo 3-T

SEQ ID NO: 541 - DNA, 44 bp, Artificial Sequence; identical to SEQ ID NO: 355
  Synthetic 940-nt-HSPA5-oligo 3-T

SEQ ID NO: 542 - DNA, 44 bp, Artificial Sequence; identical to SEQ ID NO: 358
  Synthetic 940-nt-LIG1-oligo 3-T

SEQ ID NO: 543 - DNA, 48 bp, Artificial Sequence; identical to SEQ ID NO: 359
  Synthetic 940-nt-MBD4-oligo 3-T

SEQ ID NO: 544 - DNA, 50 bp, Artificial Sequence; identical to SEQ ID NO: 360
  Synthetic 940-nt-MCM9-oligo 3-T

SEQ ID NO: 545 - DNA, 47 bp, Artificial Sequence; identical to SEQ ID NO: 361
  Synthetic 940-nt-MECP2-oligo 3-T

SEQ ID NO: 546 - DNA, 50 bp, Artificial Sequence; identical to SEQ ID NO: 368
  Synthetic 940-nt-MRE11-oligo 3-T

SEQ ID NO: 547 - DNA, 51 bp, Artificial Sequence; identical to SEQ ID NO: 369
  Synthetic 940-nt-MSH2-oligo 3-T

SEQ ID NO: 548 - DNA, 48 bp, Artificial Sequence; identical to SEQ ID NO: 371
  Synthetic 940-nt-MSH4-oligo 3-T

SEQ ID NO: 549 - DNA, 47 bp, Artificial Sequence; identical to SEQ ID NO: 372
  Synthetic 940-nt-MSH5-oligo 3-T

SEQ ID NO: 550 - DNA, 48 bp, Artificial Sequence; identical to SEQ ID NO: 378
  Synthetic 3940-nt-OGG1-oligo 3-T

SEQ ID NO: 551 - DNA, 41 bp, Artificial Sequence; identical to SEQ ID NO: 379
  Synthetic 940-nt-OLIG2-oligo 3-T

SEQ ID NO: 552 - DNA, 45 bp, Artificial Sequence; identical to SEQ ID NO: 382
  Synthetic 940-nt-PELO-oligo 3-T

SEQ ID NO: 553 - DNA, 42 bp, Artificial Sequence; identical to SEQ ID NO: 384
  Synthetic 940-nt-PMS2-oligo 3-T

SEQ ID NO: 554 - DNA, 48 bp, Artificial Sequence; identical to SEQ ID NO: 389
  Synthetic 940-nt-PMS2P5-oligo 3-T

SEQ ID NO: 555 - DNA, 41 bp, Artificial Sequence; identical to SEQ ID NO: 391
  Synthetic 940-nt-POLD4-oligo 3-T

SEQ ID NO: 556 - DNA, 48 bp, Artificial Sequence; identical to SEQ ID NO: 399
  Synthetic 940-nt-PRUNE2-oligo 3-T

SEQ ID NO: 557 - DNA, 46 bp, Artificial Sequence; identical to SEQ ID NO: 404
  Synthetic 940-nt-RPA2-oligo 3-T

SEQ ID NO: 558 - DNA, 52 bp, Artificial Sequence; identical to SEQ ID NO: 406
  Synthetic 940-nt-RPA4-oligo 3-T

SEQ ID NO: 559 - DNA, 49 bp, Artificial Sequence; identical to SEQ ID NO: 413
  Synthetic 940-nt-TAF1-oligo 3-T

SEQ ID NO: 560 - DNA, 43 bp, Artificial Sequence; identical to SEQ ID NO: 415
  Synthetic 940-nt-TP53-oligo 3-T

SEQ ID NO: 561 - DNA, 51 bp, Artificial Sequence; identical to SEQ ID NO: 420
  Synthetic 940-nt-UBB-oligo 3-T

SEQ ID NO: 562 - DNA, 43 bp, Artificial Sequence; identical to SEQ ID NO: 425
  Synthetic 940-nt-XRCC2-oligo 3-T

SEQ ID NO: 563 - DNA, 48 bp, Artificial Sequence; identical to SEQ ID NO: 428
  Synthetic 940-nt-ZNF830-oligo 3-T

SEQ ID NO: 564 - DNA, 52 bp, Artificial Sequence
  Synthetic 620-nt-Ash1l-oligo 1-T
gctggccacggtgaccggaatgttttgaaatcaccaactacccacacacacc

SEQ ID NO: 565 - DNA, 51 bp, Artificial Sequence
  Synthetic 620-nt-Btk-oligo 1-T
aggggagaatcgggatcacacaagtcaaatcaccaactacccacacacacc

SEQ ID NO: 566 - DNA, 52 bp, Artificial Sequence
  Synthetic 620-nt-Ccl5-oligo 1-T
ggtcaagccattggtcttcacttggcaaaatcaccaactacccacacacacc

SEQ ID NO: 567 - DNA, 52 bp, Artificial Sequence
  Synthetic 620-nt-Cd180-oligo 1-T
tggttaggagaagggtagagcagggcagaatcaccaactacccacacacacc

SEQ ID NO: 568 - DNA, 52 bp, Artificial Sequence
  Synthetic 620-nt-Cox8a-oligo 1-T
actgttatcctctcagaaacccgtttccaatcaccaactacccacacacacc

SEQ ID NO: 569 - DNA, 52 bp, Artificial Sequence
  Synthetic 620-nt-Cxcl15-oligo 1-T
cagaagaaatggagtccagactgacacaaatcaccaactacccacacacacc

SEQ ID NO: 570 - DNA, 52 bp, Artificial Sequence
  Synthetic 620-nt-Cxcr4-oligo 1-T
ccgccagggttctatactctgagctgtcaatcaccaactacccacacacacc

SEQ ID NO: 571 - DNA, 52 bp, Artificial Sequence
  Synthetic 620-nt-Dnmt3b-oligo 1-T
cctgctttcacctagcttggcctttgtcaatcaccaactacccacacacacc

SEQ ID NO: 572 - DNA, 52 bp, Artificial Sequence
  Synthetic 620-nt-Foxo1-oligo 1-T
cagcctccactcatcctgaaccccagagaatcaccaactacccacacacacc

SEQ ID NO: 573 - DNA, 51 bp, Artificial Sequence
  Synthetic 620-nt-Gdf5-oligo 1-T
ggggcctccccatcccagagtagactcaatcaccaactacccacacacacc

SEQ ID NO: 574 - DNA, 52 bp, Artificial Sequence
  Synthetic 620-nt-Hdac3-oligo 1-T
gctagactggtggtcaaagcctctcctgaatcaccaactacccacacacacc

SEQ ID NO: 575 - DNA, 51 bp, Artificial Sequence
  Synthetic 620-nt-Hsf1-oligo 1-T
ggaaccatgcaggcacttggcaagaccaatcaccaactacccacacacacc

SEQ ID NO: 576 - DNA, 50 bp, Artificial Sequence
  Synthetic 620-nt-Hsf2bp-oligo 1-T
gagctcctggccctgagtccaagctcaatcaccaactacccacacacacc

SEQ ID NO: 577 - DNA, 49 bp, Artificial Sequence
  Synthetic 620-nt-Hsf4-oligo 1-T
gatcccgatcccccacagtcacagtaatcaccaactacccacacacacc

SEQ ID NO: 578 - DNA, 52 bp, Artificial Sequence
  Synthetic 620-nt-Hsf5-oligo 1-T
agaggaacccactccagtcttccctaacaatcaccaactacccacacacacc

SEQ ID NO: 579 - DNA, 48 bp, Artificial Sequence
  Synthetic 620-nt-Hsfy2-oligo 1-T
atcgcccaaagtcaggaaaacaaaaatcaccaactacccacacacacc

SEQ ID NO: 580 - DNA, 52 bp, Artificial Sequence
  Synthetic 620-nt-Hsp90aa1-oligo 1-T
agctgctggaggccttccaataggaacaaatcaccaactacccacacacacc

SEQ ID NO: 581 - DNA, 52 bp, Artificial Sequence
  Synthetic 620-nt-Hsp90ab1-oligo 1-T
aagagtccagaactcaggggactgaaagaatcaccaactacccacacacacc

SEQ ID NO: 582 - DNA, 52 bp, Artificial Sequence
  Synthetic 620-nt-Hspa1a-oligo 1-T
acttaagggctaaaacttctccccgccaaatcaccaactacccacacacacc

SEQ ID NO: 583 - DNA, 52 bp, Artificial Sequence
  Synthetic 620-nt-Hspa1b-oligo 1-T
tggtgagagtgtgggactcagaccagggaatcaccaactacccacacacacc

SEQ ID NO: 584 - DNA, 52 bp, Artificial Sequence
  Synthetic 620-nt-Hspa2-oligo 1-T
cggttgcatcgaatacagaacggctgggaatcaccaactacccacacacacc

SEQ ID NO: 585 - DNA, 50 bp, Artificial Sequence
  Synthetic 620-nt-Ifna1-oligo 1-T
caaggtatggttgcatgtttctctcaaatcaccaactacccacacacacc

SEQ ID NO: 586 - DNA, 52 bp, Artificial Sequence
  Synthetic 620-nt-Ifna2-oligo 1-T
ctaaggtagaagtattgttgtggcctggaatcaccaactacccacacacacc

SEQ ID NO: 587 - DNA, 52 bp, Artificial Sequence
  Synthetic 620-nt-Ifng-oligo 1-T
gcctttgtttgtggttggggacggctgaaatcaccaactacccacacacacc

SEQ ID NO: 588 - DNA, 52 bp, Artificial Sequence
  Synthetic 620-nt-Ikbkb-oligo 1-T
gaggctgggtattgtcttctccaccaggaatcaccaactacccacacacacc

SEQ ID NO: 589 - DNA, 52 bp, Artificial Sequence
  Synthetic 620-nt-Ikbkg-oligo 1-T
aagtcataacaccgatcgctcccagcacaatcaccaactacccacacacacc

SEQ ID NO: 590 - DNA, 52 bp, Artificial Sequence
  Synthetic 620-nt-Il10-oligo 1-T
aacgtccgatatttcttgctgactcggaaatcaccaactacccacacacacc

SEQ ID NO: 591 - DNA, 52 bp, Artificial Sequence
  Synthetic 620-nt-Il12a-oligo 1-T
accatctccataaccacaagcctttgggaatcaccaactacccacacacacc

SEQ ID NO: 592 - DNA, 52 bp, Artificial Sequence
  Synthetic 620-nt-Il12b-oligo 1-T
ccacctttccccatgttttcttgttagcaatcaccaactacccacacacacc

SEQ ID NO: 593 - DNA, 52 bp, Artificial Sequence
  Synthetic 620-nt-Il3ra-oligo 1-T
tcctgtcatccgttgcaactgtactcgcaatcaccaactacccacacacacc

SEQ ID NO: 594 - DNA, 50 bp, Artificial Sequence
  Synthetic 620-nt-Il6-oligo 1-T
agacacacactccctctcccacccccaatcaccaactacccacacacacc

SEQ ID NO: 595 - DNA, 52 bp, Artificial Sequence
  Synthetic 620-nt-Irak1-oligo 1-T
tgcttgtgtgctagatctccatgcccccaatcaccaactacccacacacacc

SEQ ID NO: 596 - DNA, 43 bp, Artificial Sequence
  Synthetic 620-nt-Irak4-oligo 1-T
cctgtctgcccgcctcttgaatcaccaactacccacacacacc

SEQ ID NO: 597 - DNA, 50 bp, Artificial Sequence
  Synthetic 620-nt-Irf3-oligo 1-T
cgtttgccgcaaagcatactaggattaatcaccaactacccacacacacc

SEQ ID NO: 598 - DNA, 52 bp, Artificial Sequence
  Synthetic 620-nt-Irf5-oligo 1-T
cttgggccactctgtctcctcagccttaaatcaccaactacccacacacacc

SEQ ID NO: 599 - DNA, 52 bp, Artificial Sequence
  Synthetic 620-nt-Irf7-oligo 1-T
caggaaagcacattgggacaaggcagggaatcaccaactacccacacacacc

SEQ ID NO: 600 - DNA, 43 bp, Artificial Sequence
  Synthetic 620-nt-Jun-oligo 1-T
acttccgggggacagagcgaatcaccaactacccacacacacc

SEQ ID NO: 601 - DNA, 52 bp, Artificial Sequence
  Synthetic 620-nt-Kdm6b-oligo 1-T
ctgctacctccaaccctaacaagacaccaatcaccaactacccacacacacc

SEQ ID NO: 602 - DNA, 52 bp, Artificial Sequence
  Synthetic 620-nt-Lbp-oligo 1-T
aaccttgcaatgggatagctgtctgaacaatcaccaactacccacacacacc

SEQ ID NO: 603 - DNA, 52 bp, Artificial Sequence
  Synthetic 620-nt-Ly86-oligo 1-T
aacaggagcttgtcctaggagcccacacaatcaccaactacccacacacacc

SEQ ID NO: 604 - DNA, 52 bp, Artificial Sequence
  Synthetic 620-nt-Map3k1-oligo 1-T
agaggttactcccccgtcttggaaaggcaatcaccaactacccacacacacc

SEQ ID NO: 605 - DNA, 52 bp, Artificial Sequence
  Synthetic 620-nt-Mapk1-oligo 1-T
gtgggacagaaaccaaaaagcataacccaatcaccaactacccacacacacc

SEQ ID NO: 606 - DNA, 52 bp, Artificial Sequence
  Synthetic 620-nt-Mapk13-oligo 1-T
aagggagaggagagactagagaggagagaatcaccaactacccacacacacc

SEQ ID NO: 607 - DNA, 52 bp, Artificial Sequence
  Synthetic 620-nt-Mapk14-oligo 1-T
caggcgctcctacatgttttcaggcagaaatcaccaactacccacacacacc

SEQ ID NO: 608 - DNA, 48 bp, Artificial Sequence
  Synthetic 620-nt-Mapk15-oligo 1-T
gctttgtgctcaggttcaggggacaatcaccaactacccacacacacc

SEQ ID NO: 609 - DNA, 49 bp, Artificial Sequence
  Synthetic 620-nt-Mapk3-oligo 1-T
gcctgagccatcatacagattcaggaatcaccaactacccacacacacc

SEQ ID NO: 610 - DNA, 51 bp, Artificial Sequence
  Synthetic 620-nt-Mapk4-oligo 1-T
aagtaaaactcacagatgggaaccgagaatcaccaactacccacacacacc

SEQ ID NO: 611 - DNA, 52 bp, Artificial Sequence
  Synthetic 620-nt-Mapk6-oligo 1-T
gaatgagtgaggggtcctgggaaaactgaatcaccaactacccacacacacc

SEQ ID NO: 612 - DNA, 52 bp, Artificial Sequence
  Synthetic 620-nt-Mapk8-oligo 1-T
ttcaagggcctaccacccaccaaggagtaatcaccaactacccacacacacc

SEQ ID NO: 613 - DNA, 52 bp, Artificial Sequence
  Synthetic 620-nt-Mme-oligo 1-T
agtgctgattgcaggctctcattctctaaatcaccaactacccacacacacc

SEQ ID NO: 614 - DNA, 52 bp, Artificial Sequence
  Synthetic 620-nt-Myd88-oligo 1-T
cggggtctgcaaaaaacacgcggaacctaatcaccaactacccacacacacc

SEQ ID NO: 615 - DNA, 51 bp, Artificial Sequence
  Synthetic 620-nt-Nfkb1-oligo 1-T
agcctggggctaggtagctcccaagtgaatcaccaactacccacacacacc

SEQ ID NO: 616 - DNA, 52 bp, Artificial Sequence
  Synthetic 620-nt-Nfkb2-oligo 1-T
caaacttcctcttctgtggaaaattgccaatcaccaactacccacacacacc

SEQ ID NO: 617 - DNA, 52 bp, Artificial Sequence
  Synthetic 620-nt-Nfkbia-oligo 1-T
cgcttctcctaaatttctgcgacgacggaatcaccaactacccacacacacc

SEQ ID NO: 618 - DNA, 52 bp, Artificial Sequence
  Synthetic 620-nt-Nfkbib-oligo 1-T
cgcatttctcgtactttggctgctgaggaatcaccaactacccacacacacc

SEQ ID NO: 619 - DNA, 52 bp, Artificial Sequence
  Synthetic 620-nt-Nr2c2-oligo 1-T
gtgcccctctccttcgcgatttgagaaaaatcaccaactacccacacacacc

SEQ ID NO: 620 - DNA, 52 bp, Artificial Sequence
  Synthetic 620-nt-Pik3ap1-oligo 1-T
atagggaaggcctgcaggaaggggaaggaatcaccaactacccacacacacc

SEQ ID NO: 621 - DNA, 52 bp, Artificial Sequence
  Synthetic 620-nt-Pik3c2a-oligo 1-T
tgagcgcggactgcaattgggcttgtaaaatcaccaactacccacacacacc

SEQ ID NO: 622 - DNA, 52 bp, Artificial Sequence
  Synthetic 620-nt-Pik3c2g-oligo 1-T
tgtgggaaatcaaagaggactcttggaaaatcaccaactacccacacacacc

SEQ ID NO: 623 - DNA, 52 bp, Artificial Sequence
  Synthetic 620-nt-Pik3c3-oligo 1-T
cgggttttgaatcttctgagtgcctgtaaatcaccaactacccacacacacc

SEQ ID NO: 624 - DNA, 52 bp, Artificial Sequence
  Synthetic 620-nt-Pik3ca-oligo 1-T
ctttccgctcacatcgtgtaaacaaacaaatcaccaactacccacacacacc

SEQ ID NO: 625 - DNA, 52 bp, Artificial Sequence
  Synthetic 620-nt-Pik3r1-oligo 1-T
ctgcctctttcgaatccactagagcttcaatcaccaactacccacacacacc

SEQ ID NO: 626 - DNA, 51 bp, Artificial Sequence
  Synthetic 620-nt-Pik3r2-oligo 1-T
ccgccgatagcttccggaaattgccgaaatcaccaactacccacacacacc

SEQ ID NO: 627 - DNA, 51 bp, Artificial Sequence
  Synthetic 620-nt-Pik3r3-oligo 1-T
accccctagcctcgctcagaatggtacaatcaccaactacccacacacacc

SEQ ID NO: 628 - DNA, 52 bp, Artificial Sequence
  Synthetic 620-nt-Pik3r5-oligo 1-T
acttccagcactgcttctgcaacctctgaatcaccaactacccacacacacc

SEQ ID NO: 629 - DNA, 52 bp, Artificial Sequence
  Synthetic 620-nt-Pik3r6-oligo 1-T
tgtcccccacatttgcatcttagaccacaatcaccaactacccacacacacc

SEQ ID NO: 630 - DNA, 52 bp, Artificial Sequence
  Synthetic 620-nt-Ptpn6-oligo 1-T
aaagggtctaaaggaggggaaaggctagaatcaccaactacccacacacacc

SEQ ID NO: 631 - DNA, 52 bp, Artificial Sequence
  Synthetic 620-nt-Rela-oligo 1-T
gaggactaggcagacacctgtacctctaaatcaccaactacccacacacacc

SEQ ID NO: 632 - DNA, 51 bp, Artificial Sequence
  Synthetic 620-nt-Src-oligo 1-T
acgtcatctcatcgtgtacctcttgccaatcaccaactacccacacacacc

SEQ ID NO: 633 - DNA, 52 bp, Artificial Sequence
  Synthetic 620-nt-Stat3-oligo 1-T
tttgtgagctccttaccacgtgggaatcaatcaccaactacccacacacacc

SEQ ID NO: 634 - DNA, 52 bp, Artificial Sequence
  Synthetic 620-nt-Tab1-oligo 1-T
accccaatgccaaactagctgaagttagaatcaccaactacccacacacacc

SEQ ID NO: 635 - DNA, 43 bp, Artificial Sequence
  Synthetic 620-nt-Tab2-oligo 1-T
agtttggctcggccgctggaatcaccaactacccacacacacc

SEQ ID NO: 636 - DNA, 49 bp, Artificial Sequence
  Synthetic 620-nt-Tet2-oligo 1-T
gccaagccgctccccatttccccaaaatcaccaactacccacacacacc

SEQ ID NO: 637 - DNA, 52 bp, Artificial Sequence
  Synthetic 620-nt-Tlr2-oligo 1-T
cctcagaggttgcaacttgggctcaggaaatcaccaactacccacacacacc

SEQ ID NO: 638 - DNA, 52 bp, Artificial Sequence
  Synthetic 620-nt-Tlr4-oligo 1-T
tgttcattcgttcacgggggggatgtataatcaccaactacccacacacacc

SEQ ID NO: 639 - DNA, 52 bp, Artificial Sequence
  Synthetic 620-nt-Tnf-oligo 1-T
actgaaacccatttcttctctgtcctccaatcaccaactacccacacacacc

SEQ ID NO: 640 - DNA, 49 bp, Artificial Sequence
  Synthetic 620-nt-Traf6-oligo 1-T
agatgcccaaaagattttcaccctcaatcaccaactacccacacacacc

SEQ ID NO: 641 - DNA, 52 bp, Artificial Sequence
  Synthetic 620-nt-Vav1-oligo 1-T
gttcaaccttgctttggggccaaaactcaatcaccaactacccacacacacc

SEQ ID NO: 642 - DNA, 43 bp, Artificial Sequence
  Synthetic 620-nt-Ash1l-oligo 2-T
agagcgccgagtgcaggcaaatcaccaactacccacacacacc

SEQ ID NO: 643 - DNA, 52 bp, Artificial Sequence
  Synthetic 620-nt-Btk-oligo 2-T
agttctgacttaatgcaggtagctccccaatcaccaactacccacacacacc

SEQ ID NO: 644 - DNA, 48 bp, Artificial Sequence
  Synthetic 620-nt-Ccl5-oligo 2-T
gcatcgaggcagagtggggctgtgaatcaccaactacccacacacacc

SEQ ID NO: 645 - DNA, 52 bp, Artificial Sequence
  Synthetic 620-nt-Cd180-oligo 2-T
gaagagggccactaaaaagaagcagctgaatcaccaactacccacacacacc

SEQ ID NO: 646 - DNA, 50 bp, Artificial Sequence
  Synthetic 620-nt-Cox8a-oligo 2-T
tcagcagcagtggcgtcaggacagacaatcaccaactacccacacacacc

SEQ ID NO: 647 - DNA, 50 bp, Artificial Sequence
  Synthetic 620-nt-Cxcl15-oligo 2-T
agaaaaattccccgttagcttaccgaaatcaccaactacccacacacacc

SEQ ID NO: 648 - DNA, 52 bp, Artificial Sequence
  Synthetic 620-nt-Cxcr4-oligo 2-T
ggatgcaagtggacttacactcacactgaatcaccaactacccacacacacc

SEQ ID NO: 649 - DNA, 52 bp, Artificial Sequence
  Synthetic 620-nt-Dnmt3b-oligo 2-T
aaaagttgctgcaggtctccttccctcaaatcaccaactacccacacacacc

SEQ ID NO: 650 - DNA, 44 bp, Artificial Sequence
  Synthetic 620-nt-Foxo1-oligo 2-T
cgctgggtcaccggtgtctaaatcaccaactacccacacacacc

SEQ ID NO: 651 - DNA, 52 bp, Artificial Sequence
  Synthetic 620-nt-Gdf5-oligo 2-T
gtgcaccgtctccagtcagcagctgaaaaatcaccaactacccacacacacc

SEQ ID NO: 652 - DNA, 52 bp, Artificial Sequence
  Synthetic 620-nt-Hdac3-oligo 2-T
gcccacatcggggtcgtagaaatacgccaatcaccaactacccacacacacc

SEQ ID NO: 653 - DNA, 52 bp, Artificial Sequence
  Synthetic 620-nt-Hsf1-oligo 2-T
ggccttttagaagagtggctgaaggctgaatcaccaactacccacacacacc

SEQ ID NO: 654 - DNA, 52 bp, Artificial Sequence
  Synthetic 620-nt-Hsf2bp-oligo 2-T
atcaaaccagccgtaggcgttctgctggaatcaccaactacccacacacacc

SEQ ID NO: 655 - DNA, 48 bp, Artificial Sequence
  Synthetic 620-nt-Hsf4-oligo 2-T
gctgggtggacaggggtgttcacgaatcaccaactacccacacacacc

SEQ ID NO: 656 - DNA, 45 bp, Artificial Sequence
  Synthetic 620-nt-Hsf5-oligo 2-T
ctgagcagctcggcctcgaagaatcaccaactacccacacacacc

SEQ ID NO: 657 - DNA, 52 bp, Artificial Sequence
  Synthetic 620-nt-Hsfy2-oligo 2-T
actgtcttcatctggctctgaggcacagaatcaccaactacccacacacacc

SEQ ID NO: 658 - DNA, 52 bp, Artificial Sequence
  Synthetic 620-nt-Hsp90aa1-oligo 2-T
atctgaggtgaactcactcttacccgctaatcaccaactacccacacacacc

SEQ ID NO: 659 - DNA, 52 bp, Artificial Sequence
  Synthetic 620-nt-Hsp90ab1-oligo 2-T
gtacagcccgaacctctaacctctagacaatcaccaactacccacacacacc

SEQ ID NO: 660 - DNA, 48 bp, Artificial Sequence
  Synthetic 620-nt-Hspa1a-oligo 2-T
gctggaacacgcccacgcacgagtaatcaccaactacccacacacacc

SEQ ID NO: 661 - DNA, 48 bp, Artificial Sequence; identical to SEQ ID NO: 660
  Synthetic 620-nt-Hspa1b-oligo 2-T

SEQ ID NO: 662 - DNA, 43 bp, Artificial Sequence
  Synthetic 620-nt-Hspa2-oligo 2-T
aagcgcaagccgggacctgaatcaccaactacccacacacacc

SEQ ID NO: 663 - DNA, 52 bp, Artificial Sequence
  Synthetic 620-nt-Ifna1-oligo 2-T
tcagctcactcaggacagggatggcttgaatcaccaactacccacacacacc

SEQ ID NO: 664 - DNA, 52 bp, Artificial Sequence
  Synthetic 620-nt-Ifna2-oligo 2-T
tgaccagtagctcatcactatcagcatcaatcaccaactacccacacacacc

SEQ ID NO: 665 - DNA, 52 bp, Artificial Sequence
  Synthetic 620-nt-Ifng-oligo 2-T
agcgttcattgtctcagagctaggccgcaatcaccaactacccacacacacc

SEQ ID NO: 666 - DNA, 52 bp, Artificial Sequence
  Synthetic 620-nt-Ikbkb-oligo 2-T
aggggaggctaggacaccggtgtagttaaatcaccaactacccacacacacc

SEQ ID NO: 667 - DNA, 52 bp, Artificial Sequence
  Synthetic 620-nt-Ikbkg-oligo 2-T
ttcatcttgaagcagtgacactgtaggaaatcaccaactacccacacacacc

SEQ ID NO: 668 - DNA, 52 bp, Artificial Sequence
  Synthetic 620-nt-Il10-oligo 2-T
cccctgctgatcctcatgccagtcagtaaatcaccaactacccacacacacc

SEQ ID NO: 669 - DNA, 47 bp, Artificial Sequence
  Synthetic 620-nt-Il12a-oligo 2-T
cggagcgcgttgcaagttgtgataatcaccaactacccacacacacc

SEQ ID NO: 670 - DNA, 51 bp, Artificial Sequence
  Synthetic 620-nt-Il12b-oligo 2-T
agatgcagggagttagcgacagggaagaatcaccaactacccacacacacc

SEQ ID NO: 671 - DNA, 51 bp, Artificial Sequence
  Synthetic 620-nt-Il3ra-oligo 2-T
cgcgagtaaacagtgtgaggcagggggaatcaccaactacccacacacacc

SEQ ID NO: 672 - DNA, 52 bp, Artificial Sequence
  Synthetic 620-nt-Il6-oligo 2-T
gtggttgtcaccagcatcagtcccaagaaatcaccaactacccacacacacc

SEQ ID NO: 673 - DNA, 52 bp, Artificial Sequence
  Synthetic 620-nt-Irak1-oligo 2-T
ggctccagggcgtccatcactttgtagaaatcaccaactacccacacacacc

SEQ ID NO: 674 - DNA, 45 bp, Artificial Sequence
  Synthetic 620-nt-Irak4-oligo 2-T
gggacagaggacagcggagagaatcaccaactacccacacacacc

SEQ ID NO: 675 - DNA, 52 bp, Artificial Sequence
  Synthetic 620-nt-Irf3-oligo 2-T
gctcccaaacagtagttctgagactcgcaatcaccaactacccacacacacc

SEQ ID NO: 676 - DNA, 43 bp, Artificial Sequence
  Synthetic 620-nt-Irf5-oligo 2-T
aggcgcagtggggatgtccaatcaccaactacccacacacacc

SEQ ID NO: 677 - DNA, 52 bp, Artificial Sequence
  Synthetic 620-nt-Irf7-oligo 2-T
gatcactcactactctgtagaggggacaaatcaccaactacccacacacacc

SEQ ID NO: 678 - DNA, 52 bp, Artificial Sequence
  Synthetic 620-nt-Jun-oligo 2-T
tgtctctgccacactcagtgcaactctgaatcaccaactacccacacacacc

SEQ ID NO: 679 - DNA, 52 bp, Artificial Sequence
  Synthetic 620-nt-Kdm6b-oligo 2-T
caagcttaccagatggagttggaagtggaatcaccaactacccacacacacc

SEQ ID NO: 680 - DNA, 49 bp, Artificial Sequence
  Synthetic 620-nt-Lbp-oligo 2-T
ctgtatggacaggaacagcagccccaatcaccaactacccacacacacc

SEQ ID NO: 681 - DNA, 51 bp, Artificial Sequence
  Synthetic 620-nt-Ly86-oligo 2-T
aagccagcccaataaaaagacctccgaaatcaccaactacccacacacacc

SEQ ID NO: 682 - DNA, 45 bp, Artificial Sequence
  Synthetic 620-nt-Map3k1-oligo 2-T
cgccattttctctcgcgggctaatcaccaactacccacacacacc

SEQ ID NO: 683 - DNA, 51 bp, Artificial Sequence
  Synthetic 620-nt-Mapk1-oligo 2-T
ggcgatcgggaacgaggaaggaggacaaatcaccaactacccacacacacc

SEQ ID NO: 684 - DNA, 52 bp, Artificial Sequence
  Synthetic 620-nt-Mapk13-oligo 2-T
cccacgaactccgagatcccttttaaaaaatcaccaactacccacacacacc

SEQ ID NO: 685 - DNA, 52 bp, Artificial Sequence
  Synthetic 620-nt-Mapk14-oligo 2-T
cagatggtcttgttcagctcctgccggtaatcaccaactacccacacacacc

SEQ ID NO: 686 - DNA, 50 bp, Artificial Sequence
  Synthetic 620-nt-Mapk15-oligo 2-T
gggccagattctcagggaagccagggaatcaccaactacccacacacacc

SEQ ID NO: 687 - DNA, 52 bp, Artificial Sequence
  Synthetic 620-nt-Mapk3-oligo 2-T
gaagtcactgcacgatacttcccctgcgaatcaccaactacccacacacacc

SEQ ID NO: 688 - DNA, 49 bp, Artificial Sequence
  Synthetic 620-nt-Mapk4-oligo 2-T
ggcagcgagagaaagaaagcgggcgaatcaccaactacccacacacacc

SEQ ID NO: 689 - DNA, 50 bp, Artificial Sequence
  Synthetic 620-nt-Mapk6-oligo 2-T
acacactcacgggacttctccgactcaatcaccaactacccacacacacc

SEQ ID NO: 690 - DNA, 49 bp, Artificial Sequence
  Synthetic 620-nt-Mapk8-oligo 2-T
tcacgtccgcacaaagaaaccgcccaatcaccaactacccacacacacc

SEQ ID NO: 691 - DNA, 48 bp, Artificial Sequence
  Synthetic 620-nt-Mme-oligo 2-T
ggtgactcaccagtgggattggggaatcaccaactacccacacacacc

SEQ ID NO: 692 - DNA, 52 bp, Artificial Sequence
  Synthetic 620-nt-Myd88-oligo 2-T
gttaagcgcgaccaagggtatggagaacaatcaccaactacccacacacacc

SEQ ID NO: 693 - DNA, 52 bp, Artificial Sequence
  Synthetic 620-nt-Nfkb1-oligo 2-T
ctggctggaaattcccactgacgtcgagaatcaccaactacccacacacacc

SEQ ID NO: 694 - DNA, 52 bp, Artificial Sequence
  Synthetic 620-nt-Nfkb2-oligo 2-T
gtgcccagaggttggctatctctgagacaatcaccaactacccacacacacc

SEQ ID NO: 695 - DNA, 52 bp, Artificial Sequence
  Synthetic 620-nt-Nfkbia-oligo 2-T
caccatttgctcgtactcctcgtccttcaatcaccaactacccacacacacc

SEQ ID NO: 696 - DNA, 48 bp, Artificial Sequence
  Synthetic 620-nt-Nfkbib-oligo 2-T
cgcccctgtactgcaccacagccaaatcaccaactacccacacacacc

SEQ ID NO: 697 - DNA, 52 bp, Artificial Sequence
  Synthetic 620-nt-Nr2c2-oligo 2-T
gggaccgcgctttgttattgtttttgtcaatcaccaactacccacacacacc

SEQ ID NO: 698 - DNA, 52 bp, Artificial Sequence
  Synthetic 620-nt-Pik3ap1-oligo 2-T
ctaaagaagtagggagacaggtctcaagaatcaccaactacccacacacacc

SEQ ID NO: 699 - DNA, 45 bp, Artificial Sequence
  Synthetic 620-nt-Pik3c2a-oligo 2-T
gccccgaggaaggagtcagagaatcaccaactacccacacacacc

SEQ ID NO: 700 - DNA, 48 bp, Artificial Sequence
  Synthetic 620-nt-Pik3c2g-oligo 2-T
tggaaggcaggtccaggccagcagaatcaccaactacccacacacacc

SEQ ID NO: 701 - DNA, 52 bp, Artificial Sequence
  Synthetic 620-nt-Pik3c3-oligo 2-T
caggaagaaaccgccgctgcatcaaaacaatcaccaactacccacacacacc

SEQ ID NO: 702 - DNA, 52 bp, Artificial Sequence
  Synthetic 620-nt-Pik3ca-oligo 2-T
cctccgctggtcttccatctacccacgtaatcaccaactacccacacacacc

SEQ ID NO: 703 - DNA, 48 bp, Artificial Sequence
  Synthetic 620-nt-Pik3r1-oligo 2-T
tcggctcgcttccttcctctgcccaatcaccaactacccacacacacc

SEQ ID NO: 704 - DNA, 52 bp, Artificial Sequence
  Synthetic 620-nt-Pik3r2-oligo 2-T
ggtatcgcggaacccccaatgcaaaaggaatcaccaactacccacacacacc

SEQ ID NO: 705 - DNA, 52 bp, Artificial Sequence
  Synthetic 620-nt-Pik3r3-oligo 2-T
aaaagctcccaccgcctcctaatctttcaatcaccaactacccacacacacc

SEQ ID NO: 706 - DNA, 50 bp, Artificial Sequence
  Synthetic 620-nt-Pik3r5-oligo 2-T
cctaccggctgcagttggagcacactaatcaccaactacccacacacacc

SEQ ID NO: 707 - DNA, 52 bp, Artificial Sequence
  Synthetic 620-nt-Pik3r6-oligo 2-T
cagccaatgagctcctgaggtcctgttcaatcaccaactacccacacacacc

SEQ ID NO: 708 - DNA, 52 bp, Artificial Sequence
  Synthetic 620-nt-Ptpn6-oligo 2-T
atttcgtttcttggccacttcctcctctaatcaccaactacccacacacacc

SEQ ID NO: 709 - DNA, 51 bp, Artificial Sequence
  Synthetic 620-nt-Rela-oligo 2-T
cggcgcgctaaagtaaagccattcgccaatcaccaactacccacacacacc

SEQ ID NO: 710 - DNA, 50 bp, Artificial Sequence
  Synthetic 620-nt-Src-oligo 2-T
acctgaaggagaaactcacccaggacaatcaccaactacccacacacacc

SEQ ID NO: 711 - DNA, 48 bp, Artificial Sequence
  Synthetic 620-nt-Stat3-oligo 2-T
cgacaccgagcggggttgtacctcaatcaccaactacccacacacacc

SEQ ID NO: 712 - DNA, 49 bp, Artificial Sequence
  Synthetic 620-nt-Tab1-oligo 2-T
cgaggcggtccacacaatgccagagaatcaccaactacccacacacacc

SEQ ID NO: 713 - DNA, 45 bp, Artificial Sequence
  Synthetic 620-nt-Tab2-oligo 2-T
tccgcatcaaaatacggccacaatcaccaactacccacacacacc

SEQ ID NO: 714 - DNA, 52 bp, Artificial Sequence
  Synthetic 620-nt-Tet2-oligo 2-T
cctttctaacaaatcctacagggcagccaatcaccaactacccacacacacc

SEQ ID NO: 715 - DNA, 48 bp, Artificial Sequence
  Synthetic 620-nt-Tlr2-oligo 2-T
cacaggggtctgcaaggggaacggaatcaccaactacccacacacacc

SEQ ID NO: 716 - DNA, 44 bp, Artificial Sequence
  Synthetic 620-nt-Tlr4-oligo 2-T
cagggaggcatcatcctggcaatcaccaactacccacacacacc

SEQ ID NO: 717 - DNA, 52 bp, Artificial Sequence
  Synthetic 620-nt-Tnf-oligo 2-T
ctgtgctcatggtgtcttttctggagggaatcaccaactacccacacacacc

SEQ ID NO: 718 - DNA, 52 bp, Artificial Sequence
  Synthetic 620-nt-Traf6-oligo 2-T
actctagttcgaagccgctcactcaccgaatcaccaactacccacacacacc

SEQ ID NO: 719 - DNA, 51 bp, Artificial Sequence
  Synthetic 620-nt-Vav1-oligo 2-T
tggcctggggaagcaggttgttaagcaaatcaccaactacccacacacacc

SEQ ID NO: 720 - DNA, 50 bp, Artificial Sequence
  Synthetic 620-nt-Ash1l-oligo 3-T
ctaccccaccttcctcattctctagtggagagagagagggtggcagcctg

SEQ ID NO: 721 - DNA, 52 bp, Artificial Sequence
  Synthetic 620-nt-Btk-oligo 3-T
ctaccccaccttcctcattctctagacgtcgtgagatgccaggacttggaag

SEQ ID NO: 722 - DNA, 52 bp, Artificial Sequence
  Synthetic 620-nt-Ccl5-oligo 3-T
ctaccccaccttcctcattctctatggtcagaaccctcgacttacatggtga

SEQ ID NO: 723 - DNA, 50 bp, Artificial Sequence
  Synthetic 620-nt-Cd180-oligo 3-T
ctaccccaccttcctcattctctatgtccggagccatcccaggtccagag

SEQ ID NO: 724 - DNA, 43 bp, Artificial Sequence
  Synthetic 620-nt-Cox8a-oligo 3-T
ctaccccaccttcctcattctctatgacggcgcggaacgacca

SEQ ID NO: 725 - DNA, 52 bp, Artificial Sequence
  Synthetic 620-nt-Cxcl15-oligo 3-T
ctaccccaccttcctcattctctagatgcctaggttaaggacagccagcagg

SEQ ID NO: 726 - DNA, 52 bp, Artificial Sequence
  Synthetic 620-nt-Cxcr4-oligo 3-T
ctaccccaccttcctcattctctatcggttccatggcaacactcgctctaca

SEQ ID NO: 727 - DNA, 49 bp, Artificial Sequence
  Synthetic 620-nt-Dnmt3b-oligo 3-T
ctaccccaccttcctcattctctactcttcagagtggcggagtgcagcg

SEQ ID NO: 728 - DNA, 50 bp, Artificial Sequence
  Synthetic 620-nt-Foxo1-oligo 3-T
ctaccccaccttcctcattctctaggagcctccgagttcgtgcctaggac

SEQ ID NO: 729 - DNA, 52 bp, Artificial Sequence
  Synthetic 620-nt-Gdf5-oligo 3-T
ctaccccaccttcctcattctctataactcgttcttgaaaggagaaagccga

SEQ ID NO: 730 - DNA, 45 bp, Artificial Sequence
  Synthetic 620-nt-Hdac3-oligo 3-T
ctaccccaccttcctcattctctacggtcttggccatggtgctgg

SEQ ID NO: 731 - DNA, 45 bp, Artificial Sequence
  Synthetic 620-nt-Hsf1-oligo 3-T
ctaccccaccttcctcattctctagctagtgtcgtcgctgcccct

SEQ ID NO: 732 - DNA, 52 bp, Artificial Sequence
  Synthetic 620-nt-Hsf2bp-oligo 3-T
ctaccccaccttcctcattctctaggagacctgacctccaactaaccgccct

SEQ ID NO: 733 - DNA, 50 bp, Artificial Sequence
  Synthetic 620-nt-Hsf4-oligo 3-T
ctaccccaccttcctcattctctacattggagggatcccggggaccactg

SEQ ID NO: 734 - DNA, 43 bp, Artificial Sequence
  Synthetic 620-nt-Hsf5-oligo 3-T
ctaccccaccttcctcattctctagcggctggtcgatgagcag

SEQ ID NO: 735 - DNA, 52 bp, Artificial Sequence
  Synthetic 620-nt-Hsfy2-oligo 3-T
ctaccccaccttcctcattctctagggtatgcggtggcctctttatcaagaa

SEQ ID NO: 736 - DNA, 48 bp, Artificial Sequence
  Synthetic 620-nt-Hsp90aa1-oligo 3-T
ctaccccaccttcctcattctctacaccagggagggcacacaagcacc

SEQ ID NO: 737 - DNA, 48 bp, Artificial Sequence
  Synthetic 620-nt-Hsp90ab1-oligo 3-T
ctaccccaccttcctcattctctacgttatctccacaggctgccccgc

SEQ ID NO: 738 - DNA, 48 bp, Artificial Sequence
  Synthetic 620-nt-Hspa1a-oligo 3-T
ctaccccaccttcctcattctctaggtggtgcccaggtcgatgccgat

SEQ ID NO: 739 - DNA, 48 bp, Artificial Sequence; identical to SEQ ID NO: 738
  Synthetic 620-nt-Hspa1b-oligo 3-T

SEQ ID NO: 740 - DNA, 52 bp, Artificial Sequence
  Synthetic 620-nt-Hspa2-oligo 3-T
ctaccccaccttcctcattctctagccaacggtcgcttctcacaactctcca

SEQ ID NO: 741 - DNA, 50 bp, Artificial Sequence
  Synthetic 620-nt-Ifna1-oligo 3-T
ctaccccaccttcctcattctctagccttcttgatctgctgggcatccac

SEQ ID NO: 742 - DNA, 52 bp, Artificial Sequence
  Synthetic 620-nt-Ifna2-oligo 3-T
ctaccccaccttcctcattctctacgaggaaagcacagagtctagccattgt

SEQ ID NO: 743 - DNA, 52 bp, Artificial Sequence
  Synthetic 620-nt-Ifng-oligo 3-T
ctaccccaccttcctcattctctaggaggagaagcccagaacttctgtctca

SEQ ID NO: 744 - DNA, 49 bp, Artificial Sequence
  Synthetic 620-nt-Ikbkb-oligo 3-T
ctaccccaccttcctcattctctacaaataaaagttgcggcggccgggc

SEQ ID NO: 745 - DNA, 52 bp, Artificial Sequence
  Synthetic 620-nt-Ikbkg-oligo 3-T
ctaccccaccttcctcattctctagctaggcggctcagctttagcacaagag

SEQ ID NO: 746 - DNA, 46 bp, Artificial Sequence
  Synthetic 620-nt-Il10-oligo 3-T
ctaccccaccttcctcattctctagagcaggcagcatagcagtgct

SEQ ID NO: 747 - DNA, 52 bp, Artificial Sequence
  Synthetic 620-nt-Il12a-oligo 3-T
ctaccccaccttcctcattctctactggtggggatatatctggtcattgccg

SEQ ID NO: 748 - DNA, 52 bp, Artificial Sequence
  Synthetic 620-nt-Il12b-oligo 3-T
ctaccccaccttcctcattctctaggagagaacttaccttgctttgctgcga

SEQ ID NO: 749 - DNA, 52 bp, Artificial Sequence
  Synthetic 620-nt-Il3ra-oligo 3-T
ctaccccaccttcctcattctctatgaccacgagttagtttctgaggcaggg

SEQ ID NO: 750 - DNA, 52 bp, Artificial Sequence
  Synthetic 620-nt-Il6-oligo 3-T
ctaccccaccttcctcattctctaggcaactggatggaagtctcctgcgtgg

SEQ ID NO: 751 - DNA, 48 bp, Artificial Sequence
  Synthetic 620-nt-Irak1-oligo 3-T
ctaccccaccttcctcattctctagcggcacataacccagggtggcac

SEQ ID NO: 752 - DNA, 51 bp, Artificial Sequence
  Synthetic 620-nt-Irak4-oligo 3-T
ctaccccaccttcctcattctctatcgtggcttgagatcagccttcctcat

SEQ ID NO: 753 - DNA, 50 bp, Artificial Sequence
  Synthetic 620-nt-Irf3-oligo 3-T
ctaccccaccttcctcattctctagccacgcaacccccttctttgtcagc

SEQ ID NO: 754 - DNA, 44 bp, Artificial Sequence
  Synthetic 620-nt-Irf5-oligo 3-T
ctaccccaccttcctcattctctactgggcccttgggactttcc

SEQ ID NO: 755 - DNA, 52 bp, Artificial Sequence
  Synthetic 620-nt-Irf7-oligo 3-T
ctaccccaccttcctcattctctagaatttcccctgtggcactagctcacct

SEQ ID NO: 756 - DNA, 52 bp, Artificial Sequence
  Synthetic 620-nt-Jun-oligo 3-T
ctaccccaccttcctcattctctagtccttatccagcctgagctcaacactt

SEQ ID NO: 757 - DNA, 52 bp, Artificial Sequence
  Synthetic 620-nt-Kdm6b-oligo 3-T
ctaccccaccttcctcattctctactgtctcatggaacaagcactccagcag

SEQ ID NO: 758 - DNA, 52 bp, Artificial Sequence
  Synthetic 620-nt-Lbp-oligo 3-T
ctaccccaccttcctcattctctagcaatgtagagagcagaggtccagtccc

SEQ ID NO: 759 - DNA, 50 bp, Artificial Sequence
  Synthetic 620-nt-Ly86-oligo 3-T
ctaccccaccttcctcattctctagctcgtcctccccctgaaggagtagt

SEQ ID NO: 760 - DNA, 41 bp, Artificial Sequence
  Synthetic 620-nt-Map3k1-oligo 3-T
ctaccccaccttcctcattctctacattcgctcgtgcctgc

SEQ ID NO: 761 - DNA, 52 bp, Artificial Sequence
  Synthetic 620-nt-Mapk1-oligo 3-T
ctaccccaccttcctcattctctacacagaggagagaattaaccgccggtag

SEQ ID NO: 762 - DNA, 52 bp, Artificial Sequence
  Synthetic 620-nt-Mapk13-oligo 3-T
ctaccccaccttcctcattctctagttacagccttggagaaccagattcgcc

SEQ ID NO: 763 - DNA, 52 bp, Artificial Sequence
  Synthetic 620-nt-Mapk14-oligo 3-T
ctaccccaccttcctcattctctagaacgtgggcctctcctgcgacatcttc

SEQ ID NO: 764 - DNA, 49 bp, Artificial Sequence
  Synthetic 620-nt-Mapk15-oligo 3-T
ctaccccaccttcctcattctctacctgtgggcacaaggtggagccaac

SEQ ID NO: 765 - DNA, 45 bp, Artificial Sequence
  Synthetic 620-nt-Mapk3-oligo 3-T
ctaccccaccttcctcattctctacactccctgagccccccatgt

SEQ ID NO: 766 - DNA, 43 bp, Artificial Sequence
  Synthetic 620-nt-Mapk4-oligo 3-T
ctaccccaccttcctcattctctagcagggagccactcaccga

SEQ ID NO: 767 - DNA, 43 bp, Artificial Sequence
  Synthetic 620-nt-Mapk6-oligo 3-T
ctaccccaccttcctcattctctatcgacagagtgcagggccc

SEQ ID NO: 768 - DNA, 50 bp, Artificial Sequence
  Synthetic 620-nt-Mapk8-oligo 3-T
ctaccccaccttcctcattctctacagtcaaaacacgctccccgtgggtg

SEQ ID NO: 769 - DNA, 51 bp, Artificial Sequence
  Synthetic 620-nt-Mme-oligo 3-T
ctaccccaccttcctcattctctagaagttggaggcgactgagcagacaca

SEQ ID NO: 770 - DNA, 50 bp, Artificial Sequence
  Synthetic 620-nt-Myd88-oligo 3-T
ctaccccaccttcctcattctctatgaaggagtccagggacccggatccc

SEQ ID NO: 771 - DNA, 50 bp, Artificial Sequence
  Synthetic 620-nt-Nfkb1-oligo 3-T
ctaccccaccttcctcattctctagagagcagacagacggacggacccac

SEQ ID NO: 772 - DNA, 44 bp, Artificial Sequence
  Synthetic 620-nt-Nfkb2-oligo 3-T
ctaccccaccttcctcattctctacaggaaaccgggtcagtccc

SEQ ID NO: 773 - DNA, 45 bp, Artificial Sequence
  Synthetic 620-nt-Nfkbia-oligo 3-T
ctaccccaccttcctcattctctatggagtccaggccgctgtcgt

SEQ ID NO: 774 - DNA, 52 bp, Artificial Sequence
  Synthetic 620-nt-Nfkbib-oligo 3-T
ctaccccaccttcctcattctctatcagaaaaatgaggtggacattcgctcg

SEQ ID NO: 775 - DNA, 52 bp, Artificial Sequence
  Synthetic 620-nt-Nr2c2-oligo 3-T
ctaccccaccttcctcattctctagtgaaaggtcagagttcgtgaccccggt

SEQ ID NO: 776 - DNA, 51 bp, Artificial Sequence
  Synthetic 620-nt-Pik3ap1-oligo 3-T
ctaccccaccttcctcattctctactgggagggtggtgtcagagagcctca

SEQ ID NO: 777 - DNA, 48 bp, Artificial Sequence
  Synthetic 620-nt-Pik3c2a-oligo 3-T
ctaccccaccttcctcattctctaggatggagtgaccaaggggcacgg

SEQ ID NO: 778 - DNA, 52 bp, Artificial Sequence
  Synthetic 620-nt-Pik3c2g-oligo 3-T
ctaccccaccttcctcattctctatttgtgctgtgggtggcagagtcaatcc

SEQ ID NO: 779 - DNA, 52 bp, Artificial Sequence
  Synthetic 620-nt-Pik3c3-oligo 3-T
ctaccccaccttcctcattctctatgaaaacttcctgttccgcccatctgcc

SEQ ID NO: 780 - DNA, 43 bp, Artificial Sequence
  Synthetic 620-nt-Pik3ca-oligo 3-T
ctaccccaccttcctcattctctagagcggaggctgacaccgt

SEQ ID NO: 781 - DNA, 48 bp, Artificial Sequence
  Synthetic 620-nt-Pik3r1-oligo 3-T
ctaccccaccttcctcattctctagcctgggcttcagcggcctctgaa

SEQ ID NO: 782 - DNA, 52 bp, Artificial Sequence
  Synthetic 620-nt-Pik3r2-oligo 3-T
ctaccccaccttcctcattctctaggggcctacgatgcacgcatagtctgtg

SEQ ID NO: 783 - DNA, 49 bp, Artificial Sequence
  Synthetic 620-nt-Pik3r3-oligo 3-T
ctaccccaccttcctcattctctagcggaagccacttttgtgtccttcg

SEQ ID NO: 784 - DNA, 52 bp, Artificial Sequence
  Synthetic 620-nt-Pik3r5-oligo 3-T
ctaccccaccttcctcattctctactccagaatggtcccaacctgctctggc

SEQ ID NO: 785 - DNA, 48 bp, Artificial Sequence
  Synthetic 620-nt-Pik3r6-oligo 3-T
ctaccccaccttcctcattctctaggaggccagcaaccccaaggacca

SEQ ID NO: 786 - DNA, 52 bp, Artificial Sequence
  Synthetic 620-nt-Ptpn6-oligo 3-T
ctaccccaccttcctcattctctatccagcctgtcaaaccaatgggactctg

SEQ ID NO: 787 - DNA, 40 bp, Artificial Sequence
  Synthetic 620-nt-Rela-oligo 3-T
ctaccccaccttcctcattctctagaggcggaaatgcgcg

SEQ ID NO: 788 - DNA, 48 bp, Artificial Sequence
  Synthetic 620-nt-Src-oligo 3-T
ctaccccaccttcctcattctctagcccttcctccagccacgtctgca

SEQ ID NO: 789 - DNA, 43 bp, Artificial Sequence
  Synthetic 620-nt-Stat3-oligo 3-T
ctaccccaccttcctcattctctagcgatccggttagggctcg

SEQ ID NO: 790 - DNA, 50 bp, Artificial Sequence
  Synthetic 620-nt-Tab1-oligo 3-T
ctaccccaccttcctcattctctatacagcacgccttacacaagcggggg

SEQ ID NO: 791 - DNA, 40 bp, Artificial Sequence
  Synthetic 620-nt-Tab2-oligo 3-T
ctaccccaccttcctcattctctacaatatggcggcgcgg

SEQ ID NO: 792 - DNA, 52 bp, Artificial Sequence
  Synthetic 620-nt-Tet2-oligo 3-T
ctaccccaccttcctcattctctatcttgcttccttctgctcctctacgaaa

SEQ ID NO: 793 - DNA, 48 bp, Artificial Sequence
  Synthetic 620-nt-Tlr2-oligo 3-T
ctaccccaccttcctcattctctagcctggcatcagagctggcaggag

SEQ ID NO: 794 - DNA, 52 bp, Artificial Sequence
  Synthetic 620-nt-Tlr4-oligo 3-T
ctaccccaccttcctcattctctattttctgcaaccagtggaggccatgaga

SEQ ID NO: 795 - DNA, 48 bp, Artificial Sequence
  Synthetic 620-nt-Tnf-oligo 3-T
ctaccccaccttcctcattctctagatgtggcgccttgggccagtgag

SEQ ID NO: 796 - DNA, 52 bp, Artificial Sequence
  Synthetic 620-nt-Traf6-oligo 3-T
ctaccccaccttcctcattctctatcgtacacgctgagacttcacaaccaac

SEQ ID NO: 797 - DNA, 47 bp, Artificial Sequence
  Synthetic 620-nt-Vav1-oligo 3-T
ctaccccaccttcctcattctctattggcacaagaggacaccgtccc

(19) United States
(12) Patent Application Publication
(10) Pub. No.: US 2026/0117293 A1
(43) Pub. Date: Apr. 30, 2026

(54) Methods and kits for targeted cleavage and enrichment of Nucleic Acids for high-throughput analyses of user-defined genomic regions

(71) Applicant: UNIVERSITY OF FLORIDA RESEARCH FOUNDATION, INCORPORATED, Gainesville, FL (US)

(72) Inventors: Michael P. KLADDE, Gainesville, FL (US); Mingqi ZHOU, Gainesville, FL (US); Nancy Hisham NABILSI, Gainesville, FL (US)

(21) Appl. No.: 18/551,004

(22) PCT Filed: Mar. 16, 2022
(86) PCT No.: PCT/US2022/020624

§ 371 Date: Sep. 18, 2023

Related U.S. Application Data
(60) Provisional application No. 63/161,736, filed on Mar. 16, 2021.

(51) Int. Cl. C12Q 1/6855; C12Q 1/34; C12Q 1/48; C12Q 1/6806; C12Q 1/686

(52) U.S. Cl. CPC C12Q 1/6855; C12Q 1/34; C12Q 1/48; C12Q 1/6806; C12Q 1/686; G01N 2333/91011; G01N 2333/916

(57) ABSTRACT

Disclosed herein are methods, oligonucleotides, and kits for targeted cleavage and enrichment of nucleic acids for high-throughput analyses of user-defined genomic regions. Targeted sequence enrichment is an increasingly sought technology. Currently available methods exhibit biases and substantial proportions of off-target reads. Here, disclosed is FENGC, a versatile, multiplexed method, wherein oligonucleotide adapters and flap endonuclease direct 5′ DNA flap formation and cutting that releases target sequences with nucleotide-level precision. The target-specific oligonucleotides are designed by a novel program called FENGC oligonucleotide designer (FOLD). Further disclosed are the oligonucleotides and kits required to perform FENGC.

### GOVERNMENT SUPPORT

This invention was made with government support under Grant Numbers R01 CA155390 awarded by The National Institutes of Health and HDTRA1-16-1-0048 awarded by the Defense Threat Reduction Agency. The government has certain rights in the invention.

### REFERENCE TO ELECTRONIC SEQUENCE LISTING

The application contains a Sequence Listing which has been submitted electronically in .txt format and is hereby incorporated by reference in its entirety. Said .txt copy, created on Jun. 30, 2022, is named “10457496PC0_ST25.txt” and is 168,156 bytes in size. The sequence listing contained in this .txt file is part of the specification and is hereby incorporated by reference herein in its entirety.

### FIELD OF THE INVENTION

The invention is related to methods for capturing and amplifying a plurality of specific sequences in a multiplexed reaction.

### BACKGROUND

Targeted enrichment of DNA sequences for next-generation sequencing (NGS) is a widely used procedure in basic investigative science and medicine. Its diverse applications include medical discovery and diagnostics, genotyping, biomarker assessment, marker-assisted plant breeding, epigenetic analysis of DNA methylation, etc. Multiplexed enrichment of target sequences from a complex mixture of sequences reduces sample complexity, which enables higher sequencing coverages, improves data quality, and reduces overall cost. Enrichment of many genomic loci of interest in the same reaction in combination with sample barcoding yields further increases in NGS throughput, efficiency, and cost savings. Several distinct strategies have been developed for multiplexed sequence enrichment for NGS in genetic applications and epigenetic detection of DNA methylation, for example, 5-methylcytosine (5mC) and 5-hydroxymethylcytosine (5hmC) following bisulfite treatment. Enrichment strategies that are compatible with both genetic and epigenetic inquiries provide additional versatility.

Multiplexed PCR amplification (Chamberlain et al., 1988) is commonly used to detect microsatellite and single-nucleotide polymorphisms or variants (Hayden et al., 2008). However, PCR does not scale well beyond analysis of 10-20 regions of interest in the same reaction due to non-specific amplification, adverse primer-primer interactions, and amplification biases that lead to uneven sequencing coverage. In addition, for analysis of 5mC and 5hmC by bisulfite sequencing (Darst et al., 2010), conversion of the majority of C bases to U reduces sequence complexity (without reducing genome size) and therefore available priming specificity, further limiting the ability to perform multiplex PCR.

To avoid these problems, several approaches for sequence enrichment have been developed that do not rely on prior PCR amplification. One such method uses a restriction endonuclease to digest DNA followed by selection of fragments of a specific size range (Meissner et al., 2005; Gu et al., 2011). The enzyme MspI (CCGG) is typically used because it cuts irrespective of the methylation status of the central CG, the most commonly methylated dinucleotide in vertebrate genomes (McClelland, 1981). Isolation of MspI fragments from 40-220 bp in length represents the majority of CpG islands in the human genome (Gu et al., 2011). In principle, the resulting library of fragments could be sequenced directly to detect genetic variants; however, the approach has instead been used to examine DNA methylation in a method called reduced representation bisulfite sequencing (RRBS) (Meissner et al., 2005). The total fraction and number of represented regions (e.g., promoters, CpG islands, and their adjacent shores, etc.) is increased by isolating 70- to 320-bp fragments for enhanced RRBS (Akalin et al., 2012; Garrett-Bakelman et al., 2015) or by utilizing additional methylation-insensitive restriction endonucleases (Martinez-Arguelles et al., 2014). A drawback of RRBS, however, shared with many NGS technologies, is the purification of different-size fragments leads to biased PCR amplification of smaller fragments during NGS library preparation. Moreover, the need for restriction enzyme digestion and size selection severely limits the flexibility of target selection, and precludes analysis of custom panels of loci or “walking” along contiguous segments of a genome.

### DESCRIPTION OF THE DRAWINGS

The present embodiments are illustrated by way of example, and not by way of limitation, in the figures of the accompanying drawings.

The following figures are illustrative only, and are not intended to be limiting.

FIG. 1. Preferred flap-enabled next-generation capture (FENGC) and enrichment protocol utilizing 5′ and 1-nt 3′ flaps. Purified source DNA, with or without fragmentation by sonication, restriction enzyme digestion, or other suitable method, is used directly as input. Step 1. A plurality of double flap structures with variable-length, single-stranded DNA (ssDNA) 5′ flaps and a constant 1-nt 3′ flap (pink arrowheads) is formed by addition of a set of flap adapters 1 and 2 that anneal to 5′ and 3′ ends of each region of interest, respectively (step 1a; only one target strand depicted in light gray for simplicity). As shown, each flap adapter comprises an oligo complex consisting of a target-specific oligo 1-N or oligo 2-N base paired at their common 3′ end with a universal oligo 1 in which a 3′ A, C, G, or T remains unpaired (oligo U1-N), constituting a 1-nt 3′ flap. Flap adapter pairs comprised of oligo U1-T paired with both an oligo 1-T and an oligo 2-T are preferred because they demonstrate superior cleavage and capture efficiency (see FIG. 3 and FIG. 11). The 5′ flap endonuclease (FEN) activity of FEN1 or Thermus aquaticus DNA polymerase (hereafter, Taq) is used to remove the 5′ flaps (step 1b). Phosphodiester bond cleavage occurs one nucleotide 3′ of both 5′ flaps (red arrowheads), within duplex DNA formed by the flap adapters annealed to their respective, complementary target sites. This releases target sequences with ligatable termini, i.e., a 5′ phosphate (downstream of a 1-nt gap) and a 3′ hydroxyl. Step 2. The base constituting the 1-nt 3′ flap at the 3′ end of oligo U1-N fills each 1-nt gap and is ligated to the 5′-phosphate of each sequence of interest, as well as to the downstream sequence (not shown). The 3′ end of each target sequence is ligated to a corresponding adapter 3 complex comprised of a target-specific oligo 3-N (nested relative to oligo 2-N) with its constant 5′ end base paired with oligo U2. The 3′ hydroxyl of the oligo U2 is covalently linked to a three carbon spacer (indicated by ball and stick). Step 3. This covalent modification protects oligo U2 and 3′ ends to which it has ligated against 3′ to 5′ degradation by addition of both exonuclease I (Exo I) and Exo III. Non-protected source DNA, unligated oligo U1-N, as well as oligos 1-N, oligos 2-N, and oligos 3 are fully degraded. Step 4. The captured and dramatically enriched single-stranded target sequences are purified for the first time. At the end of the protocol, the enriched sequences are amplified with oligo U1 (serves as primer as well) and U2 primer using standard PCR or methyl-PCR (PCR following deamination of C to U) to construct NGS libraries. In this and all subsequent figures, lines connecting different DNA strands indicate complementary base pairing; the number of lines is not intended to reflect any specific number of base pairs, and the various molecules are not drawn to scale.

FIG. 2. Alternative FENGC protocol utilizing a plurality of 5′ flaps with no 3′ flap. Taq is the preferred enzyme in this protocol because it cuts 5′ flaps without a 1-nt 3′ flap with more specificity than FEN1, ultimately yielding more amplification product (see FIG. 13). Step 1. Oligo U1 (instead of U1-N) used in the alternative protocol lacks the unpaired, 3′-terminal nucleotide and therefore cannot fill the 1-nt gap created by FEN cleavage of 5′ flap-only structures. Step 2. Therefore, after cleavage, at first, only oligo U2 is ligated to the 3′ ends of the plurality of target sequences annealed to adapters 3. Step 3. A first round of digestion with Exo I and Exo III dramatically enriches the plurality of oligo U2-protected sequences of interest (for details see FIG. 1 legend). Step 4. Following a first purification, a set of flap adapters 1 comprising oligo U1-N and oligos 1-N is added and ligated to the 5′ end of each target. Step 5. A second treatment with Exo I and Exo III removes oligos 1-N and unligated oligo U1-N. Step 6. The enriched DNA sequences are subjected to a second purification and then amplification with oligo U1 and U2 primer using standard PCR or methyl-PCR to construct NGS libraries.

FIG. 3. Preferred double flap structure to direct efficient and precise 5′ flap scission. Shown is an actual oligo 1-T that hybridizes to and forms a downstream duplex at the 5′ end of its respective target sequence of interest (gray). Similarly, an oligo 2-T hybridizes to the 3′ end of the target sequence of interest (not shown). The constant 3′ tail of all oligos 1-T (and oligos 2-T) is bound to U1-T oligo, forming an upstream duplex and reconstituting a plurality of double flaps. Note that the unpaired 3′ T of the U1-T oligo ‘overlaps’ a T in all target sequences of interest (residues highlighted in pink, corresponding to pink arrowheads in FIG. 1), allowing it to be displaced upon formation of the downstream duplex, creating unpaired 1-nt 3′-T flaps. To ensure that the 3′ flaps are only 1 nt in length, in this example, a V (A, C, or G) is selected to reside at the base of the 5′ flap as shown. A V base does not pair with the first nucleotide of the 3′ tail of the oligos 1-T and oligos 2-T, i.e., an A (indicated by blue arrow).

FIG. 4. Flap adapter-directed cleavage of target 200-mer ssDNA oligos. (A) Schematic of substrate to assess cleavage of a 200-mer in structures containing a 129-nt 5′ flap but no 3′ flap. A 200-mer with A, T, G, or C (200-N oligos) at position 130 (hydrogen bonds indicated by widest vertical line) was contacted by a flap adapter comprised of a 200 oligo 1-N annealed to a constant oligo U1 (TABLE 1, Sheet 1). In the naming convention, for example, the 200-A oligo is a proxy sequence of interest with A at nt 130, which anneals to a complementary T in the 200 oligo 1-A. Taq or FEN1 cleaves the target sequence between nt 130 and nt 131 (red arrowhead), yielding a complex with a 1-nt gap (see FIG. 2, step 1b). The oligos are not drawn to scale. (B) Representative Agilent 2100 Bioanalyzer traces for cleavage of a 5′ flap with no 3′ flap. Substrates were formed by mixing 500 nM each of 200-T oligo, 200 oligo 1-T, and oligo U1 with increasing amounts of Taq as indicated (U, unitage based on polymerization activity). The cycling parameters for cleavage were 3 min at 95° C. and 20 min at 65° C., followed by 14 cycles of 30 sec at 95° C. and 10 min at 65° C. Digital peaks corresponding to the migration positions of the uncut, input 200-T oligo (orange arrow, 69 s (sec) aligned migration time) and cut 200-T oligo (green arrow, 53 s aligned migration time) are indicated, as are size markers added post cleavage (black arrows, 43 s and 113 s aligned migration times). With 2 U and 5 U Taq, prominent peaks of cut 200-mer were produced at the expense of uncut, input 200-mer.

FIG. 5. Optimization of Taq digestion of 5′ flaps, with and without a 1-nt 3′ flap. Various 5′ flap substrates were formed on 200-N oligos, cleaved with Taq, and the digestion products separated on an Agilent 2100 Bioanalyzer as in FIG. 4. The areas under the peaks of cut and uncut 200-N oligo were integrated and the percentages of digestion were calculated by (mass of cut oligo)/(mass of cut oligo+mass of uncut oligo)×100. (A) Taq dose response (unitages based on polymerization activity) in Taq buffer for 15 reaction cycles as described in the legend of FIG. 4. Reactions contained 500 nM each of 200-T oligo, 200 oligo 1-T, and oligo U1 (TABLE 1, Sheet 1). (B) Percentages of cut 200-T oligo in reactions with 500 nM each of 200-T oligo, 200 oligo 1-T, and oligo U1 incubated with 1 U Taq for the indicated numbers of reaction cycles. The first digestion cycle was 3 min at 95° C. and 20 min at 65° C., and subsequent cycles were 30 sec at 95° C. and 10 min at 65° C. (C) Comparison of digestion efficiencies of various combinations of indicated oligos incubated with 1 U Taq for 10 reaction cycles. Each Taq reaction contained 500 nM each of 200-N oligo and either oligo U1 (A+U1, C+U1, G+U1, and T+U1) or the indicated oligo U1-N(A+U1-C, -G, or -T; C+U1-A, -G; or -T; G+U1-A, -C, or -T; and T+U1-A, -C, or -G) mixed with the corresponding 200 oligo 1-N(e.g., bar 2, 200-A oligo, 200 oligo 1-A, and U1-C oligo) (TABLE 1, Sheet 1). Note the similar efficiencies of cutting all 200-mers irrespective of the absence/presence or identity of the base constituting the 1-nt 3′ flap under these conditions employing 10 cutting cycles. All experiments were done in duplicate (n=2) and are reported as the arithmetic mean±range.

FIG. 6. Dose response of FEN1 cleavage of a 129-nt 5′ flap, without a 1-nt 3′ flap. Five hundred nanomolar each of 200-T oligo, corresponding 200 oligo 1-T, and oligo U1 (TABLE 1, Sheet 1) were incubated with increasing amounts of FEN1 in FEN1 buffer for 15 reaction cycles as described in the legend of FIG. 5. The products were analyzed and percentages of cut 200-T oligo were calculated as also described in the legend of FIG. 5. Overall, a higher percentage of 200-T oligo cleavage was achieved with FEN1 compared with Taq as tested in FIG. 5. Data are reported as mean±range (n=2).

FIG. 7. Digestion efficiencies of a 129-nt 5′ flap, without a 1-nt 3′ flap, with Taq and FEN1 in different reaction buffers. Five hundred nanomolar each of 200-T oligo, corresponding 200 oligo 1-T, and oligo U1 (TABLE 1, Sheet 1) were incubated with Taq or FEN1 in the indicated buffers for 10 reaction cycles as described in the legend of FIG. 5. The enzymes used were: 1 U APEX Taq (Genesee Scientific); 1 U HotStar Taq (Qiagen); and 32 U FEN1 (New England Biolabs). The units of Taq were based on polymerization activity. The buffers used were: 1×PCR buffer (Qiagen; referred to as Taq buffer); 1×ThermoPol reaction buffer (New England Biolabs; referred to as FEN1 buffer); and the mixed buffer reactions contained final concentrations of 1×CutSmart buffer (New England Biolabs) and 1×Taq buffer or FEN1 buffer. The products were analyzed and the percentages of cut 200-T oligo were calculated as described in the legend of FIG. 5. Data are reported as mean±range (n=2; *P<0.05, **P<0.01, Student's t test). As observed in FIG. 6, higher efficiencies of 200-T oligo cutting were obtained with FEN1 compared with Taq.

FIG. 8. 5-Methylcytosine (5mC) does not inhibit 5′ flap cleavage. (A) Dose response of FEN1 cleavage of four different control unmethylated and four different C-5-methylated double flaps. These eight structures were formed by mixing 500 nM each of: 1) 80-nt oligos that had either zero or five internal 5mC residues (at nt 43, 47, 49, 53, and 54, all located within 1-10 bp from each cleaved phosphodiester bond); 2) either 80 oligo 1-A, -C, -G, or -T; and 3) either U1-A, -C, -G, or -T oligo, respectively (TABLE 1, Sheet 1). The -N suffixes in the key indicate the 3′-terminal base of each U1-N oligo, which overlaps the respective base in its 80 oligo 1-N to create four different double flaps. Reactions were incubated with 0, 0.01, 0.04, 0.2, or 1 U of FEN1 in a 20 μl reaction for 3 min at 95° C. and 20 min at 65° C., followed by 14 cycles of 30 sec at 95° C. and 10 min at 65° C. The percentages of cut 80-mer were assessed by quantitative PCR (qPCR) and calculated using the formula: % cut=100×[1−2^{-(Ct of FEN1 treated-Ct of FEN1 untreated)}]. Data are mean±SD, n=3. (B) Effective concentration achieving 50% cleavage (EC₅₀) by FEN1 obtained from the data plotted in (A). No significant differences were observed between all pairwise comparisons of cleavage of the unmethylated and methylated substrates as determined by serial one-way ANOVA tests. Therefore, multiple 5mC in the targeted strand both adjacent to the FEN1 cleavage site and within sequences contacted by FEN1 do not significantly inhibit double flap cleavage.

FIG. 9. Effect of 5′ flap length on target strand cleavage and enrichment. (A) Schematic of plasmid pGEM-3Z/601b. The selected 571-nt target region (wide dark green line) and positions of HindIII, NdeI, and DrdI restriction sites as well as their map coordinates relative to the cut HindIII end are shown. (B) Target sequence enrichment after digestion of substrates with a 1-nt 3′ flap and different 5′ flap lengths. pGEM-3Z/601b (20 ng) was linearized with HindIII and divided equally into four separate reactions, two of which were digested further with either NdeI or DrdI. Plasmid digested with both HindIII and NdeI was contacted at the 5′ end of the 572-nt target (extra nucleotide due to location of NdeI cut) with an NdeI adapter (pGEM-3Z/601b NdeI oligo 1-T and oligo U1). This serves as a no flap positive control (no added Taq) for target strand capture by ligation and amplification of the expected 618-bp product, including 46 bp added by ligation of the oligos U1 and U2 (lane 1). Plasmid digested with HindIII-DrdI and only HindIII tested FENGC with addition of Taq and the flap adapter 1 (pGEM-3Z/601b NdeI oligo 1-T and U1-T oligo) for removal of 5′ flaps of 87 nt (lane 2) and 2,453 nt (lane 3), respectively. The fourth reaction with HindIII-linearized plasmid (lane 4) but omitting Taq provides a negative control; without scission of the 2,453-nt flap, the U1-T oligo should not ligate to the 5′ end of the target DNA strand and PCR amplification should also not occur. To effect cleavage of 5′ flaps, all four reactions were incubated for 3 min at 95° C. and 20 min at 65° C., followed by 9 cycles of 30 sec at 95° C. and 10 min at 65° C. Following cleavage, a HindIII adapter (pGEM-3Z/601b HindIII oligo and oligo U2) was added to all reactions in order to ligate the oligo U2 to the common HindIII-cut 3′ end of the ssDNA 571-nt target sequence. Ligation was performed using Ampligase (Lucigen), employing conditions of 3 min at 95° C. followed by 100 cycles of 0.5 min at 94° C. and 8 min at 65° C. After subsequent bisulfite PCR (BS-PCR), the amplification products were directly electrophoresed, i.e., not purified beforehand, on a 1% agarose gel containing 0.2 μg/ml ethidium bromide and imaged with ultraviolet light on a transilluminator. The specific 618-bp product was obtained in reactions 2 and 3, indicating precise cleavage of the 87 nt and 2,453 nt 5′ flaps, respectively, as well as ligation of the liberated 571-nt target sequence to the oligos U1-T and U2, adding 47 bp post amplification. By contrast, no amplification product was observed in the negative control reaction in which Taq was omitted. Oligos are indicated in TABLE 1, Sheet 1.

FIG. 10. FENGC enrichment with Taq after cleavage adjacent to and nearby a 5mC residue within double flap structures. (A) pGEM-3Z/601b, treated with or without M.SssI and methylation cofactor S-adenosyl-L-methionine (SAM), was digested with or without the methylation-sensitive restriction endonuclease HhaI. A 1-kb molecular size marker was electrophoresed in the leftmost lane. Complete digestion of unmethylated pGEM-3Z/601b (lane 2) and complete protection against digestion of methylated pGEM-3Z/601b (lane 4), respectively, was observed. (B) Unmethylated (U) and methylated (M) pGEM-3Z/601b was linearized with HindIII and then processed by FENGC. Three different flap adapters comprised of oligos U1-T, -G, and -C that annealed to methyl test pGEM-3Z/601b oligo 1-T, -G, or —C, respectively, were used to contact and form double flap structures with the pGEM-3Z/601b target sequence. The respective oligo U1-N was ligated to the 5′ end of each target sequence after cutting by the FEN activity of Taq at the indicated phosphodiester bonds (red arrowheads; 5mC in red type). The common HindIII-cut 3′ end of each target sequence was ligated to the pGEM-3Z/601b HindIII adapter, i.e., oligo U2 bound to the pGEM-3Z/601b HindIII oligo. After ligation, unprotected fragments were degraded by incubation with Exo I and Exo III. BS-PCR was performed and the amplified products were directly visualized by 1% agarose gel electrophoresis. A 100-bp molecular size marker was electrophoresed in the leftmost lane. Given that the expected amplification product was obtained in all reactions as observed in FIG. 8, it can be concluded that 5mC does not inhibit 5′ flap cleavage. In addition, the presence of 5mC at the 5′ end of the cut target sequence, did not block ligation to the oligo U1-N nor PCR amplification. Oligos are indicated in TABLE 1, Sheet 1.

FIG. 11. Identity of the 3′-terminal nucleotide of oligo U1-N affects the performance of preferred FENGC with double flaps. (A) Site-specific mutagenesis of pGEM-3Z/601b. The first A in the NdeI site in pGEM-3Z/601b at nt 2,453 was mutated to C, G, and T. The set of four resulting plasmids, pGEM-3Z/601b-N, introduces each of the four nucleotides immediately 5′ of the site of FEN cleavage and oligo U1-N ligation, changing the base constituting the 1-nt 3′ flap. After incubation with HindIII, 0.2 ng of each of the four linearized plasmids was denatured and contacted with the pGEM-3Z/601b HindIII adapter and each of the four respective flap adapters 1 (oligo U1-N annealed to its corresponding pGEM-3Z/601b NdeI-2 oligo 1-N; TABLE 1, Sheet 1). The flap adapters directed cleavage by Taq or FEN1 between nt 2,453 and nt 2,454, using the reaction cycles described in the legend of FIG. 9. After severing the 2,453-nt 5′ flap, the 572-nt target sequence released from each of the four linearized plasmids was ligated to its respective oligo U1-N and oligo U2 at the 5′ and 3′ ends, respectively. Reactions with no Taq and no FEN1 constituted negative controls. The ligation products were treated with Exo I and Exo III, purified with AMPure XP beads (Beckman Coulter), and subjected to BS-PCR. (B) BS-PCR products from FENGC with no enzyme (lanes 1-4), 2 U Taq (based on polymerization activity; lanes 5-8), and 32 U FEN1 (lanes 9-12) were directly analyzed by 1% agarose gel electrophoresis. Note the highest yield of the expected 619-bp product (572-bp target sequence plus 47 bp universal priming sequences) was obtained when oligo 1-T and pGEM-3Z/601b NdeI-2 oligo 1-T were incubated with pGEM-3Z/601b-T (lanes 6 and 10). Also, the use of oligo 1-G not only reduced the yield of specific product but led to an amplified smear of non-specific, high-molecular-weight products (compare lane 7 with 6 and lane 11 with 10).

FIG. 12. Identity of the 3′-terminal nucleotide of the oligo U1-N also affects the performance of alternative FENGC without a 1-nt 3′ flap. (A) Schematic of 5′ flap formation. The four pGEM-3Z/601b-N plasmids described in FIG. 11A were linearized with HindIII, and 0.2 ng of each was contacted by one of the four respective flap adapters (oligo U1 and corresponding pGEM-3Z/601b NdeI-2 oligo 1-N; TABLE 1, Sheet 1) in the presence of 0 U or 2 U Taq. (B) FENGC enrichment yields. The 3′ end of each cut target sequence was first ligated to the oligo U2 of the HindIII adapter. After degradation of unprotected DNA fragments by Exo I and Exo III, the 5′ end of the target sequence was ligated to the respective adapter (oligo U1-N and corresponding oligo 1-N). Reactions without Taq (or FEN1) are used as negative controls (lanes 1-4) and with Taq (lanes 5-6). The ligation products were treated a second time with Exo I and Exo III, amplified by BS-PCR, and directly analyzed by 1% agarose gel electrophoresis. Similar results as FIG. 11 were seen, with oligo 1-T providing the highest, specific amplification yield (lane 6). Again, use of U1-G produced a non-specific smear of high-molecular-weight products.

FIG. 13. Sensitivity of plasmid sequence enrichment using the alternative FENGC procedure without a 3′ flap. Two micrograms genomic DNA (gDNA) purified from human colon cancer cell line HCT116 was spiked with the indicated masses of HindIII-linearized pGEM-3Z/601b-T and subjected to the alternative FENGC protocol as described in FIG. 2 and FIG. 12B. Sensitivity of FENGC using (A) FEN1 (32 U) in its supplied buffer and (B) FEN1 versus Taq (2 U) in Taq buffer. The amplified products were directly analyzed by 1% agarose gel electrophoresis. Note that in alternative FENGC procedure, Taq (lanes 1-3) yields superior sensitivity compared with FEN1 (lanes 4-6) because FEN1 produces multiple cleavage products.

FIG. 14. Specificity of plasmid sequence enrichment using the alternative FENGC procedure. pGEM-3Z/601b-T (0.2 ng) was subjected to alternative FENGC as described in FIG. 2 and FIG. 12B, using each of the four indicated flap adapters 1 in the presence of 32 U FEN1. The amplified products were directly analyzed by 1% agarose gel electrophoresis. The specific, 619-bp PCR product was only obtained when FEN activity and cognate flap adapter 1 (U1-T oligo and pGEM-3Z/601b NdeI-2 oligo 1-T were included (lane 6).

FIG. 15. Comparison of preferred and alternative FENGC enrichment of target sequences of interest from gDNA using the FEN activity of Taq. Two micrograms of human HCT116 gDNA were subjected to the indicated FENGC procedure using 2 U Taq with and without a 3′ flap as described in FIG. 1 and FIG. 2, respectively. Cleavage was performed for 3 min at 95° C. and 20 min at 65° C., followed by 14 cycles of 30 sec at 95° C. and 10 min at 65° C. Matched flap adapters 1-T and 2-T for 11 single-copy genes were employed to capture and enrich sequences of mean length of ˜300 nt (TABLE 1 and TABLE 2, Sheets Human ˜300 nt Targets) using BS-PCR. Reactions without Taq were used as negative controls. The amplification products were directly analyzed by 1% agarose gel electrophoresis. Both the preferred and alternative FENGC procedures with Taq yielded the specific amplification product of ˜350 bp (282-315 bp target sequences plus 47 bp of ligated oligos U1-T and U2).

FIG. 16. Comparison of Taq and FEN1 in preferred FENGC enrichment of gDNA sequences. Two micrograms of human HCT116 gDNA were processed by FENGC with a 1-nt 3′ flap as described in FIG. 1. Sequences of mean length of ˜300 nt were captured from the same 11 genes in FIG. 15 using matched flap adapters 1-T and 2-T. Reactions contained (A) no enzyme, 2 U Taq in 1×Taq buffer, or 32 U FEN1 in 1×FEN1 buffer and (B) No or 32 U FEN1 in FEN1 or Taq buffer as indicated. Reactions with no enzyme constituted negative controls (lanes 1 and 3). The ˜350 bp amplification products of BS-PCR (282-315 bp target sequences plus 47 bp of universal primers) were directly analyzed by 1% agarose gel electrophoresis. Both panels show that FEN1 yields superior sequence enrichment compared with Taq (compare lanes 2 and 4).

FIG. 17. Effect of bisulfite conversion and amplicon length on the preferred FENGC protocol. FENGC was performed as described in FIG. 1 on 2 p g or 4 p g HCT116 gDNA as indicated using 32 U FEN1. In one set of reactions, the same flap adapters 1-T and 2-T were employed to enrich the same 11 target amplicons of ˜350 bp (282-315 bp of target sequence plus 47 bp of universal primers; lanes 1-3; TABLE 1 and TABLE 2, Sheets Human ˜300 nt Targets) as in FIG. 16. In a second set of reactions, the same flap adapters 1-T were used and new flap adapters 2-T were utilized that anneal farther downstream to capture and amplify sequences of ˜500 bp (430-452 bp of target sequence plus 47 bp of universal primers; lanes 4-7; TABLE 1 and TABLE 2, Sheets Human ˜450 nt Targets). The captured sequences were amplified without (lanes 1, 2, 4, and 5) and with bisulfite conversion (lanes 3, 6, and 7), i.e., subjected to standard PCR and BS-PCR, respectively. Reactions without FEN1 were included as negative controls (lanes 1 and 4). The amplification products were directly analyzed by 1% agarose gel electrophoresis. As expected, reduced PCR yields were observed with longer amplicons (compare lanes 2 with 5 and 3 with 6) and due to the known degradation of DNA by bisulfite (compare lanes 2 with 3 and 5 with 6). An ˜500 bp BS-PCR product was not detected with 2 g gDNA input but a sufficient yield was obtained with 4 g gDNA input (compare lane 6 and 7).

FIG. 18. MAPit-FENGC of 10 promoter sequences of human DNA mismatch repair genes plus 1 human control gene amplified by standard PCR and BS-PCR. Two independent biological replicates (lanes 1-6 and lanes 7-12) of cultured human glioblastoma (GBM) L0 cells were assayed by Methyltransferase Accessibility Protocol for individual templates (MAPit) methylation footprinting followed by FENGC and BS-PCR (MAPit-FENGC). Cells were permeabilized, treated with M.CviPI to mark accessible GpC (hereafter, GC) sites in nuclear chromatin, and purified gDNA (2 μg or 4 μg as indicated) was processed by FENGC. Following target sequence scission with Taq or FEN1 and ligation capture using ˜300-nt and ˜450-nt oligos 1-T, 2-T, and 3-T (TABLE 1 and TABLE 2, Sheets Human ˜300-nt Targets and Human ˜450-nt Targets, respectively), the samples were subjected to either standard PCR or BS-PCR. The amplicons, directly analyzed by 1% agarose gel electrophoresis, are: ˜350-bp products (282-315 bp captured sequences plus ligated 47 bp universal priming sequences; lanes 1-3 and 7-9) obtained with Taq and standard PCR (lanes 1 and 7), FEN1 and standard PCR (lanes 2 and 8), and FEN1 and BS-PCR (lanes 3 and 9); ˜500-bp products (430-452 bp capture regions plus ligated 47 bp universal primers; lanes 4-6 and 10-12) obtained with Taq and standard PCR (lanes 4 and 10); FEN1 and standard PCR (lanes 5 and 11); and FEN1 and BS-PCR (lanes 6 and 12). All reactions yielded PCR products of the correct size, which were purified for NGS.

FIG. 19. High correlation of HCG and GCH methylation levels between biological replicates of MAPit-FENGC. Product libraries generated by MAPit-FENGC in FIG. 18 (lanes 3, 6, 9, and 12) were purified by AMPure XP beads and subjected to single-molecule real-time (SMRT) sequencing on a Pacific Biosciences (PacBio) Sequel instrument. High-fidelity circular consensus sequencing (CCS) reads (minimum of five sequencing passes) were aligned to the reference sequences for each of the 11 enriched promoters. Plotted are the methylation level of each HCG and GCH site from the 6 promoters that had 10 or more aligned CCS reads in the combined biological replicates for both the ˜300-nt and ˜450-nt target sequences (TABLE 3).

FIG. 20. High correlation of HCG and GCH methylation levels between two biological replicates of MAPit-FENGC and single-gene MAPit of the MLH1 promoter. For the MAPit-FENGC libraries constructed in FIG. 18 (lanes 6 and 12) and sequenced as described in FIG. 19, the methylation level of each HCG and GCH site was plotted for the 438-bp MLH1 promoter sequence (upper two panels; TABLE 2, Sheet Human ˜450-nt Targets). H5mCG and G5mCH levels were also plotted for the same 438 bp of the MLH1 promoter that overlapped a single 732-bp BS-PCR product (lower two panels) that was amplified from deaminated GBM L0 gDNA using two primers specific for MLH1 promoter sequence (TABLE 1, Sheet 1).

FIG. 21. Strong correlation of H5mCG and G5mCH levels in MAPit-FENGC for ˜300-nt and ˜450-nt target sequences processed by BS-PCR. High-fidelity PacBio Sequel CCS reads with five or more sequencing passes from the MAPit-FENGC product libraries constructed in FIG. 18, lanes 3, 6, 9, and 12 were aligned to the 11 promoter reference sequences (TABLE 2, Sheets Human ˜300-nt Targets and Human ˜450-nt Targets). The plotted values are the methylation level of each HCG and GCH site from 6 promoters with 20 or more aligned CCS reads in the combined biological replicates (TABLE 3) and in the overlapping region between the ˜300-nt and ˜450-nt target sequences.

FIG. 22. Validation of MAPit-FENGC by single amplicon BS-PCR. (A) Strong correlation between the fraction of methylation of each HCG and GCH site located within 438 bp of overlapping MLH1 sequence. PacBio CCS sequences of ˜450-nt from the 11 promoter panel (TABLE 2, Sheet Human ˜450-nt Targets) were enriched by MAPit-FENGC from GBM L0 gDNA followed by BS-PCR as presented in FIG. 18 and analyzed in FIG. 19 and FIG. 20. The BS-PCR CCS reads were obtained from a single 732-bp product amplified from the same deaminated GBM L0 DNA with primers MG03791 and MG03792, specific for MLH1 (TABLE 1, Sheet 1). (B) Nearly identical methylation levels at HCG and GCH sites within MLH1 regions that overlap between CCS reads from the 732-bp BS-PCR amplicon and the 300 bp and 438 bp MAPit-FENGC amplicons (TABLE 2, Sheets Human ˜300-nt and Human ˜450-nt Targets).

FIG. 23. FENGC sequence capture followed by deamination using enzymatic methyl conversion and amplification (EM-PCR). Genomic DNA (500 ng) from GBM L0 probed by M.CviPI was used for FENGC of 119 target sequences of ˜450 nt from promoters of human genes encoding products with functions in DNA repair and cancer (TABLE 1 and TABLE 2, Sheets Human ˜450 nt Targets). The captured sequences were purified by MinElute Kit (Qiagen), AMPure XP beads (Beckman Coulter), or NEBNext beads (NEB) as indicated, then subjected to EM-PCR (Sun et al., 2021). The amplification products were directly analyzed by 1% agarose gel electrophoresis. Based on the abundant ˜500-bp product (˜450-nt target sequences plus 47 bp universal primers), the latter two purification methods yielded favorable results.

FIG. 24. Optimization of primer concentrations for preferred FENGC followed by EM-PCR. Genomic DNA (1 μg) from HCT116 cells (not probed with M.CviPI) was input to FENGC of 119 target sequences of ˜450 nt from promoters of human genes encoding products with functions in DNA repair and cancer (TABLE 1 and TABLE 2, Sheets Human ˜450 nt Targets). Oligo U1-T (1 μl of indicated concentration) and a separate stock mixture of flap oligos 1-T and flap oligos 2-T (2 μl of indicated concentration) were first added to each reaction, with and without FEN1 as indicated. Next, without purification, separate stock mixtures of oligo U2 (1 μl of indicated concentration) and oligos 3-T (2 μl of indicated concentration) were added for ligation followed by EM-PCR. The amplification products were directly analyzed by 1% agarose gel electrophoresis. (A) Titration of primer concentrations demonstrated that including 10 μM each of oligos U1-T and U2 provides the optimal FEN1-dependent amplification of the EM-PCR product (compare lane 4 with lanes 2 and 6). (B) Specific EM-PCR product yield increased with more added flap oligos 1-T, flap oligos 2-T, and oligos 3-T. Therefore, their summed concentration should be calculated to be close to but less than the concentration of each of oligos U1-T and U2 (TABLE 12).

FIG. 25. Comparison of preferred FENGC followed by EM-PCR with DNA fragmented by SpeI digestion versus sonication. The indicated amounts of gDNA isolated from GBM L0 cells probed with M.CviPI were input to preferred FENGC directly after fragmentation by (A) SpeI digestion or sonication versus (B) sonication. The 5′ flaps of the 119 sequences of interest were cleaved by 32 U FEN1 within double flap structures with 3′ flaps comprised of one T. The EM-PCR amplification products were directly analyzed by 1% agarose gel electrophoresis. Note that sonication is superior to SpeI digestion in (A), and specific product was detected with as little as 50 ng input gDNA in (B).

FIG. 26. Uniform product lengths within purified libraries constructed by MAPit-FENGC enrichment. MAPit-FENGC with EM-PCR of 119 promoter sequences from DNA repair and cancer-associated genes of ˜450 nt in length (TABLE 1 and TABLE 2, Sheets Human ˜450 nt Targets) was performed on 500 ng of gDNA of each of the indicated cell materials. The preferred FENGC protocol (FIG. 1) was used to construct these and all libraries in subsequent figures. Two independent cultures of each cell type (NSC, human neural stem cells) were assayed as indicated by suffixes −1 and −2. Five hundred nanograms total gDNA of a 0.1%:99.9% mixture of L0:NSC gDNA were also subjected to MAPit-FENGC with EM-PCR for the 119 targets of ˜450 nt. Amplified, purified products (demarcated with arrowhead) are ˜500 bp (430-452 bp captured sequences plus 47 bp universal primers). The product libraries were purified for EM-seq (Sun et al., 2021) with 0.65×AMPure XP beads and analyzed with an Agilent TapeStation D5000 system.

FIG. 27. PacBio CCS read length distribution for sequenced MAPit-FENGC libraries. The purified EM-seq libraries prepared by MAPit-FENGC in FIG. 26 for the two biological replicates of NSC and GBM L0 were sequenced on a PacBio Sequel instrument. Percentages of different-length CCS reads are plotted as a histogram with 20-nt bins. The highest frequency of obtained reads was of the expected 500 nt, including both universal oligo sequences. Small peaks of ˜1,000 nt in the L0 histograms likely correspond to amplicon dimers that arose during ligation of sample-specific PacBio barcodes. Suffixes −1 and −2 denote each of the two independent biological replicates.

FIG. 28. PacBio CCS read length distribution for additional sequenced MAPit-FENGC libraries. The purified EM-seq libraries prepared by MAPit-FENGC in FIG. 26 for the two biological replicates of the 0.1% L0:99.9% NSC mixtures and GBM Nx18-25 were sequenced with the PacBio Sequel platform. Percentages of different-length CCS reads plotted as a histogram with 20-nt bins demonstrates the highest frequency of obtained reads was of the expected ˜500 nt, including both universal oligo sequences. Small peaks of ˜1,000 nt likely correspond to amplicon dimers that arose during ligation of sample-specific PacBio barcodes. Suffixes −1 and −2 denote each of the two independent biological replicates.

FIG. 29. MAPit-FENGC using EM-PCR results in low percentages of off-target reads. Percentages of CCS reads unmapped to the human genome, mapped to human genome but off target for the 119 regions, and on target (TABLE 4) are shown for the eight different sequenced MAPit-FENGC libraries as described in the legends of FIG. 26, FIG. 27, and FIG. 28. Suffixes −1 and −2 denote each of the two independent biological replicates.

FIG. 30. GC content has a moderate, negative correlation with the number of obtained MAPit-FENGC CCS reads. For the NSC and GBM L0 EM-seq libraries described in FIG. 26 and FIG. 27, high-fidelity CCS reads were filtered further for ≥95% conversion of HCH to HTH, i.e., not HCG or GCH, that also covered ≥95% of each reference sequence length in both biological replicates for each of the 119 MAPit-FENGC targets (TABLE 5). Plotted is the LOESS fit line of the natural logarithm transformation of the read number+1 versus the GC content for each of the 119 targets.

FIG. 31. GC content has a moderate, negative correlation with the number of obtained MAPit-FENGC CCS reads. PacBio Sequel CCS reads from the EM-seq libraries generated by MAPit-FENGC of gDNA from the duplicate Nx18-25 samples and 0.1% L0:99.9% NSC gDNA mixtures. CCS read filtering and the format of plotted data are described in the FIG. 30 legend.

FIG. 32. High reproducibility of MAPit-FENGC utilizing EM-PCR. Plotted are the correlations between the fraction of methylation of each HCG and GCH site in the two independent biological duplicates of the eight MAPit-FENGC libraries containing the enriched 119 promoter target sequences for the indicated samples described in FIG. 26, FIG. 27, and FIG. 28. Only targets with 10 or more aligned CCS reads with ≥95% conversion of HCH and ≥95% coverage of its reference sequence length in both replicates were included (TABLE 5). This equated to 64 targets for NSC, 52 targets for L0, 64 targets for Nx18-25, and 63 targets for the 0.1% L0:99.9% NSC gDNA mixtures.

FIG. 33. Strong correlation between MAPit-FENGC using EM and bisulfite conversion. Plotted is the fraction of methylation of each HCG and GCH site within 6 targets of ˜450 nt having 20 or more reads aligned in both EM-converted (TABLE 5) and bisulfite-converted samples (TABLE 3) in the combined biological replicates for the sequenced GBM L0 libraries.

FIG. 34. The POLD4 promoter, an example of a partially methylated promoter with a prominent, accessible nucleosome-free region (NFR) as detected by MAPit-FENGC with EM-seq. The GBM L0 libraries and sequenced CCS reads are as described in FIG. 26 (lanes 5 and 6) and FIG. 27, respectively. Shown is a methylscaper plot of 1,000 molecules randomly chosen from 2,139 obtained high-fidelity PacBio Sequel CCS reads with ≥5 SMRT sequencing passes, additionally filtered for ≥95% conversion, and ≥95% coverage of each reference sequence length (TABLE 5). To aid pattern recognition, the molecules were subjected to unsupervised hierarchical clustering and plotted by methylscaper {PMID: 34125875}. Each row of pixels depicts the epigenetic features of one molecule or epiallele from one cell recorded in one 447-nt CCS read from the POLD4 gene promoter, −297 to +150 relative to the transcription start site (TSS; bent arrow indicating direction of transcription). Plotted CCS reads yield a methylation pattern for HCG and GCH sites (vertical hashes at top and vertical lines crossing each panel with the distances separating them drawn to scale) as plotted in the left and right panels, respectively. Overlapping GC and CG sites, i.e., GCG are omitted to avoid ambiguity of methylation by exogenously added M.CviPI (GC specificity) and endogenous DNA methyltransferases (CG specificity), respectively. As indicated in the key at bottom, two or more consecutively methylated HCG are connected by red (left panel). Two or more consecutively accessible GCH, i.e., not bound by protein and hence accessible to and methylated by M.CviPI, are connected by yellow (right panel). Two or more consecutively unmethylated sites, either HCG or GCH, are connected by black. Gray designates border transitions between methylated and unmethylated HCG sites as well as between accessible and inaccessible GCH sites. Unaligned sequence varying from the hg38 genome reference is plotted in white. The molecules in both panels are displayed in the same top-to-bottom order, thereby linking the patterns of DNA methylation (left panel) and chromatin accessibility (right panel) in each molecule. Nucleosomes impair access of M.CviPI to 147 bp DNA, the length of DNA tightly wrapped around the histone protein core. Therefore, note in the right panel that most promoter copies exhibit two nucleosome-length protections against methylation by M.CviPI (full and partial blue ellipses labeled −1 and +1, respectively, drawn to scale), flanking a prominently accessible NFR. A fraction of NFR-bearing promoter copies harbors a short span of endogenous 5mCG (left panel), which does not correlate with the presence or absence of an NFR (right panel).

FIG. 35. The ALKBH2 promoter, a second example of MAPit-FENGC utilizing EM-seq. The GBM L0 library, sequenced CCS reads, CCS read filtering are as described in FIG. 26, FIG. 27, and FIG. 34, respectively. The majority of the 979 aligned PacBio Sequel 436-nt filtered CCS reads (TABLE 5) from −282 to +154 from L0 cells revealed 5mCG at the limit of detection, except for a low level at the TSS (left panel). In addition, a highly accessible NFR is bordered upstream by a large footprint, consistent with a variably positioned −1 nucleosome (halved blue ellipse; right panel). The NFR is prominently occupied by a relatively short, uniformly located footprint, consistent with occupancy by a sequence-specific DNA-binding factor (right panel; black rectangle at top). The CCS read filtering, content of the panels, symbols, and key at bottom are as described in the legend of FIG. 34.

FIG. 36. MAPit-FENGC with EM-PCR detects differentially methylated and differentially accessible chromatin in NSC versus GBM L0 cells. Both cell lines were treated separately with M.CviPI plus SAM methylation cofactor. Purified gDNA (500 ng) was used as input to construct the MAPit-FENGC libraries in FIG. 26 (NSC, lanes 1 and 2; GBM L0, lanes 5 and 6), using the panel of 119 promoter targets (TABLE 1 and TABLE 2, Sheets Human ˜450-nt Targets). The shown methylscaper plot has 3,460 and 3,889 450-nt filtered CCS reads from the EPM2AIP1 promoter (−143 to +307) from (A) NSC and (B) L0 cells, respectively (TABLE 5). The CCS read filtering, content of the panels, symbols, and key at bottom are as described in the legend of FIG. 34. The white rectangle represents the first portion of EPM2AIP1 protein coding sequence. In NSC (A), the EPM2AIP1 promoter has no to low 5mCG (left panel) and harbors mostly accessible, open chromatin (right panel) occupied by a sequence-specific DNA-binding factor (black rectangle at top) and a variably positioned +1 nucleosome (blue partial and full ellipses). By contrast, in GBM L0 (B), the promoter is hypermethylated, closed, and exhibits only limited accessibility within relatively short nucleosomal linker sequences, visible in a subpopulation of cells.

FIG. 37. MAPit-FENGC efficiently detects a 0.1% subpopulation of hypermethylated epialleles within a heterogeneous sample. Permeabilized GBM L0 cells and NSC were treated separately with M.CviPI and SAM, and purified gDNA was mixed in a ratio of 0.1% L0:99.9% NSC. Next, 500 ng of the gDNA mixture was processed by the preferred FENGC protocol with EM-PCR to construct the EM-seq libraries in FIG. 26 (lanes 7 and 8), yielding the high-fidelity PacBio CCS read distribution shown in FIG. 28. Shown are aligned, filtered CCS reads (1,781 total; TABLE 5) plotted by methylscaper. The CCS read filtering, content of the panels and key at bottom are as described in the legend of FIG. 34. The white rectangle represents the first portion of EPM2AIP1 protein coding sequence. Ten reads in the gDNA mixture showed dense H5mCG, consistent with originating from L0 cells (red arrowhead). A proportions test in R concluded that the observed proportion of molecules (10 hypermethylated:1,771 unmethylated epialleles) is indeed at least or greater than 0.1% (****, P<0.0001).

FIG. 38. MSHS exemplifies a gene with a remarkably accessible promoter. MAPit-FENGC was conducted on two independent replicate cultures of human non-cancerous NSC and second GBM Nx18-25 that were treated with M.CviPI. A 119-target panel of human promoters (TABLE 1 and TABLE 2, Sheets Human ˜450-nt Targets) was enriched by preferred FENGC to construct the EM-seq libraries in FIG. 26 (lanes 3 and 4), yielding the high-fidelity PacBio CCS read distribution shown in FIG. 28. CCS read filtering as well as the content of the panels and key at bottom are as described in the FIG. 34 legend. Methylscaper plots of 447-nt filtered CCS reads aligned to the MSHS promoter (−302 to +145) from (A) two independent NSC cultures (top panels; 132 and 241 reads) and (B) two independent GBM Nx18-25 cultures (bottom panels; 170 and 192 reads) (TABLE 5). Suffixes −1 and −2 denote each of the two independent biological replicates. Each HCG and GCH position (in base pairs) along the MSHS amplicon is indicated at bottom of its respective panel. The black bar indicates 147 bp, the size of a nucleosome core lacking the linker; this and other features and the distances between them are drawn to scale. A heterogeneous-sized footprint is marked by a black rectangle. Almost all MSHS promoter molecules across all 4 samples (733 of 735) had ≥10 methylated GCH sites, demonstrating highly accessible chromatin. This high degree of openness rules out incomplete cell permeabilization and chromatin probing with M.CviPI as trivial reasons for differential accessibilities observed between other loci in the same or different samples.

FIG. 39. Identification of chromatin architectures differentially methylated (H5mCG), accessible (G5mCH), or both using MAPit-FENGC with EM-seq. Generalized estimating equations were used to model the effect of cell line (human NSC versus GBM Nx18-25) on the per molecule proportions of endogenous H5mCG (number H5mCG/(number H5mCG+number HCG); left panels) and G5mCH (number G5mCH/(number G5mCH+number GCH); right panels). Only targets with ≥50 total CCS reads in the combined replicates (≥22 in any single replicate) were considered (TABLE 5), with filtering as described in the FIG. 34 legend. Errors were modeled as normally distributed, and the correlation structure was assumed exchangeable within each replicate. The geeglm function from the geepack v1.3-2 R package was used in R version 4.1.0. P values were corrected for multiple testing using the Bonferroni method with an alpha of 0.05 to control the false discovery rate (TABLE 6). Using criteria of P<0.05 and ≥0.05 differential in the proportion of either H5mCG or G5mCH between NSC and GBM Nx18-25, 57% (31/54) of the evaluated promoters showed epigenetic alterations (TABLE 6, Sheets 1 and 2). With more stringent cutoffs, the percentages of promoters with ≥0.1, ≥0.2, and ≥0.4 differentials in H5mCG or G5mCH were 26% (14/54), 13% (7/54), and 7.4% (4/54), respectively (TABLE 6, Sheets 3-5). These promoters include human (A) CD44, (B) CCN4, and (C) HIST1HB1 for which the data were rendered as violin plots for the two independent replicates (Rep 1 and 2) of NSC and GBM Nx18-25. Plotted is the proportion of H5mCG or G5mCH for each molecule (black dots), the median (horizontal line), interquartile range of methylation levels (box), and the smoothed probability density at different methylation levels (gray area).

FIG. 40. CD44 promoter exemplifies detection by targeted MAPit-FENGC with EM-seq of differential epigenetic alterations associated with gene silencing in cancer. (A) Methylscaper plots of 441-nt filtered CCS reads aligned to the CD44 promoter (−270 to +171) from NSC (left two panels; 141 reads) and GBM Nx18-25 (right two panels; 96 reads) (TABLE 5). CCS read filtering as well as the content of the panels and key at bottom are as described in the FIG. 34 legend. (B) Methylation level of each HCG (upper panel) and GCH (lower panel) motif tabulated from the filtered CCS reads plotted in (A). (C) Cumulative distribution function of NFR length, considering all aligned CCS reads from NSC and Nx18-25. The mean NFR length for NSC is 118 bp and for Nx18-25 is 72.0 bp, P<0.0001. (D) Relative expression level of CD44 in NSC and Nx18-25. Reverse transcription (RT)-qPCR was done using Taqman assay (Life Technologies, 44-449-63) with CD44-specific probe (ThermoFisher Scientific, Hs01075861_m1) for three independent biological replicates (mean±SD; **P<0.01). Taken together, compared with NSC where the CD44 promoter is relatively unmethylated and open with longer NFRs, the promoter becomes hypermethylated and closed in GBM Nx18-25, consistent with strong silencing of expression.

FIG. 41. CCN4 promoter exemplifies detection by targeted MAPit-FENGC with EM-seq of differential epigenetic alterations associated with upregulated expression in cancer. (A) Relative expression level of CCN4 transcript in NSC and GBM Nx18-25. RT-qPCR was done using Taqman assay (Life Technologies, 44-449-63) with CCN4-specific probe (ThermoFisher Scientific, Hs00180245_m1) for three biological replicates (mean±SD; *P<0.05). (B) Methylscaper plots for filtered 447-nt CCS reads of CCN4 (−279 to +168 bp) from NSC (left two panels; 1,116 molecules) and Nx18-25 (right two panels; 1,143 molecules) (TABLE 5). The CCS read filtering, content of the panels, and key at bottom are as described in the legend of FIG. 34. The white rectangle indicates the first portion of CCN4 protein coding exon 1. The bracketed subset of reads contains footprint evidence of a sequence-specific factor bound to a NFR (black rectangle). The difference in H5mCG between NSC and GBM Nx18-25 was statistically significant (TABLE 6); however, the difference in accessibility was not, probably because of an uncharacteristically high level of accessibility in molecules displaying H5mCG. The straight arrow marks a known GA to A variant (dbSNP, rs548251181) present in approximately half of the alleles from NSC and Nx18-25 (TABLE 7, Sheet Indels), which does not align to the hg38 reference genome and is plotted in white. This variant was verified by MAPit-FENGC genotyping using standard PCR amplification, i.e., without EM-seq conversion (TABLE 7). (C) Methylation level of each HCG (upper) and GCH (lower) motif from −279 to +168 bp relative to the CCN4 TSS tabulated from the filtered CCS reads plotted in (C). (D) Cumulative distribution function of NFR length, considering all aligned filtered CCS reads from NSC and Nx18-25. The mean NFR length for NSC is 89.4 bp and for Nx18-25 is 132 bp, P<0.0001. Taken together, relative to NSC, the CCN4 promoter in GBM Nx18-25 is less methylated and contains more open and longer NFRs, consistent with the strong transcriptional induction in the tumor-derived cells.

FIG. 42. HIST1H1B promoter exemplifies detection by MAPit-FENGC with EM-seq of differential epigenetic alterations in a minority subpopulation of cells, despite no significant difference in bulk transcript abundance. (A) Methylscaper plots of filtered 445-nt CCS reads of the HIST1H1B promoter (−246 to +199) from NSC (left two panels; 926 reads) and GBM Nx18-25 (right two panels; 953 reads) (TABLE 5). CCS read filtering as well as the content of the panels and key at bottom are as described in the FIG. 34 legend. The white rectangle partially covered by nucleosome +1 indicates the first portion of HIST1H1B protein coding sequence. The black rectangle marks likely binding of a sequence-specific transcription factor, whereas the gray rectangle corresponds to a relatively large footprint, but too small to be a nucleosome, that may correspond to paused RNA polymerase II. Note the range of promoter configurations (clusters marked 1-7), with less molecules populating summed clusters 1 and 2 in GBM Nx18-25 compared with NSC. In addition, the percentage of hypermethylated, inaccessible epialleles (cluster 6) was elevated in GBM versus NSC (4% versus 0.6%). (B) Methylation level of each HCG (upper panel) and GCH (lower panel) motif tabulated from the CCS reads plotted in (A). The bracket indicates the area around the TSS (−80 to +45) that shows a significant decrease in accessibility in Nx18-25 (TABLE 6, P<0.0001). (C) Relative expression of HIST1H1B in NSC and Nx18-25. RT-qPCR was done using Taqman assay (Life Technologies, 44-449-63) with HIST1H1B-specific probe (ThermoFisher Scientific, Hs01075861_m1) for three biological replicates (mean±SD). The expression levels are not statistically different. Taken together, the data exemplify the power of single-molecule MAPit-FENGC to detect epigenetic alterations in minority populations of cells. The hypermethylated and closed HIST1HIB promoter copies in GBM are highly unlikely to support active transcription but are too few in number to decrease a bulk, population-averaged measurement such as RT-qPCR.

FIG. 43. Length distribution of EM-seq high-fidelity CCS reads for ˜940-nt MAPit-FENGC libraries. Two independent biological replicates of GBM Nx18-25 cells were treated with M.CviPI. Subsequently, 800 ng or 400 ng of purified gDNA from each biological replicate was processed by preferred FENGC using oligos 1-T, 2-T, and 3-T for 45-940-nt targets (TABLE 1 and TABLE 2, Sheets Human ˜940 nt Targets). The four capture product libraries were sequenced using the PacBio Sequel platform. Shown is the length distribution of CCS reads plotted in 20-nt intervals. Suffixes −1 and −2 denote each of the two independent biological replicates.

FIG. 44. Low percentages off-target reads for MAPit-FENGC of ˜940-nt sequences of interest with EM-PCR. Shown are the percentages of high-fidelity CCS reads (≥5 SMRT sequencing passes) unmapped to the human genome, mapped to human genome but off target for the 45 regions, and on target (TABLE 8) for the 4 different GBM Nx18-25 MAPit-FENGC libraries described in the FIG. 43 legend. Suffixes −1 and −2 denote each of the two independent biological replicates.

FIG. 45. High correlation of HCG and GCH methylation levels between two independent biological replicates of MAPit-FENGC for the ˜940-nt target sequences. EM-seq high-fidelity CCS reads (≥5 SMRT sequencing passes) were filtered further for ≥95% conversion, and ≥95% coverage of the reference sequence length (TABLE 9). Plotted values are the fraction of methylation of each HCG and GCH site in 13 targets for 800 ng input and 12 targets for 400 ng input of Nx18-25 gDNA that have 14 or more CCS reads aligned in each biological replicate of each cell type (TABLE 9).

FIG. 46. High correlation of HCG and GCH methylation levels with different input amounts of gDNA for MAPit-FENGC of the ˜940-nt targets. Plotted values are the methylation level of each HCG and GCH site in 13 targets from GBM Nx18-25 cells using CCS reads filtered for ≥15× coverage in the combined biological replicates of both input gDNA amounts (TABLE 9) but otherwise as described in the FIG. 45 legend.

FIG. 47. High correlation of HCG and GCH methylation levels between two independent biological replicates of MAPit-FENGC for the ˜940-nt and ˜450-nt target sequences. Plotted are the methylation level of each HCG and GCH site in 13 targets from GBM Nx18-25 cells having ≥15× coverage in the combined biological replicates of both the ˜450-nt and ˜940-nt MAPit-FENGC samples (TABLE 5 and TABLE 9). Other CCS read filtering parameters were as described in the FIG. 45 legend.

FIG. 48. Long-read MAPit-FENGC using EM-seq phases multiple epigenetic features and reveals novel regulatory insights. The indicated lengths of promoter sequences from the locus containing the divergently transcribed EPM2AIP1 and MLH1 genes were captured and enriched by preferred FENGC from gDNA isolated from two biological replicates of M.CviPI-treated GBM Nx18-25 cells (FIG. 26, lanes 3 and 4). Sequences in (A) and (B) were among those enriched by FENGC for the 119-450-nt targets (TABLE 2, Sheet 2 and FIG. 28), whereas those in (C) were enriched for 45-940-nt targets from 800 ng gDNA (TABLE 2, Sheet 3 and FIG. 43 top). Shown and drawn to scale are methylscaper plots of molecules from the enriched regions: (A) 450 bp of EPM2AIP1, (B) 438 bp of MLH1, and (C) 937 bp of EPM2AIP1-MLH1, with all promoter coordinates indicated relative to MLH1 TSSa. White rectangles depict protein coding sequence. The black bar indicates 147 bp, the size of a nucleosome core lacking the linker; this and other features and the distances between them are drawn to scale. Patterns of methylation at CHG are plotted by methylscaper in (A) and the left panel of (C); patterns of methylation at GCH are plotted in (B) and the right panel of (C). Each HCG and GCH position (in bp) on each amplicon is indicated at bottom of each panel. CCS read filtering as well as the content of the panels and key at bottom are as described in the FIG. 34 legend. The short molecules in (A) and (B) are aligned to the long molecules in (C). Hierarchical clustering of the 937-bp molecules in (C) was weighted on the EPM2AIP1 half of the amplicon, whereas no sub-region was specified for clustering the two shorter amplicons in (A) and (B). Collectively, the three amplicons reveal three robust footprints (black rectangles numbered 1-3), corresponding to DNA-bound, sequence-specific transcription factors. The 937-bp molecules in (C), however, revealed a continuous NFR encompassing the EPM2AIP1 TSS, MLH1 TSSa, footprint 1 and footprint 3, the discovery of which would have otherwise required a third short amplicon. Moreover, strong co-occupancy of all three transcription factors and the two +1 nucleosomes was evident on the 937-bp molecules. In addition, based on the clustering of molecules from both short amplicons in (A) and (B), it is readily discerned that the +1 nucleosomes (blue ellipses) downstream of the EPM2AIP1 TSS and MLH1 TSSa occupy a continuum of positions, the EPM2AIP1+1 nucleosome more so. However, MLH1 TSSa+1 nucleosome positions were randomized when hierarchical clustering of the 937-bp molecules in (C) was weighted on the EPM2AIP1+1 nucleosome and vice versa (not shown). This strongly suggests that the positioning of the two +1 nucleosomes is regulated independently, a regulatory insight that cannot be derived from short reads/molecules.

FIG. 49. Additional examples of phasing multiple epigenetic features provided by long-read MAPit-FENGC using EM-seq. Promoter sequences of (A) 938 bp of divergently transcribed NPAT and ATM, (B) 930 bp of MSH2, and (C) 944 bp of CCN4 were captured and enriched by preferred FENGC from gDNA isolated from M.CviPI-treated GBM Nx18-25 cells (FIG. 26, lanes 3 and 4). All sequences were among those enriched for the 45-940-nt targets (TABLE 2, Sheet 3 and FIG. 43). Shown are methylscaper plots of methylation at HCG (left panels) and GCH (accessibility; right panels) of molecules from the enriched regions. The base pair position of each HCG and GCH in each amplicon is indicated at bottom of each respective panel. Promoter coordinates are indicated relative to the ATM TSS in (A) and relative to each single TSS in (B) and (C). White rectangles depict protein coding sequence. CCS read filtering as well as the content of the panels and key at bottom are as described in the FIG. 34 and FIG. 48 legends. The straight arrow in (C) marks a known GA to A variant (dbSNP, rs548251181; TABLE 7, Sheet Indels) plotted in white as described in the FIG. 41 legend. The pink rectangle denotes a 66-bp A-rich sequence (88% A) that exhibits variable-length non-alignment to the hg28 reference as well. Note that the NPAT-ATM (A) and MSH2 (B) promoters show NFRs with robust transcription factor footprints. In addition, more heterogeneously sized footprints (cyan rectangles) co-localize with the NPAT and MSH2 TSSs, possibly corresponding to paused RNA polymerase II. Furthermore, the 930-944 bp molecules from all three promoters enabled assessment of long-range nucleosome organization. The +1 nucleosome proximal to the NPAT TSS in (A) was particularly well positioned, whereas the more distal +2 and +3 nucleosomes were less so. By contrast, the long-range nucleosome organization in the MSH2 (B) and CCN4 (C) promoters was more disorganized and not apparent, respectively. Moreover, MAPit-FENGC of ˜800 bp of 5′ flanking sequence from MSH2 and CCN4 identified clear transitions between 5mCG depletion and hypermethylation.

FIG. 50. Target sequence length and GC content negatively correlate with filtered CCS read number obtained from primary mouse monocytes using MAPit-FENGC with EM-seq. Bone marrow was collected from the spines of four 4-months-old female C57BL/6J mice. Female mice were selected to examine expected epiallelic differences on the X chromosome between active and inactive gene copies. Extraneous tissues were removed from the spine, which was crushed and the homogenate filtered through a nylon mesh. Monocytes in the filtrate were enriched by a negative isolation protocol that depletes non-monocytes (Militenyi, 130-100-629). Purified monocytes were allowed to recover from the isolation procedure for 3 hr in growth medium before processing by the MAPit-FENGC protocol. For this experiment, the primers were designed by newly developed program, FENGC oligonucleotide designer (FOLD; github.com/albertoriva/FOLD). The programs searches an input file of gene names or genome coordinates for primers that avoid repeats and satisfy criteria of FIG. 1, such as locating the ‘overlapping’ residues that create 1-nt 3′ flaps. Other command-line options include, but are not limited to, increasing the length of default 500-nt sequences and percentage tolerance of departure from this specified length, specification of annotated TSS (e.g., RefSeq), and minimum and maximum primer melting temperature (Tₘ). To provide a computational solution for all desired 78 targets that satisfied all FOLD settings, target sizes were permitted to range from 474-987 nt (mean 620 nt; TABLE 1, Sheet 5 and TABLE 2, Sheet 4, Mouse ˜620-nt Targets). The resulting 78-flap adapter panel, including 77 genes with known or suspected roles in the cellular inflammatory response and 1 control promoter (Cox8a), was used for preferred FENGC enrichment. The four resulting mouse monocyte FENGC libraries were barcoded, pooled, and sequenced on a PacBio Sequel II instrument. Demultiplexed, high-fidelity CCS reads were aligned to the complete mm9 build of the mouse genome versus specific target reference sequences: 20-29% did not align to either reference, yielding 71-80% on-target reads. Overall, FENGC detected 71-75 targets (91-96%) with ≥1 read in each sample (TABLE 10 and TABLE 11). Data are plotted as the natural logarithm transformation of filtered CCS read number+1 versus (A) target length and (B) percentage GC content for each of the 78 targets.

FIG. 51. Reproducible epigenetic profiling of representative regions from primary mouse monocytes using MAPit-FENGC with EM-seq. High-fidelity CCS reads (≥5 SMRT sequencing passes; TABLE 10) were filtered further for ≥95% conversion of HCH to HTH and ≥95% coverage of each reference sequence length (TABLE 11) to avoid multiple alignments to homologous gene orthologs. Shown are methylscaper plots of methylation at HCG (left panel of each pair) and GCH (accessibility; right panel of each pair) of molecules from the enriched regions. Promoter sequences of (A) 474 bp of Hsf1, divergently transcribed with Bop1, (B) 514 bp of Btk from the X chromosome, (C) 605 bp of Pik3r3, and 576 bp of Tlr4 were captured and enriched by FENGC from gDNA isolated from M.CviPI-treated mouse monocytes. All sequences were among those enriched for the 78-620-nt targets (TABLE 1, Sheet 5 and TABLE 2, Sheet 4) by the preferred FENGC protocol. Each HCG and GCH position in base pairs along each amplicon is indicated at bottom of its respective Mouse 4 panel. The black bar indicates 147 bp, the size of a nucleosome core lacking the linker; this and other features and the distances between them are drawn to scale. Promoter coordinates at top are relative to the Hsf1 TSS in (A) and relative to each single TSS in (B), (C), and (D). The content of the panels and key at bottom are as described in the FIG. 34 legend. Note the highly consistent chromatin architectures detected at each locus (A-D) between the four different monocyte samples (Mouse 1-4). To determine rigorously if at least one mouse monocyte sample exhibited statistically significant epigenetic differences, smoothed moving averages (20-bp window) of DNA methylation and accessibility across each gene region were modeled using a mixed effects ANOVA. Testing was limited to 43 amplicons with ≥100 filtered CCS reads per sample and good diversity, i.e., absence of apparent duplicates (TABLE 11). Mean P values for H5mCG and G5mCH before correction for multiple testing were >0.90 (range 0.17-1.0) and all P values after correction were 1.0 (TABLE 11). This indicates that for each target, none of the four mouse monocyte samples had significantly different levels of either DNA methylation or chromatin accessibility. On examination of the patterns of DNA methylation and chromatin accessibility on single molecules, Hsf1 in (A) exemplifies an exceptionally open promoter in the four bone marrow-derived monocyte samples (2,333 of 2,334 molecules bearing large NFRs). As observed in FIG. 38, a locus with such a high degree of openness rules out incomplete cell permeabilization and chromatin probing as trivial bases for observed inter-locus differences in accessibility. Consistent with X inactivation, the Btk promoter from each mouse in (B) showed a cluster of highly accessible epialleles that were devoid of H5mCG and strongly anti-correlated with molecules displaying as few as two H5mCG. The Pik3r3 promoter in (C) harbored a remarkably well-positioned −1 nucleosome and incremental, preferential sliding of the +1 nucleosome, expanding/contracting the NFR mainly on one side. This is intriguing, given that most promoters, such as Tlr4 in (D) displayed movement of both the −1 and +1 nucleosomes, expanding/contracting the NFR on both sides.

FIG. 52. Reproducible epigenetic profiling of representative regions from primary mouse monocytes using MAPit-FENGC with EM-seq. Shown and drawn to scale are methylscaper plots of methylation at HCG (left panel of each pair) and GCH (accessibility; right panel of each pair) of molecules from the enriched regions. Promoter sequences of (A) 606 bp of Hsp90ab1, (B) 492 bp of Cxcr4, (C) 556 bp of Mapk15, and (D) 616 bp of Src were captured and enriched by preferred FENGC from gDNA isolated from M.CviPI-treated mouse monocytes. Promoter coordinates at top are relative to each single TSS in all panels. The FENGC target panel used to enrich sequences, CCS read filtering, and statistical test for epigenetic differences between different monocyte samples are as described in the FIG. 51 legend. As in FIG. 51, in this figure, none of the four mouse monocyte samples from each of the four targets exhibit significant epigenetic differences from the other three samples (TABLE 11). The content of the panels and key at bottom are as described in the FIG. 34 and FIG. 51 legends. As for the Tlr4 promoter in FIG. 51D, movement of both the −1 and +1 nucleosomes led to NFR contraction/expansion on both sides of the NRF in the promoters of Hsp90ab1 (A) and Cxcr4 (B). Surprisingly, the Cxcr4 promoter displayed a 36-bp zone of H5mCG (orange rectangle) in the accessible NFR, only 12 bp from a strongly footprinted TF. Compared to the above promoters, the Mapk15 gene body (C) (+1,660 to +2,215) was hypermethylated with arrays of randomly positioned nucleosomes and short, linker-length NFRs. Chromatin −571 to +45 of the TSS of Src (D) was similarly organized, consistent with low-level expression of Src in mouse monocytes {Schaum, 2018 #190}. Interestingly, a sequence in Src with strong CTCF binding site homology {Hashimoto, 2017 #188} (black rectangle) conferred clear protection against endogenous 5mCG in many molecules. Taken together with FIG. 51, the data demonstrate that MAPit-FENGC is effective at discerning epigenetic landscapes of purified primary cells, with striking inter-sample reproducibility.

TABLE 1. FENGC oligonucleotides used in this study.
TABLE 2. Characteristics of designed FENGC target sequences used in this study.
TABLE 3. CCS reads aligned to 11 human targets of ˜300 nt vs. ˜450 nt used in FENGC assay development with standard PCR vs. BS-PCR.
TABLE 4. CCS reads on- and off-target for 119-450-nt human targets in MAPit-FENGC.
TABLE 5. Filtered CCS reads aligned to 119-450-nt human targets in MAPit-FENGC using EM-seq.
TABLE 6. Differential H5mCG and G5mCH determined by MAPit-FENGC using EM-seq of ˜450-nt human targets with ≥50 filtered reads.
TABLE 7. SNPs and indels detected by FENGC using standard PCR of 119-450-nt human targets.
TABLE 8. CCS reads on- and off-target for 45-940-nt human targets in MAPit-FENGC using EM-seq.
TABLE 9. Filtered CCS reads aligned to 45-940-nt human targets in MAPit-FENGC using EM-seq.
TABLE 10. CCS reads on- and off-target for 78-620-nt mouse targets in MAPit-FENGC using EM-seq.
TABLE 11. Filtered CCS reads aligned to 78-620-nt mouse targets in MAPit-FENGC using EM-seq and P values.
TABLE 12. Recommended FENGC oligonucleotide concentrations for different numbers of targets.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference.

Generally, nomenclatures used in connection with, and techniques of, cell and tissue culture, molecular biology, immunology, microbiology, genetics, protein, and nucleic acid chemistry, and hybridization described herein are those well known and commonly used in the art. The methods and techniques of the present invention are generally performed according to conventional methods well known in the art and as described in various general and more specific references that are cited and discussed through the present specification unless otherwise indicated.

The terms “complement,” “complementary,” or “complementarity” as used herein with reference to polynucleotides (i.e., a sequence of nucleotides such as an oligonucleotide or a genomic nucleic acid) related by the base-pairing rules. The complement of a nucleic acid sequence as used herein refers to an oligonucleotide which, when aligned with the nucleic acid sequence such that the 5′ end of one sequence is paired with the 3′ end of the other, is in “antiparallel association.” For example, the sequence 5′-A-G-T-3′ is complementary to the sequence 3′-T-C-A-5′. Certain bases not commonly found in natural nucleic acids may be included in the nucleic acids of the present invention and include, for example, inosine, 7-deazaguanine, and 5-methylcytosine. Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength, and incidence of mismatched base pairs. Complementarity may be “partial” in which only some of the nucleic acids' bases are matched according to the base-pairing rules. Or, there may be “complete,” “total,” or “full” complementarity between the nucleic acids.

The terms “Flap endonuclease 1” and “FEN1” as used herein refer to a nucleolytic enzyme that acts as both 5′-3′ exonucleases and structure-specific endonucleases on specialized DNA or RNA structures that occur during the biological processes of DNA replication, DNA repair, and DNA recombination. FENs can also cleave RNA, i.e., when the oligo complex hybridizes to an RNA target (Lyamichev et al., 1993). This contributes to the removal of RNA primers in Okazaki fragments during lagging strand DNA synthesis. The endonuclease activity of FEN1 was initially identified as acting on a DNA duplex which has a single-stranded 5′ overhang on one of the strands (termed a “5′ flap,” hence the name flap endonuclease). FEN1 catalyzes hydrolytic cleavage of the phosphodiester bond at the junction of single- and double-stranded DNA.

The terms “enrichment” and “enriching” as used herein refer to capturing selective genomic regions of interest for targeted sequencing of just the coding regions, specific genes, or segments of chromosomes that are relevant to a particular experiment or disease.

The term “oligonucleotide” as used herein refers to a short polymer composed of deoxyribonucleotides, ribonucleotides, or any combination thereof. Oligonucleotides are generally between about 10, 11, 12, 13, 14, 15, 20, 25, or 30 to about 150 nucleotides (nt) in length, more preferably about 10, 11, 12, 13, 14, 15, 20, 25, or 30 to about 70 nt.

The term “source sequence” as used herein refers to a sequence in which a sequence of interest is contained. The sequence of interest is cleaved from the source sequence and enriched. The source sequence can be comprised of DNA, such as in a genome, and RNA.

The terms “Taq polymerase” and “Taq” as used herein refer to a thermostable DNA polymerase I named after the thermophilic eubacterial microorganism Thermus aquaticus, from which it was originally isolated by Chien et al. in 1976. It is frequently used in the polymerase chain reaction (PCR), a method for greatly amplifying the quantity of short segments of DNA.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless specifically stated or obvious from context, as used herein, the term “about,” or “approximately,” or symbol “˜” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Unless specifically stated or obvious from context, as used herein, the singular form “a,” “an,” and “the” include plural references. For example, the term “an oligonucleotide” or “an oligo” includes a plurality of oligos, including mixtures thereof.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by prior disclosure. Further, the dates of publication provided could be different from the actual publication dates that may need to be independently confirmed.

Unless otherwise indicated, the present disclosure is not limited to particular materials, reagents, reaction materials, manufacturing processes, or the like, as such can vary. It is also to be understood that the terminology used herein is for purposes of describing particular embodiments only and is not intended to be limiting. It is also possible in the present disclosure that steps can be executed in different sequence where this is logically possible.

### DETAILED DESCRIPTION

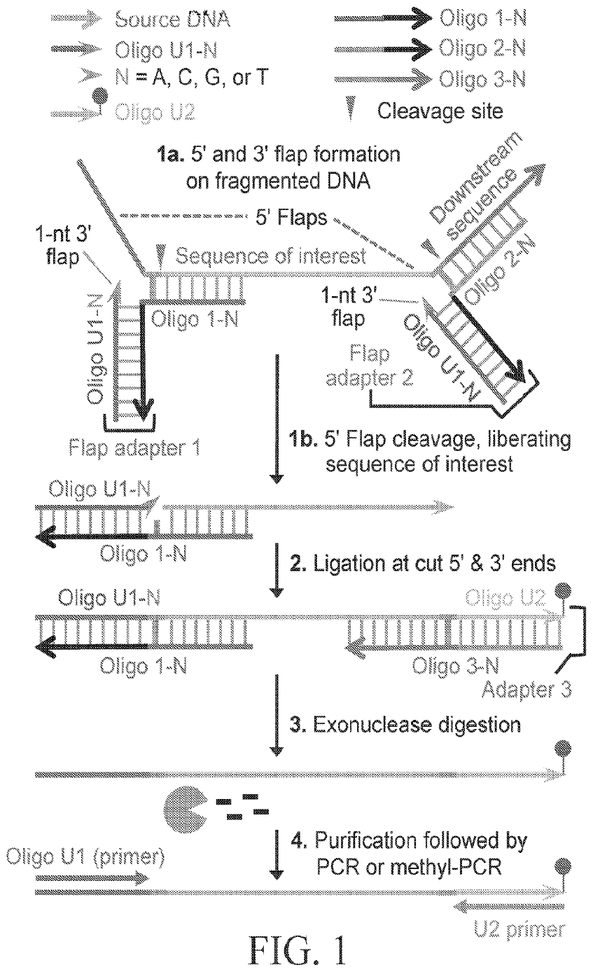

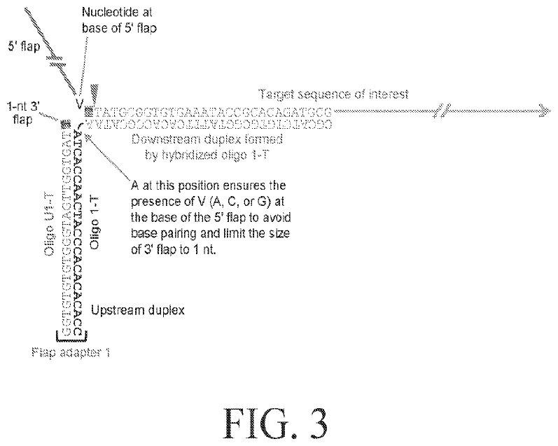

The methods disclosed herein for sequence enrichment exploit the many advantages of DNA strand capture and amplification afforded by patch ligation PCR (Varley and Mitra, 2008; Varley and Mitra, 2010), while eliminating the restrictive requirement to create capturable fragments using a select few restriction endonucleases. In this embodiment, two flap adapters, each consisting of an ˜50-mer unmodified oligo bound to a first complementary universal PCR priming oligo, have been designed to form unpaired 5′ flaps, with a preferred unpaired 1-nt 3′ flap, at both the 5′ and 3′ ends of a linear target DNA strand (FIG. 1 and FIG. 3). In some embodiments the oligos comprising the flap adapters may be shorter or longer, for example, 11 to 150 nt, contain base modifications, for example, 5mC, or some combination thereof. Cleavage of a plurality of the flap structures by the 5′ flap cleavage activity of a single thermostable enzyme, such as Thermococcus 9° N™ FEN1 (hereafter, FEN1) or Taq DNA polymerase I (hereafter, Taq) (Lyamichev et al., 1993; Finger et al., 2012), releases a plurality of single-stranded DNA (ssDNA) fragments with two defined ends. The 5′ end of each target is ligated to the first universal oligo, whereas each 3′ end is contacted by a third target-complementary oligo that positions a second universal oligo for ligation. The three oligos that hybridize to the target strands are inexpensive as they are unmodified, i.e., do not contain methylated or biotinylated bases. Only the second universal oligo is modified by covalent modification that blocks its 3′ hydroxyl group, which inconsequential to overall cost as that same universal oligo is always used. All oligos, therefore, are readily synthesized at high quality and yield as well as low cost, without needing purification. Enrichment of many target sequences can be conducted in the same reaction at a fraction of the cost of strategies based on recombinant CRISPR/Cas/gRNA or oligo hybridization and pull-down. Furthermore, high specificity is achieved because neither flap incision nor ligation tolerate DNA mismatches (Wu and Wallace, 1989; Kaiser et al., 1999; Lyamichev et al., 1999; Lyamichev et al., 1999; Hall et al., 2000; Lyamichev et al., 2000; Tsutakawa et al., 2017), and target DNA strands ligated to both universal priming sequences are preferentially PCR amplified subsequent to extensive exonuclease digestion.

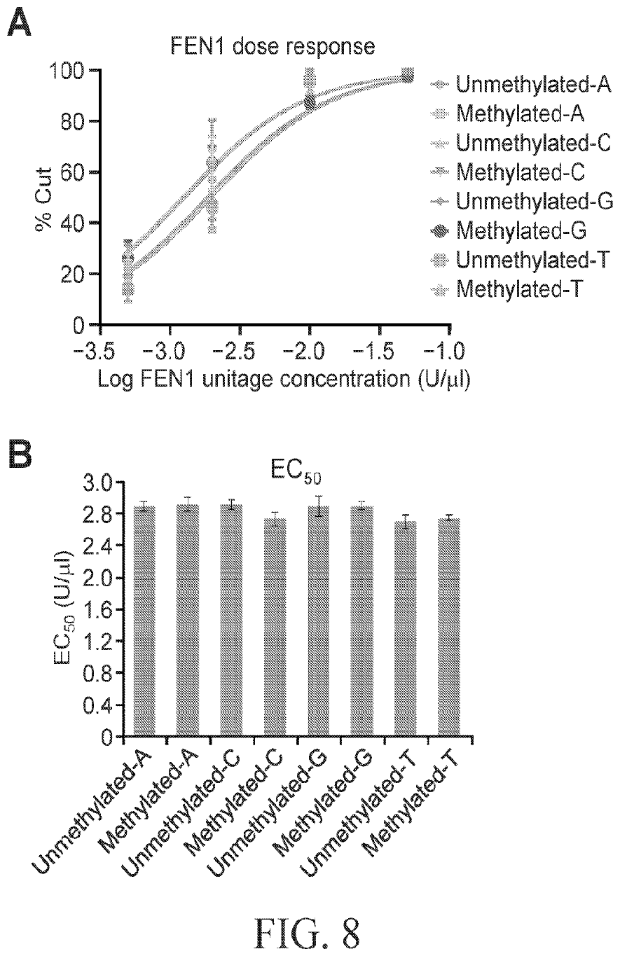

In addition to genotyping, because it was shown that endonuclease activity of FEN1 is unaffected by 5mC (FIG. 8), an aliquot of the same FENGC-enriched DNA can be processed in parallel for concurrent detection of DNA methylation and chromatin accessibility, termed MAPit-FENGC. In certain embodiments, as in bisulfite patch PCR and MAPit-patch (Varley and Mitra, 2010; Nabilsi et al., 2014), unmethylated C is converted to U post-DNA enrichment, allowing targeting oligos to be designed without regard to DNA methylation. As an improvement, though, enzymatic conversion of unmethylated C to U (Tahiliani et al., 2009; Schutsky et al., 2018) has been substituted for bisulfite treatment, largely eliminating chemical degradation of DNA (Tanaka and Okamoto, 2007). This facilitates enrichment of 940-bp-long and potentially longer DNA fragments for phasing multiple epigenetic features, e.g., nucleosomes and transcription factor footprints, on contiguous DNA molecules.

In certain embodiments the DNA cleavage and ligation reactions prior to PCR require only <1 hour of hands-on time. Furthermore, the entire preferred FENGC sequence enrichment protocol is done with serial addition of reagents and only one purification step prior to PCR amplification, minimizing loss of input genetic material. The streamlined sequence enrichment procedure therefore requires only 50 ng human gDNA and is poised for automation in clinical applications. Library preparation for FENGC genotyping and MAPit-FENGC requires two and three days, respectively. Both protocols generate a mean specificity of long, mapped sequencing reads of ˜80%.

### Applications of FENGC for Target Sequence Enrichment

Sequential Reagent Addition Protocols for FENGC Enrichment of Targeted DNA Sequences with Nucleotide-Level Precision

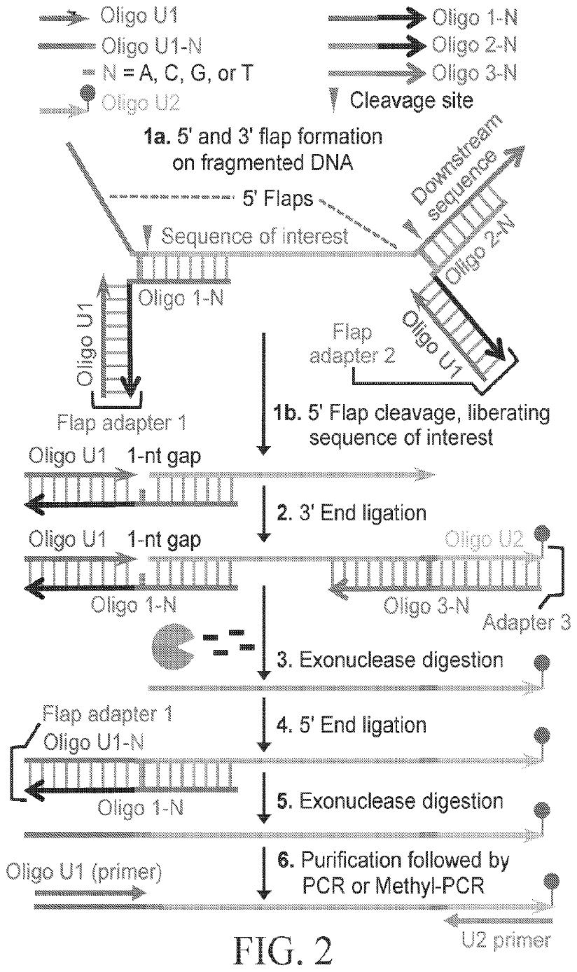

This embodiment describes protocols in which nucleic acids are sequentially modified by reagent addition in a single tube, without multiple purifications, which minimizes losses of input biological material and maintains compatibility with robotic automation. The embodiments described herein require only one or two purifications. Toward this goal, two FENGC protocols (FIG. 1 and FIG. 2) were devised. In certain embodiments, it is preferred that genomic DNA (gDNA) is first fragmented with sonication or, alternatively, with restriction enzyme digestion or another suitable method. Next, the gDNA is denatured and a plurality of single-stranded DNA (ssDNA) sequences of interest is contacted in solution at both ends with a matched pair of flap adapters (FIG. 1 and FIG. 2, step 1a; one sequence of interest shown for clarity). In the preferred FENGC protocol (FIG. 1), each flap adapter consists of either a target sequence-specific oligo 1-N or oligo 2-N, both of which have the same 3′ tail that anneals to and positions for ligation a complementary universal oligo 1-N(oligo U1-N; N is a 3′-terminal A, C, G, or T). In this naming scheme, flap adapters consist of: the U1-T oligo hybridized to an oligo 1-T (FIG. 3); the U1-A oligo hybridized to an oligo 1-A; and so on. The identities of the oligo U1-N 3′-terminal nucleotide and the first base-paired nucleotide of the target sequence within the downstream DNA duplex are the same (FIG. 3, highlighted in pink). This design creates a single-nucleotide “overlap,” enabling the nucleotide in the target sequence to compete with and displace the oligo U1-N 3′-terminal nucleotide and base pair with the corresponding, complementary nucleotide in oligo 1-N(FIG. 3, red type). This creates an unpaired 1-nt 3′ flap in addition to the unpaired 5′ flap, referred to as a double flap (Lyamichev et al., 1999). The double flap with a 1-nt 3′ flap increases the binding affinity for and cleavage rate of 5′ flaps by FENs, including thermostable Flap endonuclease 1 (FEN1) and Taq (Lyamichev et al., 1999; Lyamichev et al., 1999; Friedrich-Heineken et al., 2003). Human FEN1 can also cleave double flaps with 2-, 10-, and 20-nt 3′ flaps, although with reduced efficiency and precision (Friedrich-Heineken et al., 2003). In a preferred embodiment, the 3′ flap is restricted to 1 nt in length by ensuring that the nucleotide at the base of the 5′ flap and adjacent to the overlap nucleotide cannot base pair with the nucleotide on the opposite DNA strand in oligo 1-N. For instance, V (A, C, or G) in the 5′ flap will not base pair with the indicated A in oligo 1-T (FIG. 3).

FEN1, Taq, and related FENs incise the phosphodiester bond of the sequence of interest in double flaps with a 1-nt 3′ flap efficiently and uniformly after both the 5′ flap and the ribose of the first base pair within the downstream duplex (FIG. 1 and FIG. 2, step 1a and FIG. 3, red arrowheads). In a multiplexed reaction, this liberates a plurality of target sequences with a 5′-terminal phosphate located immediately downstream of a 1-nt gap (FIG. 1, step 1b). The 1-nt gap is filled by the oligo U1-N 3′-terminal nucleotide, which is subsequently ligated by Ampligase (Lucigen) to the 5′ ends of the plurality of target sequences (FIG. 1, step 2). By contrast, because 5′ flap incision severs each flap adapter 2 from the sequence of interest, additional adapter 3 is used to position the oligo U2 for ligation to the liberated 3′ end. The oligo U2 is synthesized with a ligatable 5′ phosphate and, at its 3′ end, five phosphorothioate bonds and the 3′-terminal hydroxyl blocked by a three-carbon spacer. These 3′ oligo modifications protect target strands to which the oligo U2 ligated against 3′ to 5′ degradation by both Exo I and Exo III (FIG. 1; step 3). By contrast, all other DNA molecules are degraded (except unligated oligo U2), dramatically enriching the targeted sequences of interest.

Note that the oligo U1-N of flap adapter 2 also ligates to the 5′ end of the downstream sequence. Therefore, a series of flap adapters, each annealing successively farther downstream, can be designed to facilitate ‘walking’ of contiguous regions.

In certain embodiments, the ligated products can be processed for genotyping, epigenetic analysis, or both. For sequence genotyping, the ligated products are purified for the first time in the reagent addition protocol and then amplified using standard PCR with the oligo U1 and U2 primer (FIG. 1, step 4). Alternatively, the purified, enriched target sequences can be subjected to bisulfite or enzymatic conversion of C to U (deamination) prior to PCR amplification, termed methyl-PCR, to detect 5mC and 5hmC at nucleotide-level resolution. In the invention herein, the PCR or methyl-PCR amplicons are subsequently ligated to barcoded hairpin adapters to create SMRTbell™ templates for multiplexed, long-read, and high-fidelity sequencing on a Pacific Biosciences (PacBio) instrument.

For genotyping and methyl sequencing on Illumina instruments, the P5 and P7 Illumina sequences are added to the 5′ end of the U1 and U2 primers by PCR amplification without and after deamination, respectively, as previously described (Varley and Mitra, 2010; Nabilsi et al., 2014). It has also recently been reported that the accuracy of Oxford Nanopore Technology sequencing can be increased to 94% by the rolling circle to concatemeric consensus (R2C2) method (Volden et al., 2018). In principle, this method could be applied to captured sequences of ˜1 kb, a limitation imposed by circularization of double-stranded DNA (Volden et al., 2018; Shore et al., 1981; Shore and Baldwin, 1983). Alternatively, high-accuracy, long-read Nanopore or PacBio sequencing can be obtained using unique molecular identifiers (UMIs) (Karst et al., 2020). The current embodiment can also be used to capture large megabase fragments followed by single-molecule nanopore sequencing (Bennett-Baker and Mueller, 2017; Gabrieli et al., 2018; Gilpatrick et al., 2020) in combination with UMIs to improve sequencing accuracy (Karst et al., 2020).

The FENGC protocol can be stopped after any step and the samples stored at −20° C. before proceeding. The hands-on time for FENGC processing of one multiplexed sample is <1 hr for standard PCR and <2 hr for methyl-PCR. The entire standard PCR protocol can be completed in two days or three days for methyl-PCR.

### EXAMPLES

### Cell Culture

Human fetal telencephalic NSC and human GBM cell lines (L0 and Nx18-25) were cultured in complete NSC medium (basal medium+proliferation supplement at a 9:1 ratio; NeuroCult™ NS-A Proliferation Kit, STEMCELL TECHNOLOGIES, 05751) supplemented with penicillin-streptomycin (1% final concentration; ThermoFisher Scientific Gibco, 15140122), 20 ng/ml human recombinant epidermal growth factor (STEMCELL TECHNOLOGIES, 78006.1), 10 ng/ml human recombinant basic fibroblast growth factor (STEMCELL TECHNOLOGIES, 78003.1), and 0.679 U/ml heparin (Sigma, H3149). For NSC culture, 10 ng/ml leukemia inhibitory factor (Millipore, LIF1050) was also added. The cells were maintained in a humidified incubator at 37° C. and 5% CO₂. A standard protocol was used for passaging the NSC{PMID: 27030542} and GBM cells {PMID: 22064695}, whereby the neurospheres were collected by centrifugation at 110 g for 5 min) every 7-10 days. The pellet was re-suspended in 0.05% (w/v) trypsin (0.53 mM EDTA, ThermoFisher Scientific, 25300062) prewarmed to 37° C. Soybean trypsin inhibitor (ThermoFisher Scientific, 17075029) was then added and gentle pipetting used to dissociate the neurospheres into single cells for re-plating.

### Ethical Approval

Animal protocols were approved by the University of Florida Institutional Animal Care and Use Committee (IACUC Protocol Nos: 201807422 & 201910745). ‘Guide for the Care and Use of Laboratory Animals’ was adhered to as prepared by the Committee for the Update of the Guide for the Care and Use of Laboratory Animals of the Institute for Laboratory Animal Research, National Research Council. Mice were closely monitored for signs of dehydration, weight loss, impaired mobility or physiological signs of underlying disorders such as labored breathing or respiratory distress.

### Animal Description and Treatment

C57BL/6J female mice were used to examine detection by MAPit-FENGC of the epigenetic mixture of transcriptionally inactivated and active copies of the X chromosome. All mice were 8-10 weeks of age upon arrival and individually housed on a 12 h dark/12 h light cycle at 19-22° C. and 30-60% humidity, with standard chow diet and water provided ad libitum. Prior to cell collection, mouse anesthetization was induced and maintained with 5.0% and 1.5% isoflurane USP (NDC 14 043-704-06, Patterson Veterinary Supply, Inc.), respectively, using an Eagle Eye Model 150 anesthesia machine (Jacksonville, FL, USA). The depth of anesthesia was monitored by the absence of pedal withdrawal reflex.

### Single-Molecule MAPit Methylation Footprinting

MAPit was done on permeabilized cells to mark accessible chromatin. In detail, two million cells were first washed with cold PBS with 0.015% (w/v) sodium azide. Cells are pelleted and washed with 500 μL ice-cold cell resuspension buffer (20 mM HEPES, pH 7.5, 70 mM MgCl₂, 0.25 mM EDTA pH 8.0, 0.5 mM EGTA pH 8.0, 0.5% (v/v) glycerol, freshly supplemented 10 mM DTT and 0.25 mM PMSF). Cells were pelleted and resuspend in 180 μL cell resuspension buffer with 0.05% (w/v) digitonin and incubated on ice for 10 min. Cell permeabilization was checked by trypan blue staining, in which 100% of cells should be stained blue before preceding to the next step. Cells were then treated as indicated with or without M.CviPI (100 U/million cells) supplemented with fresh 160 μM SAM followed by a 15 min incubation at 37° C. The reactions were stopped by addition of equal volume stop buffer (1% (w/v) SDS, 100 mM NaCl, 10 mM EDTA) and vortexed briefly at medium speed. Nuclei were treated with RNase A for 30 min at 37° C. followed by 100 μg/mL Proteinase K treatment at 50° C. overnight. Genomic DNA was extracted using phenol-chloroform-isoamyl alcohol (25:24:1, v/v) phase separation, followed by ethanol precipitation and resuspension in water.

Isolation of Monocytes from Mouse Bone Marrow

Bone marrow was collected from spines (below skull to above tail) cleaned of extraneous tissues. All subsequent steps were conducted under sterile conditions. First, the tissue was crushed at room temperature using a ceramic mortar and pestle in a sterile solution of 10 ml of phosphate-buffered saline (PBS), pH 7.2, 2 mM EDTA, 0.5% (w/v) bovine serum albumin (BSA), by mixing MACS BSA Stock Solution (Miltenyi, 130-091-376) and Biotec, autoMACS® Rinsing Solution (Miltenyi, 130-091-222) in a ratio of 1:20. Subsequently, the crushed spine was removed, and the homogenate was filtered through pre-separation filters with 30-μm nylon mesh (Miltenyi Biotec, 130-041-407,) into a 15-mL Falcon® tube. The cellular filtrate was then washed by centrifugation at 300 g for 10 min, removal of the supernatant, and resuspended in the same PBS/BSA solution for cell counting (Heska, Element HT5 Hematology Analyzer). Monocytes were then enriched using a negative isolation protocol specific to mouse bone marrow (Miltenyi Biotec, 130-100-629) according to the manufacturer's protocol. Briefly, ˜40-80 million washed cells were incubated with FcR blocking reagent and cocktail of mouse biotinylated-antibodies, washed with PBS/BSA, resuspended in degassed RPMI 1640 medium (ThermoFisher, 11875101) containing 1% (w/v) penicillin-streptomycin (ThermoFisher Gibco, 15140163), and efficiently depleted of non-target cells by addition of magnetic microbeads conjugated to mouse anti-biotin monoclonal IgG1 antibodies and passage through LS ferromagnetic columns (Miltenyi Biotec, 130-042-401). The flow through, containing highly enriched bone marrow-derived monocytes, was collected into a 1-mL centrifuge tube. To allow recovery from the collection process, a mean of 1.2 million monocytes were plated in a well on a 96-well plate and incubated in a humidified 37° C. incubator at 5% C02 in an RPMI 1640 containing 1% penicillin-streptomycin solution for 3 h before harvesting for MAPit-FENGC.

### FENGC Oligonucleotide Designer (FOLD)

The ˜620-nt panel of FENGC primers was designed by newly developed program, FOLD. The programs searches an input file of gene names or genome coordinates for primers that avoid repeats and satisfy criteria of FIG. 1, such as locating the ‘overlapping’ residues that create 1-nt 3′ flaps. Other user-defined, command-line options include, but are not limited to, increasing the length of default 500-nt sequences and percentage tolerance of departure from this specified length, specification of annotated TSS (e.g., RefSeq), and minimum and maximum primer melting temperature (Tₘ). The program is available online at github.com/albertoriva/FOLD.

### Statistics

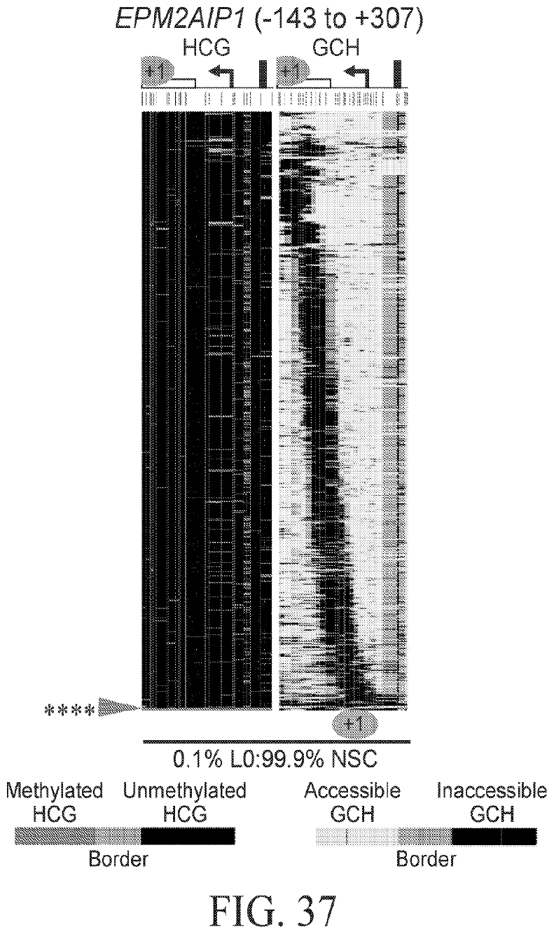

For the 0.1% L0:99.9% NSC gDNA mixture (FIG. 37), a proportions test was conducted in R using the command prop.test( ), with a null hypothesis that the observed proportion (10 hypermethylated:1781 unmethylated epialleles) is less than 0.1%. The P value of <0.0001 indicates that the proportion of hypermethylated epialleles detected was at least or greater than 0.1%.

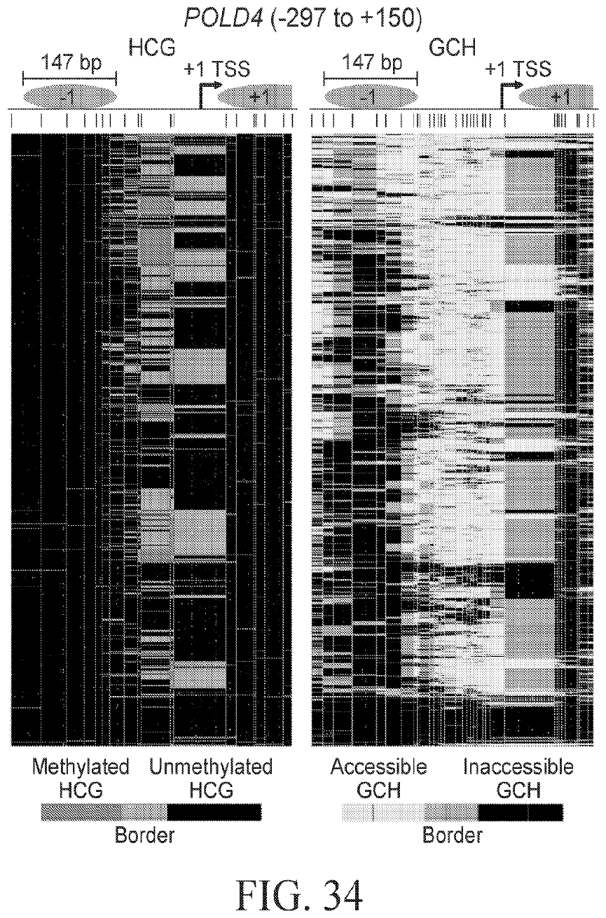

Generalized estimating equations were used to model the effect of cell line (human NSC versus GBM Nx18-25) on the per molecule proportions of endogenous H5mCG (number H5mCG/(number H5mCG+number HCG); left panels) and G5mCH (number G5mCH/(number G5mCH+number GCH); right panels). Only targets with ≥50 total CCS reads in the combined replicates (≥22 in any single replicate) were considered (TABLE 5), with filtering as described in the FIG. 34 legend. Errors were modeled as normally distributed, and the correlation structure was assumed exchangeable within each replicate. The geeglm function from the geepack v1.3-2 R package was used in R version 4.1.0. P values were corrected for multiple testing using the Bonferroni method with an alpha of 0.05 to control the false discovery rate (TABLE 6).

To determine if MAPit-FENGC detected significant epigenetic differences in at least one of the four analyzed mouse monocyte samples, smoothed moving averages (20-bp window) of DNA methylation and accessibility across each gene region were modeled using a mixed effects ANOVA. Testing was limited to 43 amplicons with ≥100 CCS reads per sample and good diversity, i.e., absence of many duplicates (TABLE 11). The model was fit using the gls function in the nlme v3.1-152 package in R version 4.1.0. Each sample was treated as a random effect and correlation along the gene region was modeled as that from an autoregression moving average model (ARMA). Autocorrelation parameters were estimated using the auto.arima function from the forecast v8.15 R package with a maximum possible value of 1 to avoid overfitting. If the differencing order was estimated as zero, then first order differences were taken of DNA methylation and accessibility. If the autoregressive and moving average parameters were both estimated as zero, then an autoregressive model of order one was used. The P values are of the interaction term of base pair and replicate which tested for differences between replicates across the gene region. P values were corrected for multiple testing using the Bonferroni method with an alpha of 0.05 to control the false discovery rate (TABLE 11).

### Example 1: FENGC Procedure for Sequence Enrichment from gDNA

In certain embodiments, flap adapters 1 and 2 as well as corresponding adapters 3 were designed to capture ˜300 nt, ˜450 nt, and ˜940 nt spanning the transcription start sites of 11, 119, and 45 human genes encoding proteins with DNA repair or cancer-associated functions, respectively (Table 1 and Table 2). In certain embodiments, flap adapters 1 and 2 as well as corresponding adapters 3 were designed to capture ˜620 nt of 78 mouse genes expressing products with functions in the cellular inflammatory response.

In certain embodiments, the preferred FENGC procedure with a 1-nt 3′ flap, the gDNA was fragmented by either of two methods: 1) Digestion with SpeI-HF (New England Biolabs) in the CutSmart Buffer (New England Biolabs) in 20 μl volume and incubation for 1 h at 37° C., followed by 20 min at 80° C.; or 2) sonication with a UCD-200 Bioruptor (Diagenode) on the high setting for 25 sec in 100 μl of sterile distilled and deionized H₂O (ddH₂O; MilliQ), followed by SpeedVac™ reduction of the volume to 20 μl. Cleavage of 5′ flaps was performed by combining 1 μl of 10 μM U1-T oligo, 2 μl of a mixture of oligos 1-T and oligos 2-T, the concentration of each depending on the number of target regions of interest (TABLE 12; calculated with formula), 1×PCR Buffer (Qiagen), 3 U APEX Taq (Genesee Scientific), and sterile ddH₂O to bring the total volume to 35 μl. For cleavage with FEN1, 32 U FEN1 and 1×FEN1 Buffer (New England Biolabs) were substituted for Taq and PCR buffer. The reactions were incubated for 3 min at 95° C. and for 20 min at 65° C., followed by 14 cycles of 30 sec at 95° C. and 65° C. for 10 min. Next, 2 μl of a mixture 10 μM of oligos 3-T, the concentration of each depending on the number of target regions of interest (TABLE 12; calculated with formula), 1 μl of 10 μM oligo U2, 1×Ampligase® reaction buffer (Lucigen), 10 U of Ampligase (Lucigen), and ddH₂O were added to bring the total volume to 45 μl. The ligation reaction conditions were identical to those employed above for FENGC of linearized plasmid. Unprotected DNA was removed by addition of 20 U of Exo I (New England Biolabs) and 100 U of Exo III (New England Biolabs), followed by incubation for 1 hr at 37° C. and for 20 min at 80° C.

Surviving DNA sequences were subjected to standard PCR, BS-PCR, or EM-PCR. For standard PCR, the captured DNA sequences (˜300-nt and ˜450-nt targets) were purified with MinElute PCR Purification Kit (Qiagen) or by addition of 1.8× volume of AMPure XP beads, and PCR was performed with HotStar Taq (Qiagen). The PCR reaction was 95° C. for 5 min, followed by 30 cycles of 94° C. for 30 sec, 57° C. for 30 sec, 72° C. for 1 min. For DNA methylation analysis of ˜300-nt and ˜450-nt targets, the eluted, enriched sequences were bisulfite converted and PCR amplified using the same conditions, except that 35 cycles were employed. For EM-PCR of ˜450-nt, ˜620-nt, and ˜940-nt targets, the captured sequences were purified by addition of 1.8× volumes of either AMPure XP beads or NEBNext beads, enzymatically converted according to the EM-seq manual (New England Biolabs), and eluted in 14-25 μl of sterile ddH₂O. PCR amplification with 500 nM each of U1 and U2 primers and HotStar Taq (Qiagen; ˜450-nt targets). 2×KAPA HiFi HotStart Uracil+ReadyMix (Roche; ˜620-nt, and ˜940-nt targets) in 50 μl for 5 min at 95° C., followed by 30 cycles of 20 sec at 98° C., 15 sec at 62° C., 30 sec at 72° C., and one final 1-min extension at 72° C. All oligos used are listed in Table 1.

For the alternate FENGC protocol without a 3′ flap, digestion of purified gDNA with SpeI-HF, 5′ flap cleavage, and first-round ligation to the 3′ end of the cut sequences of interest were as described for the preferred protocol. After the second-round ligation using the same conditions, the reactions were purified with MinElute PCR Purification Kit (Qiagen) and eluted with 14 μl of H₂O, then incubated with 1×QIAGEN PCR Buffer (Qiagen), 0.2 mM dNTP, 500 nM of U1 and U2 primers, 2 units of HotStar Taq (Qiagen) in 20 μl at 95° C. for 5 min, followed by 35 cycles of 30 sec at 94° C., 30 sec at 57° C., 1 min at 72° C. All oligos used are listed in Table 1.

### Example 2: Optimization of Precision Cleavage of ssDNA Sequences of Interest with Taq or FEN1

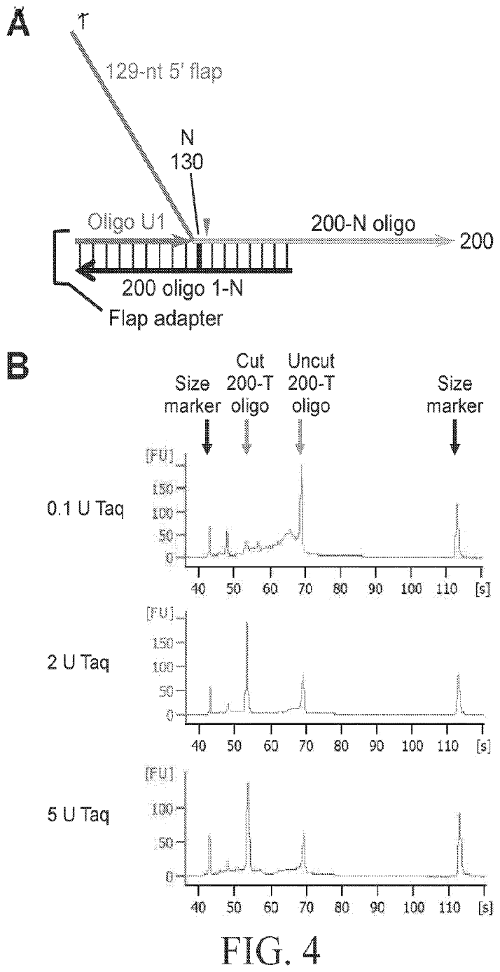

It was found that Taq and FEN1 cleave the phosphodiester bond of DNA structures with ssDNA 5′ flaps, producing a 3′-terminal hydroxyl and 5′-terminal phosphate (Lyamichev et al., 1993; Finger et al., 2012; Kaiser et al., 1999; Lyamichev et al., 1999; Lyamichev et al., 1999; Hall et al., 2000; Tsutakawa et al., 2017). To develop FENGC, the configuration of oligos used for 5′ flap formation and cleavage, the amount of needed enzyme activity, and the number of cleavage cycles were optimized. The efficiency of Taq incision of 5′ flaps containing different combinations of base-paired nucleotides at the location immediately 5′ of the scissile phosphate was tested, using annealed oligos as substrates (FIG. 4A). In this experiment, four 200-mer oligos with a sequence derived from plasmid pGEM-3Z/601 (Lowary and Widom, 1998), with either A, C, G, or T at nt 130 (200-N oligos), were used as proxy DNA sequences of interest (Table 1, Sheet 1). These oligo targets were contacted by their respective flap adapter 1, containing the oligo U1 bound to the corresponding target-complementary 200 oligo 1-N, i.e., with the nucleotide complementary to nt 130. This forms four different structures with different 5′ flaps (FIG. 4A), i.e., without a 1-nt, displaced 3′ flap.

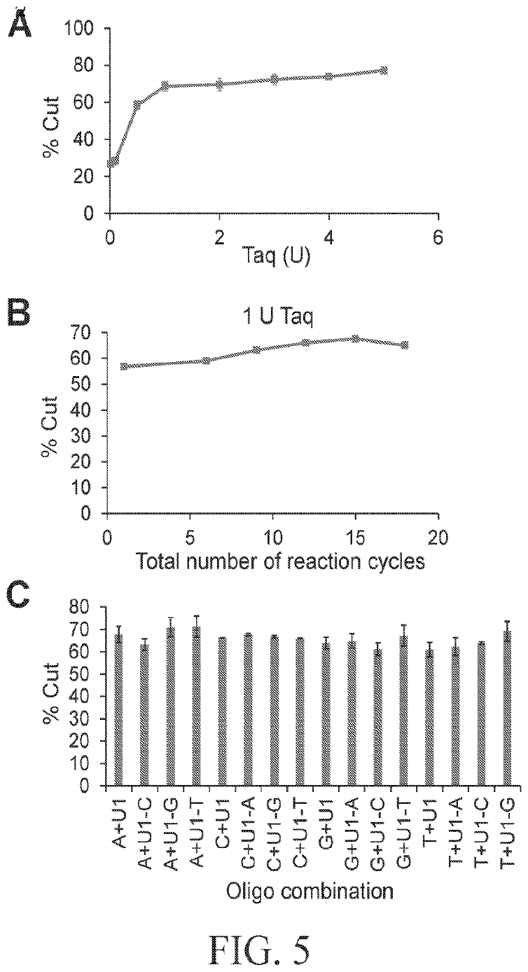

Initially, a 5′ flap substrate consisting of the 200-T oligo, its complementary flap-T oligo (with A base paired with T130 of the 200-T oligo), and the oligo U1 was used to determine the amount of Taq needed to achieve maximal 5′ flap cleavage. The cleavage efficiency was determined using the High Sensitivity DNA Chip on the Agilent 2100 Bioanalyzer (Agilent Genomics) (FIG. 4B). The areas under the peaks of cut and uncut 200-mer oligo were integrated and used to calculate the percentages of cut oligo (FIG. 5). A digestion plateau was reached with 1 unit (U; based on polymerization activity) of Taq (FIG. 5A). Using the same 5′ flap substrate and 1 U Taq, it was determined that the majority of substrate cutting was achieved with one cycle of hybridization and 5′ flap formation; additional cycles yielded a trend toward a plateau with slightly increased cutting (FIG. 5B).

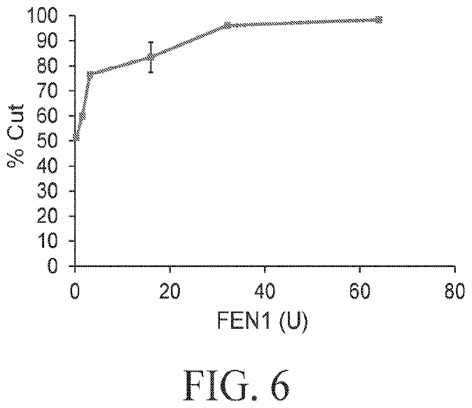

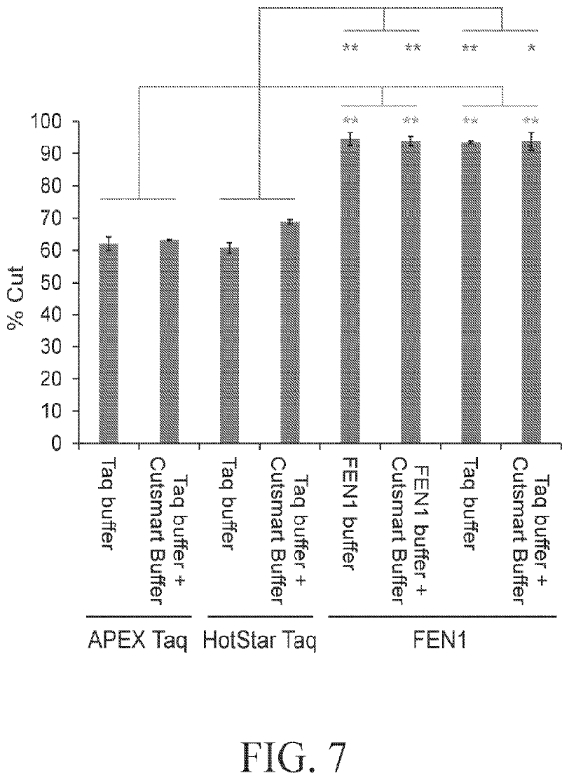

Next, the extent of digestion of 16 different 5′ flap substrates was determined (see Table 1, Sheet 1 for oligo sequences). The identity of the nucleotide immediately 5′ of the cleavage site (in the absence of a 3′ flap) on the 200-mer did not measurably affect cleavage efficiency (FIG. 5C; compare A+U1, C+U1, G+U1, and T+U1). Extending the 3′ end of each oligo U1 by 1 nt (i.e., using oligo U1-Ns instead of the oligo U1), creating an unpaired 1-nt 3′ flap, also did not measurably affect digestion by Taq under the employed reaction conditions (FIG. 5C; compare A+U1 with A+U1-C, A+U1-G, and A+U1-C, etc.). FEN1 achieved an overall higher digestion efficiency than Taq in a reaction containing the same 5′ flap structure used in FIG. 5B (200-T oligo, 200 oligo 1-T, and oligo U1) (FIG. 6). In reactions where DNA was fragmented with a restriction enzyme, the presence of less than 1×CutSmart buffer (New England Biolabs) did not affect the percentage of subsequent flap cutting by either Taq or FEN1, the latter of which again yielded the highest level of cleavage (FIG. 7).

### Example 3: Determination of Digestion Efficiency of Taq DNA Polymerase and FEN1

Flap adapters were designed to consist of oligos 1-N with 3′ tails that anneal to the oligo U1 or oligo U1-Ns. The non-annealed 5′ ends of flap adapters were designed to contact specific sequences in ssDNA target sequences of interest to form 5′ flap structures. The 200-N oligos provided the 5′ flap and cleavage site in the flap structure. For cleavage, 500 nM each of oligo U1, one of four 200 oligos 1-N, and respective one of four 200-N oligos were incubated with APEX Taq (Genesee Scientific) or HotStar Taq (Qiagen) in 1×PCR Buffer (Qiagen; referred to as Taq buffer in this study) or with thermostable FEN1 (New England Biolabs) in 1×ThermoPol Reaction Buffer (referred as FEN1 buffer in this study) in 20 μl final volume (FIG. 7). The reactions were initiated by incubation for 3 min at 95° C., then 20 min at 65° C., followed by the indicated number of cycles of 30 sec at 95° C. and 65° C. for 10 min. All oligos are listed in Table 1. For High Sensitivity DNA Chip assay, the DNA was purified with 5×AMPure XP Beads (Beckman Coulter) and loaded in Agilent 2100 Bioanalyzer system (Agilent Genomics). The amount of oligo with or without cleavage was indicated by digital peaks. The percentages of digestion was calculated by (mass of cut oligo)/(mass of cut oligo+mass of uncut oligo)×100.

### Example 4: FEN1 Cleavage of Double Flaps is Insensitive to Incorporated 5mC

DNA methylation interferes with cleavage by many restriction endonucleases (McClelland, 1981). Therefore, the effect of 5mC on the ability of FENs to bind and incise 5′ flaps was determined. According to solved X-ray co-crystallographic structures, human FEN1 contacts the phosphodiester backbone and ribose sugars of several residues in the dsDNA duplexes located upstream and downstream of a double flap (Tsutakawa et al., 2017; Tsutakawa et al., 2011). In FENGC of sequences with dense 5mC, only the upper DNA strand of the downstream duplex would be methylated because all of the utilized flap adapter oligos are unmethylated. Therefore, to examine the extent to which 5mC affects FEN1 activity, two 80-mers with the same sequence were synthesized. In one of these oligos, five 5mC residues were distributed near the predicted FEN1 cleavage and binding sites in downstream duplex within the double flap substrates formed by annealing to either a flap-A, -C, -G, or -T adapter. Each of the four unmethylated and four methylated double flap structures were cleaved with increasing amounts of FEN1 and the fraction of cut template was determined by quantitative real-time PCR. It is evident that 5mC did not exert a statistically significant effect on the cleavage efficiency of any of the double flap substrates (FIG. 8).

### Example 5: FENGC Enrichment of a Specific Plasmid Sequence after Cutting a 2,453 nt 5′ Flap

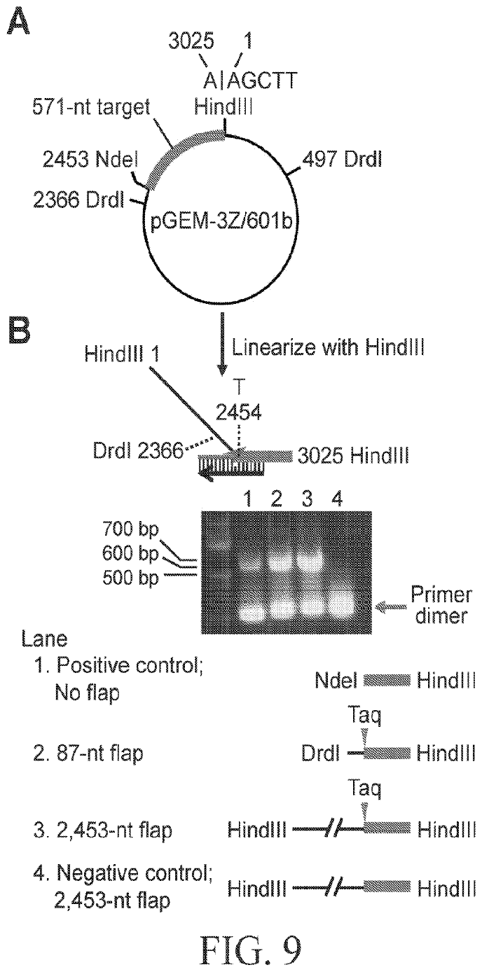

In certain FENGC reactions, the lengths of 5′ flaps will vary between different DNA targets of interest. To test the extent to which 5′ flap length impacts FENGC efficiency, Taq was used to cut substrates containing 5′ flaps of 87 nt or 2,453 nt (FIG. 9). To prepare these substrates, pGEM-3Z/601b (Dechassa et al., 2010), a modified pGEM-3Z/601, was first linearized with HindIII, localizing the 571-nt target DNA strand at one end and downstream of the 2,453 nt of the 5′ flap sequence (FIG. 9A). The first and second A residues in the HindIII site (AAGCTT) were designated as positions 3025 and 1, respectively (FIG. 9A). The linearized plasmid DNA was divided equally into four reactions. Further digestion with DrdI and NdeI shortened the 5′ flap sequence to 0 nt and 87 nt, respectively (FIG. 9B, bottom). A HindIII adapter, consisting of a pGEM-3Z/601b HindIII oligo and the oligo U2, was added to all four reactions to facilitate ligation of the oligo U2 to the common HindIII-cut 3′ end. The NdeI-HindIII cut fragment serves as a positive control for ligation and amplification, i.e., with no 5′ flap, by including an NdeI adapter (pGEM-3Z/601b NdeI oligo 1-T and oligo U1). To the remaining three reactions, a flap adapter 1 (U1-T oligo and pGEM-3Z/601b NdeI oligo 1-T) was added to test cutting of the 5′ flaps of 87 nt and 2,453 nt when Taq was also included. Ligation of the cut 571-nt target sequence to the 47 nt total of U1-T and oligo U2s followed by bisulfite conversion and PCR yielded the expected 618-bp amplification product (FIG. 9B, lane 1). Reactions with the 87-nt and 2,453-nt 5′ flaps yielded a strong product indicative of site-specific 5′ flap cleavage, ligation to the U1-T oligo (and oligo U2 to the HindIII-cut 3′ end), and subsequent PCR amplification (FIG. 9B, lanes 2 and 3). Cutting of the 2,453-nt 5′ flap was Taq dependent, as no product was detected when Taq was omitted (FIG. 9B; lane 4). It can be concluded that FENGC with Taq is able to enrich and amplify a target sequence with a 5′ flap at least as long as 2,453 nt and possibly longer.

### Example 6: FENGC Enrichment with Taq is Insensitive to 5mC at the Site of FEN Cleavage

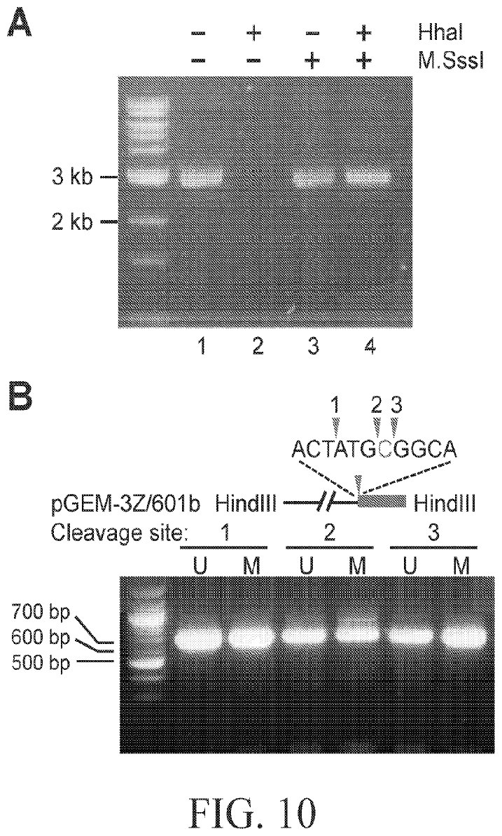

The effect of 5mC on the FENGC protocol was also tested. For this, pGEM-3Z/601b was C-5-methylated with the CpG DNA methyltransferase M.SssI in the presence of the methyl donor SAM. High-level, M.SssI-dependent methylation of the plasmid was demonstrated by inhibition of cutting by restriction enzyme HhaI (GCGC), which is sensitive to 5mC at its central CpG (FIG. 10A). FENGC was conducted on M.SssI-methylated and -untreated plasmid DNA that was linearized with HindIII (FIG. 10B, top panel). Three different flap oligo adapters, consisting of Methyl test pGEM-3Z/601b oligo 1-T, -G, or —C and the respective corresponding U1-T, -G, or -C oligo, directed cleavage of the three indicated phosphodiesters, releasing the 2,415-2,419 nt 5′ flaps from the 605-609 nt target strands. After ligation and subsequent bisulfite conversion, PCR amplification with primers U1 and U2 showed no appreciable difference in the product of FENGC enrichment using all three unmethylated and methylated substrates (FIG. 10B, gel). This demonstrates that FENs can cleave phosphodiester bonds immediately adjacent to 5mC, and therefore the efficiency of the FENGC reaction is not overtly affected by 5mC.

### Example 7: FENGC Performance of Different Flap Complexes

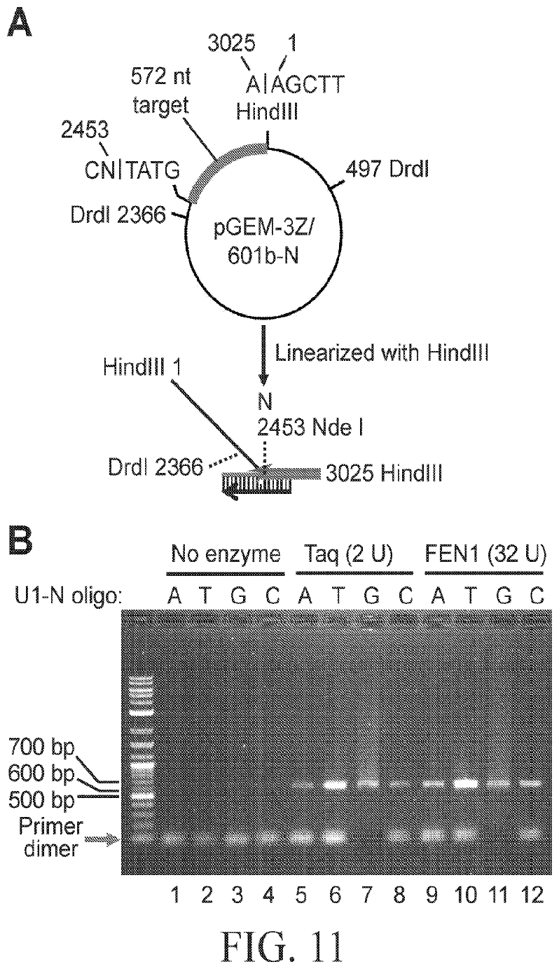

With validation of the suitability of Taq and FEN1 for cleaving 5′ flaps formed by flap adapters, the efficiency of FENGC enrichment was tested on different double flap structures, with overlapping A, C, G, or T. The first A in the NdeI site at position 2,453 of pGEM-3Z/601b was mutagenized to C, G, or T, creating a set of four plasmids (pGEM-3Z/601b-N) (FIG. 11A). As above, 0.2 ng (˜0.1 fmole) of each HindIII-linearized plasmid was denatured and annealed to the HindIII adapter and each of the four respective flap adapters, e.g., pGEM-3Z/601b NdeI-2 oligo 1-A and U1-A oligo, pGEM-3Z/601b NdeI-2 oligo 1-C and U1-C oligo, etc. In each flap structure, the identity of the variant base at position 2,453 and the 1-nt 3′ flap are the same. Cleavage with either Taq or FEN1 of all four plasmids, followed by ligation, bisulfite conversion, and PCR with the U1 and U2 primers yielded the correct 619-bp products (FIG. 11B). Use of the flap adapter-G, however, also generated a high-molecular-weight smear consisting of repeats of primers U1-G and oligo U2s as determined by DNA sequencing. FENGC reactions with the U1-T and flap 1-T oligos showed the highest, specific yield, and therefore this configuration was adopted as the preferred protocol.

### Example 8: Alternative FENGC Procedure without an Overlapping 1-Nt 3′ Flap

It was found that FEN1 and Taq also cut 5′ flap structures that lack a 1-nt 3′ flap, leaving a 1-nt gap (Lyamichev et al., 1999; Lyamichev et al., 1999). In certain embodiments the 5′ flap formation utilizes a flap adapter that contains the oligo U1, without the extra 3′ nucleotide of the oligo U1-N. By this no 3′ flap protocol (FIG. 2), target sequence cutting was accomplished as in the preferred procedure (FIG. 1 and FIG. 2, steps 1a-1b). However, after strand cutting, the 3′ end of each of the plurality of target DNA strands is first ligated to the oligo U2 (FIG. 2, step 2), followed by digestion with both Exo I and Exo III (FIG. 2, step 3). Next, the 5′ end of the DNA target strand is ligated to the corresponding oligo U1-N as dictated by the complementary nucleotide in the gap (FIG. 2, step 4). After a second round of incubation with Exo I and Exo III (FIG. 2, step 5), the plurality of target sequences is purified and amplified by standard PCR or methyl-PCR after deamination (FIG. 2, step 6).

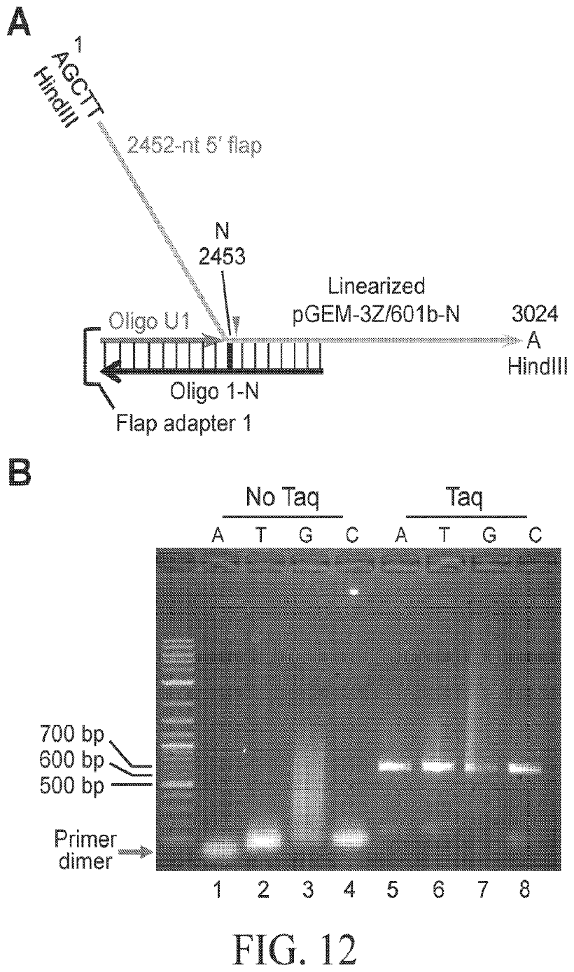

This 5′ flap only procedure was applied to the four pGEM-3Z/601b-N plasmids described in FIG. 11. In this experiment, four different flap structures were formed on the HindIII-linearized and denatured plasmids using flap adapters containing oligo U1 (not U1-N). Therefore, after cleavage of the target strand 5′ end between nt 2,453 and nt 2,454 (FIG. 12A), the 1-nt gap precludes ligation of the oligo U1 as shown in FIG. 2, step 1b. Therefore, the HindIII adapter, Ampligase, and ATP were added in order to ligate the 3′ end-protected oligo U2 to the target strand 3′ end (FIG. 2, step 2). After completion of the alternative FENGC protocol, agarose gel electrophoresis verified production of the expected 619-bp amplification product only in reactions containing Taq (FIG. 12B), as was observed when substrates with a 1-nt 3′ flap were employed in FIG. 11B. In particular, in this no 3′ flap FENGC protocol, the reactions with no Taq also displayed a range of high-molecular-weight products (FIG. 12B) similar to those observed in the Taq and FEN1 reactions in FIG. 11B.

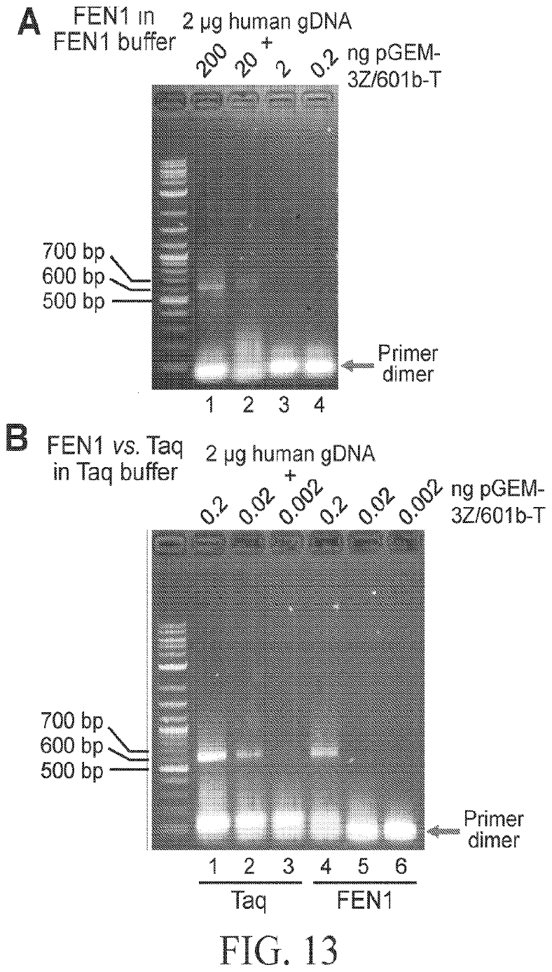

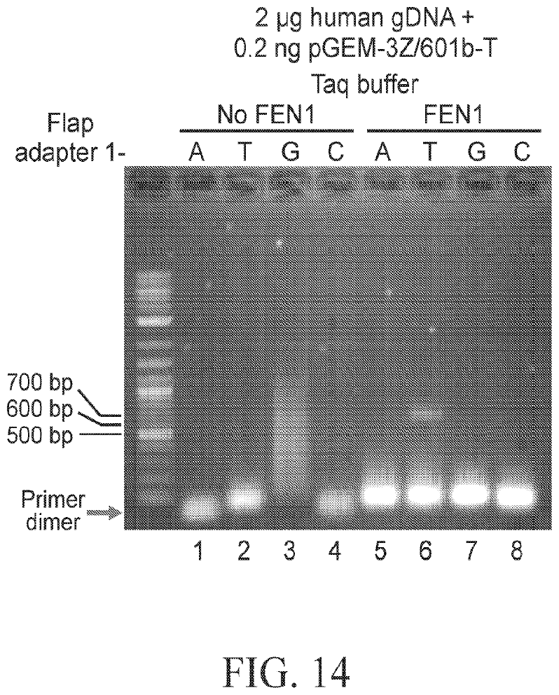

The sensitivity of FENGC was tested using substrates with only a 5′ flap (FIG. 13). In these experiments, 2 p g of human gDNA was mixed with a 10-fold dilution series (0.002 ng to 200 ng) of HindIII-linearized plasmid pGEM-3Z/601b-T, representing human genome equivalent copy numbers of 1 to 100,000. Oligo U1 and pGEM-3Z/601b NdeI-2 oligo 1-T comprising the flap adapter were added to form a 5′ flap with no 3′ flap with the target sequence. Thermostable FEN1 cleaves the target DNA strand of such structures at multiple sites, according to the manufacturer (New England Biolabs). Consistent with this, after executing the alternative FENGC protocol, an amplification product was not observed until 20 ng of spike-in plasmid, equivalent to 10,000 copies (FIG. 13A, lanes 1 and 2 compared with lanes 3 and 4). Interestingly, FENGC with FEN1 and only a 5′ flap improved in Taq buffer in that an abundant PCR product was visible with 100× less spike-in (0.2 ng pGEM-3Z/601-T; 100 copy equivalents) (FIG. 13B, lane 4). By contrast, a trace PCR product of the correct size was obtained in the reaction containing Taq in its supplied buffer and only 0.002 ng plasmid, a molar equivalent of 1 copy (FIG. 13B, lane 3). Furthermore, FENGC in Taq buffer in reactions containing 2 μg human gDNA plus 0.2 ng pGEM-3Z/601-N spike-in produced the 619-bp amplification product only when FEN1 and the pGEM-3Z/601-T flap oligo were supplied (FIG. 14; lane 6 compared with lanes 1-5, 7, and 8). This demonstrates high specificity for 5′ flap cleavage, a T at the 3′ end of the oligo U1 in order to fill the gap and base pair with the complementary A, and ligation of the U1-T oligo to the 5′ end of the FEN1-cut target sequence. Taken together, FENGC of 5′ flaps without a 1-nt 3′ flap achieved the highest sensitivity in reactions using oligo 1-T and Taq in its supplied buffer.

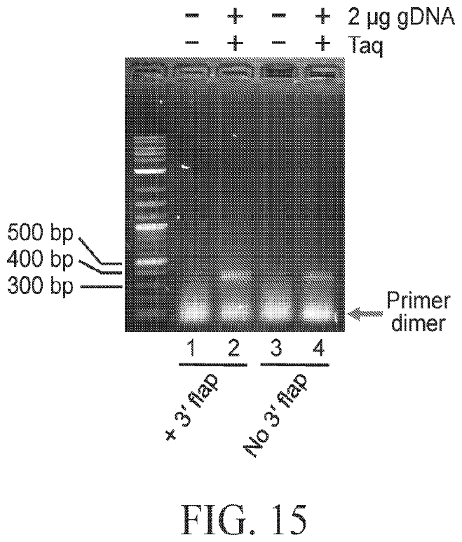

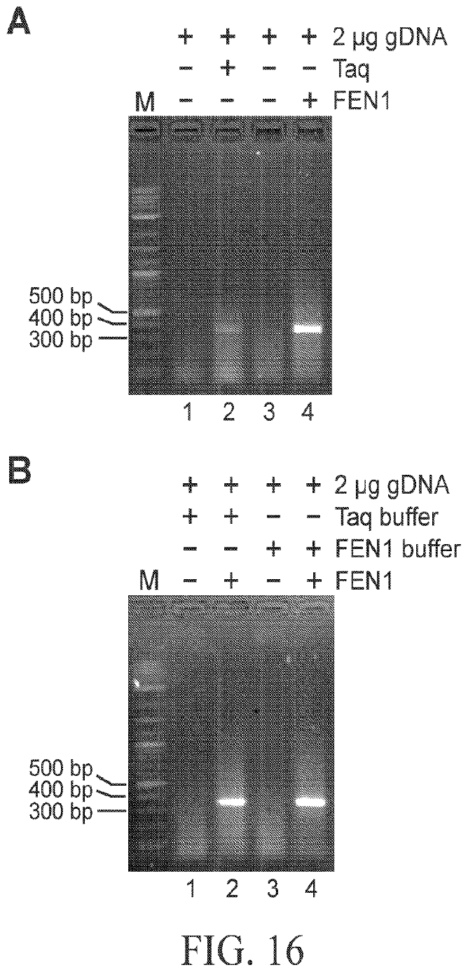

Next, enrichment of human sequences using the two FENGC procedures, one with and one without a 1-nt 3′ flap, were compared in parallel. To do so, gDNA from the human colon carcinoma cell line HCT116, which has a mostly euploid genome (Mouradov et al., 2014), was used as input. Sequences were enriched from approximately −200 to +100 of the transcription start sites (TSSs) of 10 human DNA mismatch repair genes and 1 human control gene with open chromatin (Table 2). Captured sequences of the expected lengths of ˜300 nt, ligated to the 47 nt of ligated universal oligos, were amplified by standard PCR dependent on Taq in both FENGC reactions, demonstrating the efficacy of both protocols (FIG. 15; lanes 2 and 4 versus lanes 1 and 3). Moreover, a side-by-side comparison of preferred FENGC with Taq versus FEN1 on double flap structures showed superior enrichment yield for the same 11 human sequences with FEN1 (FIG. 16A), and FEN1 performed well in the manufacturer-supplied buffers for both enzymes (FIG. 16B). Therefore, the FENGC strategy employing the double flap and FEN1 in its manufacturer supplied buffer was selected for all subsequent experiments.

### Example 9: Compatibility of FENGC with Bisulfite Sequencing to Detect DNA Methylation

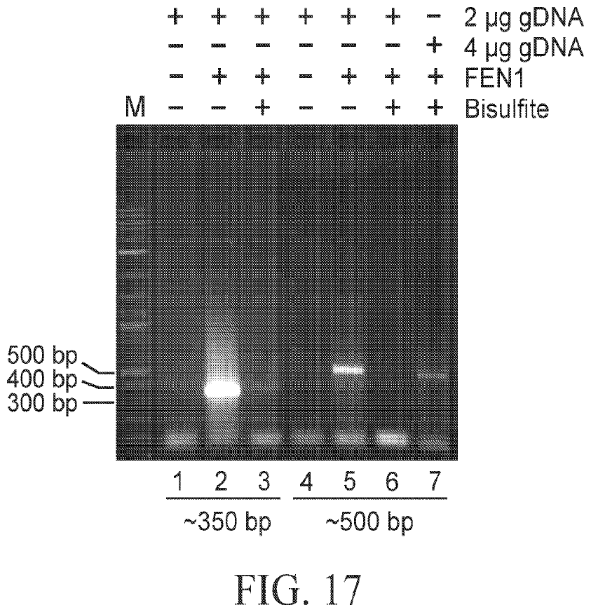

To determine if 5mC could be detected by FENGC, sequences from the same 10 human DNA mismatch repair genes plus 1 human control gene were captured from 2 μg HCT116 DNA, treated with and without sodium bisulfite, and PCR amplified. Two sets of PCR amplicons were examined, with lengths ˜350 bp and ˜500 bp, anchored at the same 5′ end (Table 2) (FIG. 17). As expected, the FENGC enrichment yield with and without bisulfite treatment was higher for the set of shorter amplicons than the longer set (FIG. 17, compare lane 2 with 5 and lane 3 with 6), reflecting the well-known PCR bias toward shorter amplicons. This result also underscores the advantage of matching sequence lengths as afforded by FENGC. In addition, bisulfite deamination of both sets of captured sequences dramatically decreased amplification yield (FIG. 17, compare lane 2 with 3 and lane 5 with 6); an amplified ˜500-bp product was only observed when the input gDNA was doubled to 4 μg (FIG. 17, compare lane 6 with 7). In addition, variable amplification yields were observed with different bisulfite conversion kits (data not shown). The lower PCR product yield is consistent with the degradation of as much as 99.9% of input DNA during the deamination reaction (Tanaka and Okamoto, 2007). Therefore, the quantities of captured DNA available for PCR are extremely low after bisulfite conversion, e.g., only ˜1 pg within 1 μg human gDNA for 10 targets of ˜300 nt.

### Example 10: MAPit-FENGC, a Versatile Assay for Targeted Epigenetic Analysis

In certain embodiments, FENGC is combined with single-molecule MAPit methylation footprinting. In the resulting MAPit-FENGC assay, cells are permeabilized to allow the GpC DNA methyltransferase M.CviPI (New England Biolabs) or other suitable DNA methyltransferases to enter and diffuse into nuclei to methylate accessible GpC sites in the case of M.CviPI (Xu et al., 1998) or C in other contexts in chromatin (Nabilsi et al., 2014; Jessen et al., 2006; Gal-Yam et al., 2006; Lin et al., 2007; Pardo et al., 2011; Kelly et al., 2012). In some aspects, nuclei may first be isolated and treated with a DNA methyltransferase. After stopping the methylation reaction, gDNA was purified and subjected to FENGC followed by bisulfite conversion as in FIG. 17. Because Gp5mC (hereafter, G5mC) can be discerned from endogenous 5mCpG (hereafter, 5mCG), MAPit-FENGC simultaneously detects chromatin accessibility and DNA methylation. Moreover, the assay is freed from the constraints of target selection imposed by restriction endonucleases, a limitation of previously described MAPit-patch (Nabilsi et al., 2014).

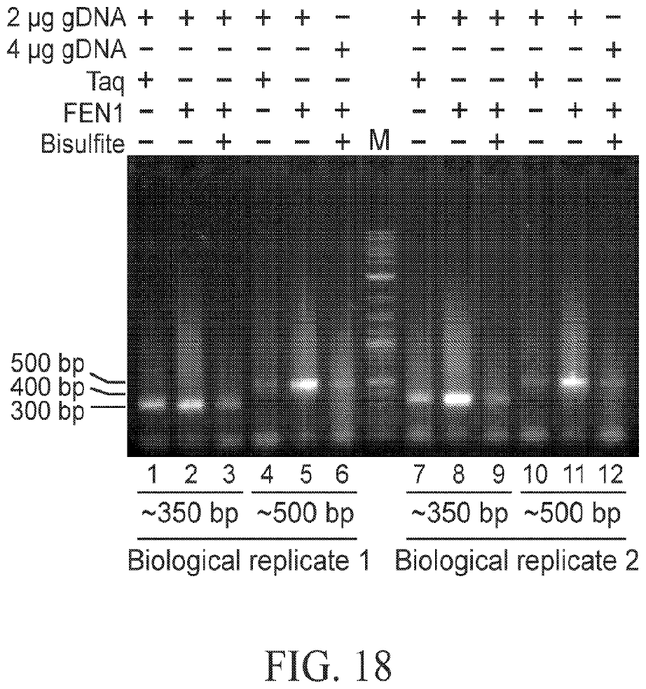

To test MAPit-FENGC, the same 11 FENGC amplification products described above were obtained from gDNA isolated from M.CviPI-treated human glioblastoma (GBM) cell line L0{PMID 24105770}. The MLH1 promoter was included to serve as a positive control with a known chromatin structure (Lin et al., 2007). FENGC with Taq or FEN1 was used to capture targets with lengths of ˜300 nt and ˜450 nt (TABLE 1, Sheets 2 and 3 and TABLE 2, Sheets 1 and 2), which were subjected to amplification by standard PCR or BS-PCR. Specific amplification products of the expected sizes inclusive of the 47 bp of the universal oligo sequences were observed in two independent biological replicates (FIG. 18, ˜350 bp, lanes 1-3 and 7-9; ˜500 bp, lanes 4-6 and 10-12).

Long-read, high-fidelity sequencing is the most informative in epigenetic assays in that it provides single-molecule data, i.e., avoids population averaging, and preserves phasing, the relationship between multiple features along each sequencing read. Therefore, the amplification products were subjected to long-read, circular consensus sequencing (CCS) on a Pacific Biosciences (PacBio) Sequel instrument (Eid et al., 2009). PacBio currently provides the most accurate sequencing platform because single molecules with lengths of up to 10 kb are sequenced at least five times or passes. The PacBio Sequel II instrument has a capacity of 8 M single molecules.

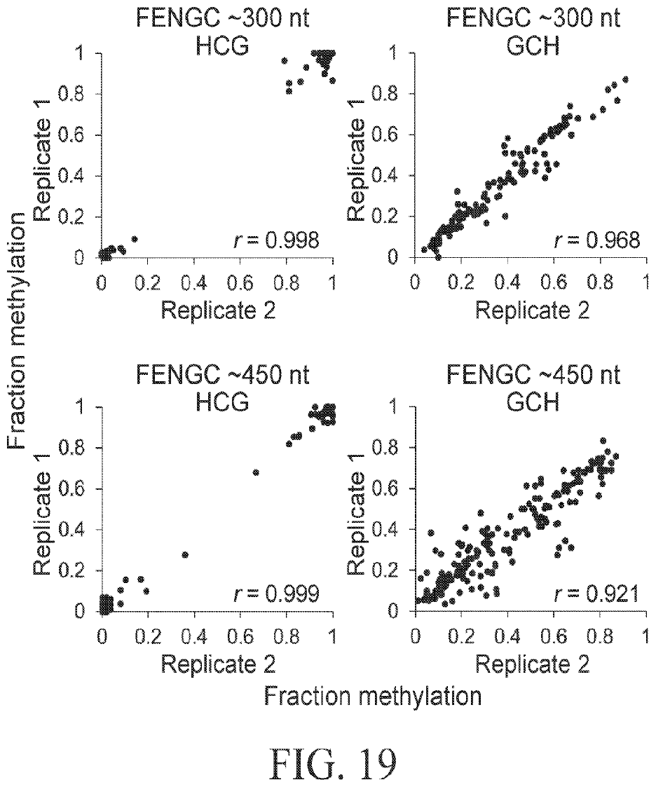

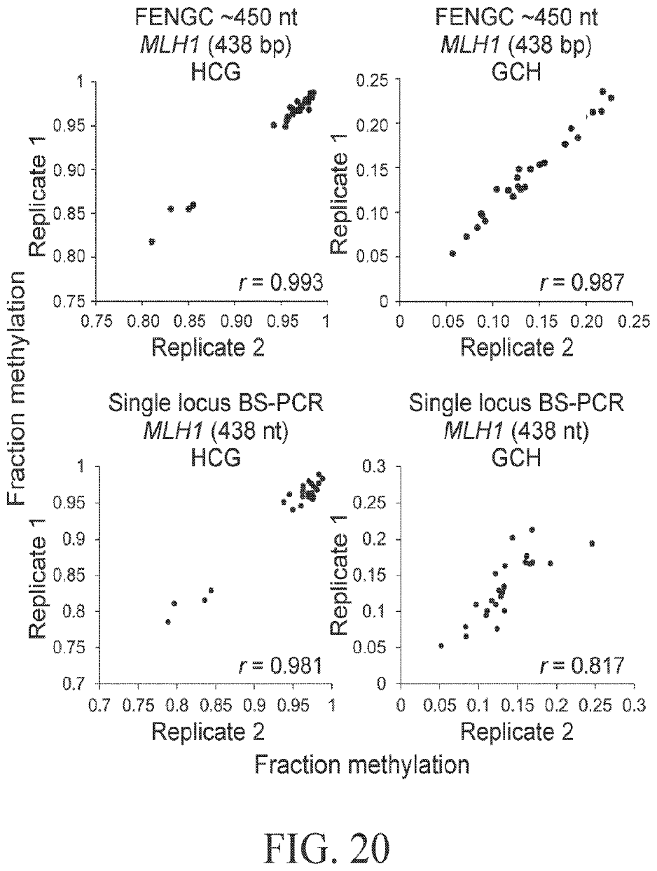

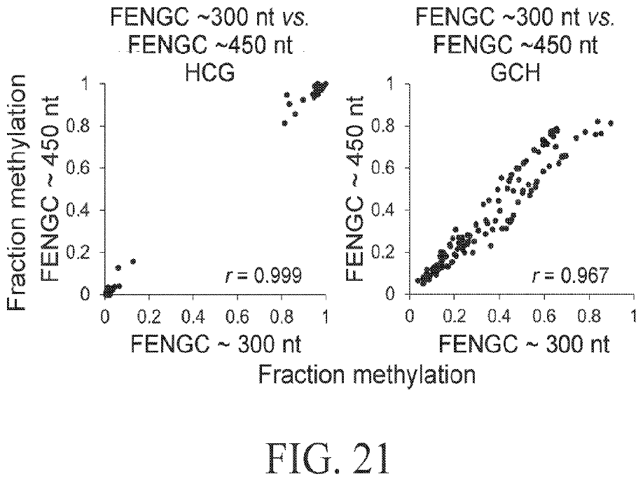

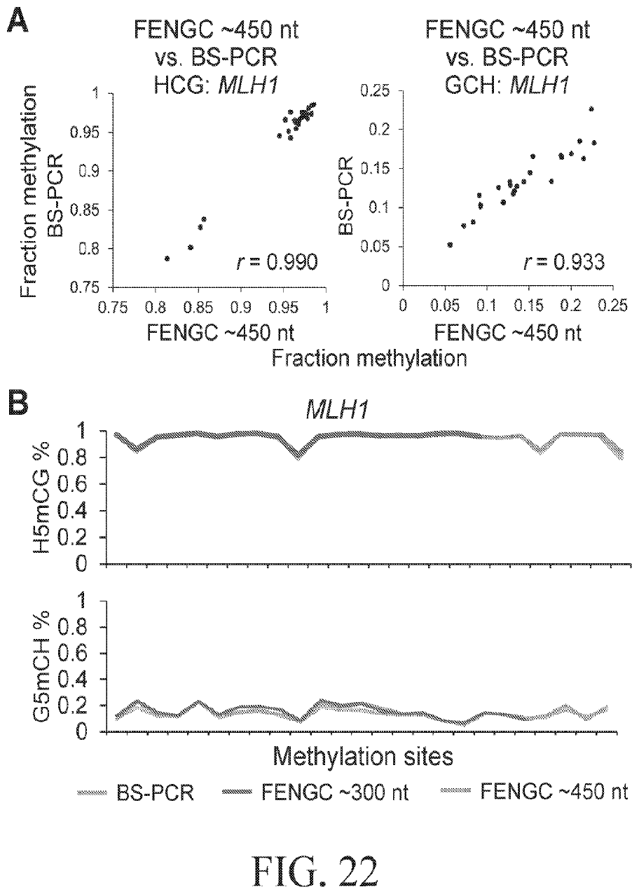

After aligning the PacBio Sequel reads to their reference sequences as previously described (Nabilsi et al., 2014), all FENGC amplification reactions detected at least 9 of 11 targets (82%) with at least 1 read (Table 3). Few reads were detected for MSH3 and MSH6, most likely due to their high GC content, a well-established NGS phenomenon. For the remaining 9 targets, both Taq and FEN1 captured more than 20 reads with standard PCR when combining two biological replicates, demonstrating that both enzymes can be used in FENGC. For the BS-PCR samples, there were 6 targets with more than 20 reads when combining two biological replicates for both the ˜350-nt and ˜500-nt captured sequences. These 6 targets were used to calculate the fraction of G5mCH and H5mCG (GCG were excluded due to overlap of GpC and CpG) in reads with ≥5 sequencing passes and ≥95% conversion at non-GCH and non-HCG sites, i.e., HCH. The correlation coefficients for methylation of all HCG and GCH sites between the two biological replicates were >0.92 for all conditions, indicating high reproducibility of MAPit-FENGC results (FIG. 19). Interestingly, the sequenced FENGC products of MLH1 showed higher correlations between biological replicates than bisulfite sequencing of a single PCR amplicon, i.e., not obtained by FENGC (FIG. 20). This is perhaps related to improved amplification with universal primers as opposed to gene-specific primers (Varley and Mitra, 2008). Importantly, the correlations of HCG and GCH methylation levels between the FENGC products of different lengths were also very high (FIG. 21). FENGC and direct BS-PCR of the MLH1 promoter also showed excellent agreement (FIG. 22). In summary, FENGC efficiently enriches specific target sequences from gDNA obtained from chromatin samples probed with exogenous DNA methyltransferases, such as M.CviPI, that are subsequently treated with bisulfite and PCR amplified.

### Example 11: MAPit (Methyltransferase Accessibility Protocol for Individual Templates) Methylation Footprinting

MAPit was performed on cells permeabilized with digitonin to mark accessible GpC sites in chromatin. In detail, two million cells were first washed with cold phosphate buffered saline containing 0.015% (w/v) sodium azide (Nabilsi et al., 2014). Cells were pelleted and washed with 500 μL ice-cold cell resuspension buffer (20 mM HEPES, pH 7.5, 70 mM MgCl₂, 0.25 mM EDTA, pH 8.0, 0.5 mM EGTA, pH 8.0, 0.5% (v/v) glycerol, freshly supplemented with 10 mM DTT and 0.25 mM PMSF. Cells were next pelleted and resuspend in 180 μL cell resuspension buffer with 0.05% (w/v) digitonin and incubated on ice for 10 min. A 1 μL aliquot of cells was stained with trypan blue to verify 100% permeabilization before proceeding. The cell suspension was then divided in half, treated with and without M.CviPI (100 U/million cells), and supplemented with fresh 160 μM SAM, followed by incubation for 15 min at 37° C. Methylation reactions were stopped by addition of an equal volume of stop buffer (1% (w/v) sodium dodecyl sulfate, 100 mM NaCl, 10 mM ethylenediaminetetraacetic acid (EDTA)) and vortexed briefly at medium speed. RNase A was added to 10 μg/mL for 1 hr at 37° C. followed by 100 μg/mL proteinase K treatment overnight at 50° C. GDNA was extracted using phenol-chloroform-isoamyl alcohol (25:24:1 (v/v)) phase separation, followed by ethanol precipitation, and resuspension in ddH₂O.

### Example 12: Enhanced FENGC Efficacy with Enzymatic-Based Detection of 5mC

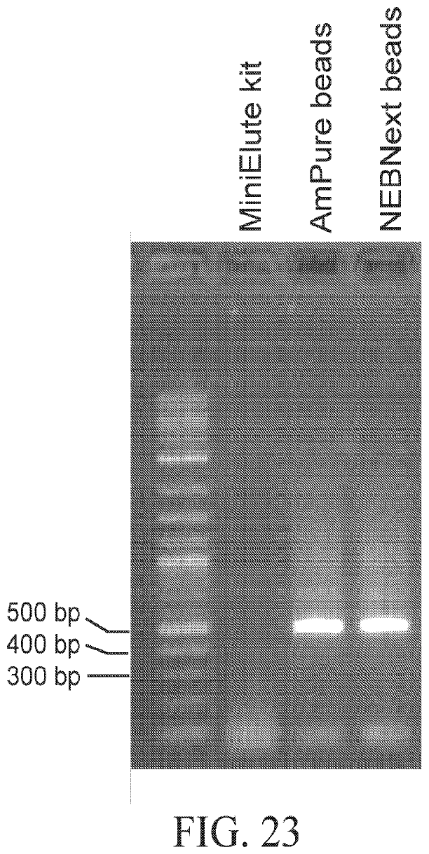

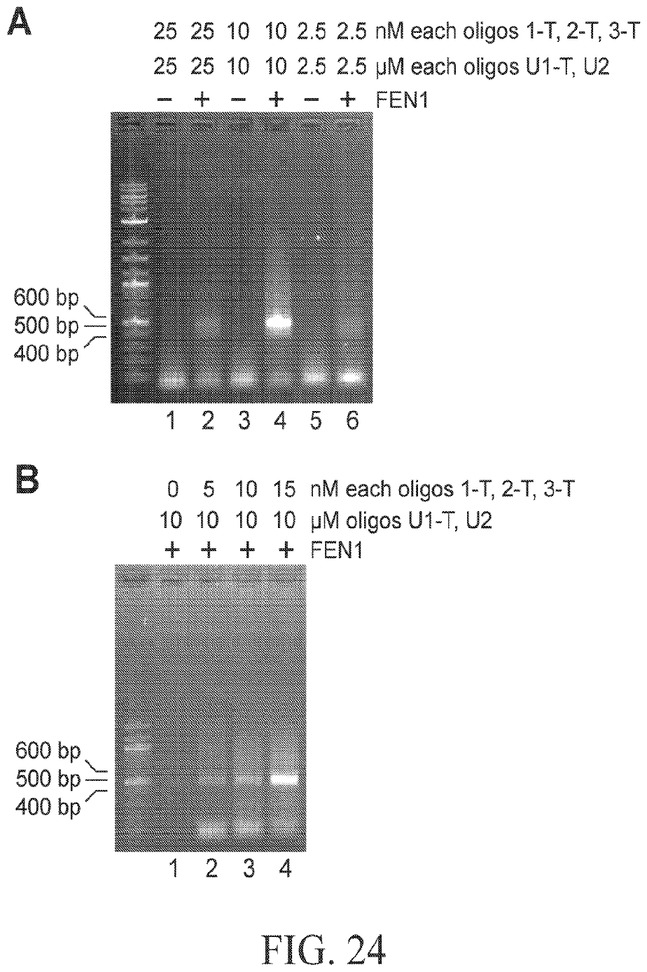

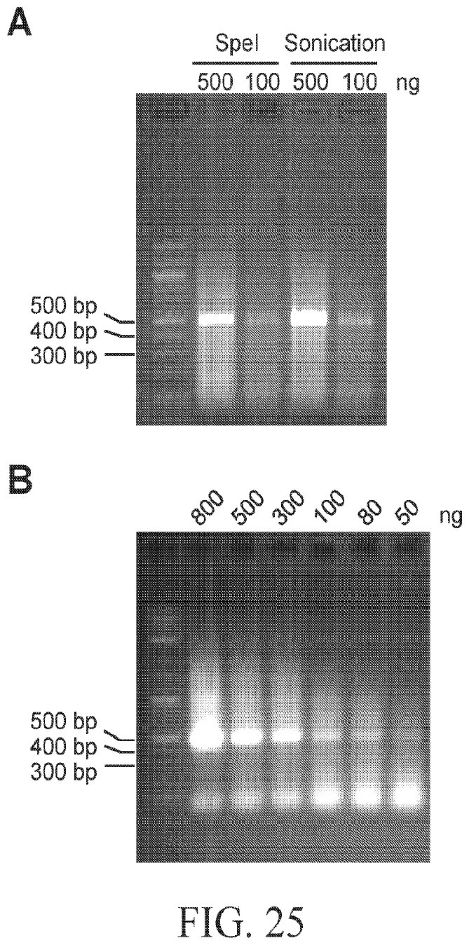

Given the extensive DNA degradation and hence decreased sensitivity of all bisulfite-based methods for detection of DNA methylation, the extent to which a nondestructive method of C to U conversion would improve the sensitivity of MAPit-FENGC was tested. This method, Enzymatic Methyl-seq™ (EM-seq; New England Biolabs), uses an α-ketoglutarate-dependent ten-eleven translocation 2 (TET2) enzyme to oxidize 5mC to 5-hydromethyl-C (neb.com/products/e7120-nebnext-enzymatic-methyl-seq-kit #Citations %20&%20Technical %20Literature)(Sun et al., 2021; Zhang et al., 2013; Yu et al., 2012), which is coupled to glucosylation by T4 phage β-glucosyltransferase (Josse and 15 Kornberg, 1962; Tomaschewski et al., 1985; Schutsky et al., 2017). The resulting glucosyl-5-hydroxymethylcytosine modification protects against subsequent C to U enzymatic deamination by APOBEC (Schutsky et al., 2018; Schutsky et al., 2017). Also, the number of distinct target sequences captured and amplified from the same L0 gDNA used to generate FIG. 18 was increased to 119 targets spanning the transcription start sites (TSSs) of 74 genes with the Gene Ontology term “metabolic process” and filtered for “DNA repair,” 42 genes associated with cancer, and 3 control genes (Table 1, Sheet 3 and TABLE 2, Sheet 2). These targets ranged from 430 nt to 452 nt in size. EM-seq-converted sequences were robustly enriched by FENGC, using 500 ng gDNA (digested with SpeI to decrease the size of 5′ flaps) and post-exonuclease purification with either AMPure XP beads or NEBNext beads (FIG. 23). Purification with the MinElute PCR Purification Kit (Qiagen) was not successful for methyl-PCR. The optimal amount of oligos comprising the flap adapters to include for FENGC followed by EM-seq is 1 μl each of a 10 μM stock solution of U1-T and U2 (FIG. 24A). In addition, the summed total of flap oligos 1-T, flap oligos 2-T, and oligos 3-T to include should approach but be less than the amount of oligo U1-T or oligo U2 (FIG. 24B and Table 12, calculated with formula). Sonication of gDNA to a mean length of 1 kb to decrease 5′ flap size also enriched target sequences as well as SpeI digestion, and is preferred as it avoids cutting SpeI-containing target sequences (FIG. 25A). In addition, in contrast to bisulfite conversion, EM-seq conversion produced a detectable amplification product of the expected size with as low as 50 ng of sonicated DNA input (FIG. 25B).

### Example 13: MAPit-FENGC with Enzymatic-Based Detection of 5mC Yields a High Proportion of On-Target Sequence Reads

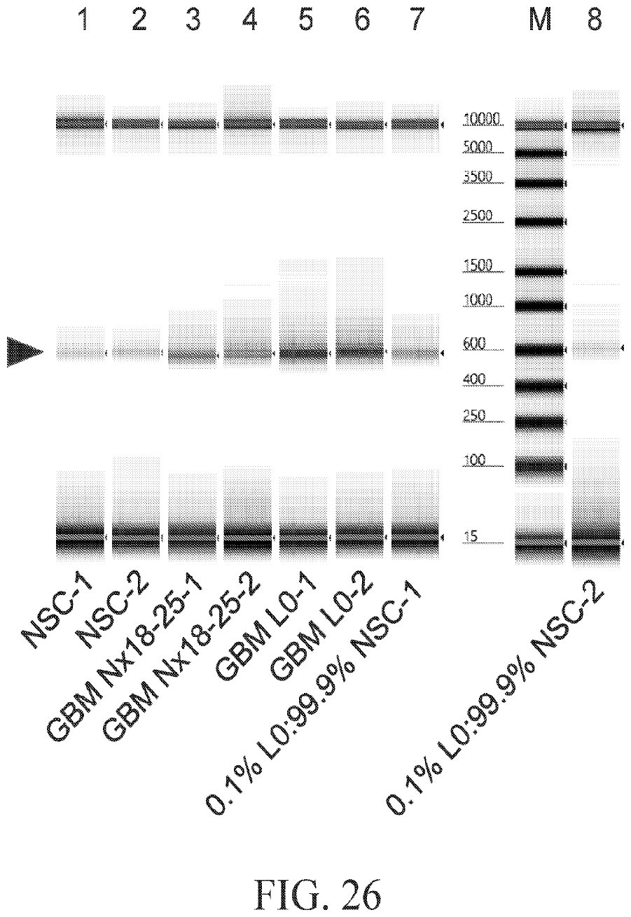

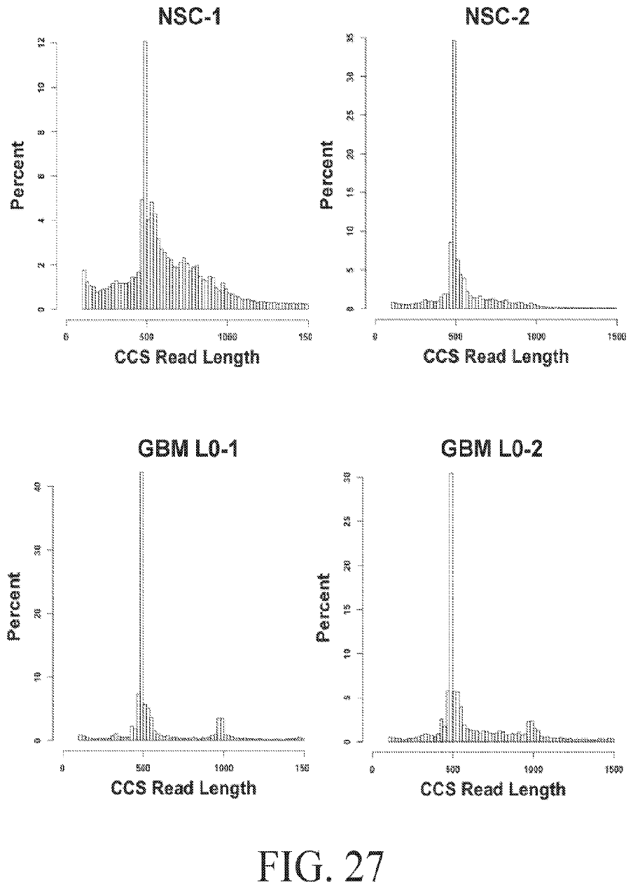

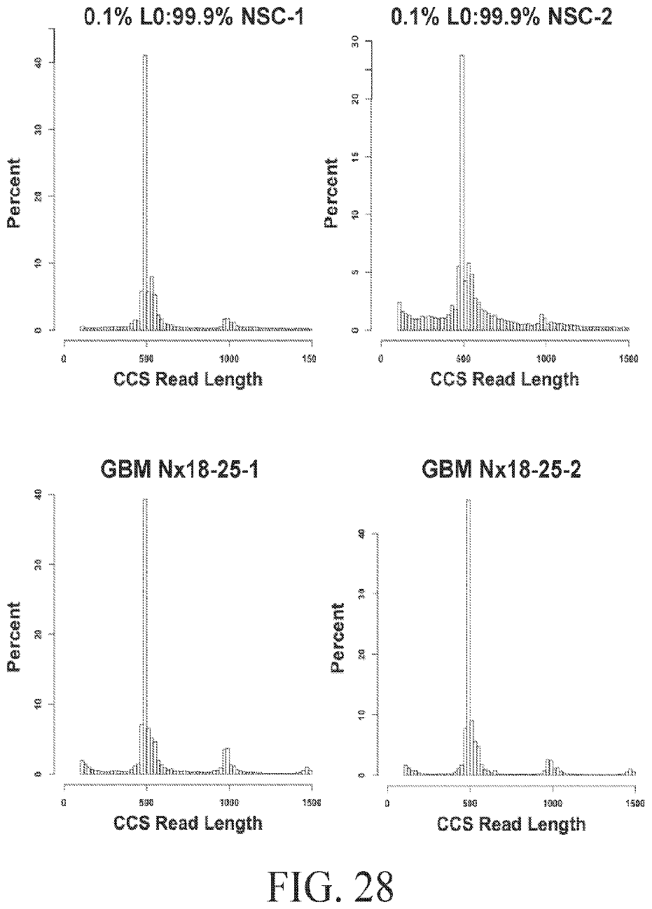

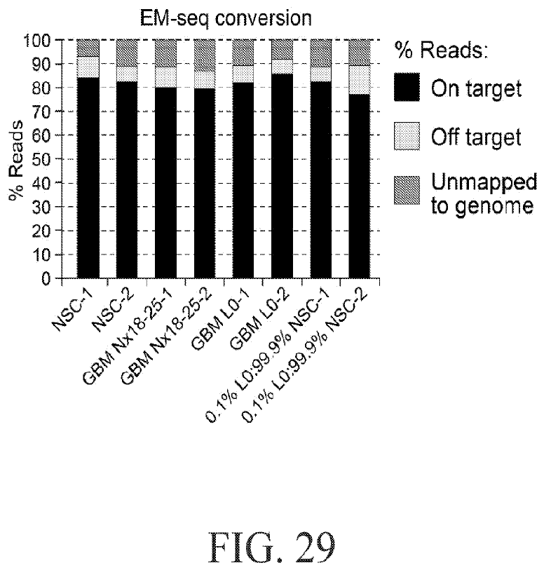

MAPit-FENGC analysis was conducted in duplicate on two independent cultures of NSC, GBM Nx18-25, and GBM L0. The 119 gene targets were captured from sonicated gDNA purified from each cell line itself as well as a mixture of 0.1% L0:99.9% NSC, subjected to EM-seq conversion, PCR amplified, and purified with AMPure XP beads. The amplified, purified products were of high quality and uniformity in length as gauged by the Agilent TapeStation D5000 system (FIG. 26). In the distribution of high-fidelity PacBio CCS reads, the highest proportion were 480-500 nt in length, consistent with the amplification target size (FIG. 27 and FIG. 28). The small peak at approximately 960-1,000 bp is consistent with self-ligation of amplified products during the PacBio barcoding process. CCS reads were mapped to both the whole human genome (hg38) and target reference sequences (FIG. 29; Table 4).

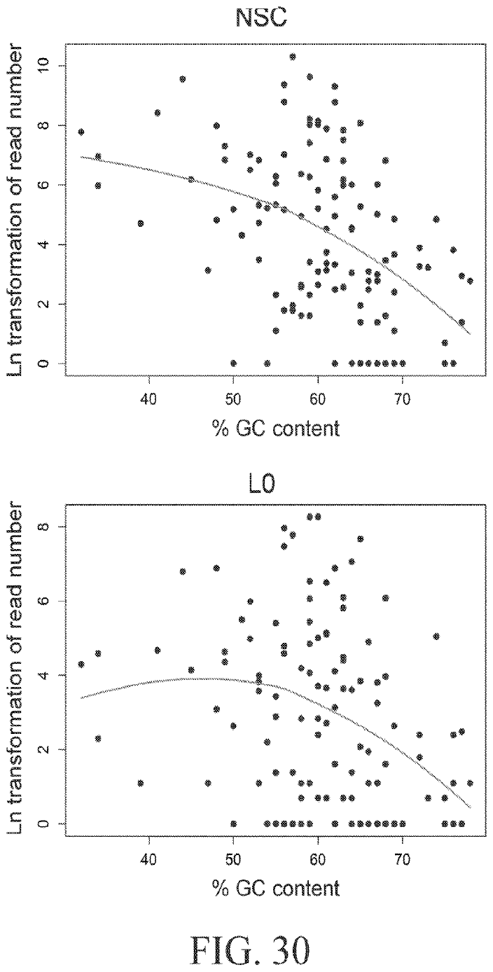

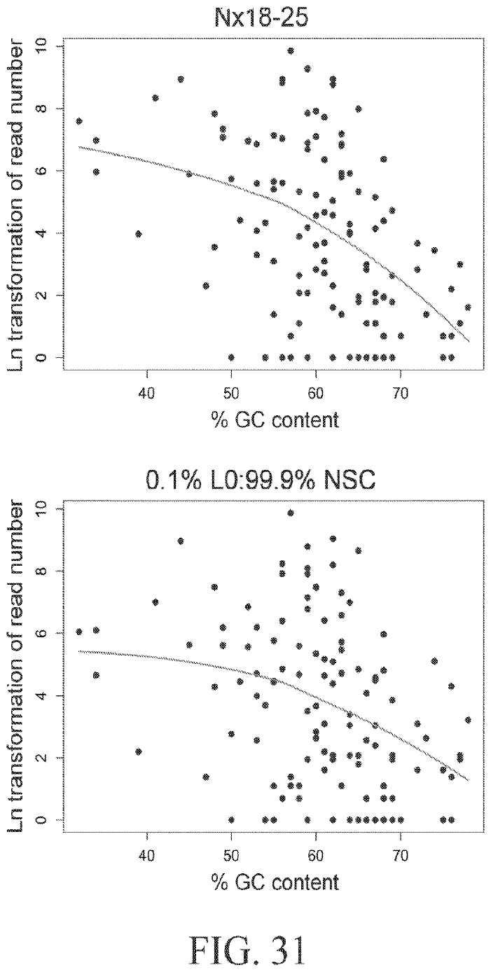

For the two independent biological replicates of L0, for example, percentages of unmapped, off-target, and on-target reads obtained from each of the sequenced libraries were 8-11%, 7-8%, and 82-86%, respectively. There were 105 targets (88%) detected, with at least one read in the combined data from the two biological replicates (Table 4). Reads with ≥5 sequencing passes, >95% HCH conversion to HTH, and aligning to >95% of the length of each reference sequence were analyzed to avoid duplicated alignment to gene homologs with high sequence similarity. After this filtering, all L0 reads were uniquely aligned and 92 targets (77%) were represented (Table 5). As reported previously for bisulfite patch PCR and MAPit-patch (Varley and Mitra, 2010; Nabilsi et al., 2014), filtered read number was negatively correlated with GC content for all of the sequenced libraries (FIG. 30 and FIG. 31).

### Example 14: MAPit-FENGC Efficiently Detects and Localizes 5mCG, Nucleosomes, and DNA-Bound Transcription Factors

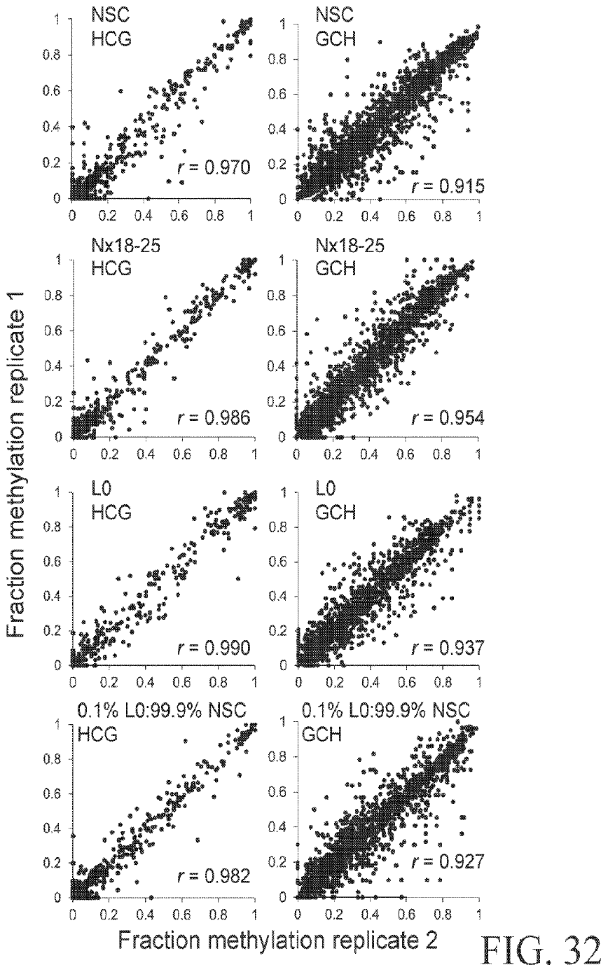

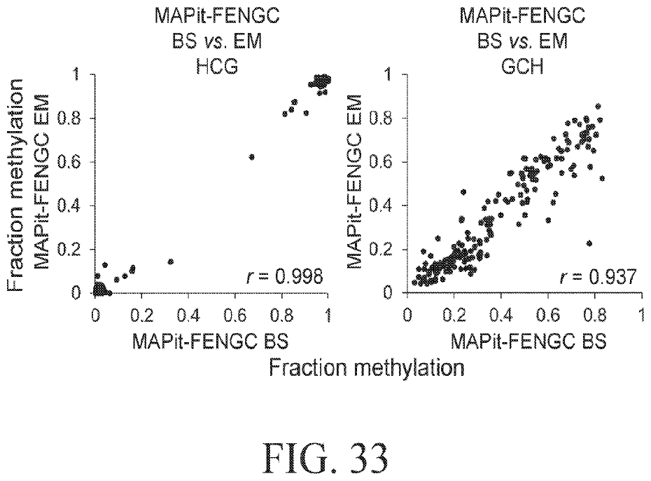

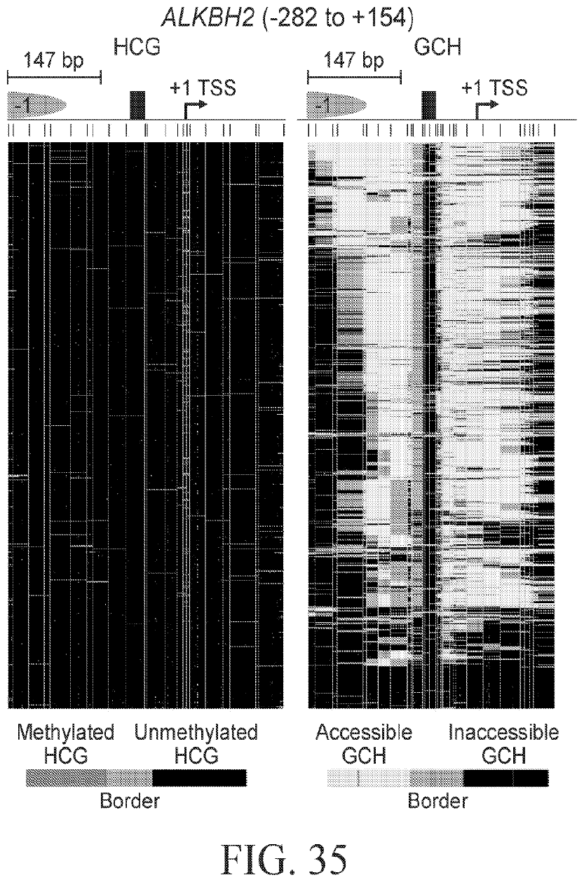

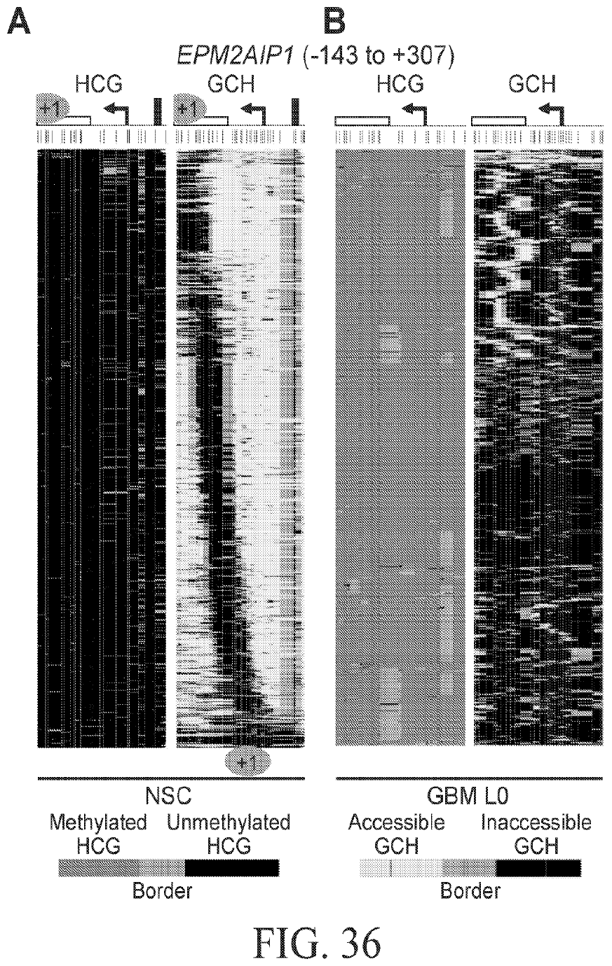

For the fractions of H5mCG (DNA methylation) and G5mCH (chromatin accessibility), the correlation coefficients between the two independent biological replicates of each sequenced sample were very high, above 0.91 (FIG. 32). The results from FENGC targets processed by BS-PCR versus EM-seq conversion PCR (EM-PCR) were compared for six targets with a minimum of 36 sequencing reads in both data sets (FIG. 33). The fractions of methylation of all HCG and GCH sites on the six amplicons from these two data sets were highly consistent, indicating that EM-seq can be substituted for BS-seq in MAPit-FENGC. Three representative examples of MAPit-FENGC sequence reads using EM-seq as plotted with methylscaper {PMID 34125875} are shown in FIG. 34, FIG. 35, and FIG. 36. In these images, each row of pixels represents the pattern of HCG methylation (left; red) and GCH accessibility (right; yellow) on one chromatin copy or molecule (read) in the original GBM L0 cells. All molecules are presented in both panels in the same top-to-bottom order.

The POLD4 promoter from L0 harbors two positioned nucleosomes (designated −1 and +1) flanking a prominent NFR of variable length at the TSS, i.e., present on a large proportion of epialleles (FIG. 34, right panel). Variable-length spans of H5mCG also reside in the linker DNA between the −1 and +1 nucleosomes (FIG. 34, left panel). Open chromatin and the absence of DNA methylation at the TSS are features that correlate well with active transcription.

In the second example, also from L0, all sequenced copies of the region encompassing the ALKBH2 TSS are essentially unmethylated, that is, the level of 5mCG is at the detection limit, except for modest 5mCG at the TSS (FIG. 35, left panel). In addition, most promoter copies house a large region of accessibility to M.CviPI of >147 bp (147 bp DNA wraps around and contacts the histone octamer in nucleosomes), indicative of a prominent NFR (FIG. 35, right panel). Large, variable-length regions of inaccessibility or footprints at the most upstream end of the ALKBH2 promoter likely correspond to portions of differentially positioned nucleosomes (halved blue ellipse representing the population average). By contrast, a relatively small and robust footprint occupies the NFR just upstream of the TSS. Due to its short length of ˜22 bp and uniform position, this footprint most likely corresponds to occupancy by a sequence-specific, non-histone regulatory factor, perhaps a transcriptional activator that orchestrates nucleosome eviction from the TSS.

The third promoter, EPM2AIP1, exemplifies a locus that is differentially methylated in two different cell types (FIG. 36). In cultured human neural stem cells (NSC), the region around the TSS of EPM2AIP1 is highly accessible, except for a variably positioned +1 nucleosome and a likely DNA-bound, sequence-specific transcriptional activator just upstream of the TSS (FIG. 36A, right panel) and essentially unmethylated (FIG. 36A, left panel). By contrast, in cultured GBM L0 cells, the region around the TSS of EPM2AIP1 exhibited high, aberrant levels of H5mCG (FIG. 36B, left panel) and largely inaccessible chromatin, except for accessible, relatively short nucleosomal linkers (FIG. 36B, right panel), consistent with transcriptional silencing (data not shown). The above three examples illustrate that MAPit-FENGC employing methylation detection by EM-seq is a powerful strategy for detecting multiple epigenetic features at high resolution on single molecules.

### Example 15: MAPit-FENGC has High Sensitivity, Identifying 1 in 1,000 Hypermethylated Epialleles

To examine the sensitivity of MAPit-FENGC in the detection of gDNA derived from abnormal cells, 0.1% L0 gDNA was spiked into that of human NSC, i.e., 0.1% L0:99.9% NSC. The 119 products were enriched as described above in FIG. 26, lanes 7 and 8. The read lengths distribution (FIG. 28), percentages of mapped CCS reads (FIG. 29), and correlations of the fractions H5mCG and G5mCH between the two independent biological replicates (FIG. 32) were also high as observed with MAPit-FENGC of L0 described above in FIG. 27, FIG. 29, and FIG. 32. With more than three times more high-fidelity CCS reads compared with L0, this 0.1% L0:99.9% NSC sample detected 109 targets (92%) with ≥1 read (Table 4). After more stringent filtering of CCS reads for ≥5 sequencing passes, ≥95% HCH conversion, and ≥95% coverage of the length of each target reference sequence, 103 targets (87%) were detected (Tables 4 and 5). MAPit-FENGC successfully detected the dense H5mCG in the 450 bp area encompassing the EPM2AIP1 TSS derived from the 0.1% L0 gDNA spike-in (10 of 1,781 molecules), with high statistical significance (P<0.0001) (FIG. 37). By contrast, the remaining molecules in the mixture showed a pattern of GCH accessibility similar to that of NSC and therefore were likely derived from that sample. These data demonstrate that FENGC is highly sensitive, quantifiable, and reproducible for the detection of epigenetic features.

### Example 16: Efficient Detection of Differential Epigenetic Alterations by MAPit-FENGC

The ability of MAPit-FENGC to detect differential epigenetic signatures was tested further by assaying independent duplicate cultures of non-cancerous NSC and a different GBM primary cell line, Nx18-25, using the same 119 target-gene panel. Libraries for the 119 captured products were generated as described above (FIG. 26, lanes 1-4) and yielded the expected length distributions of high-fidelity PacBio CCS read lengths (FIG. 27 and FIG. 28). The percentages of mapped CCS reads were high (Tables 4 and 5). Among these samples, Nx18-25 had similar read numbers compared with the 0.1% L0 spike-in sample, and also showed the same number of detected targets (109 or 92%). In addition, an identical number of targets (103 or 87%) was represented among high-quality reads having ≥5 sequencing passes, additionally filtered for ≥95% HCH conversion, and covering ≥95% of each reference sequence (Tables 4 and 5). While 2.3× as many reads were obtained from NSC as compared to Nx18-25, the numbers of targets detected (mean 110 or 93%) and targets covered by filtered reads (mean 105 or 87%) were similar (Tables 4 and 5).

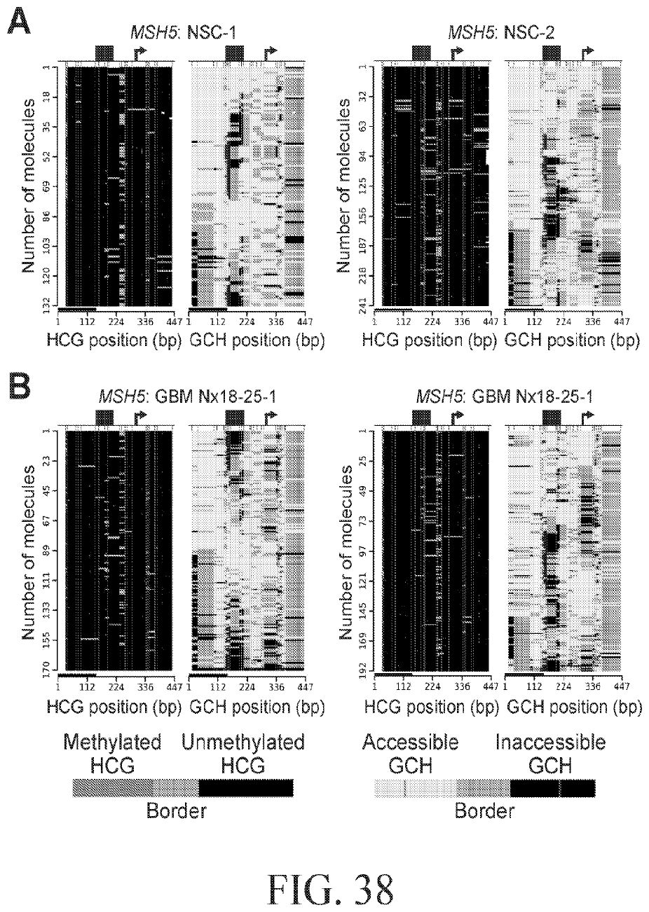

Epigenetic differences between NSC and GBM Nx18-25 were assessed by calculating P values for 54 targets with ≥50 total CCS reads in both replicates, but no less than 22 reads in either of the two replicates (TABLE 6). Using criteria of P<0.05 and ≥5% differential in the level of either H5mCG or G5mCH between NSC and GBM Nx18-25, 57% (31/54) of the evaluated promoters showed epigenetic alterations (TABLE 6, Sheets 1 and 2). Applying more stringent cut-offs, the percentages of promoters with ≥0.1, ≥0.2, and ≥0.4 differentials in H5mCG or G5mCH were 26% (14/54), 13% (7/54), and 7.4% (4/54), respectively (TABLE 6, Sheets 3-5). The quantitative nature of these results is supported by the highly correlated (r>0.91) H5mCG and G5mCH levels between individual replicates (FIG. 32). In addition, 733 of 735 of MSHS promoter molecules across all 4 samples had 10 methylated GCH sites (FIG. 38). This rules out incomplete cell permeabilization and chromatin probing as trivial reasons for the differential accessibilities observed between loci in the same or different samples in TABLE 6 and as further discussed below.

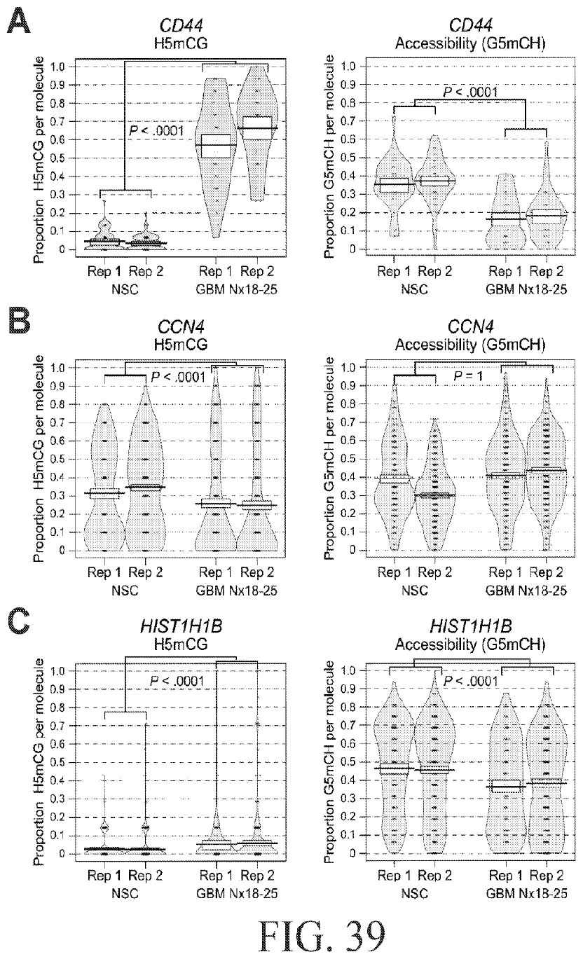

Three representative genes from the NSC and Nx18-25 data sets were chosen to illustrate the reproducibility and power of MAPit-FENGC to reveal differential epigenetic landscapes. These promoters include human CD44, CCN4, and HIST1HB1 for which the data were rendered as violin plots for the two independent replicates (Rep 1 and 2) of NSC and GBM Nx18-25 (FIG. 39). Plotted is the proportion of H5mCG or G5mCH for each molecule (black dots), the median (horizontal line), interquartile range of methylation levels (box), and the smoothed probability density at different methylation levels (gray area). These plots, similar to the H5mCG and G5mCH correlation plots (FIG. 32), further illustrate the quantitative nature and reproducibility of MAPit-FENGC results.

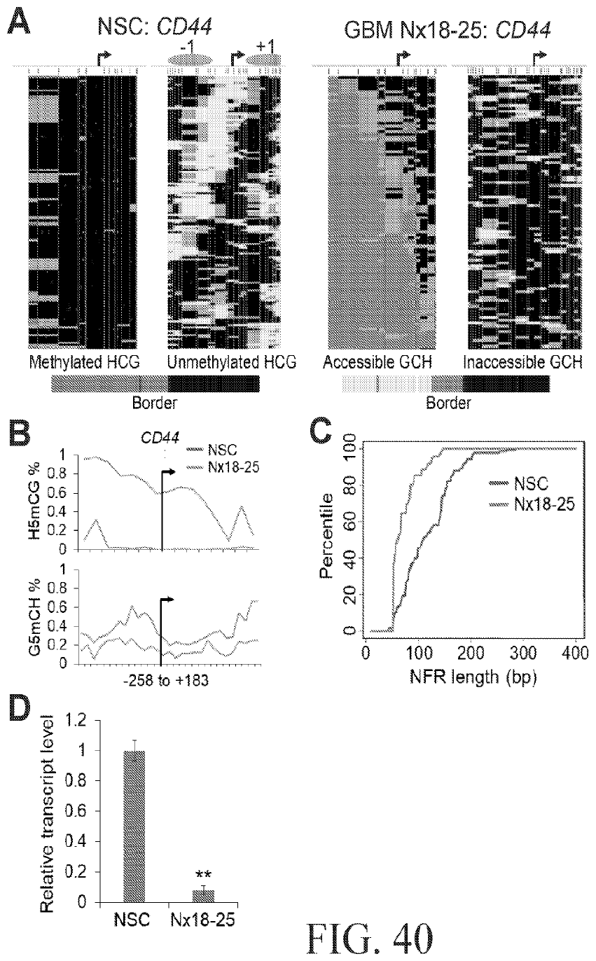

CD44 encodes a transmembrane glycoprotein with functions in cell adhesion, proliferation, and apoptosis (Naor et al., 1997; Naor, 2016). It has also been reported to be a marker for astrocyte-restricted precursor cells (Liu et al., 2004). High expression of CD44 in GBM tissue has been particularly linked to the mesenchymal subtype (Phillips et al., 2006; Verhaak et al., 2010) and GBM cancer stem cells (Anido et al., 2010; Fu et al., 2013). MAPit-FENGC of non-cancerous NSC showed undetectable H5mCG in the vicinity of the CD44 TSS, with limited H5mCG accumulating farther upstream (FIG. 40A, first panel and FIG. 40B, upper panel). The majority of promoter copies from NSC harbored an NFR flanked by nucleosomes, which appeared to move in register to occupy multiple positions (FIG. 40A, second panel and FIG. 40B, lower panel). In striking contrast, in GBM Nx18-25, H5mCG had apparently spread to different extents across most promoter epialleles, leading to relatively promoter hypermethylation that correlated with a dramatic reduction in accessibility (FIG. 40A, third and fourth panels and FIG. 40B, lower panel), which manifests as shortened NFRs compared with NSC (FIG. 40C). The revealed epigenetic signatures of CD44 are consistent with the observed strong transcriptional silencing in Nx18-25 compared with NSC (FIG. 40D).

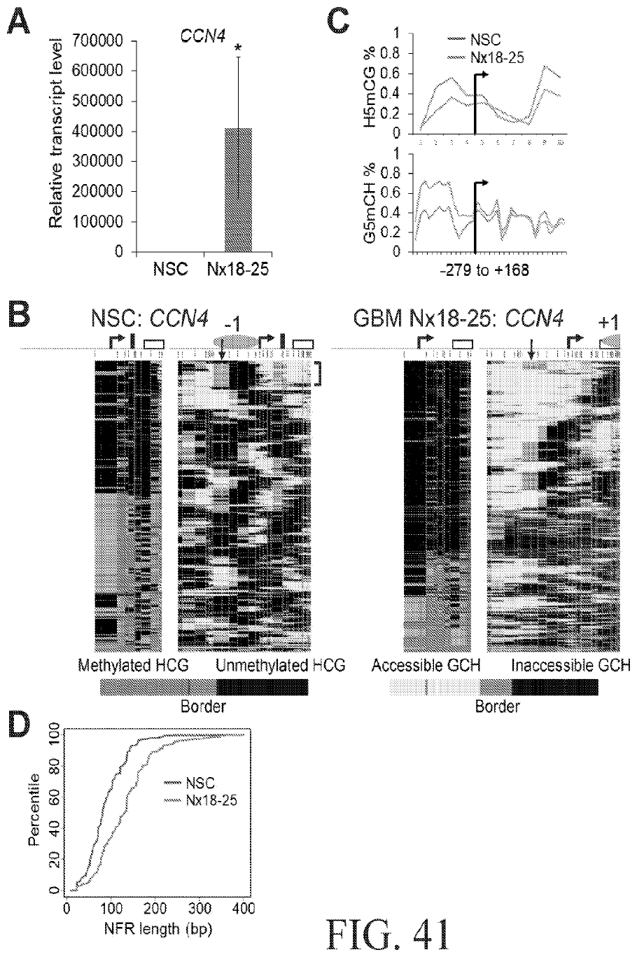

CCN4, also known as WNT1-Inducible Signaling Pathway Protein 1 (WISP1), has been shown to contribute to the tumorigenesis and progression of a wide array of human cancers (Gaudreau et al., 2019; Liu et al., 2019; Deng et al., 2019). More importantly, CCN4 gene expression has been reported to be upregulated in GBM compared with normal tissues (Jing et al., 2017). Indeed, the transcript level of CCN4 was markedly enhanced in GBM Nx18-25 as opposed to the undetectable level in NSC (FIG. 41A). MAPit-FENGC of the CCN4 promoter from NSC showed about half of the cells contained a broken span of H5mCG immediately downstream of the TSS (FIG. 41B, first panel and FIG. 41C, upper panel). Reads derived from these and many other cells contained random, small spans of accessibility, demonstrating occupancy by randomly positioned nucleosomes (FIG. 41A, second panel). In addition, a cluster of reads at the top had a visible −1 nucleosome; the bracketed subset of these reads contained footprint evidence of a DNA-bound, sequence-specific factor (black rectangle). Consistent with the dramatic increase in CCN4 transcription in Nx18-25 compared with NSC seen in FIG. 41A, the number of CCN4 promoter molecules with H5mCG was depleted and there were increases in accessibility and NFR length upstream of the TSS (FIG. 41B, compare the third with first panel as well as fourth with second panel; FIG. 41C, and FIG. 41D).

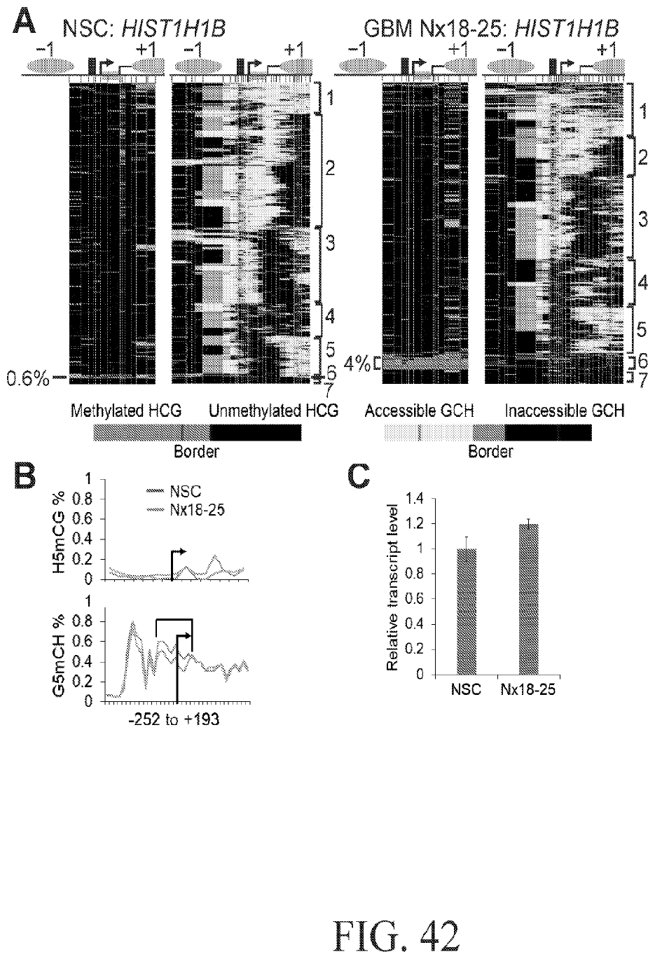

The gene HIST1H1B encodes the linker histone protein H1.5, which is involved in maintaining higher-order chromatin structure as well as regulating DNA repair and cell proliferation (Albig et al., 1997; Sancho et al., 2008; Happel and Doenecke, 2009). By MAPit-FENGC, the HIST1H1B promoter from NSC was largely devoid of H5mCG, except for 0.6% that were hypermethylated and relatively inaccessible across the whole analyzed region (FIG. 42A, first panel, cluster 6 and FIG. 42B, upper panel). In Nx18-25, the fraction of epialleles populating cluster 6 increased to ˜4% (FIG. 42A, compare panel 3 with 1). Interestingly, in both cell types, the −1 nucleosome was uniformly positioned, whereas the +1 nucleosome was slid downstream to various extents (FIG. 42A, second and fourth panels). On close examination, overall, accessibility decreased at the HIST1H1B TSS (FIG. 42A, compare second and fourth panels, sum of clusters 1 and 2; FIG. 42B, bottom panel, bracket). Despite these observed alterations in promoter accessibility and apparent epigenetic silencing of 4% of epialleles in Nx18-25, there was no significant difference in transcript abundance between NSC and Nx18-25 (FIG. 42D). Therefore, MAPit-FENGC is able to identify frequent as well as rare epigenetic differences between biological samples, and the profiled epigenetic heterogeneity provides key insights into gene regulatory mechanisms.

### Example 17: Detection of Genetic Alterations by FENGC

For genotyping, target DNA sequences captured and enriched by FENGC are sequenced directly, without deamination. To demonstrate feasibility, the same 119 target promoter sequences were captured from NSC and Nx18-25 gDNA by FENGC, amplified with standard PCR, barcoded, and then directly sequenced on a PacBio Sequel instrument. Ninety-seven (82%) targets were detected with at least 1 read in at least one condition (Table 4, Sheet 2). Among these 97 regions, 54 single-nucleotide polymorphisms (SNPs) and 18 indels were identified, in which 9 SNPs and 2 indels were GBM-specific (Table 7). Three GBM-specific variants affected CG or GC sites. There were 43 SNPs and 16 indels already recorded in dbSNP (http://www.ncbi.nlm.nih.gov/SNP/). The C-to-A substitution in the 5′ upstream flanking region of the CDH1 gene at chr16:68737131 was identified as GBM cell-specific, and indeed has been labeled as a risk-factor with clinical significance (rs16260). The SNP A allele was identified within 0% of reads in NSC and 37% of reads in Nx18-25. Among the 11 SNPs and 2 indels not yet included in dbSNP, a T-to-A substitution in the 5′ upstream flanking region of the DDB2 gene at chr11:47214657 was also GBM-specific. The SNP A allele was observed in 2.7% of reads in NSC and 21% of reads in Nx18-25. The ability to identify both alleles of known polymorphisms and novel variants demonstrates the high genotyping sensitivity of FENGC that is reproducible across samples as well as its potential for application in clinical diagnosis of genetic disorders. In addition, no SNPs or indels were identified in the three promoters chosen above to exemplify the identification of differential DNA methylation and chromatin accessibility between NSC and Nx18-25 GBM by MAPit-FENGC (FIG. 40, FIG. 41, and FIG. 42). This demonstrates bonafide epigenetic alterations between these cell lines, which, by definition, require the absence of mutations.

### Example 18: MAPit-FENGC Captures and Detects Epigenetic Features on ˜940-Nt Products and Delineates Long-Range Regulatory Relationships

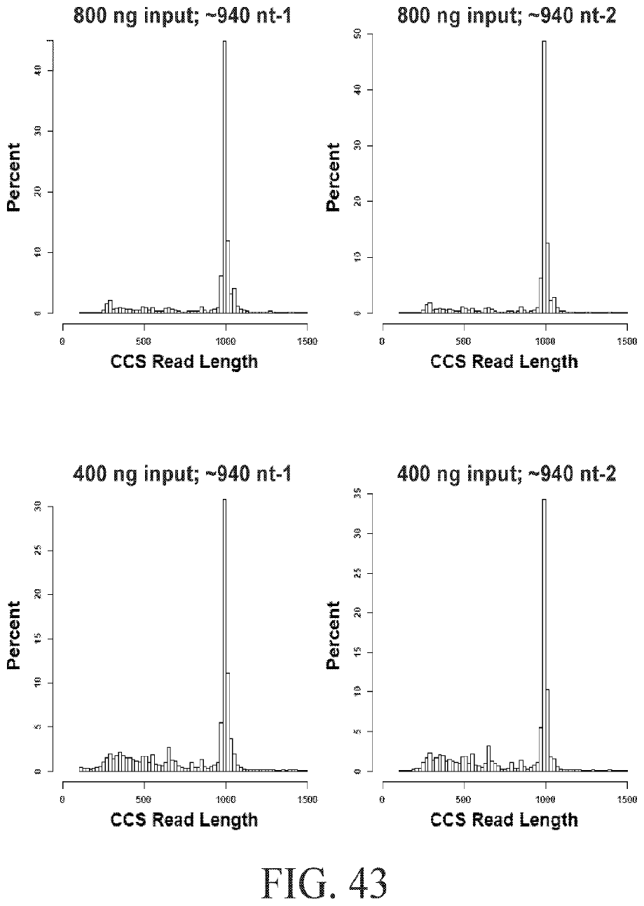

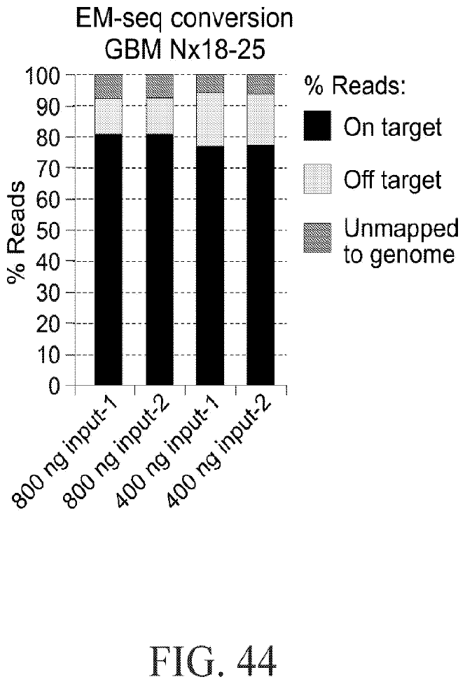

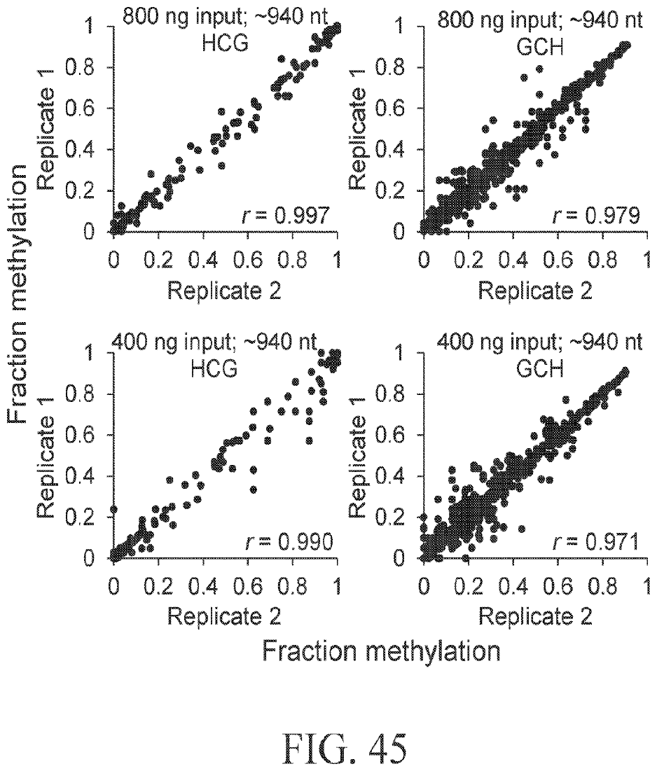

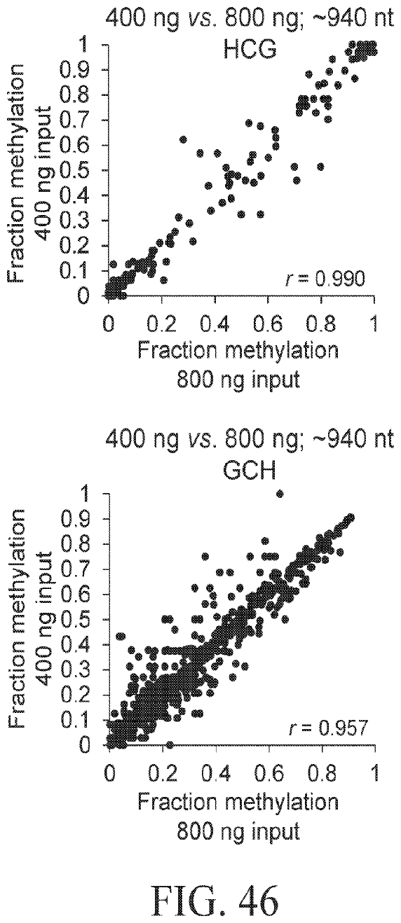

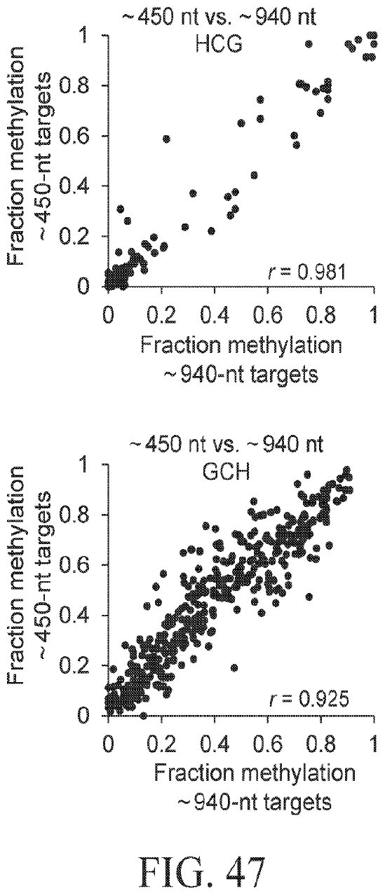

Long-read sequencing allows examination of epigenetic landscapes at a distance, i.e., relationships between individual regulatory modules such as multiple positioned nucleosomes and cis-acting sequences bound by transcription factors. MAPit-FENGC was therefore applied to the primary GBM cell line, Nx18-25, for 45 targets with lengths of ˜940 nt (Table 1, Sheet 4; Table 2, Sheet 3). Two gDNA input amounts, 800 ng and 400 ng, were tested from two biological replicates. The distribution of obtained CCS read lengths showed that most of the captured and amplified products were mostly of the expected ˜990 nt (˜940-nt targets plus 47-nt PCR primers; FIG. 43). Total percentages of on-target ˜940 bp reads for the 800 ng and 400 ng input samples were similar to the above ˜450 bp products in FIG. 29, approximately 81% and 77%, respectively (FIG. 44; Table 8). Less than 8% of reads did not map to the human genome and, among the reads aligned to human genome, the off-target percentage was ˜12% for 800 ng gDNA input and 18% for 400 ng gDNA input. There were 38 (84%) targets detected by ≥1 read for both of the replicates for each input DNA mass (Table 8). The coverage, however, of each target was variable (Table 9). Nevertheless, H5mCG and G5mCH levels were highly correlated between the two biological replicates for both the same (FIG. 45) and different input gDNA amounts (FIG. 46), as well as between the overlapped regions between the ˜940-nt and ˜450-nt targets (FIG. 47).

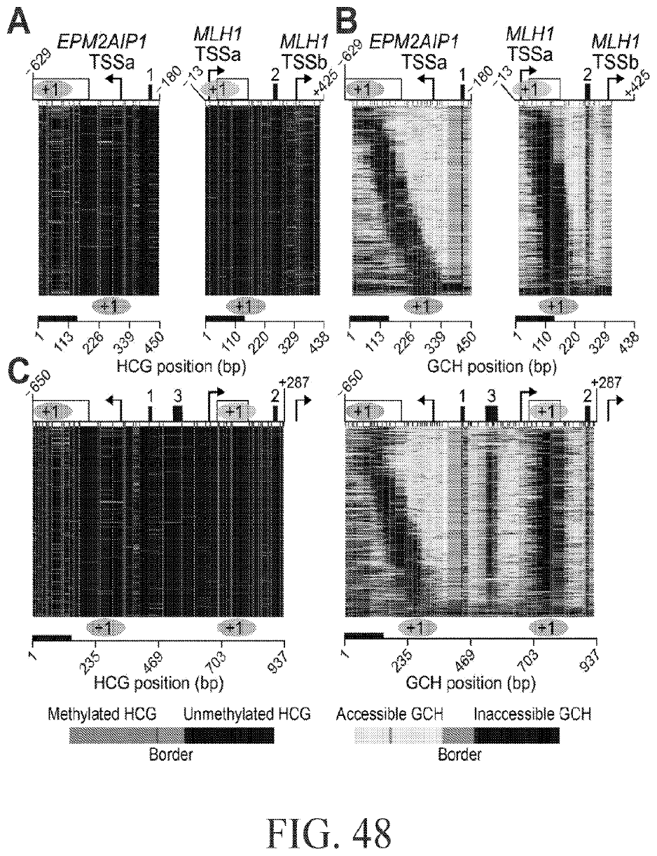

MAPit-FENGC reads of 937 bp containing the divergent TSSs from both the EPM2AIP1 and MLH1 promoters were compared with the reads from two overlapping, shorter products of 438 bp and 450 bp obtained from Nx18-25 gDNA (FIG. 48). A very low level of H5mCG was detected along the entire promoter in these cells (FIG. 48A and FIG. 48C, left panel). The 450 bp of overlap harboring the EPM2AIP1 TSS showed a variably positioned +1 nucleosome and therefore range of NFR lengths, and a short footprint (labeled 1) likely corresponding to occupancy by a sequence-specific transcriptional activator (FIG. 48B, left panel and FIG. 48C, right panel). Both of these features were seen in NSC as well (FIG. 36A). By contrast, in the overlapping MLH1 sequences, the +1 nucleosome occupied a much more constrained range of positions (FIG. 48B, right panel), and occupancy of a second sequence-specific transcription factor was identified upstream of MLH1 TSSb (labeled 2) (FIG. 48B and FIG. 48C, right panels). A third, robust transcription factor footprint (labeled 3) was detected upstream of MLH1 TSSa on the 937-bp amplicon (FIG. 48C, right panel). Robust co-occupancy of all three TFs and the two +1 nucleosomes was evident on these long EPM2AIP1-MLH1 molecules (FIG. 48C, right panel).

In addition to detecting additional molecular information, these long reads provided an opportunity to examine the extent to which multiple epigenetic features are coordinated or co-regulated. For example, the hierarchical organization of reads from both shorter amplicons clearly shows that the positions of both +1 nucleosomes range from farther to closer to each TSS (FIG. 48B). However, on the 937-bp amplicon, this ordered organization is no longer apparent for the MLH1 TSSa+1 nucleosome when the reads are hierarchically clustered on the EPM2AIP1 TSS+1 nucleosome (FIG. 48C, right panel) and vice versa (data not shown). This indicates that these two nucleosomes shift independently of each other. In sum, the longer amplicon netted an additional TF footprint and deduction of independent, dynamic mobilization of two nucleosomes, novel regulatory insights precluded by short reads.

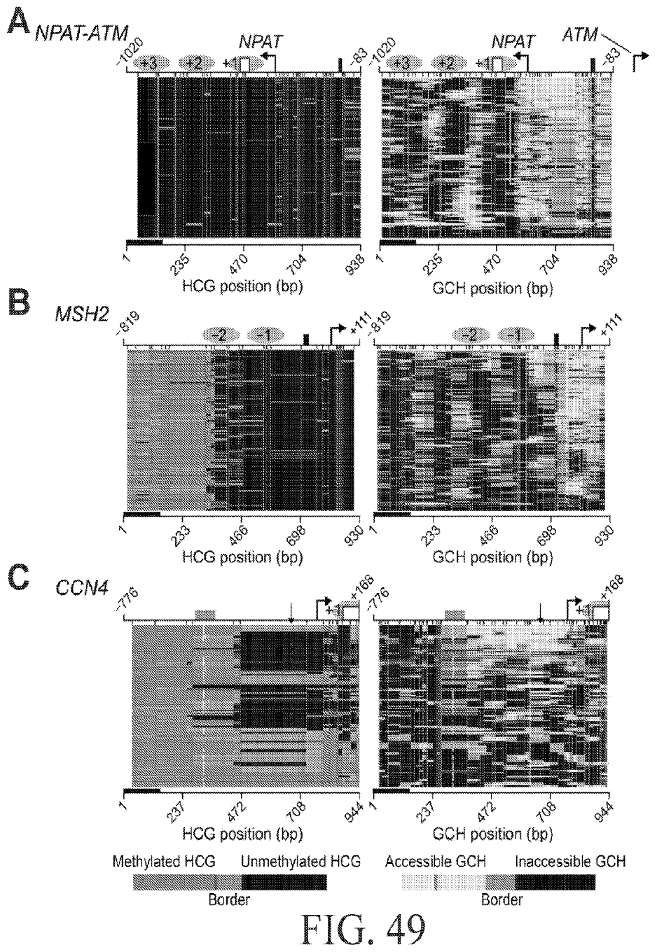

MAPit-FENGC of other ˜940-nt-targets from Nx18-25 cells availed additional organizational features compared to their shorter counterparts. The NFR of the divergent NPAT-ATM promoter showed a robust transcription factor footprint, a heterogeneously sized footprint (cyan rectangle) at the NPAT TSS, and a well-positioned +1 nucleosome followed by progressively less well-positioned nucleosomes +2 and +3 (FIG. 49A). By contrast, the upstream MSH2 promoter nucleosomes were much more disorganized (FIG. 49B), and the NFR of a sizeable number of molecules was punctuated with ˜55-bp footprints at the TSS (cyan rectangle), possibly corresponding to paused RNA polymerase II. MAPit-FENGC of a longer CCN4 promoter fragment from Nx18-25 (FIG. 49C) than assayed in FIG. 41B revealed NFR expansion to ˜400 bp on a subset of molecules, but no upstream positioned nucleosomes were discernable. Furthermore, MAPit-FENGC of ˜800 bp of 5′ flanking sequence from MSH2 (FIG. 49B) and CCN4 (FIG. 49C) identified clear transitions between 5mCG depletion and hypermethylation.

### Example 19: MAPit-FENGC Reproducibly Informs Epigenetic Architectures within Primary Cells

Having successfully applied FENGC to detect epigenetic and genetic variation in cultured NSC and GBM, the epigenetic arm of the protocol was tested on primary monocytes isolated from the bone marrows of four female mice. These cells were treated with M.CviPI and 600 ng gDNA from each sample was processed with primers targeting 78 promoters of cellular inflammatory response genes (Table 1, Sheet 5; Table 2, Sheet 4). For this experiment, the primers were designed by newly developed program, FENGC oligo designer (FOLD). To provide a computational solution for all 78 targets that avoided repetitive sequences and satisfied other rigorous command-line settings, target sizes were permitted to range from 474-987 nt (Table 2, Sheet 4).

High-quality CCS reads from the four libraries were mapped to the complete mm9 build of the mouse genome and the specific target reference sequences: 20-29% did not align to either reference or were removed by filtering (≥95% HCH conversion and ≥95% alignment), yielding 80-71% on-target reads. FENGC detected 71-75 targets (91-96%) with ≥1 read in each sample (Tables 10 and 11).

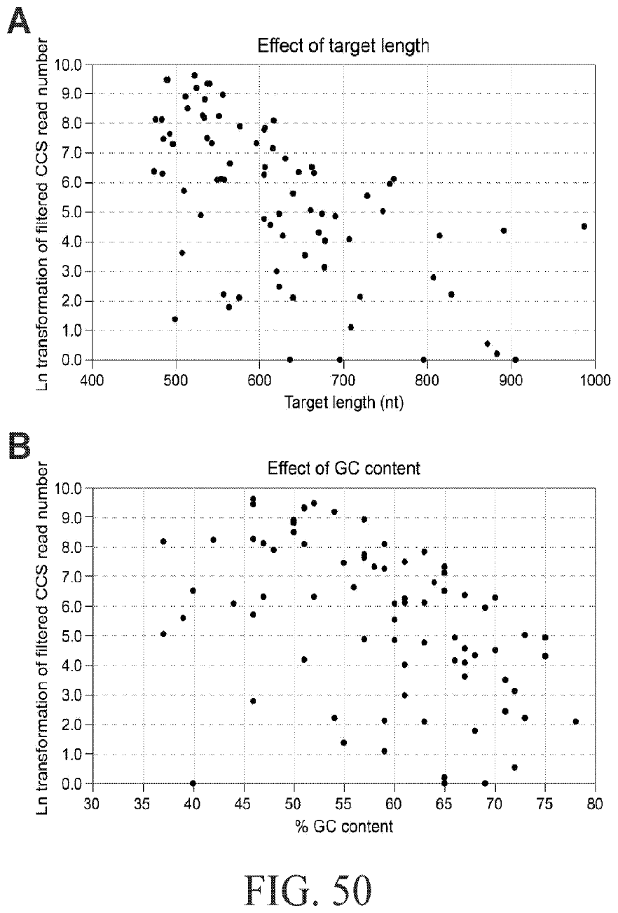

A mixed effects ANOVA was used to determine the extent to which the levels of DNA methylation and chromatin accessibility determined by MAPit-FENGC across each target were statistically different in at least one bone marrow-derived monocyte sample (Table 11). This testing was based on the total percentage of H5mCG or G5mCH per molecule and limited to 43 amplicons with ≥100 CCS reads per sample and good diversity, i.e., absence of duplicates apparent on visual inspection. CCS read numbers obtained from these targets showed negative correlations with target sequence length and GC content that ranged from 37-70% GC content and 474-760 nt, respectively (FIG. 50). Mean P values for H5mCG and G5mCH before correction for multiple testing were >0.90 (range 0.17-1.0) and all P values after correction with the Bonferroni method were 1.0. This indicates that for each target, none of the four mouse monocyte samples had significantly different levels of DNA methylation or chromatin accessibility (Table 11).

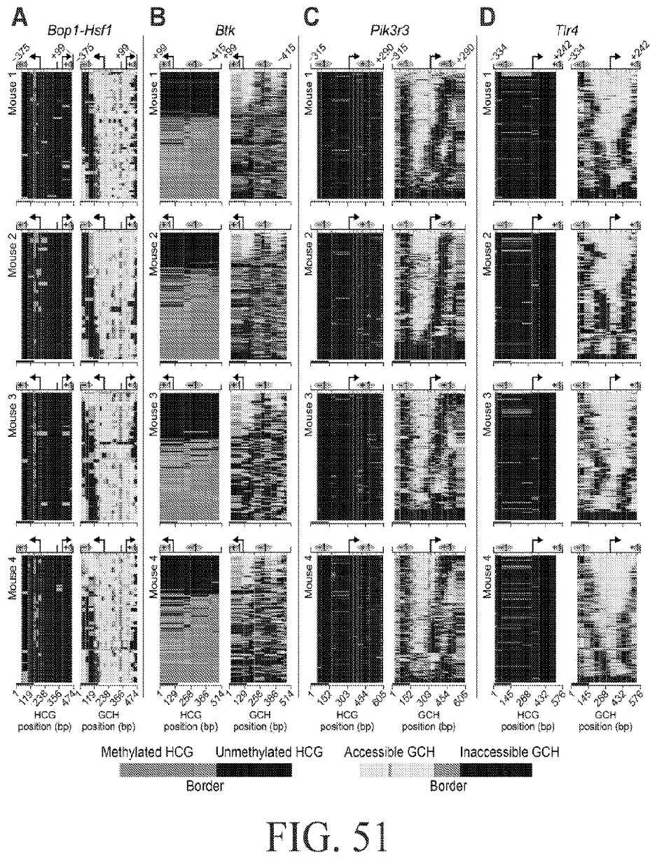

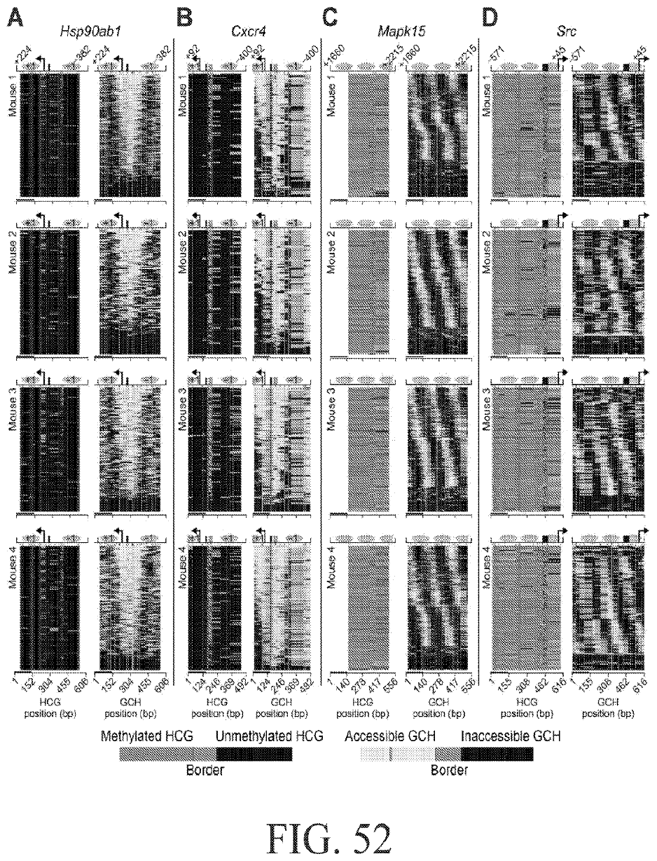

The reproducibility of chromatin architecture between independent mice is evident in single-molecule plots of H5mCG and G5mCH from eight representative loci that also illustrate interesting chromatin biology (FIG. 51 and FIG. 52). Hsf1 exemplifies a promoter in bone marrow-derived monocytes that is exceptionally open, with 2,333 of 2,334 molecules showing relatively large NFRs (range 223-445 bp; FIG. 51A). Again, such a locus indicates that observed changes in accessibility between different loci within a sample or at a specific locus between different samples are not attributable to variable cell permeabilization or M.CviPI activity.

Therefore, over the population of Btk promoter molecules from the X chromosome of the four female mice samples, a mean maximal accessibility of 46% (range 43-50%) occurred at the TSS within NFRs up to 195 bp long, localized almost exclusively to epialleles bearing 0% H5mCG (FIG. 51B). By contrast, consistent with X inactivation, long NFRs were highly depleted from epialleles with ≥2 H5mCG.

Among other intriguing loci, the Pik3r3 promoter harbored a remarkably well-positioned −1 nucleosome and incremental, preferential sliding of the +1 nucleosome, expanding or contracting NFR length in individual cells mainly on the side of the NFR downstream of the TSS (FIG. 51C). By comparison, NFR contraction/expansion occurred on both sides of the NRF in the promoters of Tlr4 (FIG. 51D), Hsp90ab1 (FIG. 52A), Irf7 (data not shown), and Cxcr4 (FIG. 52B) due to movement of both the −1 and +1 nucleosomes. Surprisingly, the Cxcr4 promoter displayed a 36-bp zone of H5mCG (orange rectangle) in the accessible NFR, only 12 bp from a strongly footprinted TF.

Compared to the above promoters, the Mapk15 gene body (+1,660 to +2,215) was heavily methylated with arrays of randomly positioned nucleosomes with short, linker-length NFRs (FIG. 52C). Chromatin −571 to +45 of the Src TSS was similarly organized (FIG. 52D), consistent with low-level expression of Src in mouse monocytes {Schaum, 2018 #190}. Interestingly, a sequence in Src with strong CTCF binding site homology {Hashimoto, 2017 #188} conferred clear protection of 50 bp against endogenous 5mCG in many cells.

Single-amplicon MAPit was used as an independent method to evaluate the chromatin structures of the Btk, Cxcr4, Hsp90ab1, and Tlr4 targets. Identical patterns of chromatin accessibility and DNA methylation were seen (data not shown), validating the MAPit-FENGC results. Taken together, the data demonstrate that MAPit-FENGC is effective at discerning epigenetic landscapes of purified primary cells, with striking inter-sample reproducibility.

In sum, FENGC permits facile, multiplexed, and cost-effective capture and enrichment of cohorts of user-defined sequences for either genotyping or detection of DNA methylation and chromatin accessibility in a single experiment. The high on-target coverage of long sequencing reads provides an unprecedented and exquisite level of molecular detail for applications in basic science and medicine.

### REFERENCES

1. Mamanova L, Coffey A J, Scott C E, Kozarewa I, Turner E H, Kumar A, Howard E, Shendure J, Turner D J. Target-enrichment strategies for next-generation sequencing. Nat Methods. 2010; 7(2):111-8. doi: 10.1038/nmeth.1419. PubMed PMID: 20111037.
2. Myllykangas S, Ji H P. Targeted deep resequencing of the human cancer genome using next-generation technologies. Biotechnol Genet Eng Rev. 2010; 27:135-58. PubMed PMID: 21415896; PMCID: PMC4340661.
3. Kozarewa I, Armisen J, Gardner A F, Slatko B E, Hendrickson C L. Overview of target enrichment strategies. Cuff Protoc Mol Biol. 2015; 112:7 21 1-3. doi: 10.1002/0471142727.mb0721s112. PubMed PMID: 26423591.
4. Chamberlain J S, Gibbs R A, Ranier J E, Nguyen P N, Caskey C T. Deletion screening of the Duchenne muscular dystrophy locus via multiplex DNA amplification. Nucleic Acids Res. 1988; 16(23):11141-56. doi: 10.1093/nar/16.23.11141. PubMed PMID: 3205741; PMCID: PMC339001.
5. Hayden M J, Nguyen T M, Waterman A, Chalmers K J. Multiplex-ready PCR: a new method for multiplexed SSR and SNP genotyping. BMC Genomics. 2008; 9:80. doi: 10.1186/1471-2164-9-80. PubMed PMID: 18282271; PMCID: PMC2275739.
6. Frommer M, McDonald L E, Millar D S, Collis C M, Watt F, Grigg G W, Molloy P L, Paul C L. A genomic sequencing protocol that yields a positive display of 5-methylcytosine residues in individual DNA strands. Proc Natl Acad Sci USA. 1992; 89(5):1827-31. doi: 10.1073/pnas.89.5.1827. PubMed PMID: 1542678; PMCID: PMC48546.
7. Darst R P, Pardo C E, Ai L, Brown K D, Kladde M P. Bisulfite sequencing of DNA. Curr Protoc Mol Biol. 2010; Chapter 7:Unit 7 9 1-17. PubMed PMID: 20583099.
8. Meissner A, Gnirke A, Bell G W, Ramsahoye B, Lander E S, Jaenisch R. Reduced representation bisulfite sequencing for comparative high-resolution DNA methylation analysis. Nucleic Acids Res. 2005; 33(18):5868-77. doi: 10.1093/nar/gki901. PubMed PMID: 16224102; PMCID: PMC1258174.
9. Gu H, Smith Z D, Bock C, Boyle P, Gnirke A, Meissner A. Preparation of reduced representation bisulfite sequencing libraries for genome-scale DNA methylation profiling. Nat Protoc. 2011; 6(4):468-81. doi: 10.1038/nprot.2010.190. PubMed PMID: 21412275.
10. McClelland M. The effect of sequence specific DNA methylation on restriction endonuclease cleavage. Nucleic Acids Res. 1981; 9(22):5859-66. doi: 10.1093/nar/9.22.5859. PubMed PMID: 6273810; PMCID: PMC327569.
11. Akalin A, Garrett-Bakelman F E, Kormaksson M, Busuttil J, Zhang L, Khrebtukova I, Milne T A, Huang Y, Biswas D, Hess J L, Allis C D, Roeder R G, Valk P J, Lowenberg B, Delwel R, Fernandez H F, Paietta E, Tallman M S, Schroth G P, Mason C E, Melnick A, Figueroa M E. Base-pair resolution DNA methylation sequencing reveals profoundly divergent epigenetic landscapes in acute myeloid leukemia. PLoS Genet. 2012; 8(6):e1002781. doi: 10.1371/journal.pgen.1002781. PubMed PMID: 22737091; PMCID: PMC3380828 Illumina.
12. Garrett-Bakelman F E, Sheridan C K, Kacmarczyk T J, Ishii J, Betel D, Alonso A, Mason C E, Figueroa M E, Melnick A M. Enhanced reduced representation bisulfite sequencing for assessment of DNA methylation at base pair resolution. J Vis Exp. 2015(96):e52246. doi: 10.3791/52246. PubMed PMID: 25742437; PMCID: PMC4354670.
13. Martinez-Arguelles D B, Lee S, Papadopoulos V. In silico analysis identifies novel restriction enzyme combinations that expand reduced representation bisulfite sequencing CpG coverage. BMC Res Notes. 2014; 7:534. doi: 10.1186/1756-0500-7-534. PubMed PMID: 25127888; PMCID: PMC4141122.
14. Sun Z, Vaisvila R, Hussong L M, Yan B, Baum C, Saleh L, Samaranayake M, Guan S, Dai N, Correa I R, Jr., Pradhan S, Davis T B, Evans T C, Jr., Ettwiller L M. Nondestructive enzymatic deamination enables single-molecule long-read amplicon sequencing for the determination of 5-methylcytosine and 5-hydroxymethylcytosine at single-base resolution. Genome Res. 2021. doi: 10.1101/gr.265306.120. PubMed PMID: 33468551; PMCID: PMC7849414.
15. Lyamichev V, Brow M A, Dahlberg J E. Structure-specific endonucleolytic cleavage of nucleic acids by eubacterial DNA polymerases. Science. 1993; 260(5109):778-83. doi: 10.1126/science.7683443. PubMed PMID: 7683443.
16. Chien A, Edgar D B, Trela J M. Deoxyribonucleic acid polymerase from the extreme thermophile Thermus aquaticus. J Bacteriol. 1976; 127(3):1550-7. doi: 10.1128/JB.127.3.1550-1557.1976. PubMed PMID: 8432; PMCID: PMC232952.
17. Varley K E, Mitra R D. Nested Patch PCR enables highly multiplexed mutation discovery in candidate genes. Genome Res. 2008; 18(11):1844-50. doi: 10.1101/gr.078204.108. PubMed PMID: 18849522; PMCID: PMC2577855.
18. Varley K E, Mitra R D. Bisulfite Patch PCR enables multiplexed sequencing of promoter methylation across cancer samples. Genome Res. 2010; 20(9):1279-87. doi: 10.1101/gr.101212.109. PubMed PMID: 20627893; PMCID: PMC2928506.
19. Finger L D, Atack J M, Tsutakawa S, Classen S, Tainer J, Grasby J, Shen B. The wonders of flap endonucleases: structure, function, mechanism and regulation. Subcell Biochem. 2012; 62:301-26. doi: 10.1007/978-94-007-4572-8_16. PubMed PMID: 22918592; PMCID: PMC3728657.
20. Wu D Y, Wallace R B. Specificity of the nick-closing activity of bacteriophage T4 DNA ligase. Gene. 1989; 76(2):245-54. doi: 10.1016/0378-1119(89)90165-0. PubMed PMID: 2753355.
21. Kaiser M W, Lyamicheva N, Ma W, Miller C, Neri B, Fors L, Lyamichev V I. A comparison of eubacterial and archaeal structure-specific 5′-exonucleases. J Biol Chem. 1999; 274(30):21387-94. doi: 10.1074/jbc.274.30.21387. PubMed PMID: 10409700.
22. Lyamichev V, Brow M A, Varvel V E, Dahlberg J E. Comparison of the 5′ nuclease activities of Taq DNA polymerase and its isolated nuclease domain. Proc Natl Acad Sci USA. 1999; 96(11):6143-8. doi: 10.1073/pnas.96.11.6143. PubMed PMID: 10339555; PMCID: PMC26849.
23. Lyamichev V, Mast A L, Hall J G, Prudent J R, Kaiser M W, Takova T, Kwiatkowski R W, Sander T J, de Arruda M, Arco D A, Neri B P, Brow M A. Polymorphism identification and quantitative detection of genomic DNA by invasive cleavage of oligonucleotide probes. Nat Biotechnol. 1999; 17(3):292-6. doi: 10.1038/7044. PubMed PMID: 10096299.
24. Hall J G, Eis P S, Law S M, Reynaldo L P, Prudent J R, Marshall D J, Allawi H T, Mast A L, Dahlberg J E, Kwiatkowski R W, de Arruda M, Neri B P, Lyamichev V I. Sensitive detection of DNA polymorphisms by the serial invasive signal amplification reaction. Proc Natl Acad Sci USA. 2000; 97(15):8272-7. doi: 10.1073/pnas.140225597. PubMed PMID: 10890904; PMCID: PMC26937.
25. Lyamichev V I, Kaiser M W, Lyamicheva N E, Vologodskii A V, Hall J G, Ma W P, Allawi H T, Neri B P. Experimental and theoretical analysis of the invasive signal amplification reaction. Biochemistry. 2000; 39(31):9523-32. doi: 10.1021/bi0007829. PubMed PMID: 10924149.
26. Tsutakawa S E, Thompson M J, Arvai A S, Neil A J, Shaw S J, Algasaier S I, Kim J C, Finger L D, Jardine E, Gotham V J B, Sarker A H, Her M Z, Rashid F, Hamdan S M, Mirkin S M, Grasby J A, Tainer J A. Phosphate steering by Flap Endonuclease 1 promotes 5′-flap specificity and incision to prevent genome instability. Nat Commun. 2017; 8:15855. doi: 10.1038/ncomms15855. PubMed PMID: 28653660; PMCID: PMC5490271.
27. Nabilsi N H, Deleyrolle L P, Darst R P, Riva A, Reynolds B A, Kladde M P. Multiplex mapping of chromatin accessibility and DNA methylation within targeted single molecules identifies epigenetic heterogeneity in neural stem cells and glioblastoma. Genome Res. 2014; 24(2):329-39. doi: 10.1101/gr.161737.113. PubMed PMID: 24105770; PMCID: PMC3912423.
28. Tahiliani M, Koh K P, Shen Y, Pastor W A, Bandukwala H, Brudno Y, Agarwal S, Iyer L M, Liu D R, Aravind L, Rao A. Conversion of 5-methylcytosine to 5-hydroxymethylcytosine in mammalian DNA by MLL partner TET1. Science. 2009; 324(5929):930-5. doi: 10.1126/science.1170116. PubMed PMID: 19372391; PMCID: PMC2715015.
29. Schutsky E K, DeNizio J E, Hu P, Liu M Y, Nabel C S, Fabyanic E B, Hwang Y, Bushman F D, Wu H, Kohli R M. Nondestructive, base-resolution sequencing of 5-hydroxymethylcytosine using a DNA deaminase. Nat Biotechnol. 2018. doi: 10.1038/nbt.4204. PubMed PMID: 30295673; PMCID: PMC6453757.
30. Tanaka K, Okamoto A. Degradation of DNA by bisulfite treatment. Bioorg Med Chem Lett. 2007; 17(7):1912-5. doi: 10.1016/j.bmcl.2007.01.040. PubMed PMID: 17276678.
31. Friedrich-Heineken E, Henneke G, Ferrari E, Hubscher U. The acetylatable lysines of human Fen1 are important for endo- and exonuclease activities. J Mol Biol. 2003; 328(1):73-84. doi: 10.1016/s0022-2836(03)00270-5. PubMed PMID: 12683998.
32. Volden R, Palmer T, Byrne A, Cole C, Schmitz R J, Green R E, Vollmers C. Improving nanopore read accuracy with the R2C2 method enables the sequencing of highly multiplexed full-length single-cell cDNA. Proc Natl Acad Sci USA. 2018; 115(39):9726-31. doi: 10.1073/pnas.1806447115. PubMed PMID: 30201725; PMCID: PMC6166824.
33. Shore D, Langowski J, Baldwin R L. DNA flexibility studied by covalent closure of short fragments into circles. Proc Natl Acad Sci USA. 1981; 78(8):4833-7. doi: 10.1073/pnas.78.8.4833. PubMed PMID: 6272277; PMCID: PMC320266.
34. Shore D, Baldwin R L. Energetics of DNA twisting. I. Relation between twist and cyclization probability. J Mol Biol. 1983; 170(4):957-81. doi: 10.1016/s0022-2836(83)80198-3. PubMed PMID: 6315955.
35. Shore D, Baldwin R L. Energetics of DNA twisting. II. Topoisomer analysis. J Mol Biol. 1983; 170(4):983-1007. doi: 10.1016/s0022-2836(83)80199-5. PubMed PMID: 6644817.
36. Stenberg J, Dahl F, Landegren U, Nilsson M. PieceMaker: selection of DNA fragments for selector-guided multiplex amplification. Nucleic Acids Res. 2005; 33(8):e72. doi: 10.1093/nar/gni071. PubMed PMID: 15860769; PMCID: PMC1087790.
37. Karst S M, Ziels R M, Kirkegaard R H, Sørensen E A, McDonald D, Zhu Q, Knight R, Albertsen M. Enabling high-accuracy long-read amplicon sequences using unique molecular identifiers with Nanopore or PacBio sequencing. bioRxiv. 2020:645903. doi: 10.1101/645903.
38. Bennett-Baker P E, Mueller J L. CRISPR-mediated isolation of specific megabase segments of genomic DNA. Nucleic Acids Res. 2017; 45(19):e165. doi: 10.1093/nar/gkx749. PubMed PMID: 28977642; PMCID: PMC5737698.
39. Gabrieli T, Sharim H, Fridman D, Arbib N, Michaeli Y, Ebenstein Y. Selective nanopore sequencing of human BRCA1 by Cas9-assisted targeting of chromosome segments (CATCH). Nucleic Acids Res. 2018; 46(14):e87. doi: 10.1093/nar/gky411. PubMed PMID: 29788371; PMCID: PMC6101500.
40. Gilpatrick T, Lee I, Graham J E, Raimondeau E, Bowen R, Heron A, Downs B, Sukumar S, Sedlazeck F J, Timp W. Targeted nanopore sequencing with Cas9-guided adapter ligation. Nat Biotechnol. 2020. doi: 10.1038/s41587-020-0407-5. PubMed PMID: 32042167.
41. Lowary P T, Widom J. New DNA sequence rules for high affinity binding to histone octamer and sequence-directed nucleosome positioning. J Mol Biol. 1998; 276(1):19-42. doi: 10.1006/jmbi.1997.1494. PubMed PMID: 9514715.
42. Tsutakawa S E, Classen S, Chapados B R, Arvai A S, Finger L D, Guenther G, Tomlinson C G, Thompson P, Sarker A H, Shen B, Cooper P K, Grasby J A, Tainer J A. Human flap endonuclease structures, DNA double-base flipping, and a unified understanding of the FEN1 superfamily. Cell. 2011; 145(2):198-211. doi: 10.1016/j.cell.2011.03.004. PubMed PMID: 21496641; PMCID: PMC3086263.
43. Dechassa M L, Sabri A, Pondugula S, Kassabov S R, Chatterjee N, Kladde M P, Bartholomew B. SWI/SNF has intrinsic nucleosome disassembly activity that is dependent on adjacent nucleosomes. Mol Cell. 2010; 38(4):590-602. doi: 10.1016/j.molcel.2010.02.040. PubMed PMID: 20513433; PMCID: PMC3161732.
44. Mouradov D, Sloggett C, Jorissen R N, Love C G, Li S, Burgess A W, Arango D, Strausberg R L, Buchanan D, Wormald S, O'Connor L, Wilding J L, Bicknell D, Tomlinson I P, Bodmer W F, Mariadason J M, Sieber O M. Colorectal cancer cell lines are representative models of the main molecular subtypes of primary cancer. Cancer Res. 2014; 74(12):3238-47. doi: 10.1158/0008-5472.CAN-14-0013. PubMed PMID: 24755471.
45. Xu M, Kladde M P, Van Etten J L, Simpson R T. Cloning, characterization and expression of the gene coding for a cytosine-5-DNA methyltransferase recognizing GpC. Nucleic Acids Res. 1998; 26(17):3961-6. Epub 1998/08/15. PubMed PMID: 9705505; PMCID: 147793.
46. Jessen W J, Hoose S A, Kilgore J A, Kladde M P. Active PHOS chromatin encompasses variable numbers of nucleosomes at individual promoters. Nat Struct Mol Biol. 2006; 13(3):256-63. doi: 10.1038/nsmb1062. PubMed PMID: 16491089.
47. Gal-Yam E N, Jeong S, Tanay A, Egger G, Lee A S, Jones P A. Constitutive nucleosome depletion and ordered factor assembly at the GRP78 promoter revealed by single molecule footprinting. PLoS Genet. 2006; 2(9):e160. doi: 10.1371/journal.pgen.0020160. PubMed PMID: 17002502; PMCID: PMC1574359.
48. Lin J C, Jeong S, Liang G, Takai D, Fatemi M, Tsai Y C, Egger G, Gal-Yam E N, Jones P A. Role of nucleosomal occupancy in the epigenetic silencing of the MLH1 CpG island. Cancer Cell. 2007; 12(5):432-44. doi: 10.1016/j.ccr.2007.10.014. PubMed PMID: 17996647; PMCID: PMC4657456.
49. Pardo C E, Darst R P, Nabilsi N H, Delmas A L, Kladde M P. Simultaneous single-molecule mapping of protein-DNA interactions and DNA methylation by MAPit. Curr Protoc Mol Biol. 2011; Chapter 21:Unit 21 2. doi: 10.1002/0471142727.mb2122s95. PubMed PMID: 21732317; PMCID: PMC3214598.
50. Kelly T K, Liu Y, Lay F D, Liang G, Berman B P, Jones P A. Genome-wide mapping of nucleosome positioning and DNA methylation within individual DNA molecules. Genome Res. 2012; 22(12):2497-506. doi: 10.1101/gr.143008.112. PubMed PMID: 22960375; PMCID: PMC3514679.
51. Lay F D, Kelly T K, Jones P A. Nucleosome Occupancy and Methylome Sequencing (NOMe-seq). Methods Mol Biol. 2018; 1708:267-84. doi: 10.1007/978-1-4939-7481-8_14. PubMed PMID: 29224149.
52. Deleyrolle L P, Reynolds B A. Isolation, expansion, and differentiation of adult Mammalian neural stem and progenitor cells using the neurosphere assay. Methods Mol Biol. 2009; 549:91-101. doi: 10.1007/978-1-60327-931-4_7. PubMed PMID: 19378198.
53. Eid J, Fehr A, Gray J, Luong K, Lyle J, Otto G, Peluso P, Rank D, Baybayan P, Bettman B, Bibillo A, Bjornson K, Chaudhuri B, Christians F, Cicero R, Clark S, Dalal R, Dewinter A, Dixon J, Foquet M, Gaertner A, Hardenbol P, Heiner C, Hester K, Holden D, Kearns G, Kong X, Kuse R, Lacroix Y, Lin S, Lundquist P, Ma C, Marks P, Maxham M, Murphy D, Park I, Pham T, Phillips M, Roy J, Sebra R, Shen G, Sorenson J, Tomaney A, Travers K, Trulson M, Vieceli J, Wegener J, Wu D, Yang A, Zaccarin D, Zhao P, Zhong F, Korlach J, Turner S. Real-time DNA sequencing from single polymerase molecules. Science. 2009; 323(5910):133-8. doi: 10.1126/science.1162986. PubMed PMID: 19023044.
54. Benjamini Y, Speed T P. Summarizing and correcting the GC content bias in high-throughput sequencing. Nucleic Acids Res. 2012; 40(10):e72. doi: 10.1093/nar/gks001. PubMed PMID: 22323520; PMCID: PMC3378858.
55. Zhang L, Szulwach K E, Hon G C, Song C X, Park B, Yu M, Lu X, Dai Q, Wang X, Street C R, Tan H, Min J H, Ren B, Jin P, He C. Tet-mediated covalent labelling of 5-methylcytosine for its genome-wide detection and sequencing. Nat Commun. 2013; 4:1517. doi: 10.1038/ncomms2527. PubMed PMID: 23443545; PMCID: PMC3679896.
56. Yu M, Hon G C, Szulwach K E, Song C X, Zhang L, Kim A, Li X, Dai Q, Shen Y, Park B, Min J H, Jin P, Ren B, He C. Base-resolution analysis of 5-hydroxymethylcytosine in the mammalian genome. Cell. 2012; 149(6):1368-80. doi: 10.1016/j.cell.2012.04.027. PubMed PMID: 22608086; PMCID: PMC3589129.
57. Josse J, Kornberg A. Glucosylation of deoxyribonucleic acid. III. α- and β-Glucosyl transferases from T4-infected Escherichia coli. J Biol Chem. 1962; 237:1968-76. PubMed PMID: 14452558.
58. Tomaschewski J, Gram H, Crabb J W, Ruger W. T4-induced α- and β-glucosyltransferase: cloning of the genes and a comparison of their products based on sequencing data. Nucleic Acids Res. 1985; 13(21):7551-68. doi: 10.1093/nar/13.21.7551. PubMed PMID: 2999696; PMCID: PMC322070.
59. Schutsky E K, Nabel C S, Davis A K F, DeNizio J E, Kohli R M. APOBEC3A efficiently deaminates methylated, but not TET-oxidized, cytosine bases in DNA. Nucleic Acids Res. 2017; 45(13):7655-65. doi: 10.1093/nar/gkx345. PubMed PMID: 28472485; PMCID: PMC5570014.
60. Naor D, Sionov R V, Ish-Shalom D. CD44: structure, function, and association with the malignant process. Adv Cancer Res. 1997; 71:241-319. doi: 10.1016/s0065-230x(08)60101-3. PubMed PMID: 9111868.
61. Naor D. Editorial: interaction between hyaluronic acid and its receptors (CD44, RHAMM) regulates the activity of inflammation and cancer. Front Immunol. 2016; 7:39. doi: 10.3389/fimmu.2016.00039. PubMed PMID: 26904028; PMCID: PMC4745048.
62. Liu Y, Han S S, Wu Y, Tuohy T M, Xue H, Cai J, Back S A, Sherman L S, Fischer I, Rao M S. CD44 expression identifies astrocyte-restricted precursor cells. Dev Biol. 2004; 276(1):31-46. doi: 10.1016/j.ydbio.2004.08.018. PubMed PMID: 15531362.
63. Phillips H S, Kharbanda S, Chen R, Forrest W F, Soriano R H, Wu T D, Misra A, Nigro J M, Colman H, Soroceanu L, Williams P M, Modrusan Z, Feuerstein B G, Aldape K. Molecular subclasses of high-grade glioma predict prognosis, delineate a pattern of disease progression, and resemble stages in neurogenesis. Cancer Cell. 2006; 9(3):157-73. doi: 10.1016/j.ccr.2006.02.019. PubMed PMID: 16530701.
64. Verhaak R G, Hoadley K A, Purdom E, Wang V, Qi Y, Wilkerson M D, Miller C R, Ding L, Golub T, Mesirov J P, Alexe G, Lawrence M, O'Kelly M, Tamayo P, Weir B A, Gabriel S, Winckler W, Gupta S, Jakkula L, Feiler H S, Hodgson J G, James C D, Sarkaria J N, Brennan C, Kahn A, Spellman P T, Wilson R K, Speed T P, Gray J W, Meyerson M, Getz G, Perou C M, Hayes D N, Cancer Genome Atlas Research N. Integrated genomic analysis identifies clinically relevant subtypes of glioblastoma characterized by abnormalities in PDGFRA, IDH1, EGFR, and NF1. Cancer Cell. 2010; 17(1):98-110. doi: 10.1016/j.ccr.2009.12.020. PubMed PMID: 20129251; PMCID: PMC2818769.
65. Anido J, Saez-Borderias A, Gonzalez-Junca A, Rodon L, Folch G, Carmona M A, Prieto-Sanchez R M, Barba I, Martinez-Saez E, Prudkin L, Cuartas I, Raventos C, Martinez-Ricarte F, Poca M A, Garcia-Dorado D, Lahn M M, Yingling J M, Rodon J, Sahuquillo J, Baselga J, Seoane J. TGF-β receptor inhibitors target the CD44^{high}/Id1^{high} glioma-initiating cell population in human glioblastoma. Cancer Cell. 2010; 18(6):655-68. doi: 10.1016/j.ccr.2010.10.023. PubMed PMID: 21156287.
66. Fu J, Yang Q Y, Sai K, Chen F R, Pang J C, Ng H K, Kwan A L, Chen Z P. TGM2 inhibition attenuates ID1 expression in CD44-high glioma-initiating cells. Neuro Oncol. 2013; 15(10):1353-65. doi: 10.1093/neuonc/not079. PubMed PMID: 23877317; PMCID: PMC3779037.
67. Gaudreau P O, Clairefond S, Class C A, Boulay P L, Chrobak P, Allard B, Azzi F, Pommey S, Do K A, Saad F, Trudel D, Young M, Stagg J. WISP1 is associated to advanced disease, EMT and an inflamed tumor microenvironment in multiple solid tumors. Oncoimmunology. 2019; 8(5):e1581545. doi: 10.1080/2162402X.2019.1581545. PubMed PMID: 31069142; PMCID: PMC6492985.
68. Liu Y, Song Y, Ye M, Hu X, Wang Z P, Zhu X. The emerging role of WISP proteins in tumorigenesis and cancer therapy. J Transl Med. 2019; 17(1):28. doi: 10.1186/s12967-019-1769-7. PubMed PMID: 30651114; PMCID: PMC6335850.
69. Deng W, Fernandez A, McLaughlin S L, Klinke D J, 2nd. WNT1-inducible signaling pathway protein 1 (WISP1/CCN4) stimulates melanoma invasion and metastasis by promoting the epithelial-mesenchymal transition. J Biol Chem. 2019; 294(14):5261-80. doi: 10.1074/jbc.RA118.006122. PubMed PMID: 30723155; PMCID: PMC6462510.
70. Jing D, Zhang Q, Yu H, Zhao Y, Shen L. Identification of WISP1 as a novel oncogene in glioblastoma. Int J Oncol. 2017; 51(4):1261-70. doi: 10.3892/ijo.2017.4119. PubMed PMID: 28902353.
71. Albig W, Meergans T, Doenecke D. Characterization of the H1.5 gene completes the set of human H1 subtype genes. Gene. 1997; 184(2):141-8. doi: 10.1016/s0378-1119(96)00582-3. PubMed PMID: 9031620.
72. Sancho M, Diani E, Beato M, Jordan A. Depletion of human histone H1 variants uncovers specific roles in gene expression and cell growth. PLoS Genet. 2008; 4(10):e1000227. doi: 10.1371/journal.pgen.1000227. PubMed PMID: 18927631; PMCID: PMC2563032.
73. Happel N, Doenecke D. Histone H1 and its isoforms: contribution to chromatin structure and function. Gene. 2009; 431(1-2):1-12. doi: 10.1016/j.gene.2008.11.003. PubMed PMID: 19059319.
74. Knight P, Gauthier M L, Pardo C E, Darst R P, Kapadia K, Browder H, Morton E, Riva A, Kladde M P, Bacher R. Methylscaper: an R/shiny app for joint visualization of DNA methylation and nucleosome occupancy in single-molecule and single-cell data. Bioinformatics. 2021 Jun. 14; 37(24):4857-9. doi: 10.1093/bioinformatics/btab438. Epub ahead of print. PMID: 34125875; PMCID: PMC8665741.
Fortin J M, Azari H, Zheng T, Darioosh R P, Schmoll M E, Vedam-Mai V, Deleyrolle L P, Reynolds B A. Transplantation of Defined Populations of Differentiated Human Neural Stem Cell Progeny. Sci Rep. 2016 Mar. 31; 6:23579. doi: 10.1038/srep23579. PMID: 27030542; PMCID: PMC4814839.
Azari H, Millette S, Ansari S, Rahman M, Deleyrolle L P, Reynolds B A. Isolation and expansion of human glioblastoma multiforme tumor cells using the neurosphere assay. J Vis Exp. 2011 Oct. 30; (56):e3633. doi: 10.3791/3633. PMID: 22064695; PMCID: PMC3227195.

What is claimed is:

1. An oligonucleotide complex for enriching at least one sequence of interest from a source sequence comprising flap oligonucleotides 1-N and 2-N each hybridized to a universal oligonucleotide 1-N(U1-N) or universal oligonucleotide 1 (U1) to form a first and second flap adapter, wherein the 5′ ends of the flap oligonucleotides 1-N and 2-N are sufficiently complementary to anneal to separate ends of the sequence of interest to facilitate cleavage of the sequence of interest from the source sequence.

2. The oligonucleotide complex of claim 1, wherein oligonucleotides 1-N and 2-N are comprised of a common 3′ tail portion that anneals to a universal oligonucleotide, either U1 or a respective U1-N.

3. The oligonucleotide complex of claim 1, wherein oligonucleotide U1-N or U1 is sufficiently complementary to anneal to a 3′ tail portion of flap oligonucleotide 1-N and flap oligonucleotide 2-N.

4. The oligonucleotide complex of claim 1, wherein an unpaired 3′ flap on oligonucleotide U1-N is at least one nucleotide in length and wherein no 3′ flap is present on oligonucleotide U1.

5. The oligonucleotide complex of claim 3, wherein the unpaired 3′ flap on oligonucleotide U1-N is one deoxynucleotide or dideoxynucleotide in length and can be A, C, G, or T.

6. The oligonucleotide complex of claim 3, wherein the 3′ flap end comprises at least one nucleotide and the most 5′ nucleotide of the 3′ flap matches the nucleotide at point of cleavage of the source sequence where the flap oligonucleotides 1-N and 2-N are annealed at and downstream of the target sequence, respectively, wherein, optionally, the 3′ flap cannot pair with the 3′ tail of the flap oligonucleotides 1-N and 2-N.

7. (canceled)

8. The oligonucleotide complex of claim 1, wherein the first and second flap adapters create cleavage sites for a structure-specific endonuclease, and wherein, optionally, the endonuclease is a flap endonuclease or Taq.

9. (canceled)

10. The oligonucleotide complex of claim 1, wherein the 5′ cleaved end is ligated to a first universal oligonucleotide (U1-N) and the 3′ cleaved end is ligated to a second universal oligonucleotide (U2) hybridized to an oligonucleotide 3-N thereby forming a DNA strand comprising an arrangement of U1-N-Sequence of interest-U2.

11. The oligonucleotide complex of claim 10, wherein the ligated universal oligonucleotides U1-N, U2, or both are modified to protect against degradation by one or more exonucleases.

12. The oligonucleotide complex of claim 10, wherein upon subjecting the DNA strand to one or more exonucleases an enriched single strand of DNA comprising an arrangement of U1-N-Sequence of interest-U2 is produced.

13. A kit for enriching at least one sequence of interest from a source sequence comprising; flap oligonucleotide 1-N stock, flap oligonucleotide 2-N stock, oligonucleotide 3-N stock, universal oligonucleotide 1-N(oligo U1-N), universal oligonucleotide 2 (U2), U1 primer (oligonucleotide U1), and U2 primer.

14. (canceled)

15. The kit of claim 13, wherein for each target sequence of interest there are (i) flap oligonucleotides 1-N and 2-N that are comprised of a 5′ end that is target sequence specific and a 3′ end that is sufficiently complementary to the oligonucleotide U1-N or U2, and, optionally, (ii) an oligonucleotide 3-N that is comprised of a 3′ end that is target sequence specific and a 5′ end that is sufficiently complementary to the oligonucleotide U2.

16. (canceled)

17. The kit of claim 13, wherein oligonucleotide U2 is synthesized with a ligatable 5′ phosphate and, at its 3′ end, one or more chemical moieties that protect against exonuclease degradation.

18. The kit of claim 13, wherein (i) oligonucleotide U2 is synthesized with five phosphorothioate bonds at the 3′ end and wherein a three-carbon spacer is covalently attached to the 3′-terminal hydroxyl; (ii) wherein the U1-N is synthesized with a ligatable 3′-terminal hydroxyl and, at its 5′ end, one or more chemical moieties that protect against exonuclease degradation; and/or (iii) wherein the 5′ end of oligonucleotide U1-N is synthesized with five phosphorothioate bonds and either covalently attached to a three-carbon spacer or without a 5′ phosphate.

19. (canceled)

20. (canceled)

21. A method for enriching a sequence of interest from a source sequence for sequencing comprising:
I. obtaining a source DNA,
II. denaturing the source DNA,
III. annealing a pair of flap oligonucleotides 1-N and 2-N to a first and second region, respectively, of the denatured source sequence, each flap oligonucleotide 1-N and 2-N hybridized to a universal oligonucleotide 1-N(U1-N), wherein the first region comprises a portion of the sequence of interest and wherein the second region is downstream of the sequence of interest,
IV. cleaving the denatured source DNA using a structure-specific endonuclease to produce a cleaved sequence of interest comprising 5′ and 3′ cut ends,
V. ligating the 5′ cut end to the oligonucleotide U1-N such that the sequence of interest comprises a ligated U1-N hybridized with the flap oligonucleotide 1-N,
VI. ligating the 3′ cut end to an oligonucleotide U2 hybridized to an oligonucleotide 3-N, wherein a 5′ portion of the oligonucleotide 3-N is sufficiently complementary to U2 and a 3′ tail portion is sufficiently complementary to a portion of the sequence of interest such that the sequence of interest comprises a ligated oligonucleotide U2 hybridized to oligonucleotide 3-N, and
VII. subjecting the sequence of interest from step (V), step (VI), or both, to one or more exonucleases to produce an enriched single strand of DNA comprising an arrangement of U1-N-Sequence of interest-U2.

22. (canceled)

23. The method of claim 21, wherein ligating comprises ligating the 5′ end of the sequence of interest to the 3′ end of oligonucleotide oligo U1-N and the 3′ end of the sequence of interest to the 5′ end of oligonucleotide oligo U2 followed by exonuclease digestion.

24. (canceled)

25. The method of claim 21, wherein the method further comprises subjecting a source sequence to DNA methyltransferase prior to step I.

26. The method of claim 22, further comprising purifying the U1-N-Sequence of interest-U2 or amplifying the enriched single strand of DNA comprising an arrangement of U1-N-Sequence of interest-U2 by standard PCR, BS-PCR, EM-PCR, both standard PCR and BS-PCR, or both standard PCR and EM-PCR, using U1 and U2 primers.

27. The method of claim 21, wherein the structure-specific endonuclease comprises a flap endonuclease or Taq polymerase.

28. (canceled)

29. (canceled)

30. A method for enriching a sequence of interest from a source sequence comprising:
I. obtaining a source DNA,
II. denaturing the source DNA,
III. annealing a pair of flap oligonucleotides 1-N and oligonucleotide 2-N to a first and second region, respectively, of the denatured source sequence, each flap oligonucleotides 1-N and 2-N hybridized to a universal oligonucleotide-1 (U1), wherein the first region comprises a portion of the sequence of interest and wherein the second region is downstream of the sequence of interest,
IV. cleaving the denatured source DNA using a structure-specific endonuclease to produce a cleaved sequence of interest comprising 5′ and 3′ cut ends,
V. ligating the 3′ cut end to an oligonucleotide oligo U2 hybridized to an oligonucleotide 3-N, wherein a 5′ portion of the oligonucleotide 3-N is complementary to oligo U2 and a 3′ tail portion is complementary to a portion of the sequence of interest such that the sequence of interest comprises a ligated oligonucleotide U2 hybridized to oligonucleotide 3,
VI. subjecting the sequence of interest from step (V), to one or more exonucleases to produce a single strand of DNA comprising an arrangement of Sequence of interest-U2,
VII. annealing to the enriched sequence of interest-U2 to a flap oligonucleotide 1-N hybridized to a universal oligonucleotide 1-N(U1-N),
VIII. ligating oligonucleotide U1-N to the 5′ end of the sequence of interest-U2,
IX. subjecting the sequence of interest-U2 of step VIII to one or more exonucleases to produce a single strand of DNA comprising an arrangement of U1-N-Sequence of interest-U2.

31. (canceled)

32. (canceled)

33. (canceled)

34. (canceled)

35. (canceled)

36. (canceled)
